(19) United States
(12) Patent Application Publication
(10) Pub. No.: US 2026/0002213 A1
(43) Pub. Date: Jan. 1, 2026

(54) Reagents and Methods for Identifying Pregnancy Risk

(71) Applicant: EWinger, Inc., San Francisco, CA (US)

(72) Inventors: Edward E Winger, San Francisco, CA (US); Jane L Reed, Klamath, OR (US)

(21) Appl. No.: 19/042,529

(22) Filed: Jan. 31, 2025

Related U.S. Application Data
(60) Provisional application No. 63/549,392, filed on Feb. 2, 2024.
(60) Provisional application No. 63/550,113, filed on Feb. 6, 2024.
(60) Provisional application No. 63/550,123, filed on Feb. 6, 2024.
(60) Provisional application No. 63/676,053, filed on Jul. 26, 2024.

(51) Int. Cl. C12Q 1/6883

(52) U.S. Cl. CPC C12Q 1/6883; C12Q 2600/118; C12Q 2600/158; C12Q 2600/178

(57) ABSTRACT

This disclosure relates to miRNA-related reagents and methods for identifying health risks during pregnancy. In preferred embodiments, the miRNAs are selected from those disclosed herein that in a pregnant human being indicate an increased risk of developing a placental bed disorder as compared to the control sample. In some preferred embodiments, such miRNAs are selected from the group consisting of hsa-miR-150-5p, hsa-let-7g-5p, hsa-miR-16-5p, hsa-miR-29a-3p, hsa-let-7f-5p, hsa-miR-15b-5p, hsa-let-7i-5p, hsa-miR-26b-5p, hsa-miR-223-3p, hsa-miR-21-5p, hsa-miR-23a-3p, hsa-miR-142-3p, hsa-miR-19b-3p, hsa-let-7a-5p, hsa-miR-22-3p, hsa-miR-29c-3p, hsa-miR-342-3p, hsa-miR-25-3p, hsa-miR-24-3p, and hsa-miR-17-5p.

### RELATED APPLICATIONS

This application claims priority to U.S. Ser. No. 63/549,392 filed on Feb. 2, 2024; U.S. Ser. No. 63/550,113 filed on Feb. 6, 2024; U.S. Ser. No. 63/550,123 filed on Feb. 6, 2024; and, U.S. Ser. No. 63/676,053 filed on Jul. 26, 2024, each of which is incorporated in its entirety into this application.

### FIELD OF THE DISCLOSURE

This disclosure relates to miRNA-related reagents and methods for identifying health risks during pregnancy.

### BACKGROUND INFORMATION

Winger and Reed identified a group of maternal cell microRNAs that are differentially expressed in early pregnancy between pregnant women destined to experience healthy pregnancies from those destined to experience pregnancies compromised by disorders of the placental bed. The supporting studies were conducted on samples drawn from a single clinic serving a non-diverse population (Winger E E, Reed J L et al. Am J Reprod Immunol. 2014 November; 72(5):515-26.; Winger E E, Reed J L et al. J Reprod Immunol. 2015 August; 110:22-35). When further studies incorporated patients from racially diverse groups were compared to a control group derived from the original clinic population, the predictive power of the assay was less robust. We now recognize that optimum panel microRNAs vary strongly with the racial population being studied (Winger E E, Reed J L et al. PLoS One. 2020 Aug. 13; 15(8):e0236805. doi: 10.1371/journal.pone.0236805. eCollection 2020. PMID: 32790689).

The diagnostic technique devised by Winger and Reed is based on collection of maternal peripheral blood during the first and early second trimester of pregnancy. Typically, the liquid elements of blood are discarded saving the cellular portion for microRNA extraction. MicroRNA comprise a large class of polynucleotides that regulate post transcriptional gene expression. Quantified sample levels are compared with corresponding microRNAs in a control group from one or more women who ultimately experience healthy pregnancies. Panels of microRNAs are constructed which are differentially expressed between patients destined healthy pregnancy and those destined to pregnancy disorders. MicroRNAs and their respective expression levels utilized in the initial studies were derived from an essentially non-Black population. (Winger E E, Reed J L et al. PLoS One. 2017 Jul. 10; 12(7):e0180124. doi: 10.1371/journal.pone.0180124. eCollection 2017).

Significant differences in risk of compromised birth between Black women and non-Black women are well recognized. The diagnostic technique is likewise affected. The patient comparison panel heretofore did not recognize differences in racial composition. The microRNA selected and the cutoff values used to discriminate risk levels are different between the non-Black individuals initially tested and panels devised limited to self-identified Black individuals. Data will be presented comparing microRNAs that are differentially expressed between patients destined to healthy and compromised pregnancy outcomes in Black and non-Black patients. The data are compared and demonstrate marked differences between the two racial groups. There has been considerable speculation attributing cause to both environmental and genetic factors. However, most studies support the importance of environmental factors. Matching of racial characteristics of patient and control group for improvement in prediction of pregnancy compromise offers a significant improvement in the robustness of the assay. It is important to recognize that while expression levels of microRNA are genetically determined they are also regulated in real time by physiologic requirements causing their expression to be related to both genetics and environment. In our initial studies, self-identified race has been used in the determination of risk groups.

The inventors recognize that use of self-identification of race is of limited utility for defining patient groups. Correct identification of a patient's racial group is necessary in order optimal pregnancy predictive power of the test to be achieved. However, correct identification of the patient's racial group is not always possible by visual inspection or medical record assessment. An objective means for assignment of patients and members of a control group to a common group is an unmet need. Conventional means of group identification may not provide insight into differences in future behavior of an individual such as the development of a pregnancy disorder. The inventors have devised a method of patient group assignment that relies on use of quantitative and objective criteria.

Data will be presented that demonstrate differences in the specific microRNAs that are differentially expressed between patients at risk from those who will experience healthy pregnancy between Black and non-Black patients and, by extension, between other racial groups. Improved prediction of outcomes accompanies the use of a racially matched control group. Mixed race patients offer a further challenge because the biologic behavior of microRNAs making comparison group assignment ambiguous. Expression of microRNAs may guide the analyst in assignment of the patients to a control group that shares a common expression pattern of microRNA.

Archeological findings aided more recently by genetics has revised our understanding of the distinctions between human populations in particular genetic differences between Blacks and non-Blacks. Accompanying the evolution of Homo sapiens within the species, intermingling of distinct but related species of Homo has made significant contributions to the genetic pool of Homo sapiens through the process of introgression (Dannemann M, Kelso J. Am J Hum Genet. 2017 Oct. 5; 101(4):578-589). Retention of limited portions of introgressed Neanderthal genetic material have aided in survival in environments not encountered in the earlier sub-Saharan African development of Homo sapiens. Neanderthals adapted to the colder northern European climates over several hundreds of thousands of years dwarfing the shorter periods of genetic adaption within migrating Homo sapiens groups. Introgression enabled African emigrants more rapid adaption to European conditions through acquisition of Neanderthal genes (Gittelman R M et al. Curr Biol. 2016 Dec. 19; 26(24):3375-3382).

It is now recognized that a significant source of diversity amongst modern humans originates from the variable admixtures of archaic human species. Neanderthals contribute from 1 to 3 percent to the genome of present-day Eurasians (Additional contributions from Denisovans are also known particularly in Asian cohorts.) The resulting specific contributions are not homogeneous throughout Eurasian groups and have significant geographic variation. There is considerable variation in the specific genetic contribution from introgression in individuals while the overall percentage may be the same in two individuals which may account for differences in pregnancy outcomes amongst individuals of any single group. Non-migrating Homo sapiens, those that remained in Africa, show little or no Neanderthal or Denisovan introgression. The result is a dichotomy in genetics between sub-Saharan Africans remaining in Africa and those that migrated out of Africa (Silvert, M., et al., 2009. Genetics, 203, pp. 881-891).

Extensive variation in responses to immune challenge between individuals of African and European descent attributable to Neanderthal introgression have been identified (Quach, H et al. Cell, 167(3), pp. 643-656). An environment dense in infectious challenges calls for a strong inflammatory responsiveness. African ancestry predicts a stronger inflammatory bias than corresponding European populations (Nédélec, Y. et al. Cell, 167(3), pp. 657-669).

Genes entering the Homo sapiens genome by Neanderthal introgression subsequently undergo purifying selection. Immune genes are most conserved. Toll-like receptors (TLR1, TLR6 and TLR10), OAS1/2/3 (oligoadenylate synthase), TNFAIP3, STAT1 and STAT3 in modern non-Black humans carry distinct archaic haplotypes introgressed from Neanderthal and Denisovan genomes. Prominent amongst introgressed genetic elements are innate immunity genes as they are directly involved in preservation in the newly encountered environment, (Dannemann, M et al. bioRxiv, p. 022699), (Sams, A. J et al. Genome biology, 17(1), pp. 1-15), (Deschamps, M, et al. 2016. The American Journal of Human Genetics, 98(1), pp. 5-21); (Quach, H. and Quintana-Murci, L, 2017. Journal of Experimental Medicine, 214(4), pp. 877-894). Some of the differentially expressed genes and enhancers observed between modern human races may have been differentially introgressed from Neanderthal ancestors. Some of these differentially expressed genes might account for immune and metabolic and pregnancy outcome differences observed between races. We analyzed top 20 differentially expressed maternal immune cell microRNAs between pregnant Blacks and Non-Blacks in the early first trimester limited to only to those microRNAs that regulate known Neanderthal introgressed enhancer SNPs. Examining a Neanderthal gene database (Neanderthal genes S7 of the reference: Silvert M, Quintana-Murci L, Rotival M. Am J Hum Genet. 2019 Jun. 6; 104(6):1241-12500). We found that fifty percent (10 of the of our top 20) microRNAs most differentially expressed microRNAs between pregnant Black and Non-Blacks were also found to regulate gene sets controlled by Neanderthal introgressed enhancer SNPs Gene sets selected for analysis were “Regulation of cell motility” (GO:2000145) and “Regulation of cell migration” (GO:0030334), biologic processes are involved with trophoblast migration in early pregnancy (Table 30). These microRNA/gene associations were determined herein using mirDIP 4.1 (mirDIP reference: Tokar T, Pastrello C, Rossos A E M, Abovsky M, Hauschild A C, Tsay M, Lu R, Jurisica I. mirDIP 4.1-integrative database of human microRNA target predictions. Nucleic Acids Res. 2018 Jan. 4; 46(D1):D360-D370. doi: 10.1093/nar/gkx1144. PMID: 29194489; PMCID: PMC5753284).

The most statistically significant pathway calculated from the 22 Neanderthal introgressed genes regulated by our top microRNAs most differentially expressed between pregnant Black and Non-Blacks microRNAs was “Regulation of gene expression by Hypoxia-inducible Factor” (Table 31). Hypoxia-inducible Factor-1 alpha (HIF-1 alpha) is critically important in early pregnancy as it binds to the hypoxia response element in VEGF at the time of pregnancy implantation enabling anaerobic metabolism in the low oxygen environment of the implantation site (Daikoku T et al. J Biol Chem. 2003 Feb. 28; 278(9):7683-91).

In addition to HIF-1-alpha regulation pathway, Neanderthal introgressed genes differentially expressed between modern human races may be involved with other population differences in pregnancy outcome. IL-33 metabolism may be involved with improved thermogenesis and cold tolerance in northern environments, and there is evidence that introgressed genes related to these pathways may predominate in modern Europeans but not Africans. (Odegaard J I, et al. Perinatal Licensing of Thermogenesis by IL-33 and ST2. Cell. 2016 Aug. 11; 166(4):841-854). Interestingly, in addition to cold tolerance, IL-33 may also act as an ‘alarmin’ in early pregnancy to alert the immune system of potential tissue stress or damage. In early pregnancy, cleaved forms of IL-33 may activate immune cells expressing ST2, ILC2s and regulatory T cells (Tregs), enhancing the TH2 effector response, improving trophoblast invasion and pregnancy outcomes. (Romero R, et al.—a longitudinal study. J Matern Fetal Neonatal Med. 2018 February; 31(4):418-432; Sheng Y R, Hu W T, Shen H H, Wei C Y, Liu Y K, Ma X Q, Li M Q, Zhu X Y. Cell Mol Life Sci. 2022 Mar. 4; 79(3):173.). Given that Europeans carry these Neanderthal introgressed genes and African Blacks generally do not (Plunkett J et al. Ann Med. 2008; 40(3):167-95), it is possible that genetic Introgression may partially explain some racial differences we see between Blacks and non-Blacks in early pregnancy. These differences may be used to better understand the management of pregnancy disease. In summary, it is possible that differences in degree of Neanderthal introgression may offer insight into differences in pregnancy outcome between modern populations, and new tools for treatment approaches.

The correct orchestration of immune responses is central to implantation and facilitation of the long gestation of a successful pregnancy in eutherian mammals. Inflammation is necessary for implantation but must be followed by an orderly switch to an anti-inflammatory state permissive of pregnancy maintenance (Griffith, O. W., et al. Proceedings of the National Academy of Sciences, 114(32), pp. E6566-E657).

Newly pregnant women are at risk of maldevelopment of the placental bed which, in turn, leads to the development of preeclampsia and preterm delivery. These conditions are exemplary of a larger group that have become known as disorders of the placental bed. The formation of the placental bed is largely determined in early pregnancy. After disorders of the placental bed become clinically apparent, generally after about twenty weeks of pregnancy, they become more difficult to manage. Identification of women who are destined to develop these conditions permit intervention early in pregnancy, during the initial period of placental bed maturation, when intervention may alter placental bed development.

Placental dysfunction is widely recognized as the mediator of the manifestations of diseases arising from placental bed maldevelopment. Well-documented in preeclampsia is the excessive release of vasoactive substances such as sFlt1 (soluble VEGF receptor) that, in turn, sequester endothelial factors that maintain endothelial cells throughout the mother's vascular system. While the placenta is the key mediator of clinical disease, the placenta is, itself, the target of the prior and more silent dysfunction arising in the placental bed. Conventionally, prediction of clinical manifestations has rested upon detection of placental dysfunction. More appropriately, diagnostic efforts directed to the earlier events taking place within the placental bed should be more informative and permit earlier diagnosis and therapy.

Racially distinctive expression of microRNAs in patients destined to placental bed dysfunction will be shown. Expression of microRNAs that are expressed between healthy (control) patients and patients destined to disorders of the placental bed may be different between different racial groups both in level of expression as well as in specific microRNAs. Racially divergent expression of microRNAs suggests differential regulation of metabolic pathways. For example, differential regulation of insulin pathways, fat metabolism and thermogenesis suggest the importance of those pathways in individuals where specific microRNAs regulating these pathways are expressed differently between healthy patients and those destined to pregnancy disorders. Interventions directed at regulation of these pathways may, therefore, be of specific importance.

In the United States, over 14% of Black women experience premature birth compared to just 9% of non-Black women (Joyce A. Martin et al., “Births: Final Data for 2018,” National Vital Statistics Reports 68, no. 13 (2019). Recognition of population risk differences in pregnancy is necessary if effective prevention is to be achieved. Low dose aspirin administration, for example, has been shown to mitigate maladaptive maturation within the placental bed when provided early (Rolnik D L et. N Engl J Med. 2017 Aug. 17; 377(7):613-622). However, non-selective administration of aspirin and other therapies, are associated with adverse effects that outweigh benefit if administered routinely to low-risk populations. (Whitlock E P et al, Ann Intern Med. 2016 Jun. 21; 164(12):826-35. doi: 10.7326/M15-2112). Population-based risk assessment is essential if timely prevention is to be achieved.

This disclosure provides improvements to previously described methods by improving the selection of microRNA from which the control population is developed. This disclosure is based on the surprising observation that the microRNA panel used for comparison provides superior differentiation between predicted pregnancy outcome when the microRNA selected for the control panel are matched for a patient characteristic (e.g., ethnicity, for example Black vs. non-Black individuals) (a “Patient Cluster”). The use of biologic criteria to select groupings may provide more specific, non-socially devised selection that may suggest appropriate intervention.

The disclosure provides methods that improve the conventional use of a control group by additionally limiting comparison of patients to a control group belonging to a common patient cluster. Because the term “cluster” may be defined by the expression levels of one or more microRNAs, it is not limited to a specified characteristic such as ethnicity (e.g., race (white, Black, Asian, etc.)), nationality, ethnicity, environmental exposure such as economic status, or disease state because microRNA expression is affected by all of these factors.

### BRIEF DESCRIPTION OF DRAWINGS

FIG. 1. compares ROC curves for pregnancy outcome prediction in Blacks and non-Blacks (see scoring calculations in Tables 9, 10, 11 and 12, respectively). MicroRNA pregnancy risk panels differ between Black and non-Black racial groups (see Table 13). This finding demonstrates an unmet need for a woman's biological Cluster grouping (race) to be known for optimal microRNA scoring prediction.

FIG. 2 illustrates “self-identified” race populations and compares their ROC curves for pregnancy outcome prediction. ROC curves calculated using microRNA pregnancy outcome prediction scores taken from data in Tables 21, 22, 23, and 24, respectively.

FIG. 3 illustrates “microRNA designated” race populations and comparing their ROC curves for pregnancy outcome prediction. ROC curves calculated using microRNA pregnancy outcome prediction scores taken from data in Tables 25, 26, 27, and 28 respectively.

FIG. 4 presents step one of Cluster analysis procedure using the NCSS statistical program: Patients are listed into a spreadsheet with group ID, (“A” or “B”) listed in rows and associated MicroRNA levels listed in columns.

FIG. 5 presents step two of Cluster analysis procedure: The spreadsheet from step one is pasted into the data entry section of the NCSS program.

FIG. 6 presents step three of Cluster analysis procedure: K-Means Clustering analysis method is selected from the NCSS program.

FIG. 7 presents step four of Cluster analysis procedure: Plot variables for K-means clustering are entered into the NCSS program.

FIG. 8 presents step five of Cluster analysis procedure: Report parameters are selected for the NCSS program.

FIG. 9 presents step six of Cluster analysis procedure: Plot features are selected for the NCSS program.

FIG. 10 presents step seven of Cluster analysis procedure: The cluster data from the spreadsheet is saved into the NCSS program so that future data can be interpreted using the current clusters.

FIG. 11 presents step eight of Cluster analysis procedure: The “Run” button is selected and a Cluster analysis report is generated using the NCSS program.

FIG. 12 presents step nine of Cluster analysis procedure: The cluster group designations are generated for each sample (Cluster “1” or “2” or neither 1 nor 2, which is “3”).

FIGS. 13 and 14 present step nine and ten of Cluster analysis procedure: Once cluster designations are defined from the training set, a Cluster designation for an unknown sample (sample “X”) can be generated by entering its microRNA value into a new row of the NCSS spreadsheet.

FIG. 15 presents the K-means Cluster Analysis Report summary page.

FIG. 16 presents the K-Means Cluster Analysis Report, F-Ratio section.

FIG. 17 presents thew K-Means Cluster Analysis Report Computer-generated random seed Distance data.

FIG. 18 presents the K-Means Cluster Analysis Report Distance data for Cluster 1 and 2.

FIG. 19 presents a K-Means Cluster Analysis Report for Cluster 3 with a Cluster Plot diagram.

FIGS. 20 and 21 presents the K-Means Cluster Analysis Report input summaries data.

FIG. 22 presents the top 20 differentially expressed MicroRNAs (Black compared to non-Blacks) that are tightly involved with the regulation of Focal Adhesion Pathway at multiple steps.

### SUMMARY OF DISCLOSURE

This disclosure provides improvements to the methods for predicting pregnancy complications as are currently known in the art (e.g. Winger, Reed: U.S. Pat. No. 10,323,282 B2 issued on Jun. 18, 2019). The disclosure provides microRNA (miRNA)-based tests and protocols for identifying and/or treating pregnant human beings at risk for a placental bed disorder during pregnancy, as well as reagents and/or kits relating to the same. In some embodiments, this disclosure provides reagents and methods for identifying reagents and methods for identifying at least two characteristic groups in a patient population on the basis of microRNA expression in maternal immune cells, wherein one characteristic group is associated with a reproductive disorder or risk of such a disorder, comprising the steps of a) quantifying at least one microRNA from a biological sample derived from maternal immune cells, and b) segregating the patient population into groups on the basis of expression of at least one microRNA, wherein the microRNA are selected from a group of microRNAs that are differentially expressed between patients who will experience healthy pregnancy and those who experience a placental bed disorder.

In some embodiments, this disclosure optionally provides therapeutic intervention in those patients identified at risk of a placental bed disorder. Differences in patient origin such as race constitute differences that may obscure identification of patients at increased risk of a placental bed disorder. The improvement that is identified and claimed is improvement in comparison of the patient to a control group by identifying the group, such as race group, and limiting comparison between similar groups.

### DETAILED DESCRIPTION

Previous studies compares microRNA expression of two groups of pregnant women: (1) those that develop a pregnancy disorder; and, (2) others that have healthy delivery. The comparison is performed using microRNA “cut-offs” with respect to the expression of particular microRNAs. Armed with the list of microRNAs differentially expressed between the groups, patients with unknown pregnancy risk can be assigned to one of the two categories, Healthy or Unhealthy delivery. Receiver Operating Character (ROC) curves are then developed using such information to generate panels of microRNA to comprise the panels. This disclosure further such methods by extending the composition of such microRNA expression panels by first sorting both patient and control samples into “clusters” identified using a second panel of one or more microRNAs (“cluster microRNA”). This is because the microRNAs associated with a pregnancy-related disorder can vary within certain groups of patients (i.e., “patient cluster” or “cluster”). It is not always possible to identify patients of a particular cluster through medical records or self-reporting (e.g., self-identification as of a particular race) but this can be carried out using a panel of microRNAs that can identify such patients are belonging to a particular cluster (e.g., by shared microRNA expression patterns). Thus, the methods disclosed herein first classify a patient within a particular cluster and then carry out microRNA expression screening of that clustered patient with a panel of microRNAs shown to be differentially expressed within that cluster of patients as compared to another patient cluster in which that panel is not associated with the risk of developing a pregnancy-related disorder. In preferred embodiments, the second panel includes or consists of at least one microRNA that is not known to be associated with the development and/or existence of a pregnancy-related disorder and/or pregnancy outcome. The cluster microRNAs can be associated with any other non-pregnancy disorder-related characteristic of a group. Thus, while any second panel of microRNAs can be used, the cluster microRNAs preferably include those not associated with the pregnancy prognosis. Thus, in this cluster step, patients are clustered (or grouped) using a panel of microRNAs that identifies one or more patient groups of like types (i.e., sharing at least one definable characteristic such as, e.g., genetic status, non-pregnancy disorder-related disease state, race (e.g., African American (or “Black”), American Indian, caucasian), and/or the like. In some embodiments, this disclosure provides optimal MicroRNA pregnancy outcome prediction panels differ between patient populations “clusters” (e.g., racial groups). Correct identification of a patient's cluster group is necessary for optimal pregnancy predictive power of the test to be achieved. For example, in some embodiments, the prediction that a patient is or is not at risk of a pregnancy-related disorder can be improved by defining a patient as within a particular cluster (e.g., race 1) and then screening that patient using a microRNA panel associated with the risk of a pregnancy-related disorder in that particular cluster, thereby providing the most specific pregnancy-related risk assessment.

The phrase “Great Obstetrical Syndromes” is generally used interchangeably with the term “placental bed disorder” “referring to the most common complications of pregnancy. These include preeclampsia, gestational diabetes, preterm labor, preterm birth, abruptio placentae, premature rupture of the placental membranes, miscarriage, implantation failure, incompetent cervix, stillbirth, intrauterine growth restriction, and fetal death. In pursuit of a pathogenetic designation for these syndromes, the phrase “Disorders of the Placental Bed” has been coined. (Reference: Pijnenborg, Robert, Brosens Ivor, Romero, Roberto, Placental Bed Disorders, Basic Science and its Translation to Obstetrics, Cambridge University Press, 2010, ISBN 978-0-521-51785-0).

MicroRNAs (microRNA) comprise a class of non-coding RNAs of about a 22-24 bases. They integrate disparate genetic elements into collaborative metabolic and signaling pathways. MicroRNAs form networks that supervise coordinated expression of mRNAs that guide and maintain, in turn, cell identity and buffer cell systems against changing conditions. MicroRNAs are known to be involved in embryonic development and have attracted great interest in the diagnosis and monitoring of various conditions including cancer, autoimmune, inflammatory and neurologic diseases (DePlanell-Saguor, et al. Analytica Chimica Acta. 2011; 699(2): 134-152). In previous studies, we determined that first trimester peripheral blood mononuclear cell (PBMC) microRNA provides sensitive and specific prediction of preeclampsia and preterm birth when sampled within a range of 4-14 weeks gestation (Winger et al. Early first trimester peripheral blood cell microRNA predicts risk of preterm delivery in pregnant women: Proof of concept. PLoS One. 2017 Jul. 10; 12(7):e0180124), (Winger et al. Peripheral blood cell microRNA quantification during the first trimester predicts preeclampsia: Proof of concept. PLoS One. 2018 Jan. 2; 13(1):e0190654). As used herein, specific microRNAs may be identified by their prefix mir- and a corresponding numeric identifier, such as mir-155. Sequences within an RNA transcript targeted by microRNAs may lie anywhere within the transcript. However, sequences within the 3′ untranslated region are most common. MicroRNA nomenclature comprises a three-letter prefix “mir” followed by a number assigned generally in order of the description of the microRNA. In one convention, when the “R” is lower case, the sequence refers to the pre-microRNA while when upper case is employed (miR), the mature form is indicated. Variants where the sequences vary by one or two bases may be designated by the letters “a” and “b”. Occasionally, pre-microRNAs located within separate regions of the genome result in an identical mature microRNA. These microRNAs are distinguished by a numeric suffix (e.g., “miR-123-1” and “miR-123-2”). When two microRNAs originate from opposite arms of the same pre-microRNA hairpin they are designated with the suffix-3p or -5p according to whether the 3′ or 5′ strand is used. As used herein, the numeric code, e.g., “mir-123” shall include its variants such as mir-123-1, mir123-2, and the -3p and -5p variants. As used herein the term “pri-microRNA” shall mean the RNA targeted by the Drosha-Pasha complex; the term “pre-microRNA” shall mean the product of the cleavage by the Drosha-Pasha complex; and no distinction shall be made between sequences between the parent nomenclature for example mir-123 and any more selective sequence for example mir-123-5p and other than by description within the text. Pre-microRNA may be generated by alternate pathways (non-canonic pathways) resulting in the same pre-microRNA sequence. Specific microRNA abbreviations may also include an additional prefix identifying the species of origin, such as “hsa” for Homo sapiens. MicroRNAs typically comprise approximately 18-25 nucleotides, in some embodiments, about 22 nucleotides. Nomenclature for microRNAs as used herein may be found in miRBase (www.mirbase.org), the entries of which represent the predicted hairpin portion of the microRNA transcript. As used in this specification, variations and/or equivalents in the sequences listed in mirbase.org are recognized. These include isomirs (i.e., isomiRs are miRNAs variants that originate from microRNA loci as consequence of specific processes as exoribonucleases or nucleotidyl transferase activity, RNA editing, SNPs or imprecise cleavage by the ribonucleases Drosha and Dicer), alleles (i.e., genomic variations in a listed microRNA or its pri-microRNA or pre-microRNA), and the like, and that, in certain embodiments, the methods, reagents and kits of this disclosure comprising such microRNA equivalents are intended to be included therein. Modifications to the pri-, pre- and mature sequence that do not alter the nucleic acid sequence are also included. These include modifications to bases such as the addition of methyl groups as in addition of methyl to the adenine at position 6. Other modifications are well known. MicroRNAs are also grouped into families. MicroRNAs within a family often share common evolutionary paths, regulate common pathways and are often functionally redundant. For example, a well described microRNA family known as Let-7 defines a group of microRNAs with common functions and sequences that vary by a few bases but retain similar functions. Other microRNA families with members are the mir-15/-16 family, mir-17 family, mir-19 family, mir-29 family and there are many others (reference: HCNC database supported by National Human Genome Research Institute (NHGRI) grant U24HG003345 URL: https://www.genenames.org/data/genegroup/#!/group/476 last accessed Jun. 13, 2023) Such microRNA families are regarded as interchangeable with respect to microRNA specifically claimed. Although the primary embodiments described herein are directed to humans, one of skill in the art will appreciate that, in some embodiments, the methods provided in this disclosure can be applied to other species. Mirtrons are the result of an alternate pathway of synthesis of microRNAs substituting splicing for Drosha-Pasha cleavage.

As used herein, the term “cluster” as applied to human subjects indicates a group of individuals expressing one or more characteristics in common whether the characteristic is predefined or to be discovered during the process of analysis. Clustering may rely upon the use of prior designated characteristics, or for example, self-identification within a racial group such as Black or non-Black. Other means of microRNA panel grouping may include, for example, the diagnosis of diabetes, autoimmunity, hypertension, kidney disease, obesity, age, history of preeclampsia, etc. In the case of race grouping, a “self-identified race” may be used to assign individuals sharing that characteristic to a common cluster. However, cluster analysis or cluster assignment does not require predefined group characteristics. It doesn't need to group data points into any predefined groups, which means that it can also be an “unsupervised learning” process. In “unsupervised learning”, characteristics are derived from the data, usually by use of computer algorithms which discover underlying patterns or data groups without any predefined labels. This later technique is well understood as a method of discovering relationships between groups while supervised analysis is better suited for making predictions. Cluster analysis is ultimately a statistical process. Software can assign membership with statistical parameters such as specific inclusion and exclusion parameters with respect to cluster assignment. An example of the use of statistics is the use of standard deviation (SD). A subject may be included within a cluster by use of an upper limit of SD a statistically defined cluster center. The inventors utilize microRNA expression levels as clustering characteristics. Sequence variations within said microRNAs may be used to distinguish clusters. In a preferred embodiment, microRNA that have limited differential expression in prognosis of patients with respect to pregnancy outcome may be well-suited to a define a patient cluster. It is understood, however, that all microRNAs, regardless of their prognostic capacity, may be used for cluster definition. Wherein microRNAs may be useful in distinguishing clusters and also are differentially expressed in prognosis, artificial intelligence may be employed in their usage. K-means clustering is the preferred method used herein. The use of such microRNAs may be of particular utility in defining clusters significant in determining pregnancy outcome. In a specific type of analysis, two characteristics are utilized to segregate individuals known as Principal Component Analysis where the most and second most varied data to segregate members into clusters (See ref: A Step-by-Step Explanation of Principal Component Analysis (PCA) URL: https://builtin.com/data-science/step-step-explanation-principal-component-analysis, last accessed Jul. 23, 2023).

The disclosure provides methods that improve the conventional use of a control group by additionally limiting comparison of patients to a control group belonging to a common patient cluster. Because the term “cluster” may be defined by the expression levels of one or more microRNAs, it is not limited to a specified race (white, Black or Asian), nationality, ethnicity or environmental exposure such as economic status because microRNA expression is affected by all of these factors.

In a preferred embodiment, clusters will be defined by microRNAs expressed by subgroups of women in their first or second trimester of pregnancy in peripheral blood (maternal immune cells). In lieu of using self-identified race to identify patients and control groups with common characteristics, the expression levels of microRNAs may be used. Table 13 (Tables 13A and 13 B) demonstrates differential expression of microRNA between patients self-identified as Black or non-Black. In preferred embodiments, microRNAs selected for discrimination between races include one or more microRNAs of top 20 microRNAs given in Table 14 with a p value for prediction of <0.05.

In some preferred embodiments, the use of one or more microRNAs selected from Table 13 can be used to sort patients into clusters belonging to one of the two racial clusters. Clusters defined by expression levels one or more microRNAs may be determined by a variety of methods. While visual inspection of graphical display based on microRNA expression, it may be preferable to employ a software-based method to define clusters for example the NCSS 2023 statistical software program. In particular, the software provides analysis using K-means for Patient Clustering. In addition, Principal Component Analysis provides additional clustering techniques. The latter method sorts patient samples by principal components. Conventionally at least two components demonstrating the greatest variance amongst expressed in samples are used to sort samples into clusters. However, in preferred embodiments, this disclosure provides methods of selecting principal components by an additional factor, their biologic function. Selecting microRNAs that regulate pathways known to participate in reproductive pathology as principal components may provide patient clusters amenable to directed intervention.

As used herein, a “self-identified race” represents the conventional understanding of racial groups. Other equivalent phrases may include race, physical appearance, skin color, origin such as sub-Saharan Africa, history of migration such as Celtics, a Germanic group migrating to Great Britain. A person may “self-identify” as Black, non-Black, white, or Asian, for example.

As used herein, the term “Black” shall mean Homo sapiens who remained in sub-Saharan Africa following the diaspora of a group of Homo sapiens out of Africa estimated at approximately 50,000 to 100,000 years ago. Other racial groups such as Asian or Native American can also be used to define clusters. Homo sapiens may also be designated as either “African or non-African” or interchangeably, “Black or non-Black”. Here the term African shall represent Homo sapiens that remained in Africa following the diaspora around 50 to 100,000 years ago.

This disclosure provides microRNA-based tests and identifies patients who might benefit from treatment. As shown herein, in some preferred embodiments, a ratio (“HC Ratio”) for an individual microRNA can be calculated and used to identify microRNAs of interest. As mentioned above, the HC ratio is preferably calculated by using as the numerator, the mean microRNA signal (i.e., expression) for a “compromised pregnancy outcome” population minus the mean microRNA signal level (i.e., expression) for a “healthy pregnancy outcome” population (in other words, subtracting the mean microRNA signal level for a “healthy pregnancy outcome” population from the mean microRNA signal for a “compromised pregnancy outcome” population), and using as the denominator the average of the standard deviations (SD) of the “healthy pregnancy outcome” mean signal level and the “compromised pregnancy outcome” mean signal level. The HC ratio is preferably calculated from application of the following formula: numerator: mean microRNA signal (compromised) minus mean microRNA signal (healthy); denominator: the average of the sum of the standard deviations of the mean signals for each the compromised and healthy signals divided by two (2).

The individual microRNAs identified with high HC ratios are shown herein to distinguish the two populations, for example, those with a placental bed disorder (e.g., preeclampsia) from those women destined to have healthy pregnancy outcome. In a preferred embodiment the HC ratio shall be equal or greater than about any of 1.0, 1.1, 1.2, 1.3, 1.4, or 1.5, and is most preferably equal to or greater than the absolute value of 1.5 (Tables 2 and 3). ROC curves may be calculated. In preferred embodiments, as shown herein, for microRNAs that demonstrate a high ratio, the “associated criterion value” at the Youden index J point of the ROC calculation can be used to determine the cut-off value used to determine patient risk of developing a placental bed disorder (MedCalc Statistical Software version 19.0.7: MedCalc Software bvba, Ostend, Belgium; https://www.medcalc.org; 2019). Other cut-off points may be calculated by those of ordinary skill in the art. In some embodiments, when a patient's measured microRNA signal is greater than this predetermined cut-off value, the patient is deemed to be at “higher risk” of experiencing a placental bed disorder. Those women in whom “higher risk” is assessed may then be considered for intervention such as by pharmaceutical agents.

As used herein, the term “placental bed disorder” refers to conditions that can arise during pregnancy that typically have deleterious effects during pregnancy and post-partum, such as hypertension and increased risk of cardiovascular disease, and includes but is not limited to preeclampsia, preterm birth, HELLP Syndrome (a complication of pregnancy characterized by hemolysis, elevated liver enzymes, and a low platelet count), gestational diabetes, miscarriage, implantation failure, intrauterine growth retardation (IUGR) or fetal growth restriction, and Premature Rupture of the Membranes (PROM.). Within this disclosure, the term “placental site” shall refer to the discrete area of the maternal endometrium in direct contact with the implanting feto-placental unit, which is coextensive with the placenta.

The methods, reagents and kits disclosed herein may also be as described in U.S. Pat. No. 10,323,282 B2 issued on Jun. 18, 2019; PCT/US2012/061994 filed on Oct. 25, 2012; U.S. Ser. No. 13/284,739 filed on Oct. 28, 2011; U.S. Ser. No. 61/767,669 filed on Feb. 21, 2013; and/or U.S. Ser. No. 61/456,063 filed on Nov. 1, 2010; each of which being incorporated herein into this application in their entireties. In addition, the methods, reagents and kits disclosed herein may also be as described in reference paper: Winger E E, Reed J L et al. Am J Reprod Immunol. 2014 November; 72(5):515-26, and reference paper: Winger E E, Reed J L et al. J Reprod Immunol. 2015 August; 110:22-35, each of which being incorporated herein into this application in their entireties.

As used herein, the term “about” when referring to a value or to an amount of mass, weight, volume, concentration is meant to encompass variations of some preferred embodiments of ±20%, in some preferred embodiments ±10%, in some preferred embodiments ±5%, in embodiments ±1, in some preferred embodiments ±0.5%, and in some preferred embodiments ±0 0.1% from the specified amount, as such variations are appropriate to perform the disclosed methods. Further, sequences, such as microRNA sequences and their precursors may vary but still fall within the definition thereof. These variations may comprise alleles that are expressed in the genome. Variations are also recognized that the result of post translational variations such as by deletions or additions of bases at either the 5′ or 3′ end of the polynucleotide usually by enzymatic activity.

Preeclampsia, as an example of a placental bed disorder, affects at least 2-3% of all pregnancies and is a major cause of maternal and perinatal morbidity and mortality (Knight, et al. eds. on behalf of MBRRACEUK. Saving lives, improving mothers' care—lessons learned to inform future maternity care from the UK and Ireland confidential enquiries into maternal deaths and morbidity December 2009. Oxford: National Perinatal Epidemiology Unit, University of Oxford; 2014). The condition is clinically recognized after about 20 weeks of gestation with the new appearance of hypertension and proteinuria. In countries with limited access to medical care, it is estimated that the disorder is responsible annually for greater than 60,000 deaths worldwide (World Health Org. 2005. World health report: Make every mother and child count. Geneva: World Health Org. URL:http://www.who.int/whr/2005/whr2005-en.pdf. Last accessed Jul. 24, 2017). In developed countries, therapeutic intervention is often concluded with early delivery. While this intervention protects the mother, it results in significant morbidity and mortality to the neonate. (Friedman et al. Neonatal outcome after preterm delivery for preeclampsia. Am J Obstet Gynecol. 1995; 172:1785-1792). Early diagnosis has been a goal permitting intervention at an early time point (Bujold, et al. Prevention of preeclampsia and intrauterine growth restriction with aspirin started in early pregnancy: a meta-analysis. Obstet Gynecol. 2010. August; 116(2 Pt 1):402-414).

While certain microRNA-based tests and treatment protocols for preeclampsia have been developed, there is a need in the art for additional (e.g., more accurate and/or condition-relevant and at an earlier time point in pregnancy) microRNA-based tests and treatment protocols for placental bed disorders, including preeclampsia. These improvements recognize that comparison of patient and healthy control group microRNAs is improved wherein the patient and control group share common backgrounds such as race. Preferred embodiments of tests and treatment protocols are provided in this disclosure wherein patients are sorted into clusters by their microRNA expression levels.

Within this disclosure, the term “non-placental biological sample” shall mean maternal cells (preferably maternal immune cells) and derivatives thereof not collected from the placental site, for example the peripheral blood, of a subject (for example a pregnant human being). A non-placental biological sample (preferably maternal immune cells) may be derived from an individual being investigated for the propensity or likelihood of developing a placental bed disorder, or having a placental bed disorder, preferably during the first and second trimesters of a pregnant woman. A period of about 6 months prior to the initiation of pregnancy is also included. As used herein, the term “subject” refers to any mammal, including both human and other mammals. A “control subject” is an individual(s) of comparable characteristics such as age, sex, race and/or condition (e.g., pregnant) who does not have a placental bed disorder, and/or related condition(s) and/or pathology leading to said placental bed disorder and are not known to be at risk of developing a placental bed disorder. The term “control sample” shall mean a non-placental biological sample of a control subject, taken from the same source, such a peripheral blood, and collected under the same or comparable conditions including time of acquisition as a patient sample comprising cells of the non-placental biological sample. In some embodiments, the term “control sample” as used herein may represent the mathematical mean of multiple samples from control individuals wherein a number of samples considered sufficient by an individual of ordinary skill in the art are collected. Additional statistical parameters may be derived from said samples such as standard deviation of the mean. Said additional statistical parameters may be used for purposes of comparison of a patient test result with control samples to estimate the probability that the patient's test result represents an abnormal finding and, thereby suggesting that the patient is suffering from preeclampsia or related condition or risk of said condition. For purposes of simplicity the term may also be used in another way wherein a plurality of comparable, temporally separate, samples are collected and assayed from a single individual and compared with one another such that a first sample or a particular subsequent sample are compared as though the first is a control for the second, permitting assessment of a change in condition potentially as a function of the clinical state, stage of pregnancy or as a result of therapeutic intervention. Preferably, the subjects to whom the methods described herein are applied are human beings, most preferably pregnant human beings. As used herein, the phrase “expression profile” shall mean one or more microRNAs that are differentially expressed between healthy, and individuals destined for compromised pregnancy including birth. These microRNAs may be used for comparison between controls and patients.

Suitable techniques for isolating cells from non-placental biological sample (preferably maternal immune cells) can include isopycnic density-gradient centrifugation or monoclonal antibody paramagnetic bead conjugates, for example, as are well-known known in the art as well as any other suitable techniques that are available to those of ordinary skill in the art. In some embodiments, this disclosure provides methods comprising providing a non-placental biological sample (preferably maternal immune cells). Such a non-placental biological sample can be derived from cells of the biologic sample (preferably maternal immune cells) such as, for example, peripheral blood (e.g., whole blood), the buffy coat thereof (i.e., the fraction of an anticoagulated peripheral blood sample that contains most of the white blood cells and platelets following centrifugation of the blood), bone marrow. Maternal mononuclear cells may also be isolated as taught by Boyum (Scand J Immunol 17:429-436 (1983). In a preferred embodiment, for example, a sample derived from a peripheral blood and/or bone marrow can include any leukocyte population(s), for example, monocytes, lymphocytes, granulocyte, platelets, and/or stem cells may be segregated by means well known in the art permits selective quantification of microRNAs within that cell population. Further, for example, cell subpopulations (e.g., T cells, B cells) can be individually interrogated following their selective isolation by techniques such as, for example, flow cytometric sorting following interaction with fluorescently labeled monoclonal antibody combinations that are capable of discreetly characterizing the individual subclasses. It is understood by those of ordinary skill in the art that the microRNA content of a sample enriched for peripheral blood cells (e.g., the buffy coat) is representative of the microRNA content of the mononuclear cells in that sample because the microRNA content of peripheral blood cells is vastly greater than that of plasma. Thus, in preferred embodiments, a buffy coat specimen or even a whole blood specimen is essentially equivalent to a mononuclear cell specimen so long as the specific microRNA quantified is in sufficient excess of the microRNA in non-cellular components of the sample that said quantification provides clinically equivalent results as those derived from purified cells such as peripheral blood mononuclear cells (PBMCs). Various methods for detection of microRNA, for example by the polymerase chain reaction (PCR) are known. Preferably the method of Chen et al. is suitable. The method is described (http://www3.appliedbiosystems.com/cms/groups/mcb-marketing/documents/generaldocuments/cms-040548.pdf downloaded May 11, 2010). Primers and reagents may be selected for individual microRNAs from those described in product overview (http://www3.appliedbiosystems.com/cms/groups/mcb-marketing/documents/generaldocuments/cms-068884.pdf downloaded May 11, 2010).

Commercial reagents and kits may be configured to recover short RNA polynucleotides of microRNA length. Commercial reagents with accompanying instructions are widely available. It is understood herein that detection of microRNA may include detection of the presence or absence of a specific microRNA within a non-placental biological sample, and more preferably its quantification. The methods may produce semi-quantitative or quantitative results. It is understood that relative quantification wherein comparative levels between the sample of the patient is related to the level in a control or other sample particularly wherein sequential samples are assayed. Any detection method well known to those skilled in the art falls within the scope of the invention. Hybridization, preferably where a polynucleotide complimentary to the target polynucleotide is labeled, may be used to detect the target strand, in particular wherein nucleic acid probes complementary to specific microRNAs are attached to a solid phase as in a microarray plate. Polymerase chain reaction (PCR) using labeled probes, electrophoresis, and/or sequencing of target strands, or other detection strategy may be employed.

In some embodiments, RNA can be extracted from cells of the non-placental biological sample (preferably maternal immune cells) according to well-known techniques. Blood collected can be drawn into tubes comprising an anticoagulant such as heparin or EDTA and maintained at room temperature preferably for approximately 24 hours prior to isolation of cells. Buffy coat may also be used wherein sample tubes are spun at a sufficient speed and for a sufficient time to separate blood visibly into three components (plasma, buffy coat layer and red cell layer) and the buffy coat layer isolated by pipette (often 10-15 minutes). RNA sampling and extraction: cells or sorted cell populations (<1×10⁷ viable cells) are collected in 1 ml Trizol (Invitrogen) and stored at −80° C. until use. Total RNA can be isolated according to standard techniques, such as using the Tri/zol reagent/protocol (Invitrogen) and/or RNeasy Mini Kit (Qiagen) (e.g., at room temperature with the QIAcube automated robot (Qiagen)). Total RNA yield can be assessed using the Thermo Scientific NanoDrop 1000 micro-volume spectrophotometer (absorbance at 260 nm and the ratio of 260/280 and 260/230), and RNA integrity assessed using, e.g., the Agilent's Bioanalyzer NANO Lab-on-Chip instrument (Agilent). MicroRNAs may be quantitated by any suitable technique including but not limited to quantitative real time PCR (qPCR using, e.g. SYBR Green, a TaqMan probe, locked nucleic acid probe (Vester, et al. Nature Methods, 7: 687-692 (2004), microRNA arrays, next generation sequencing (NGS) techniques (e.g., TruSeq kits (Illumina); (Baker et al. Biochemistry, 43:13233-13241 (2010)), multiplex microRNA profiling assays.

In some embodiments, the expression of various microRNAs in a non-placental biological sample (preferably maternal immune cells) of an individual can be collected and assembled to provide a microRNA signature for that individual. Analysis and/or comparison of a microRNA signature of a non-placental biological sample may be compared with a corresponding microRNA signature derived from a control sample and/or a database representative of a control sample. Mathematical approaches to analysis of data and methods for comparison are well known to those skilled in the art.

The methods for quantifying or semi-quantifying microRNA(s) are well-known in the art. These include but are not limited to nucleic acid hybridization techniques well-known in the art for example performed using a solid phase support comprising specific, bound polynucleotides complementary to the target microRNA sequence. RNA isolated from a biologic sample may be reversed transcribed into DNA and conjugated with a detectable label and thence contacted with the anchored probes under hybridizing conditions and scanned by a detection system permitting discrete quantification of signals. It is understood that probe sequences may also be complementary to target sequences comprising single-nucleotide polymorphisms (“SNPs”). Moreover, it is understood that probe sequences may be complementary to pre-microRNA and pri-microRNA regions of specific microRNAs. Techniques comprising the polymerase chain reaction (PCR), preferably those incorporating real-time techniques, wherein amplification products are detected through labeled probes or utilizing non-specific dye amplicon-binding dyes such as Cyber Green™. For instance, RNA may be extracted from cells isolated cells by extraction according to instructions from the manufacturer (Qiagen catalogue 763134). microRNA such as mir-146a-5p may be detected and quantified by polymerase chain reaction (PCR) by the method described by Chen et al (vide supra). Sequencing methods may also be utilized for quantification as well as for identification of isomers and alleles as defined utilizing methods well known to those with ordinary expertise in the art.

In some embodiments, an individual identified as being at risk for a placental bed disorder (or as having a placental bed disorder) may be treated by a therapeutic intervention that can prevent, slow, or eliminate the placental bed disorder. Exemplary therapeutic intervention(s) can include any one or more of immunotherapy (e.g., administration of a immunosuppressant and/or anti-inflammatory drug such as intravenous immunoglobulin (IVIG), corticosteroids, Neupogen™, anticoagulant(s) (e.g., heparin(s) such as low molecular weight versions such as Lovenox™), statin(s), progesterone, antibiotic(s), metformin, Cerclage, intralipids, “natural” therapies (e.g., omega-3 and/or fish or krill oil preparations, and the like), dietary changes and/or restrictions, bedrest regimens, and the like. In some embodiments, the appropriate therapeutic intervention can be selected using various in vitro cell markers of maternal immune cells (any maternal (non-fetal) immune cells or subset thereof, e.g., of peripheral blood mononuclear cells (PBMCs)). In further embodiments, immune cells are isolated prior to microRNA quantification and the specific microRNAs are quantified within the individual cell types. Isolation may be done by flow cytometry or by paramagnetic beads conjugated to appropriate cell-type selective probes. It is also understood that in situ hybridization of microRNA probes may be used for both quantification and identification of the site of expression such as in a tissue for example a lymph node.

In some embodiments, quantification of various microRNAs and patterns of microRNA change (e.g., at least one of the microRNAs) may be listed. MicroRNA expression levels and/or at least one or more equivalent(s) measurement (s) thereof in maternal cells at various time points prior to and following immunotherapeutic intervention may be performed. These microRNA “signatures” can direct the clinical diagnosis and/or treatment. This disclosure also contemplates that the methods, reagents and kits described herein can be used to assess other clinical conditions beyond placental bed disorders and/or different immunotherapeutic interventions.

In preferred embodiments, said quantification of microRNAs can be used to define patient clusters. These microRNAs may include hsa-let-7a-5p, hsa-let-7f-5p, hsa-let-7g-5p, hsa-let-7i-5p, hsa-miR-150-5p, hsa-miR-15b-5p, hsa-miR-16-5p, hsa-miR-19b-3p, hsa-miR-21-5p, hsa-miR-223-3p, hsa-miR-23a-3p, hsa-miR-26b-5p, hsa-miR-29a-3p, hsa-miR-29c-3p, hsa-miR-142-3p, hsa-miR-24-3p, hsa-miR-342-3p, hsa-miR-17-5p, hsa-miR-22-3p and hsa-miR-25-3p (Table 14). Some additional embodiments may use hsa-miR-582-5p, hsa-miR-6737-3p, hsa-miR-193-3p and hsa-miR-223-5p to define patient clusters (Table 19). Their use simplifies complex diagnostic strategies into a single procedure and provides information heretofore unavailable. In some embodiments, the methods described herein can include detecting expression of the microRNAs (and/or symptoms of a placental bed disorder) before, during and/or after such therapeutic intervention and treatment can be adjusted according to such expression.

Additional information may be derived from comparison of differential expression related to specific microRNAs within the control panel. Differential expression of specific microRNAs may suggest specific abnormalities in pathways regulated by panel comprised microRNAs. Thus, differential expression of a microRNA within the control panel may suggest an abnormality related to the specific pathway regulated by groups of microRNAs (Table 17). It is recognized that such findings might direct specific therapeutic interventions. In the current invention, differential expression of microRNAs between racially distinct groups may suggest different interventions such as identified by principal component analysis.

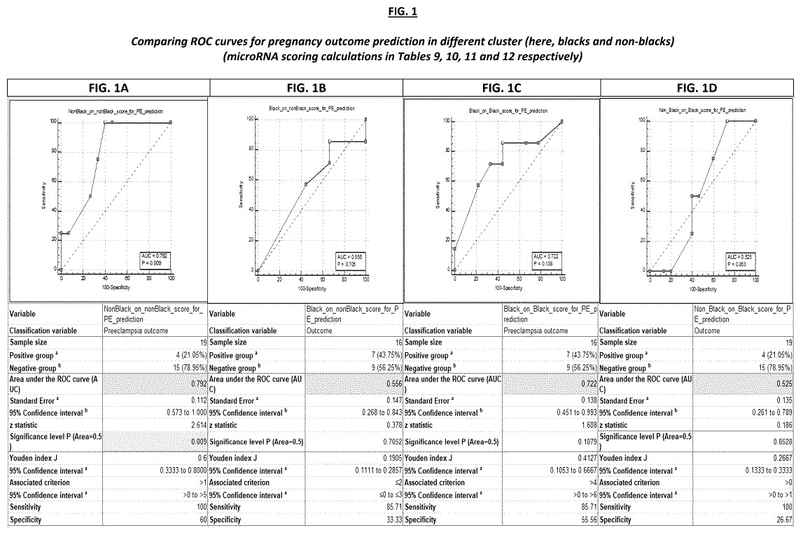

The methods described herein can comprise quantification of one or more individual microRNAs from the non-placental biological sample and quantifying the individual microRNAs and comparing the expression levels of microRNA(s) in the test sample to the expression levels of the corresponding microRNA in a control sample(s). The methods, reagents and kits, especially with respect to the second step (i.e., steps b) of Embodiments 1 and 2 below) disclosed herein may also be as described in U.S. Pat. No. 10,323,282 B2 issued on Jun. 18, 2019; U.S. Pat. No. 11,268,147 B2 issued on Mar. 8, 2022; U.S. Pat. No. 12,012,635 B2 issued on Jun. 18, 2024; PCT/US2012/061994 filed on Oct. 25, 2012; U.S. Ser. No. 13/284,739 filed on Oct. 28, 2011; U.S. Ser. No. 61/767,669 filed on Feb. 21, 2013; and/or U.S. Ser. No. 61/456,063 filed on Nov. 1, 2010); each of which being incorporated herein into this application in their entireties. The specification discloses herein provides an improvement wherein subject and control panel individuals are derived from the same or closely related group, e.g. such as the same race. In the current invention, differential expression of microRNAs between racially distinct groups may suggest need for race-specific pregnancy risk panels for optimal predictive power achievement in the clinic. As described, microRNA panels for pregnancy risk assessment may vary between different races. If a pregnancy panel is developed on one race and applied to another race (for example, a Black panel is used on a non-Black patient), the ability to predict pregnancy outcome becomes less robust. Correct identification of a patient's racial group is necessary in order optimal pregnancy predictive power of the test to be achieved. However, correct identification of the patient's racial group is not always possible by visual inspection or medical record assessment. In many cases, a patient may be of mixed race or have unknown parentage. In other cases, a patient may not volunteer their family history. In other cases, an individual may simply not have this information available to them. The modification eliminates differences in control panel members and subject that may not comprise the best microRNAs for comparison of the two (see Tables 9-12 and FIG. 1). Mismatch of subject and control group is shown in FIG. 1 where mismatched groups perform significantly less well than those that are matched (such as when a Black patient uses a non-Black microRNA panel).

Identification of patients belonging to microRNA response groups is associated with improved efficacy, prognosis and utility of particular therapeutic intervention(s). Moreover, quantitative levels of certain microRNAs and patterns of change within microRNAs may predict patient response group(s) and post-therapy levels may have additional predictive value. In some embodiments, expression profiles may consist of the entirety of all known microRNAs incorporated into or onto a microarray chip, bead or other solid support typically used in expression analysis. Selection of microRNAs to be configured into a control panel may be selected from Tables 2 and 3 wherein microRNAs are selected with the highest HC ratios, preferably 1.5 or greater and are derived from the same population group (e.g., such as the same race).

Any of several methods may be used for quantification or semi-quantification. Determination of an expression profile may be performed by quantitative or semi-quantitative determination of a panel of microRNAs in patients affected by a condition to be assessed and in individuals without said condition. Alternatively, determination of an expression profile that may be used to determine progress of a condition may be determined in a similar manner wherein comparison is made by quantitative or semi-quantitative differences between the two time points. Separate expression profiles may be determined in a similar manner wherein the two time points are separated by a therapeutic intervention. In a similar manner individual expression profiles may be determined at different time points particularly during the course of pregnancy including time points within 6 months preceding or following pregnancy. Panels of microRNAs to be assessed selected a priori or these may comprise large collections intended to include all currently known microRNAs such as in a microarray. The determination may be carried out by any means for determining the expression profiles of nucleic acids (e.g., microRNAs).

Additional information may be derived from comparison of differential expression related to specific microRNAs within the control panel. Differential expression of specific microRNAs may suggest specific abnormalities in pathways regulated by panel comprised microRNAs. Thus, differential expression of a microRNA within the control panel may suggest an abnormality related to the specific pathway regulated by the specific microRNA. It is recognized that such findings might direct specific therapeutic interventions. In the current invention, differential expression of microRNAs between racially distinct groups may suggest different interventions. Principal component analysis sorting by microRNAs of specific interest identifies subjects and controls by biologic characteristics related to a disease/condition.

In preferred embodiments, this disclosure provides the following embodiments:
1. A method comprising:
   a) classifying a pregnant human as a member of a particular patient cluster by quantifying the expression of a first panel of microRNAs (miRNAs) in one or more biological samples of the pregnant human being as compared to that of a control sample of the particular patient cluster;
   b) comparing the expression of a second panel miRNAs associated with an increased risk of developing a placental bed disorder in the particular patient cluster, wherein a difference in the expression in a test biological sample of the pregnant human being relative to a control biological sample of the particular patient cluster indicates the pregnant human being is at risk of developing a placental bed disorder; and,
   c) treating the pregnant human being identified in step b) as being at risk of developing a placental bed disorder using immunotherapy.
2. A method comprising:
   a) cluster identification by classifying a pregnant human being as a member of a first patient cluster but not at least one second patient cluster by quantifying the expression of a first panel of microRNAs in a biological sample of the pregnant human being as compared to that of a second patient cluster, the first panel of microRNAs consisting of one or more miRNAs that are not necessarily known to be associated with a pregnancy-related disorder in either the first or second clusters but known to be differentially expressed in patients of the first and second clusters; wherein differential expression of the first panel of microRNAs by the pregnant human being as compared to the second patient cluster identifies the patient as a member of the first patient cluster;
   b) identifying a risk for a pregnancy-related disorder by: determining the expression of a second panel of micro RNAs in a biological sample of the pregnant human being identified in step a) as a member of the first patient cluster, the second panel of microRNAs consisting of one or more microRNAs known to be associated with a pregnancy-related disorder in the first patient cluster but not patients of a second patient cluster, comparing the expression by the pregnant human being of the second panel of microRNAs to the expression of the second panel of microRNAs of a pregnancy-related disorder control sample, wherein differential expression of the second panel of miRNAs in the pregnant human being as compared to the control sample indicates an increased risk of the pregnant human being developing a placental bed disorder; and,
   c) optionally, treating the pregnant human being identified in b) as being at risk of developing a placental bed disorder using immunotherapy.
3. The methods above wherein expression of the miRNAs in the pregnant human being is higher than that of the control sample of the particular patient cluster, thereby identifying the pregnant human being a member of that particular patient cluster.
4. The methods above wherein expression of the miRNAs in the pregnant human being is lower than that of the control sample of the particular patient cluster, thereby identifying the pregnant human being a member of that particular patient cluster.
5. The methods above wherein the miRNAs of the first panel are selected from the group consisting of hsa-let-7g-5p, hsa-miR-150-5p, hsa-let-7f-5p, hsa-let-7i-5p, hsa-miR-15b-5p, hsa-miR-16-5p, hsa-miR-26b-5p, hsa-miR-29a-3p, hsa-let-7a-5p, hsa-miR-19b-3p, hsa-miR-21-5p, hsa-miR-223-3p, hsa-miR-23a-3p, hsa-miR-25-3p, hsa-miR-17-5p, hsa-miR-22-3p, hsa-miR-24-3p, hsa-miR-342-3p, hsa-miR-142-3p, and hsa-miR-29c-3p.
6. The methods above wherein the biological sample is obtained during the first trimester of pregnancy.
7. The methods above wherein the placental bed disorder is selected from the group consisting of preeclampsia, preterm birth, HELLP Syndrome, gestational diabetes, miscarriage, implantation failure, fetal growth restriction, and premature rupture of the membranes (P.R.O.M.).
8. The methods above, wherein the placental bed disorder is preeclampsia.
9. The methods above wherein the control biological sample is representative of a pregnant human being without a placental bed disorder.
10. The methods above wherein the biological sample is derived from peripheral blood.
11. The methods above wherein the biological sample comprises mononuclear cells.
12. The methods above, further comprising the additional step of isolating mononuclear cells from the biological sample.
13. The methods above wherein the biological sample is peripheral blood.
14. The methods above, further comprising the step of extracting miRNA-comprising RNA from the biological sample.
15. The methods above comprising calculating a ratio (HC ratio) of expression of said at least one miRNA of the first and/or second panels, wherein said ratio comprises: a numerator equal to the difference between the mean value of expression of the at least one miRNA in the first population and the mean value of the second population and the denominator comprises the average of the two standard deviations of the values for the first and second populations; wherein the at least one miRNA is selected from the group consisting of the miRNAs presented herein as being capable of distinguishing the first and second populations.
16. The methods above wherein said miRNA exhibits a signal consistency of at least about 85%; a mean signal strength of at least 5.0; and a p value of less than 0.05 (p<0.05).
17. The methods above wherein the at least one miRNA exhibits a HC ratio of greater than or equal to about any of 1.0. 1.1, 1.2, 1.3, 1.4, or 1.5; or greater than or equal to about 1.3.
18. The methods above comprising the use of a reagent for quantifying a panel miRNAs, the panel comprising miRNAs selected from the group consisting of hsa-let-7g-5p, hsa-miR-150-5p, hsa-let-7f-5p, hsa-let-7i-5p, hsa-miR-15b-5p, hsa-miR-16-5p, hsa-miR-26b-5p, hsa-miR-29a-3p, hsa-let-7a-5p, hsa-miR-19b-3p, hsa-miR-21-5p, hsa-miR-223-3p, hsa-miR-23a-3p, hsa-miR-25-3p, hsa-miR-17-5p, hsa-miR-22-3p, hsa-miR-24-3p, hsa-miR-342-3p, hsa-miR-142-3p, and hsa-miR-29c-3p.
19. The methods above using a component of claim 17 wherein said component is selected from the group consisting of a nucleic acid amplification primer, a pair of nucleic acid amplification primers, and an oligonucleotide probe corresponding to at least one of said miRNAs.
20. The methods above using a microarray, solid support, or collection of solid supports, comprising a panel of miRNAs selected from the group consisting of hsa-let-7g-5p, hsa-miR-150-5p, hsa-let-7f-5p, hsa-let-7i-5p, hsa-miR-15b-5p, hsa-miR-16-5p, hsa-miR-26b-5p, hsa-miR-29a-3p, hsa-let-7a-5p, hsa-miR-19b-3p, hsa-miR-21-5p, hsa-miR-223-3p, hsa-miR-23a-3p, hsa-miR-25-3p, hsa-miR-17-5p, hsa-miR-22-3p, hsa-miR-24-3p, hsa-miR-342-3p, hsa-miR-142-3p, and hsa-miR-29c-3p; and/or a binding partner for at least one of said miRNAs; and/or the a microarray, solid support, or collection of solid supports per se.
21. The methods and/or microarray, solid support, or collection of solid supports above comprising a nucleic acid amplification primer, a pair of nucleic acid amplification primers, and/or an oligonucleotide probe corresponding to at least one of said miRNAs.
22. The methods and/or solid support or collection of solid supports above wherein said solid support is a bead or collection of beads, respectively.
23. A kit comprising a component, microarray, solid support, or collection of solid supports above, optionally further including instructions for use; and/or methods using the same.
   Other embodiments are also contemplated herein as will be understood by those of ordinary skill in the art.

All patents and references whether conventionally cited in the literature or addressed through internet links herein are incorporated in entirety by reference. All technical and scientific terms used within this description shall have the same meaning as commonly understood by those or ordinary skill in the art disclosed herein except where otherwise specifically defined. Following longstanding practice patent law conventions, the terms “a’, and “the” refer to “one or more” when used in this application including the claims. Thus, for example, reference to “a polynucleotide” includes a plurality of such polynucleotides, and so forth.

### EXAMPLES

### Example 1

As mentioned above, differentially expressed genes and enhancers observed between modern human races may account for immune and metabolic and pregnancy outcome differences observed between races. Analysis of the top 20 differentially expressed microRNAs between pregnant Blacks and non-Blacks (C1C2 microRNAs) was limited to only to those microRNAs that regulate known Neanderthal introgressed enhancer single nucleotide polymorphisms (SNPs). Table 31 presents Reactome pathways analysis (Reactome v84) that was performed on top 10 C1C2 microRNAs most differentially expressed microRNAs between pregnant Black and non-Black (Table 32 herein) that also regulate known Neanderthal introgressed enhancer SNPs. The data presented in Table 32 is presented as C1C2 ratio rankings (non-Black versus Black). 175 microRNAs not as useful for pregnancy outcome prediction were selected (HC ratio <1.0) and only samples from healthy pregnancies were assessed to avoid conflation with pregnancy prediction microRNAs. The mean and standard deviation for 175 microRNA expression rankings were calculated using three healthy pregnant Black patients and six healthy pregnant non-Black patients (see population details Table 1). The numerator comprised the difference between the mean expression rank of the three patient “Black” group population (Cluster 2) minus the mean expression rank for the six patient “non-Black” group population (Cluster 1) and the denominator comprised the average of the two standard deviations of the values for the three Black and six non-Black individuals. These C1C2 Ratios for each of the 175 microRNAs were then sorted from highest to lowest. MicroRNAs with the highest C1C2 ratios >2.3 with p value 50.05 were considered to have the greatest ability to assess racial type. A set of statistically significant pathways were found. Interestingly, many of these pathways are known to be critical in the formation of placental bed in early pregnancy. Hypoxia-inducible Factor-1 alpha (HIF-1alpha), for example, binds to the hypoxia response element in VEGF at the time of pregnancy implantation enabling anaerobic metabolism in the low oxygen environment of the implantation site. (Daikoku T et al. J Biol Chem. 2003 Feb. 28; 278(9):7683-91).

In preferred embodiments, the microRNA/gene associations are determined using mirDIP 4.1 (See Supplementary Table A, below) (mirDIP reference: Tokar T, Pastrello C, Rossos A E M, Abovsky M, Hauschild A C, Tsay M, Lu R, Jurisica I. mirDIP 4.1-integrative database of human microRNA target predictions. Nucleic Acids Res. 2018 Jan. 4; 46(D1):D360-D370. doi: 10.1093/nar/gkx1144. PMID: 29194489; PMCID: PMC5753284). Gene sets selected for analysis were “Regulation of cell motility” (GO:2000145) and “Regulation of cell migration” (GO:0030334). These are biologic processes are involved with trophoblast migration in early pregnancy.

The most statistically significant pathway calculated from the Neanderthal introgressed genes regulated by our top C1C2 (population identifying) microRNAs using Reactome® software was “Regulation of gene expression by Hypoxia-inducible Factor” (Table 31). Hypoxia-inducible Factor-1 alpha (HIF-1 alpha) is important in early pregnancy. It binds to the hypoxia response element in VEGF at the time of pregnancy implantation enabling anaerobic metabolism in the low oxygen environment of the implantation site. (Daikoku T et al. J Biol Chem. 2003 Feb. 28; 278(9):7683-91).

In addition to HIF-1-alpha regulation pathway, Neanderthal introgressed genes may be involved with other population differences in pregnancy outcome. Il-33 metabolism may be involved with improved thermogenesis and cold tolerance in northern environments, and there is evidence that introgressed genes related to these pathways may predominate in modern Europeans but not Africans. (Odegaard J I, et al. Perinatal Licensing of Thermogenesis by IL-33 and ST2. Cell. 2016 Aug. 11; 166(4):841-854). Interestingly, in addition to cold tolerance, IL-33 may also act as an ‘alarmin’ in early pregnancy to alert the immune system of potential tissue stress or damage. In early pregnancy, cleaved forms of IL-33 may activate immune cells expressing ST2, ILC2s and regulatory T cells (Tregs), enhancing the TH2 effector response, improving trophoblast invasion and pregnancy outcomes. (Romero R, et al.—a longitudinal study. J Matern Fetal Neonatal Med. 2018 February; 31(4):418-432; Sheng Y R, Hu W T, Shen H H, Wei C Y, Liu Y K, Ma X Q, Li M Q, Zhu X Y. Cell Mol Life Sci. 2022 Mar. 4; 79(3):173.). Given that Europeans carry these Neanderthal introgressed genes and African Blacks generally do not (Plunkett J et al. Ann Med. 2008; 40(3):167-95), it is possible that genetic Introgression may partially explain some racial differences we see between Blacks and non-Blacks in early pregnancy. These differences may be used to better understand the management of pregnancy disease. In summary, it is possible that differences in degree of Neanderthal introgression may offer insight into differences in pregnancy outcome between modern populations, and new tools for treatment approaches.

The methods for predicting risk of a pregnancy-related disorder in a human being disclosed herein, including previously known methods for screening (e.g., those described in Winger and Reed U.S. Pat. No. 10,323,282B2) provide improved pregnancy outcome prediction when microRNA panel is matched for patient cluster (e.g., race). To this point, this disclosure relates to methods using differential expression of microRNAs between patient clusters (e.g., racially distinct groups or ethnic groups) may suggest need for race specific pregnancy risk panels for optimal predictive power achievement in the clinic. As described, microRNA panels for pregnancy risk assessment may vary between different clusters (e.g., ethnic group). If a pregnancy panel is developed on one cluster (e.g., race) and applied to another cluster (e.g., race (for example, a Black panel is used on a non-Black patient)), the ability to predict pregnancy outcome becomes less robust. Correct identification of the Patient Cluster of a patient (e.g., ethnic group) is necessary in order optimal pregnancy predictive power of the test to be achieved. However, correct identification of the patient's Patient Cluster (e.g., ethnic group) is not always possible by visual inspection or medical record assessment. In many cases, a patient may be of mixed cluster (e.g., race or have unknown parentage). In other cases, a patient may not volunteer their family history. In other cases, an individual may simply not have this information available. In other cases, a patient may self-identify as a member of one cluster or group but carry key biological features of another cluster or group according to microRNA testing. These cases and others create an unmet need for a universal method for population membership assignment that eliminates guesswork. A method for correct subgroup designation at the time of pregnancy risk testing is an important unmet need that is fulfilled by the methods disclosed herein.

Homogenous groups can be defined by common expression of microRNAs that are distinctly different between patient populations to be distinguished (i.e., patient clusters). Table 32 displays differences in microRNA expression between exemplary clusters (here, Black and non-Black individuals) ordered by the differences in the expression between the two self-identified clusters (here, ethnic origin). One or more of these microRNAs can be used to assign cluster membership (Black, non-Black, for example). Tables 6-12 and FIG. 1 demonstrate the importance of correct patient cluster assignment thereby demonstrating the unmet need. Once the correct pregnancy risk panel is selected, optimal pregnancy risk prediction can be achieved.

### Example 2

### A. Embodiment A, Step 1. Phenotypic Clusters: Development of a Predictive Test for Cluster (e.g., Ethnicity) Assignment in the First Trimester of Pregnancy Developed on “Self-Identified” Ethnic Groups

In a preferred embodiment, two microRNA lists were determined, one list showing the microRNAs best able to discriminate between the Healthy and Compromised pregnancy outcomes in self-identified non-Blacks, another list showing the microRNAs best able discriminate between the Healthy and Compromised pregnancy outcomes in self-identified Blacks (see Tables 2A, 2B, 3A and 3B). MicroRNA expression levels were quantified by microarray from maternal peripheral blood cells of first trimester pregnant women up to 13 weeks pregnant. 471 microRNAs were compared. The non-Black population consisted of five Healthy and three Compromised pregnancies and the Black population consisted of three Healthy and six Compromised pregnancies. The study was a retrospective analysis using frozen maternal blood samples and clinical data from patient charts (Table 1). Microarray Analysis was performed according to the general procedures given in the papers (Winger E E, Reed J L, Ji X. First trimester PBMC microRNA predicts adverse pregnancy outcome. Am J Reprod Immunol 2014, doi:10.1111/aji.12287 and Winger E E, Reed J L, Ji X, Gomez-Lopez N, Pacora P, Romero R. MicroRNAs isolated from peripheral blood in the first trimester predict spontaneous preterm birth. PLoS One. 2020 Aug. 13; 15(8):e0236805. doi: 10.1371/journal.pone.0236805. PMID: 32790689; PMCID: PMC7425910.).

For each self-identified racially-defined population (non-Black and Black), an HC ratio was first calculated for each microRNA, then the microRNAs were then ordered by the absolute value of the HC ratio. A high HC ratio corresponds to the ability of a microRNA to predict pregnancy risk in the racial population group being measured. Tables 2A and 2B represents data from the non-Black population and Tables 3A and 3B represents data from the Black population. To generate the data presented in Tables 2A and 2B, a population of eight (8) non-Black patients in the first trimester of pregnancy were analyzed by microarray (5 healthy and 3 compromised outcome). An HC ratio was calculated for each microRNA, then the microRNAs were then ordered by the absolute value of the HC ratio. As described in the “Detailed description” the HC ratio consists of a numerator comprising the difference between the mean value of the “compromised” population minus the mean value of the “healthy” population and the denominator comprises the average of the two standard deviations of the values for healthy and compromised individuals. Means and standard deviations were calculated for each microRNA from patient samples with “healthy” outcomes and “compromised” outcomes. The individual miRNAs identified with highest ratios (≥1.5) are shown herein to best discriminate between the healthy and compromised pregnancy outcomes (Column H). Of the 471 microRNAs, 50 had HC ratios 1.5. MicroRNAs with these ratios were considered to demonstrate significant differences between patients destined to healthy and compromised pregnancies. To generate the data presented in Tables 3A and 3B, a population of nine (9) Black patients in the first trimester of pregnancy were analyzed by microarray (3 healthy and 6 compromised outcome). An HC ratio was calculated for each microRNA, then the microRNAs were then ordered by the absolute value of the HC ratio. As described in the “Detailed description,” the HC ratio consists of a numerator comprising the difference between the mean value of the “compromised” population minus the mean value of the “healthy” population and the denominator comprises the average of the two standard deviations of the values for healthy and compromised individuals. Means and standard deviations were calculated for each microRNA from patient samples with “healthy” outcomes and “compromised” outcomes. The individual miRNAs identified with highest ratios (21.5) are shown herein to best discriminate between the healthy and compromised pregnancy outcomes (Column Q). Of the 471 microRNAs 28 had H/C ratios ≥1.5. MicroRNAs with these ratios were considered to demonstrate significant differences between patients destined to healthy and compromised pregnancies. The HC ratio consists of a numerator comprising the difference between the mean value of the “Compromised” population minus the mean value of the “Healthy” population and the denominator comprising the average of the two standard deviations of the values for “Healthy” and “Compromised” individuals. Means and standard deviations are calculated for each microRNA from patient samples with “Healthy” outcomes and “Compromised” outcomes. The individual miRNAs identified with highest ratios (absolute value 21.5) were designated by “XX” in the tables. MicroRNAs designated “XX” are considered to be the top candidate microRNAs for pregnancy outcome prediction for each racial group.

### B. Embodiment A, Step 2: Development of Patient Cluster-Specific microRNA Panels (Here, for Black and Non-Black Populations) Using C1C2 Value Calculation Method

Of the 471 microRNAs sorted by HC ratio for each race (Tables 2A-B and Tables 3A-B), microRNAs were further selected to use for race prediction (separate from pregnancy outcome prediction). 175 microRNAs not useful for pregnancy outcome prediction were selected as candidates for cluster (here, ethnicity) assessment. Only MicroRNAs with HC ratio <|1.0| were selected for ethnicity assessment to avoid conflation with pregnancy outcome prediction microRNAs. Using these top 175 candidate microRNA for race assessment, C1C2 calculations were determined for each microRNA to assess those most differentially expressed between patient clusters (here, Black and non-Black populations). The C1C2 value was calculated for each microRNA by taking the difference between the mean microRNA expression of one ethnic group (Black population, “Cluster 2”) minus the mean microRNA expression level for the other ethnic group (here, the non-Black population, “Cluster 1”) as the numerator. The denominator was determined by calculating the average of the two standard deviations for Cluster 1 and Cluster 2. Then an absolute value was calculated. MicroRNAs were then sorted from highest to lowest C1C2 ratio. The top twenty C1C2 microRNAs were found to be statistically predictive of cluster in the first trimester of pregnancy by Fishers Exact Test as seen in Table 13A (p value<0.05; Graphpad online software URL: https://www.graphpad.com/quickcalcs/contingency1/last accessed Jul. 9, 2023). MicroRNAs with the highest C1C2 calculations with p value 50.05 were considered to have the greatest ability to assess race between Blacks and non-Blacks in the first trimester of pregnancy.

### C. Embodiment A, Step 3: ROC Curve Calculations for Each microRNA to Further Determine its Ability to Predict Patient Cluster (in this Example, Ethnicity)

Once the top 20 C1C2 microRNA candidates for race assessment were identified (from Step 2), a ROC curve was calculated for each of these top 20 microRNA (step 3). This step has two goals: (1) to heighten selection for top microRNA candidates for cluster identification and, (2) to determine microRNA expression level cut off designations for use in a “cluster” (group identification) prediction panel. Similar to score methods described for creation of a pregnancy risk panel detailed in our previous patent disclosures (microRNA Score method using ROC curve data described in: Winger, Reed, U.S. Pat. No. 10,323,282 B2 issued on Jun. 18, 2019, an ROC curve for cluster identification can be calculated for each microRNA for use in a scoring system for outcome category (in our current case, the “outcome” is ethnicity assessment). The Youden J point Associated Criterion Value is determined from for each microRNA ROC curve analysis using Medcalc® software (MedCalc Statistical Software version 19.0.7 (MedCalc Software bvba, Ostend, Belgium; https://www.medcalc.org; 2019). The Youden Index J Associated Criterion value for each microRNA is used as its positive/negative cut-off for race assessment. The chosen microRNA panel and associated microRNA cut-off points can then be applied to a validation set of individual pregnant patients of unknown ethnicity to assess their cluster (here, ethnic type) in early pregnancy. MicroRNA expression levels below the cut-off value for each of the microRNAs in the panel are assigned a score of “1”. The sums of the individual microRNA scores for each sample are calculated and are designated as a cluster assessment score (here, “non-Black-group Assessment Scores”) to be applied to a validation set population. Similar to methods used to improve microRNA prediction in the '282 patent, it is also understood that a plurality of microRNAs could be simultaneously analyzed to enhance predictive power using a microRNA panel.

### D. Embodiment B: Identification of Patient Group “Clusters” by microRNA Expression for Ethnic Assignment without Knowledge of “Self-Identified” Ethnicity

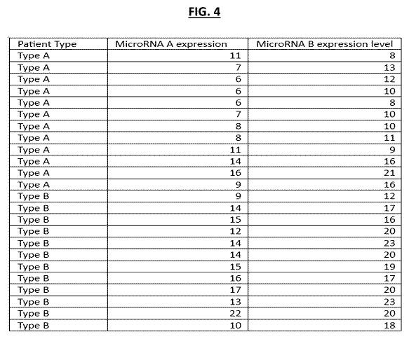

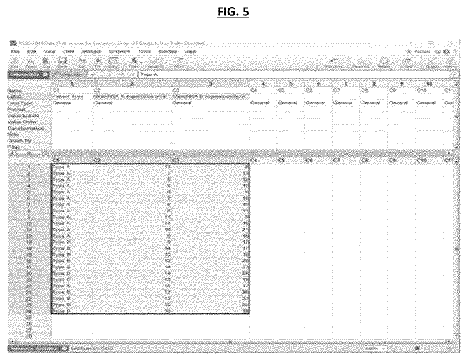

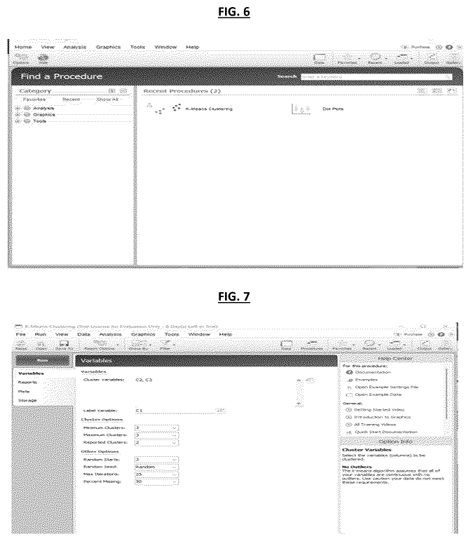

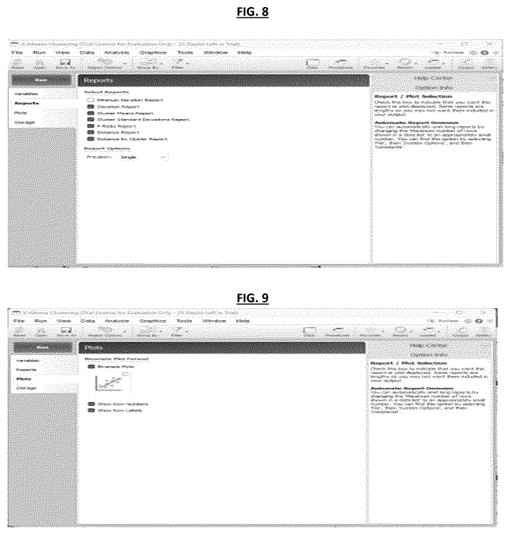

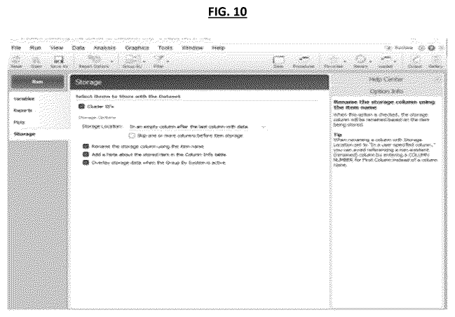

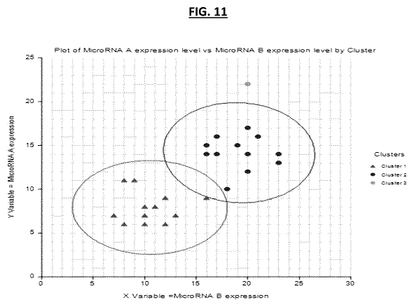

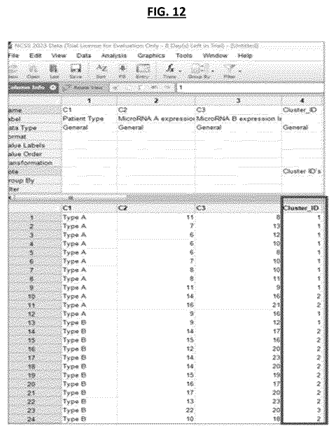

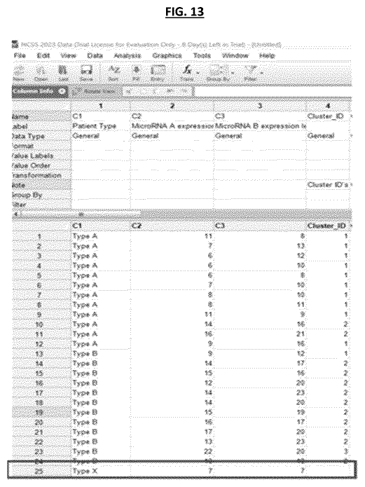

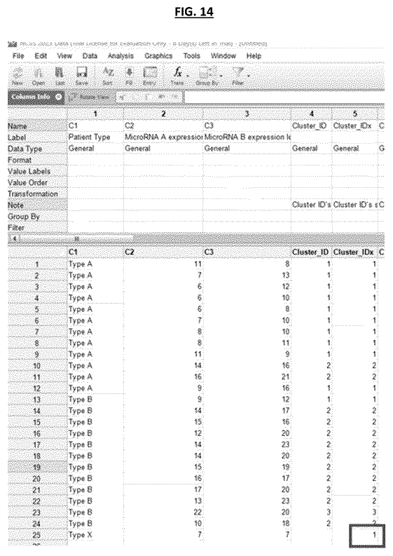

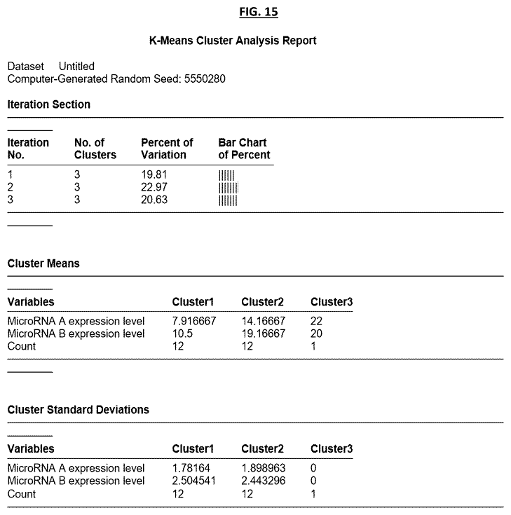

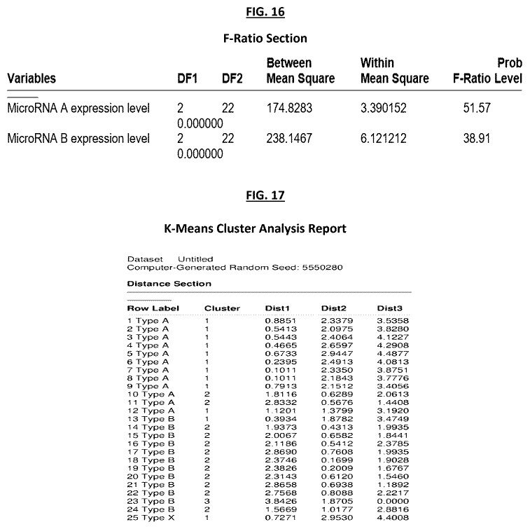

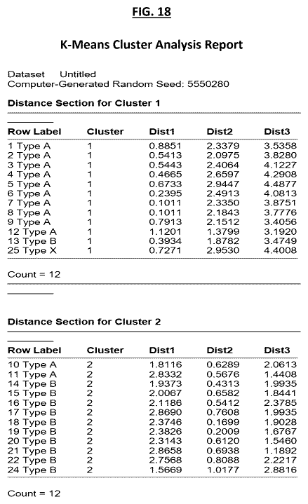

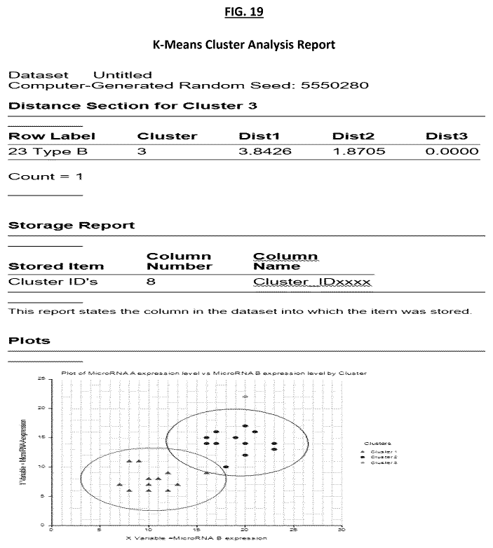

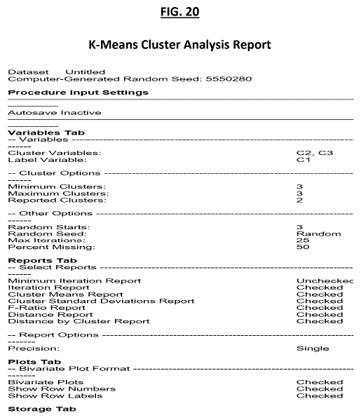

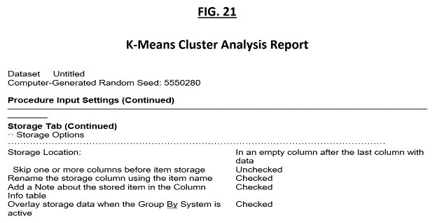

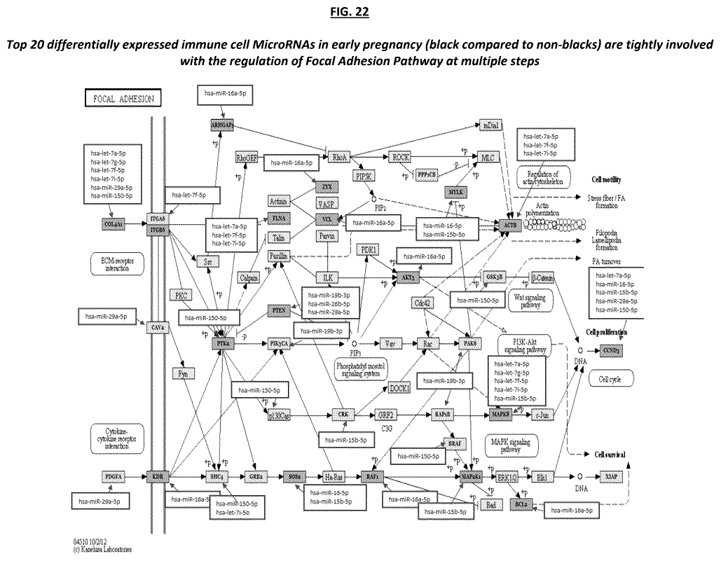

Racial Group or “Cluster-type” assignment can also be performed without initial knowledge of patient self-identified ethnicity. Where a plurality of microRNAs is used without knowledge of cluster (e.g., ethnicity or other phenotype), a software package such as NCSS statistical program alone (NCSS 2023 Statistical Software (2023). NCSS, LLC. Kaysville, Utah, USA, ncss.com/software/ncss) selecting K-Clustering may be used as an alternate method to determine a patient's placement into one Cluster or other Clusters. The program can generate two or more clusters based on microRNA expression levels. A demonstration of steps required to use the NCSS Cluster program for use with microRNA defined cluster groups is provided in FIGS. 4-21. Once Clusters are identified, the same program can be used to identify the most appropriate Cluster assignment to be used in the assay. Principal Component Analysis may also be used.

### Example 3: Pregnancy Monitoring and Treatments

A. Comparing microRNA Change Over Sequential Blood Draws in the First Trimester: Healthy Vs Unhealthy Pregnancy Outcomes and Racial Differences

In addition to using microRNAs for race-type prediction in early pregnancy, we also compared the change in sequential maternal blood cell microRNA levels in early pregnancy in Healthy and Unhealthy pregnancy to see how the race group and outcome group microRNA panels compared overtime. We started with two patients with preterm birth (“Unhealthy” outcome) and one patient with Full term delivery (“Healthy” outcome). Mean gestational age of first blood draw for the three patients was 56.7 days pregnant (7.2 weeks gestational age). The mean gestational age of second blood draw was 74.3 days pregnant (10.6 weeks gestational age). We used similar microRNA array quantification methods to those of our previous study (Winger E E, Reed J L, PLoS One. 2020 Aug. 13; 15(8):e0236805. doi: 10.1371/journal.pone.0236805. PMID: 32790689; PMCID: PMC7425910). However, in the current array, over 1,240 microRNAs were analyzed instead of 852 used in the former study. In this current experiment, it was found that, of the top 30 most increasing and decreasing microRNAs of approximately 1,240 microRNAs (2.4%), there were 21 microRNAs both increased in the Unhealthy outcome, and decreased in the Healthy patients, suggesting that opposite behavior of maternal cell microRNA in Healthy vs Unhealthy pregnancies in the early first trimester (Table 15). Most interestingly, it was found that 14 of these differentially expressed microRNAs in Healthy vs Unhealthy pregnancy matched the top twenty C1C2 race-predictive microRNAs expressed in early pregnancy (Tables 13A, 13B). Although C1C2 microRNAs were selected in samples from healthy pregnancies and included microRNAs with a low HC ratio <1.0 (less predictive of pregnancy risk within a group), interestingly, we found many of these microRNAs still exhibited differential pregnancy prediction ability between races. This suggests that these 14 overlapping microRNAs (microRNAs predictive of pregnancy risk within one race group but not predictive of pregnancy risk within another race group), may be regulated by neanderthal introgressed genes. Comparison of microRNA groups between the two race studies are listed in as an “x” in columns B, C and D of Table 16. Neanderthal introgressed enhancer SNP gene related microRNAs are listed in E. Many of these comparison microRNA groupings overlap. Given the shared microRNAs observed between the two studies, the shared microRNAs may have a significant upstream role in pregnancy outcome regulation and pathology. It should be noted that, in general, Black populations demonstrate increased rates of preterm birth over other racial populations. (Joyce A. Martin et al., “Births: Final Data for 2018,” National Vital Statistics Reports 68, no. 13 (2019). This observation may be key to understanding why the racial pregnancy differences associated with C1C2 microRNAs may overlap with the polar opposite behavior of these same microRNAs seen between Healthy and Compromised pregnancy in homogeneous race groups. These microRNAs may be key markers to assessing pregnancy risk. The identification of these microRNAs patterns so early in pregnancy may also open the door to new pregnancy treatments. For example, increasing levels of the top twenty C1C2 race-assessment microRNAs identified by first trimester pregnancy testing may also to alert for early treatment benefit in early pregnancy using immunotherapy (e.g., using intravenous IgG (IVIg), aspirin, and/or behavioral modifications). This information is especially important as preventative therapies have been shown to be most effective if given in the early first trimester (during placental bed formation) a time when microRNA levels can be used to monitor treatment effectiveness.

In addition to being a potential marker for treatment benefit, microRNA testing may help monitor therapy that has already been instigated. Previously published data demonstrates that pregnancy therapies can modulate microRNA expression levels to more healthy pregnancy pattern, potentially improving pregnancy outcomes (Winger, E. E., Reed, J. L. and Ji, X., 2015. Journal of Reproductive Immunology, 110, pp. 22-35). In this study, the mean IVIg related microRNA response in preeclampsia (7 patients) to the mean IVIg-related microRNA response in healthy pregnancy (7 patients). The mean microRNA IVIg response was calculated using the mean microRNA sequential changes of healthy patients before and after IVIg (mean day of first blood draw 41.4±18.8 days post-implantation; mean day of second blood draw 65.7±21.4 days post-implantation) and compared with similar patients not using IVIg (3 healthy pregnancies). A similar IVIg comparison was performed for the preeclampsia pregnancies (mean day of first blood draw 48.0±40.3 days post-implantation; mean day of second blood draw 76.4±40.2 days post-implantation) and compared with similar patients not using IVIg (2 preeclampsia pregnancies). When the results (with and without IVIg) for each outcome group were sorted from largest difference to smallest difference, the “microRNA IVIg response” by specific microRNAs were associated with different pregnancy outcomes. The “microRNA IVIg responses” between the preeclampsia and the healthy pregnancies were converse. This suggested that immunologic intervention can modulate microRNA expression levels toward those found in healthy women.

Immune modulation can improve pregnancy outcome in at risk pregnancies with dysregulated immune regulatory T cells. It is known that FoxP3+ regulatory T cells play an important role in maintenance of immune homeostasis (Bluestone J A et al. Nature. 2010; 464:1293-300. https://doi.org/10.1038/nature08933). MicroRNAs are also tightly implicated in the regulation of these T regulatory cells (Zhou X et al. J Exp Med. 2008; 205:1983-91. https://doi.org/10.1084/jem.20080707). In addition, it is known that low circulating CD4+ T regulatory cells predict miscarriage risk (Winger, E. E. and Reed, J. L., 2011. Low circulating CD4+ CD25+ Foxp3+ T regulatory cell levels predict miscarriage risk in newly pregnant women with a history of failure. American Journal of Reproductive Immunology, 66(4), pp. 320-328.). Again, maternal immune cell microRNA testing in early pregnancy may offer another tool for efficient pregnancy risk management based on its potential role in T regulatory cell regulation.

### B. MicroRNA Pathways Analysis to Identify Pathology

Prognostic differences between Blacks and non-Black pregnant women are well known. Certainly, environmental influences such as poor diet, low education and unhealthy environment may be partly responsible for higher pregnancy risk observed in Black women. However, when diet, education and education are controlled for, Black women consistently experience higher preterm birth rates (14%) than other racial groups (9%), and the cause may be partly genetic (Joyce A. Martin et al., “Births: Final Data for 2018,” National Vital Statistics Reports 68, no. 13 (2019). MicroRNAs work in groups to drive gene expression. Differentially expressed microRNAs between Black and non-Black patients may be interrogated in various online programs disclosing specific gene-mediated pathways regulating placental bed formation. These pathologies may potentially be modulated and treated if identified early enough in pregnancy.

Of the top 20 differentially expressed C1C2 microRNAs between healthy Blacks and non-Blacks in early pregnancy (Table 13A), the most statistically significant KEGG pathway found using analyzed using DIANA miRPath v.2.0: (Web server issue) was MAPK signaling pathway (hsa04010), PI3K-Akt signaling pathway (hsa04151), Prostate cancer (hsa05215), and Focal adhesion (hsa04510) (Table 17) (Reference for KEGG pathways tool: Kanehisa, et al. KEGG for taxonomy-based analysis of pathways and genomes. Nucleic Acids Res. 51, D587-D592 (2023). These pathways control cell growth, proliferation and cell migration, all processes known to be involved with trophoblast migration at the placental bed site. (Knöfler M. Critical growth factors and signalling pathways controlling human trophoblast invasion. Int J Dev Biol. 2010; 54(2-3):269-80. doi: 10.1387/ijdb.082769mk. PMID: 19876833; PMCID: PMC2974212). Again, any known pathologies may potentially be modulated and treated if identified early in pregnancy.

### Example 4. Developing a microRNA Panel for Duster Membership Prediction

This disclosure provides methods for overcoming difficulties associated with assigning patients to control groups matched for racial background. Comparison of patients to controls is improved wherein control groups and patients are matched for racial background. Difficulties arise when identification of racial background of either the patient or members of the control group is unclear. Patients of mixed racial background are of particular concern. The biologic behavior of an individual may be more closely related to one or the other race or a combination of both. The present invention does not rely upon self-identified race but, instead, relies upon biologic characteristics. The instant invention assigns patients and the controls to which they are compared to a common group or cluster by their shared expression levels of microRNAs. These groups share biologic characteristics rather than factors limited to self-identified racial membership. This invention is directed to distinction of two racial groupings, Blacks and non-Blacks by their common expression levels of microRNAs. Black women appear to demonstrate distinct microRNA patterns from other racial groups (e.g. Asian, Hispanic, white etc.) that have been examined by inventors. It is speculated that the differences identified may be related to the introgression of genetic elements from Neanderthals that are not found in individuals of sub-Saharan origin.

This disclosure provides at least three methods that aid in assignment of patient and controls to a common patient cluster. The first is conventionally used assigning patients by self-identified characteristic (e.g., ethnicity (e.g., race), disease state (e.g., diabetes)). While this method improves results over methods that do not employ assignment to a cluster, it suffers from inaccurate patient reporting as well as the problem of assignment in cases relating to ethnicity, mixed heritage. A second method identifies biologic characteristics, namely expression levels of microRNAs that are differentially expressed by different patient clusters (e.g., the racial categories Black and non-Black). Differential expression is useful wherein a sufficient numbers of controls individuals are compared permitting identification of strongly differentially expressed microRNAs. A third method disclosed herein assigns patients to a patient cluster by comparison to control patients using the clustering algorithm programs disclosed herein that assigning patients to clusters without self-identification (“Patient Cluster”).

This disclosure provides methods for identifying microRNAs that are differentially expressed in patients within a particular patient cluster (e.g., Black and non-Black patients). In a preferred embodiment, all candidate control patients (from whom control samples are derived) are in their first trimester of pregnancy and destined to healthy pregnancy outcome.

Selection of a microRNA Panel to Assess Patient Cluster Type

Step 1: Patients populations of interest are first selected for analysis by self-identified characteristic (e.g., ethnicity or disease state) divided into at least two groups. For instance, patients can be categorized into “Group A” (e.g., self-identified as Black) and “Group B” (e.g., self-identified as non-Black patients and corresponding controls). All individuals are in their first trimester of pregnancy and destined (by history) to be have healthy pregnancy outcomes. Peripheral blood is collected during the first trimester, preferably at about 8 weeks pregnancy.

Step 2: Maternal blood is collected during the first trimester of pregnancy, preferably at 8-10 weeks pregnancy, in 10 ml vacutainer with EDTA.

A sample embodiment for Cluster type score determination is illustrated in Tables 18, 19 20. This embodiment illustrates selection of pregnancy race-type for individuals in population of first trimester pregnant patients using a microRNA quantification alone. First, four populations of first trimester pregnant women were compared: Blacks, whites, Hispanic and Asian/Amerind. MicroRNA quantification was performed on maternal buffy coat blood specimens obtained between 11±1.3 weeks gestation from 265 pregnant women. These 265 samples were randomized to two groups: 193 samples in a training set and 72 samples in validation set (see Table 18 for population details). Each of the samples was analyzed using a panel of 45 microRNAs previously selected by their differential expression on analysis between patients destined to compromised pregnancy and healthy pregnancies (Winger E E, Reed J L et al. PLoS One. 2020 Aug. 13; 15(8): e0236805. doi: 10.1371/journal.pone.0236805. PMID: 32790689; PMCID: PMC7425910. Samples were reverse transcribed and rtPCR performed according to a similar protocol as described in this published study. Selection of microRNAs for race assessment panel was performed using the training set using microRNA percent signalling as a guide. MicroRNA “percent signalling” is the percentage of samples that demonstrate detectable signal out of the total number of samples being measured (#samples that exhibit a detectable signal (Ct >30) out of total number of samples in the training set). In this embodiment, first microRNAs that demonstrate at least 1.25 times increased or decreased percent signal in one race over all other races in healthy pregnancy are first considered as candidate microRNA markers for race prediction (top 10% of 45 microRNAs). These candidate microRNAs are then subjected to Fisher's exact T test analysis for statistical significance. Those microRNAs that reach statistical significance p<0.2 using Fisher's Exact test are selected for the final score panel: miR-581-5p, miR-6737-3p, miR-193-3p and miR-223-5p (see p-values, Table 19, Column F). The other 41 microRNAs were deemed to demonstrate no meaningful predictive value for race in this particular experiment. Next, a scoring system is constructed based on the relative predictive weight given by each of the top four microRNAs selected. In this example embodiment, all samples belong to Cluster Type 1 (non-Black) by default unless microRNA panel identifies the particular sample belonging to the Cluster 2 group (Black) based on these four microRNAs, (miR-582-p being the strongest with p<0.0001 and miR-223-5p being the weakest with p<0.18). The presence or absence of the microRNA signal are used to score a sample as “Cluster 1 type” point of “Cluster 2 type” using each microRNA in sequence based on its statical strength. Cluster 2 is then determined in two simple steps. First step, all blood samples that demonstrate a hsa-miR-582-5p signal are automatically designated into Cluster 2 (Black race type). Second, all blood samples that demonstrate a miR-6737-3p signal without a co-existing miR-193-3p signal and/or a 223-3p signal are also designated into Cluster 2 (Black race type). See Table 20 for details. It should be noted that the designated Cluster Type (Black or non-Black type) does not necessarily match the self-identified race identified by the patient. For example, it is possible that a mixed-race obstetric patient (with a white mother and Black father, for example) may self-identify as “Black” however they may biologically follow the pregnancy risk pattern observed of a “white” race patient. In this case, the patient would benefit from using the non-Black race microRNA Pregnancy Risk test instead of the Black one for optimal pregnancy risk assessment (see ROC curves “Black” and “non-Black” versus “Self-identified” and “MicroRNA-identified” test comparisons illustrated in Table 29).

### BRIEF DESCRIPTION OF TABLES

In Table 1 below, two populations of first trimester pregnant women were compared: a non-Black population and a Black population. The non-Black population consisted of 5 “Healthy” and 3 Unhealthy” pregnancies and the Black population consisted of 3 “Healthy” and 6 “Unhealthy” pregnancies. The study was a retrospective analysis using frozen maternal blood samples and clinical data from patient charts. MicroArray analysis was performed according to the general procedures given in the papers (Winger E E, Reed J L, Ji X. Am J Reprod Immunol 2014, doi:10.1111/aji.12287 and Winger E E, Reed J L et al. PLoS One. 2020 Aug. 13; 15(8):e0236805. doi: 10.1371/journal.pone.0236805. PMID: 32790689; PMCID: PMC7425910.).

In Table 2A and 2B a population of 8 non-Black patients in the first trimester of pregnancy were analyzed by microarray (5 healthy and 3 compromised outcome) (Table A is the shortened version. See full table in Table 2B). An HC ratio was calculated for each microRNA, then the microRNAs were then ordered by the absolute value of the HC ratio. Means and standard deviations were calculated for each microRNA from patient samples with “healthy” outcomes and “compromised” outcomes. The individual miRNAs identified with highest absolute value of the HC ratios (≥|1.5|) are shown herein to best discriminate between the healthy and compromised pregnancy outcomes (Column H). Of the 471 microRNAs 50 had |HC ratios|≥1.5. MicroRNAs with these ratios were considered to demonstrate significant differences between patients destined to healthy and compromised pregnancies.

Table 3A and 3B presents HC ratio calculations for pregnancy outcome prediction using 471 microRNAs from pregnant Black patients. A population of 9 Black patients in the first trimester of pregnancy were analyzed by microarray (3 healthy and 6 compromised outcome). (See full version of Table 3A in Table 3B). An HC ratio was calculated for each microRNA, then the microRNAs were then ordered by the absolute value of the HC ratio. Means and standard deviations were calculated for each microRNA from patient samples with “healthy” outcomes and “compromised” outcomes. The individual miRNAs identified with highest absolute value of the ratios (≥|1.5|) are shown herein to best discriminate between the healthy and compromised pregnancy outcomes (Column Q). Of the 471 microRNAs 28 had |HC ratios|≥1.5. MicroRNAs with these ratios were considered to demonstrate significant differences between patients destined to healthy and compromised pregnancies.

Table 4 shows four of the top 25 HC ratio microRNAs for non-Black persons that predict adverse pregnancy outcome with a statistically significant ROC curve p value of <0.05 (taken from the 471 microRNAs that were interrogated in the non-Black cluster in Table 2). When the patient's microRNA signal level is above the cut-off point set at the ROC curve's associated criterion value taken at the Youden J point (MedCalc Software bvba, Ostend, Belgium) the patient is deemed to be at “increased risk” of a developing a pregnancy disorder. It is also understood that a plurality of microRNAs could be simultaneously analyzed to enhance predictive power.

Table 5 shows that of the 471 microRNAs that were interrogated in the Black population (Table 3) twelve of the top 25 HC ratio microRNAs for non-Blacks predict adverse pregnancy outcome with a statistically significant ROC curve p value of <0.05. When the patient's microRNA signal level is above the cut-off point set at the ROC curve's associated criterion value taken at the Youden J point (MedCalc software) the patient is deemed to be at “increased risk” of a developing a pregnancy disorder. It is also understood that a plurality of microRNAs could be simultaneously analyzed to enhance predictive power.

Table 6 presents population details for the training and validation sets for an “unmet need” demonstration (biological race) at time of pregnancy prediction testing. Seventy-one samples from the first trimester pregnant women were randomized to two groups: 36 samples in a validation set and 35 samples in validation set. In this study, microRNA quantification was performed on maternal buffy coat blood specimens obtained between 11±1.3 weeks gestation.

Table 7 presents the AUC-ROC for each microRNA in the training set of the non-Black sub-population, calculated with its associated p values and an associated Youden Index J Associated Criterion Value using Medcalc software. Each of the samples was analyzed using a panel of 30 microRNAs previously selected by their differential expression on microarray analysis between patients destined to compromised pregnancy and healthy pregnancies (Winger, et al., 2014. Am J Reprod. Immunol 2014; 72:515±526.) Samples were reverse transcribed and rtPCR performed according to the protocol in our previous study (Winger et al., 2015. J Reprod Immunol). The Youden Index J Associated Criterion Value for each microRNA was used as its positive/negative cut-off for the “non-Black” panel. The panel and calculated cut-off points were later applied to the validation set. Ct values below the cut-off value for each of the microRNAs in the panel were assigned a score of “1”. The sums of the individual microRNA scores for each sample were calculated and were designated as “non-Black risk scores” to be applied to a validation set population.

Table 8 presents the AUC-ROC for each microRNA in the training set of the Black sub-population calculated with its associated p values and its associated Youden Index J Associated Criterion Value using Medcalc software. The Youden Index J Associated Criterion Value for each microRNA was used as its positive/negative cut-off. The panel and calculated cut-off points were then applied to the validation sets for each population. Ct values below the cut-off value for each of the microRNAs in the panel were assigned a score of “1”. The sums of the individual microRNA scores for each sample were calculated and were designated as “Black group risk scores” to be applied to a validation set population.

Table 9 presents data relating to non-Black population using non-Black score for pregnancy outcome prediction (See ROC curve calculations in FIG. 1A). For each microRNA in the training set of the non-Black sub-population, an AUC-ROC was calculated with its associated p values and associated Youden Index J Associated Criterion Value using Medcalc software (Table 7). The Youden Index J Associated Criterion Value for each microRNA was used as its positive/negative cut-off. The panel and calculated cut-off points were then applied to a similar non-Black validation set sub-population. Ct values measured by the designated cut-off value for each of the microRNAs in the panel were assigned a score of “1”. The sums of the individual microRNA scores for each sample were calculated and were designated as “Risk Scores” to be applied to each patient. Resulting ROC curves for pregnancy outcome prediction can be seen in FIG. 1A.

Table 10 presents data regarding a Black population using the non-Black score for pregnancy outcome prediction. (ROC curve can be seen in FIG. 1B). For each microRNA in the training set of the non-Black sub-population, an AUC-ROC was calculated with its associated p values and associated Youden Index J Associated Criterion Value using Medcalc software (Table 7). The Youden Index J Associated Criterion Value for each microRNA was used as its positive/negative cut-off. The panel and calculated cut-off points were then applied to a Black validation set population for comparison purposes. Ct values below the cut-off value for each of the microRNAs in the panel were assigned a score of “1”. The sums of the individual microRNA scores for each sample were calculated and were designated as “Risk Scores” to be applied to each patient.

Table 11 presents data regarding a Black population using Black score for pregnancy outcome prediction (See ROC curve calculations in FIG. 1 C) For each microRNA in the training set Black sub-population, an AUC-ROC was calculated with its associated p values and associated Youden Index J Associated Criterion Value using Medcalc software (Table 8). The Youden Index J Associated Criterion Value for each microRNA was used as its positive/negative cut-off. The panel and calculated cut-off points were then applied to a similar Black validation set sub-population. Ct values below the cut-off value for each of the microRNAs in the panel were assigned a score of “1”. The sums of the individual microRNA scores for each sample were calculated and were designated as “Risk Scores” to be applied to each patient.

Table 12 presents data regarding a non-Black population using Black score for pregnancy outcome prediction. (Resulting ROC curve for pregnancy outcome prediction can be seen in FIG. 1D.) For each microRNA in the training set of the Black sub-population, an AUC-ROC was calculated with its associated p values and associated Youden Index J Associated Criterion Value using Medcalc software (Table 8). The Youden Index J Associated Criterion Value for each microRNA was used as its positive/negative cut-off. The panel and calculated cut-off points were then applied to a non-Black validation set population for comparison purposes. Ct values below the cut-off value for each of the microRNAs in the panel were assigned a score of “1”. The sums of the individual microRNA scores for each sample were calculated and were designated as “Risk Scores” to be applied to each patient.

Tables 13 A and B (Table 13A is the shortened version of Table 13 B) present the differential expression rank of individual microRNAs between pregnant Black and non-Black women with Healthy pregnancy outcome. The C1C2 rankings in non-Black versus Black using microRNA microarray reading. 175 microRNAs that are not useful for pregnancy outcome prediction were selected (HC ratio <1.0) and only samples from healthy pregnancies were assessed, in this example, to avoid conflation with pregnancy prediction microRNAs. The mean and standard deviation for 175 microRNA expression rankings were calculated using three healthy pregnant Black patients and six healthy pregnant non-Black patients (see population details Table 1). The numerator comprised the difference between the mean expression rank of the three patient “back” group population (Cluster 2) minus the mean expression rank for the six patient “non-Black” group population (Cluster 1) and the denominator comprised the average of the two standard deviations of the values for the three Black and six non-Black individuals, then the absolute value is calculated. These C1C2 values for each of the 175 microRNAs were then sorted from highest to lowest. MicroRNAs with the highest C1C2 values >2.3 with p value ≤0.05 were considered to have the greatest ability to assess racial type for microRNA panel designation decision.

Similar to methods used for pregnancy outcome prediction listed (detailed Patient Score method using ROC data described in Winger, Reed, U.S. Pat. No. 10,323,282 B2 issued on Jun. 18, 2019, a ROC curve could be calculated for each microRNA to predict racial group of a patient instead of pregnancy outcome (only “Healthy” pregnancy outcomes used here) as shown in Table 14. Of the 175 microRNAs that were interrogated in the two racial populations, 20 microRNAs were found to have C1C2 calculation >2.3. Of those microRNAs with C1C2 calculation >2.3, all were thereby deemed useful individually for race prediction based on having a ROC curve p value <0.05. A ROC curve's associated criterion value (cut-off point”) taken at the Youden J point can be used to predict the odds of a patient being a member of a particular pregnancy race group. When the patient's microRNA signal level below the cut-off point set at the Youden J point Associated Criterion Value, the patient is deemed carry an “increased chance” of being a member of given pregnancy “Cluster” group (for example, member of a given racial population). The Youden J point Associated Criterion Value can be determined from ROC curve analysis using Medcalc® software (MedCalc Statistical Software version 19.0.7 (MedCalc Software bvba, Ostend, Belgium; https://www.medcalc.org; 2019) upon analysis of quantification of individual microRNA in patients being in one racial group versus another. Like with pregnancy outcome prediction, it is also understood that a plurality of microRNAs could be simultaneously analyzed to enhance predictive power.

In Table 15, columns A and B represent differences in expression level in sequential first trimester blood draws of 3 individual patients (2 Unhealthy pregnancies mean values (Column A), one Healthy pregnancy (Column B). Mean gestational age of first blood draw is 56.7 days pregnant (7.2 weeks GA) for 30 of 1,240 microRNAs quantified by microarray. The mean gestational age of second blood draw is 74.3 days pregnant (10.6 weeks GA). The change in the Unhealthy versus the Healthy patient was studied. It was determined that the top 30 microRNA expression levels that increased in the Unhealthy outcome, decreased in the Healthy patients suggesting that biomarker potential exists for these microRNAs. Such similar differential sequential microRNA expression patterns were observed in our previous patent disclosures (Winger, Reed, U.S. Pat. No. 10,323,282 B2 issued on Jun. 18, 2019) however our current experiment uses a larger microarray (approx. 300+ additional microRNAs) than our previous experiments.

Table 16 compares the features of top marker-candidate MicroRNAs. The table demonstrates microRNAs that share significant features: (Column B) xx=Member of Twenty MicroRNA group with largest differences in expression levels between first trimester Black and non-Black patients as seen in Table 13 (Column C) x=microRNA that express most diminishing (bottom 2.4% of 1,240 microRNAs) expression levels in first trimester pregnant patient sequential blood draws with healthy outcomes as seen in Table 15 and (Column D) x=MicroRNA that express most increasing (top 2.4% of 1,240 microRNAs) expression levels in first trimester pregnant patients sequential blood draws with Unhealthy outcomes as seen in Table 15. MicroRNAs represented in multiple columns demonstrate greater diagnostic potential.

Table 17 presents the KEGG pathways associated the most differentially expressed microRNAs between Blacks and non-Black populations in the first trimester of pregnancy. Top KEGG pathways associated the most differentially expressed microRNAs between early pregnant Blacks and Non-Blacks (top C1C2microRNAs from Table 13). The most statistically significant pathways were found to be MAPK signaling pathway (hsa04010), PI3K-Akt signaling pathway (hsa04151), Prostate cancer (hsa05215), MAPK signaling pathway (hsa04010) and Focal adhesion (hsa04510). These pathways are associated with growth, proliferation and migration. Pathway analysis performed using DIANA miRPath v.2.0: (Web server issue).

Table 18 presents a comparison of four populations of first trimester pregnant women with healthy pregnancy outcomes were compared: Blacks, whites, Hispanic and Asian/Amerind for analysis. MicroRNA quantification was performed on maternal buffy coat blood specimens obtained between 11±1.3 weeks gestation from 265 pregnant women. These 265 samples were randomized to two groups: 193 samples in a training set and 72 samples in validation set. Each of the samples was analyzed using a panel of 45 microRNAs selected in Winger E E, Reed J L et al. PLoS One. 2020 Aug. 13; 15(8):e0236805. doi: 10.1371/journal.pone.0236805. PMID: 32790689; PMCID PMC7425910. Samples were reverse transcribed and rtPCR performed according to a similar protocol as described in this published study.

Table 19 presents a selection of microRNAs for an ethnicity (specifically here, race) assessment panel using a training set. MicroRNA “percent signal” is the percentage of samples with Healthy pregnancy outcome that demonstrate detectable signal out of the total number of samples being measured (#samples that detectable signal/total #samples). This was quantified for four groups pregnant women: Blacks, whites, Hispanic and Asian/Amerind (columns). MicroRNA quantification was first performed on maternal buffy coat blood specimens obtained between 11±1.3 weeks gestation from 265 pregnant women with healthy pregnancy outcome. These 265 samples were randomized to two groups: 193 samples in a training set and 72 samples in validation set. Each of the samples was analyzed using a panel of 45 microRNAs used in the Winger/Reed study (Winger E E, Reed J L et al. PLoS One. 2020 Aug. 13; 15(8):e0236805. doi: 10.1371/journal.pone.0236805. PMID: 32790689; PMCID: PMC7425910. First, microRNAs that demonstrate at least 1.25 times increased or decreased % signal in one race over all other races in the training set are considered potential candidate markers for race assessment in the validation set (top 10% of 45 microRNAs measured in the experiment). These candidate microRNAs are then subjected to Fisher's exact T test analysis for statistical significance in the right-hand column of the table for final marker selection (Column F). Those microRNAs that confirm a minimum degree of statistical significance for racial differentiation ability of p<0.2 using Fisher's Exact test were included in the final score panel. In this embodiment, the microRNAs that met the criteria were miR-581-5p, miR-6737-3p, miR-193-3p and miR-223-5p. The other 41 microRNAs were deemed to demonstrate no meaningful predictive value for race in this particular experiment.

Table 20 illustrates the application of an example score system for determination of Cluster type (ethnicity or race group) based on top four microRNAs selected in patients with Healthy pregnancy outcomes seen in Table 19. In this embodiment, the top four microRNAs in first trimester pregnant sample with Healthy outcomes were determined to be statistically predictive of Black race versus non-Black race pregnancy type using Fishers exactT-test using the training set (Table 19, column F). These microRNAs were then used to predict pregnancy ethnicity type (Patient Cluster 1 “Black” versus Patient Cluster 2 “non-Black”) using the Validation set samples of similar first trimester Healthy samples blinded to self-identified race. A scoring system is then applied based on the relative predictive weight “Patient Cluster-type” (ethnicity prediction) given by each of the top four microRNAs selected in Table 19. In this example embodiment, all samples belong to Patient ClusterType 1(non-Black race) by default unless microRNA panel identifies the particular sample belonging to the Patient Cluster 2 group (Black race type) based on these four microRNAs. (miR-582-p being the strongest with p<0.0001 and miR-223-5p being the weakest with p<0.18) the presence or absence of the microRNA signal are used to score a sample as “Patient Cluster 1 type” point of “Patient Cluster 2 type” using each microRNA in sequence of its statical strength. Patient Cluster type 1 is designated as “non-Black (non-African origin) race type and Patient Cluster 2 is designated as Black (African-origin). Cluster 2 is then determined in two simple steps. First step, all blood samples that demonstrate a hsa-miR-582-5p signal are automatically designated into Patient Cluster 2 (Black race type). Second, all blood samples that demonstrate a miR-6737-3p signal without a co-existing miR-193-3p signal and/or a 223-3p signal are also designated into Patient Cluster 2 (Black race type). It should be noted that the designated Cluster Type (Black or non-Black type) does not necessarily match the self-identified race identified by the patient. For example, it is possible that a mixed-race obstetric patient (with a white mother and Black father, for example) may self-identify as “Black” however they may biologically follow the pregnancy risk pattern observed of a “white” race patient. In this example, the self-identified “Black” patient would benefit from using the non-Black race microRNA Pregnancy Risk test instead of the Black one for optimal pregnancy risk assessment. (See ROC curves “Black” and “non-Black” versus “self-identified” and “microRNA-identified” test comparisons illustrated in Table 29).

Table 21 demonstrates application of non-Black microRNA pregnancy prediction panel to a “self-identified” non-Black validation set population based on a validation set microRNA signal cut-offs (Associated Criterion values) listed in Table 7.

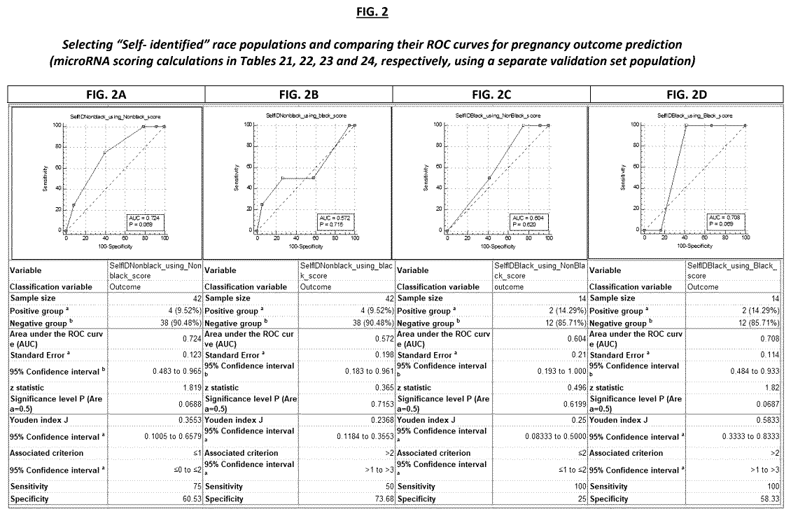

Table 22 demonstrates application of Black microRNA pregnancy prediction panel to a “self-identified” non-Black validation set population based on microRNA signal cut-offs (see FIG. 2B)

Table 23 demonstrates application of non-Black microRNA pregnancy prediction panel to a “self-identified” Black/mixed/unknown race validation set population based on microRNA signal cut-offs listed in FIG. 1 (FIG. 2C).

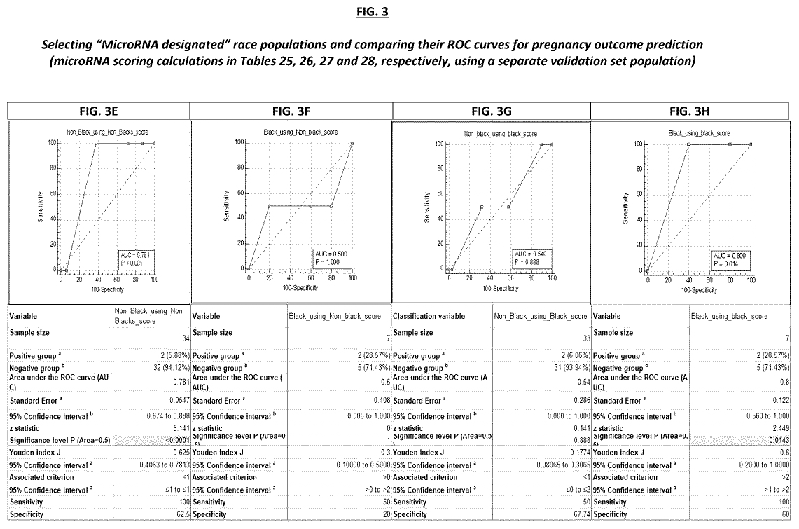

Table 24 demonstrates application of Black microRNA pregnancy prediction panel to a “self-identified” Black and mixed/unknown race validation set population based on microRNA signal cut-offs listed in Table 8 (see FIG. 3D).

Table 25 demonstrates application of non-Black microRNA pregnancy prediction panel to a “microRNA designated” non-Black validation set population based on microRNA signal cut-offs listed in Table 7 (see FIG. 3E).

Table 26 demonstrates application of non-Black microRNA pregnancy prediction panel to a “microRNA designated” Black validation set population based on microRNA signal cut-offs listed in Table 7 (see FIG. 3F).

Table 27 demonstrates application of Black microRNA pregnancy prediction panel to a “microRNA designated” non-Black validation set population based on microRNA signal cut-off listed in Table 8 (see FIG. 3G).

Table 28 demonstrates application of Black microRNA pregnancy prediction panel to a “microRNA designated” Black validation set population based on microRNA signal cut-off listed in Table 8.

Table 29 compares ROC curves for pregnancy outcome prediction using “self-identified” race versus “microRNA designated” race in validation set populations. Conclusion: The addition of the “microRNA population designation” step increases the ability of the microRNA pregnancy outcome panel to predict pregnancy risk. It is noted that a similar table scoring approach using to that used in Tables 21-28 is described are in Winger, Reed: Patent Application Pub. No. 20120107825.

Table 30 provides an analysis of the top 20 differentially expressed immune cell microRNAs between pregnant Blacks and Non-Blacks in early pregnancy (see Table 13) top 22 genes with associated pathways in common (see Table 31).

Table 31 describes Reactome Pathways Analysis of 22 Neanderthal introgressed genes (from Table 30) regulated by our top 10 C1C2 race identifying microRNAs (from Table 32) in early pregnancy.

Table 32 shows C1C2 ratio calculations for race assessment taken from healthy first trimester pregnancy samples in Blacks and non-Blacks using 175 microRNAs with low HC ratios <1.0 (low pregnancy outcome prediction ability).

TABLE 1
Two patient clusters (e.g., populations) of first trimester pregnant
women: non-Black and Black for top microRNA selections

| | | | | | | | | | |
|---|---|---|---|---|---|---|---|---|---|
| | | | | Matern. | Gest age | Deliv. | Birth- | Delivery | Pregnancy |
| Patient# | Outcome | Race | BMI | age | (wks) | (wks) | weight (g) | method | outcome |

Non-Black population

| | | | | | | | | | |
|---|---|---|---|---|---|---|---|---|---|
| 1 | Healthy | Asian | 24 | 32 | 9.9 | 41 | 4253 | CS | Full term |
| | | | | | | | | | Healthy |
| 2 | Heathy | White | 31.5 | 45 | 4.9 | 39 | 3175 | CS | Full term |
| | | | | | | | | | Healthy |
| 3 | Healthy | White | 26.3 | 34 | 8.3 | 40 | 3317 | Vaginal | Full term |
| | | | | | | | | | Healthy |
| 4 | Healthy | Asian | NA | 40 | NA | 40 | 3771 | Vaginal | Full term |
| | | | | | | | | | Healthy |
| 5 | Healthy | White | 25.1 | 40 | 6 | 35 | 1814 × 3 | CS | Triplets |
| | | | | | | (triplets) | | | healthy |
| 6 | Unhealthy | White | 24.1 | 46 | 9.4 | 37 | 2411 | CS | Preeclampsia |
| | | | | | | | | | IUGR |
| 7 | Unhealthy | White | 36.6 | 51 | 6 | 36 | 3288 | Vaginal | Preterm PROM |
| 8 | Unhealthy | White | NA | 40 | 5.6 | 40 | 2693 | Vaginal | IUGR |

Black population

| | | | | | | | | | |
|---|---|---|---|---|---|---|---|---|---|
| 1 | Healthy | Black | 26.7 | 41.3 | 12.6 | 39.5 | 2900 | Vaginal | Full term |
| | | | | | | | | | healthy |
| 2 | Healthy | Black | 29.7 | 28.8 | 12.1 | 39.1 | 3030 | Vaginal | Full term |
| | | | | | | | | | healthy |
| 3 | Healthy | Black | 44.4 | 32.5 | 13 | 39.3 | 2835 | CS | Full term |
| | | | | | | | | | healthy |
| 4 | Unhealthy | Black | 28.4 | 35.6 | 12.6 | 26.4 | 540 | Vaginal | Early Preterm/ |
| 5 | Unhealthy | Black | 31.1 | 36 | 12.3 | 31.8 | 1410 | CS | Preterm/ |
| | | | | | | | | | Preeclampsia |
| 6 | Unhealthy | Black | 36.3 | 34.3 | 12.9 | 26.7 | 560 | CS | Early Preterm/ |
| | | | | | | | | | Preeclampsia |
| 7 | Unhealthy | Black | 32 | 31.9 | 13 | 30 | 1510 | CS | Preterm/IUGR/ |
| | | | | | | | | | Preeclampsia |
| 8 | Unhealthy | Black | 32.9 | 33 | 13.3 | 34.5 | 1900 | CS | Late preterm/ |
| | | | | | | | | | Preeclampsia |
| 9 | Unhealthy | Black | 47.8 | 42.6 | 12.7 | 38 | 3175 | Vaginal | Preeclampsia |

TABLE 2A
HC ratio calculations for pregnancy outcome prediction using 471 microRNAs from pregnant non-Blacks (See full version of the table, Table 2B)

| | | | | | | (Column G) | | (Column I) |
|---|---|---|---|---|---|---|---|---|
| | | | | (Column E) | | \|HC ratio = | | Shared |
| | | (Column C) | | Mean | | Compromised | (Column H) | microRNA |
| (Column A) | (Column B) | Mean Healthy | (Column D) | Compromised | (Column F) | minus Healthy/ | XX = \|HC | with self- |
| Ratio order | 471 MicroRNAs | NON-BLACK | SD | NON-BLACK | SD | (mean SD)xx\| | Ratio\| ≥1.5 | identified |
| 1 | hsa-miR-374b- | −1.60715 | 0.322687 | −0.09985 | 0.021748 | 8.752301 | xx | |
| 2 | hsa-miR-26a-5p | 0.170792 | 0.071153 | −0.45202 | 0.175539 | 5.049328 | xx | |
| 3 | hsa-miR-18a-5p | −3.26451 | 1.261435 | −0.00607 | 0.090208 | 4.821451 | xx | |
| 4 | hsa-miR-652-3p | −1.41289 | 0.12101 | 0.124716 | 0.551146 | 4.575151 | xx | |
| 5 | hsa-miR-374a- | −1.90775 | 0.60976 | 0.12177 | 0.326151 | 4.33699 | xx | |
| 6 | hsa-miR-505-3p | −1.9521 | 0.442561 | −0.1063 | 0.412806 | 4.315818 | xx | |
| 7 | hsa-miR-185-5p | −2.16459 | 0.653478 | −0.0186 | 0.382906 | 4.141289 | xx | |
| 8 | hsa-miR-454-3p | −1.54513 | 0.571763 | 0.031249 | 0.223257 | 3.965633 | xx | |
| 9 | hsa-miR-320a | −1.22427 | 0.214056 | 0.142961 | 0.48603 | 3.905906 | xx | |
| 10 | hsa-miR-371a- | 0.701674 | 0.301323 | −0.39416 | 0.27359 | 3.812181 | xx | |
| 11 | hsa-miR-373-5p | 0.84198 | 0.301323 | −0.16059 | 0.27359 | 3.487735 | xx | |
| 12 | hsa-miR-502-3p | −1.68346 | 0.499359 | 0.153467 | 0.685722 | 3.100091 | xx | |
| 13 | hsa-miR-665 | −0.56973 | 0.301323 | 2.471473 | 1.736099 | 2.985347 | xx | |
| 14 | hsa-miR-23b-3p | −1.98163 | 0.796097 | −0.28221 | 0.360738 | 2.938037 | xx | |
| 15 | hsa-miR-18b-5p | −1.77082 | 1.062443 | −0.02518 | 0.135332 | 2.914819 | xx | x |
| 16 | hsa-miR-27a-3p | −0.45964 | 0.241587 | 0.076702 | 0.132851 | 2.864801 | xx | |
| 17 | hsa-miR-636 | 0.691477 | 0.301323 | −0.12959 | 0.27359 | 2.856322 | xx | |
| 18 | hsa-miR-199a- | −2.70055 | 0.744756 | −0.30239 | 1.107134 | 2.589966 | xx | |
| 19 | hsa-miR-361-5p | −1.99971 | 1.108255 | −0.24863 | 0.293447 | 2.498507 | xx | |
| 20 | hsa-miR-324-5p | −1.32801 | 0.708594 | 0.470691 | 0.792009 | 2.397307 | xx | |
| 21 | hsa-miR-31-5p | −1.62409 | 1.169749 | 0.074898 | 0.265859 | 2.366923 | xx | |
| 22 | hsa-miR-18b-3p | 0.518778 | 0.301323 | −0.16059 | 0.27359 | 2.363383 | xx | x |
| 23 | hsa-miR-654-5p | 0.233717 | 0.301323 | −0.43965 | 0.27359 | 2.342516 | xx | |
| 24 | hsa-miR-195-5p | −1.58982 | 1.045509 | −0.04603 | 0.27603 | 2.336349 | xx | |
| 25 | hsa-miR-151a- | −1.28125 | 0.50867 | 0.028563 | 0.615574 | 2.330121 | xx | |
| 26 | hsa-miR-625-5p | −3.22121 | 1.421968 | −0.51567 | 0.923 | 2.30753 | xx | |
| 27 | hsa-miR-29c-5p | −1.29847 | 0.720562 | 0.062287 | 0.475367 | 2.275644 | xx | |
| 28 | hsa-miR-551b- | −1.97114 | 0.724857 | −0.03584 | 0.980063 | 2.270256 | xx | |
| 29 | hsa-miR-1260a | 0.641061 | 0.64046 | −0.40701 | 0.295787 | 2.238884 | xx | |
| 30 | hsa-miR-363-3p | −2.24293 | 1.570397 | 0.176148 | 0.61072 | 2.218201 | xx | |
| 31 | hsa-miR-148a- | −3.79779 | 2.329934 | −0.14621 | 0.972439 | 2.211493 | xx | |
| 32 | hsa-miR-20b-5p | −0.5185 | 0.369618 | 0.089044 | 0.19079 | 2.168229 | xx | |
| 33 | hsa-miR-425-5p | −2.41456 | 1.273586 | −0.44601 | 0.551627 | 2.157059 | xx | |
| 34 | hsa-miR-151a- | −0.66459 | 0.185858 | −0.24671 | 0.203855 | 2.144527 | xx | |
| 35 | hsa-miR-141-3p | −0.9686 | 0.812812 | 0.715403 | 0.883999 | 1.984897 | xx | |
| 36 | hsa-miR-136-5p | −1.68987 | 1.900498 | 1.560481 | 1.608627 | 1.852514 | xx | |
| 37 | hsa-miR-28-5p | −0.27926 | 0.11338 | 0.11878 | 0.338506 | 1.761664 | xx | |
| 38 | hsa-miR-765 | −1.48205 | 0.301323 | 0.934661 | 2.55059 | 1.6948 | xx | |
| 39 | hsa-miR-29a-5p | 0.755766 | 1.103799 | −0.39416 | 0.27359 | 1.669722 | xx | |
| 40 | hsa-miR-93-5p | −0.72144 | 0.498703 | −0.08329 | 0.271401 | 1.657286 | xx | |
| 41 | hsa-miR-660-5p | −2.06409 | 1.62302 | −0.11524 | 0.7365 | 1.651905 | xx | |
| 42 | hsa-miR-197-3p | 0.2892 | 0.473592 | −0.28561 | 0.238731 | 1.613891 | xx | |
| 43 | hsa-miR-25-5p | 0.736077 | 1.240848 | −0.4636 | 0.27359 | 1.584314 | xx | |
| 44 | hsa-miR-628-3p | 0.613403 | 1.00398 | −0.39416 | 0.27359 | 1.577316 | xx | |
| 45 | hsa-miR-548am- | −2.18846 | 2.973847 | 0.414261 | 0.378366 | 1.552839 | xx | |
| 46 | hsa-miR-200c-3p | −0.59039 | 0.440325 | 0.606822 | 1.121883 | 1.532718 | xx | |
| 47 | hsa-miR-590-5p | −2.1 | 1.680134 | 0.297732 | 1.478738 | 1.518092 | xx | |
| 48 | hsa-miR-193a- | 0.665019 | 0.294569 | −1.04804 | 1.977531 | 1.507905 | xx | |
| 49 | hsa-miR-484 | −2.81891 | 3.548787 | −0.0326 | 0.168595 | 1.499073 | xx | |
| 50 | hsa-miR-301a- | −4.54215 | 4.817259 | −0.22431 | 1.05668 | 1.470168 | xx | |
| 51 | hsa-miR-376c | −1.92647 | 0.849715 | −0.23956 | 1.519033 | 1.424308 | | |
| 52 | hsa-miR-17-3p | −3.21421 | 4.18144 | 0.299228 | 1.288626 | 1.284605 | | |
| 53 | hsa-miR-132-3p | −2.76973 | 3.69422 | −0.03171 | 0.58296 | 1.280291 | | |
| 54 | hsa-miR-181c-5p | −2.67784 | 4.097739 | 0.218005 | 0.501485 | 1.259278 | | |
| 55 | hsa-miR-30c-5p | −2.08234 | 1.817395 | −0.49097 | 0.724291 | 1.252212 | | |
| 56 | hsa-miR-30e-5p | −0.70063 | 0.977341 | 0.290181 | 0.625471 | 1.236347 | | |
| 57 | hsa-miR-342-5p | −0.27094 | 0.19094 | 0.208918 | 0.594619 | 1.221693 | | |
| 58 | hsa-miR-362-5p | −3.03915 | 4.647199 | 0.18562 | 0.648234 | 1.217945 | | |

TABLE 2B
HC ratio calculations for pregnancy outcome prediction using 471 microRNAs from pregnant non-Blacks (full version)

| | | | | | | | | (Column I) |
|---|---|---|---|---|---|---|---|---|
| | | | | | | | | Shared |
| | | | | | | (Column G) | | microRNA |
| | | | | (Column E) | | \|HC ratio = | | with self- |
| | | (Column C) | | Mean | | Compromised | (Column H) | identified |
| (Column A) | (Column B) | Mean Healthy | (Column D) | Compromised | (Column F) | minus Healthy/ | XX = \|HC | Black |
| Ratio order | 471 MicroRNAs | NON-BLACK | SD | NON-BLACK | SD | (mean SD)\| | Ratio\| ≥1.5 | persons |
| 1 | hsa-miR-374b-5p | −1.60715 | 0.322687 | −0.09985 | 0.021748 | 8.752301 | xx | |
| 2 | hsa-miR-26a-5p | 0.170792 | 0.071153 | −0.45202 | 0.175539 | 5.049328 | xx | |
| 3 | hsa-miR-18a-5p | −3.26451 | 1.261435 | −0.00607 | 0.090208 | 4.821451 | xx | |
| 4 | hsa-miR-652-3p | −1.41289 | 0.12101 | 0.124716 | 0.551146 | 4.575151 | xx | |
| 5 | hsa-miR-374a-5p | −1.90775 | 0.60976 | 0.12177 | 0.326151 | 4.33699 | xx | |
| 6 | hsa-miR-505-3p | −1.9521 | 0.442561 | −0.1063 | 0.412806 | 4.315818 | xx | |
| 7 | hsa-miR-185-5p | −2.16459 | 0.653478 | −0.0186 | 0.382906 | 4.141289 | xx | |
| 8 | hsa-miR-454-3p | −1.54513 | 0.571763 | 0.031249 | 0.223257 | 3.965633 | xx | |
| 9 | hsa-miR-320a | −1.22427 | 0.214056 | 0.142961 | 0.48603 | 3.905906 | xx | |
| 10 | hsa-miR-371a-5p | 0.701674 | 0.301323 | −0.39416 | 0.27359 | 3.812181 | xx | |
| 11 | hsa-miR-373-5p | 0.84198 | 0.301323 | −0.16059 | 0.27359 | 3.487735 | xx | |
| 12 | hsa-miR-502-3p | −1.68346 | 0.499359 | 0.153467 | 0.685722 | 3.100091 | xx | |
| 13 | hsa-miR-665 | −0.56973 | 0.301323 | 2.471473 | 1.736099 | 2.985347 | xx | |
| 14 | hsa-miR-23b-3p | −1.98163 | 0.796097 | −0.28221 | 0.360738 | 2.938037 | xx | |
| 15 | hsa-miR-18b-5p | −1.77082 | 1.062443 | −0.02518 | 0.135332 | 2.914819 | xx | x |
| 16 | hsa-miR-27a-3p | −0.45964 | 0.241587 | 0.076702 | 0.132851 | 2.864801 | xx | |
| 17 | hsa-miR-636 | 0.691477 | 0.301323 | −0.12959 | 0.27359 | 2.856322 | xx | |
| 18 | hsa-miR-199a-5p | −2.70055 | 0.744756 | −0.30239 | 1.107134 | 2.589966 | xx | |
| 19 | hsa-miR-361-5p | −1.99971 | 1.108255 | −0.24863 | 0.293447 | 2.498507 | xx | |
| 20 | hsa-miR-324-5p | −1.32801 | 0.708594 | 0.470691 | 0.792009 | 2.397307 | xx | |
| 21 | hsa-miR-31-5p | −1.62409 | 1.169749 | 0.074898 | 0.265859 | 2.366923 | xx | |
| 22 | hsa-miR-18b-3p | 0.518778 | 0.301323 | −0.16059 | 0.27359 | 2.363383 | xx | x |
| 23 | hsa-miR-654-5p | 0.233717 | 0.301323 | −0.43965 | 0.27359 | 2.342516 | xx | |
| 24 | hsa-miR-195-5p | −1.58982 | 1.045509 | −0.04603 | 0.27603 | 2.336349 | xx | |
| 25 | hsa-miR-151a-3p | −1.28125 | 0.50867 | 0.028563 | 0.615574 | 2.330121 | xx | |
| 26 | hsa-miR-625-5p | −3.22121 | 1.421968 | −0.51567 | 0.923 | 2.30753 | xx | |
| 27 | hsa-miR-29c-5p | −1.29847 | 0.720562 | 0.062287 | 0.475367 | 2.275644 | xx | |
| 28 | hsa-miR-551b-3p | −1.97114 | 0.724857 | −0.03584 | 0.980063 | 2.270256 | xx | |
| 29 | hsa-miR-1260a | 0.641061 | 0.64046 | −0.40701 | 0.295787 | 2.238884 | xx | |
| 30 | hsa-miR-363-3p | −2.24293 | 1.570397 | 0.176148 | 0.61072 | 2.218201 | xx | |
| 31 | hsa-miR-148a-3p | −3.79779 | 2.329934 | −0.14621 | 0.972439 | 2.211493 | xx | |
| 32 | hsa-miR-20b-5p | −0.5185 | 0.369618 | 0.089044 | 0.19079 | 2.168229 | xx | |
| 33 | hsa-miR-425-5p | −2.41456 | 1.273586 | −0.44601 | 0.551627 | 2.157059 | xx | |
| 34 | hsa-miR-151a-5p | −0.66459 | 0.185858 | −0.24671 | 0.203855 | 2.144527 | xx | |
| 35 | hsa-miR-141-3p | −0.9686 | 0.812812 | 0.715403 | 0.883999 | 1.984897 | xx | |
| 36 | hsa-miR-136-5p | −1.68987 | 1.900498 | 1.560481 | 1.608627 | 1.852514 | xx | |
| 37 | hsa-miR-28-5p | −0.27926 | 0.11338 | 0.11878 | 0.338506 | 1.761664 | xx | |
| 38 | hsa-miR-765 | −1.48205 | 0.301323 | 0.934661 | 2.55059 | 1.6948 | xx | |
| 39 | hsa-miR-29a-5p | 0.755766 | 1.103799 | −0.39416 | 0.27359 | 1.669722 | xx | |
| 40 | hsa-miR-93-5p | −0.72144 | 0.498703 | −0.08329 | 0.271401 | 1.657286 | xx | |
| 41 | hsa-miR-660-5p | −2.06409 | 1.62302 | −0.11524 | 0.7365 | 1.651905 | xx | |
| 42 | hsa-miR-197-3p | 0.2892 | 0.473592 | −0.28561 | 0.238731 | 1.613891 | xx | |
| 43 | hsa-miR-25-5p | 0.736077 | 1.240848 | −0.4636 | 0.27359 | 1.584314 | xx | |
| 44 | hsa-miR-628-3p | 0.613403 | 1.00398 | −0.39416 | 0.27359 | 1.577316 | xx | |
| 45 | hsa-miR-548am-5p | −2.18846 | 2.973847 | 0.414261 | 0.378366 | 1.552839 | xx | |
| 46 | hsa-miR-200c-3p | −0.59039 | 0.440325 | 0.606822 | 1.121883 | 1.532718 | xx | |
| 47 | hsa-miR-590-5p | −2.1 | 1.680134 | 0.297732 | 1.478738 | 1.518092 | xx | |
| 48 | hsa-miR-193a-5p | 0.665019 | 0.294569 | −1.04804 | 1.977531 | 1.507905 | xx | |
| 49 | hsa-miR-484 | −2.81891 | 3.548787 | −0.0326 | 0.168595 | 1.499073 | xx | |
| 50 | hsa-miR-301a-3p | −4.54215 | 4.817259 | −0.22431 | 1.05668 | 1.470168 | xx | |
| 51 | hsa-miR-376c | −1.92647 | 0.849715 | −0.23956 | 1.519033 | 1.424308 | | |
| 52 | hsa-miR-17-3p | −3.21421 | 4.18144 | 0.299228 | 1.288626 | 1.284605 | | |
| 53 | hsa-miR-132-3p | −2.76973 | 3.69422 | −0.03171 | 0.58296 | 1.280291 | | |
| 54 | hsa-miR-181c-5p | −2.67784 | 4.097739 | 0.218005 | 0.501485 | 1.259278 | | |
| 55 | hsa-miR-30c-5p | −2.08234 | 1.817395 | −0.49097 | 0.724291 | 1.252212 | | |
| 56 | hsa-miR-30e-5p | −0.70063 | 0.977341 | 0.290181 | 0.625471 | 1.236347 | | |
| 57 | hsa-miR-342-5p | −0.27094 | 0.19094 | 0.208918 | 0.594619 | 1.221693 | | |
| 58 | hsa-miR-362-5p | −3.03915 | 4.647199 | 0.18562 | 0.648234 | 1.217945 | | |
| 59 | hsa-miR-93-3p | 0.476009 | 0.744535 | −0.12959 | 0.27359 | 1.189638 | | |
| 60 | hsa-miR-191-5p | 0.357756 | 0.749988 | −0.24944 | 0.27359 | 1.18642 | | |
| 61 | hsa-miR-520d-3p | 2.807154 | 4.35775 | 0.091103 | 0.27359 | 1.172901 | | |
| 62 | hsa-miR-595 | −2.70478 | 0.996221 | −0.46495 | 2.844517 | 1.166354 | | |
| 63 | hsa-miR-1207-5p | 0.359069 | 0.765137 | −0.34594 | 0.470627 | 1.141014 | | |
| 64 | hsa-miR-15a-5p | −0.06169 | 0.524259 | 0.559804 | 0.566737 | 1.139315 | | |
| 65 | hsa-miR-1915-3p | 0.782997 | 1.386829 | −0.12181 | 0.211692 | 1.132049 | | |
| 66 | hsa-miR-194-3p | 0.728563 | 0.85356 | 0.091103 | 0.27359 | 1.131101 | | |
| 67 | hsa-miR-99a-5p | −3.11797 | 4.587682 | −0.12451 | 0.707117 | 1.130718 | | |
| 68 | hsa-miR-221-5p | −3.1991 | 4.418635 | −0.2049 | 0.938723 | 1.117788 | | |
| 69 | hsa-miR-27b-3p | −0.37291 | 0.149835 | −0.16609 | 0.222148 | 1.111977 | | |
| 70 | hsa-miR-548a-5p | 0.274849 | 0.301323 | −0.03952 | 0.27359 | 1.093633 | | |
| 71 | hsa-miR-645 | 0.336471 | 0.301323 | 0.022099 | 0.27359 | 1.093633 | | |
| 72 | hsa-miR-485-3p | 0.215643 | 0.301323 | −0.09873 | 0.27359 | 1.093633 | | |
| 73 | hsa-miR-429 | 0.366114 | 0.301323 | 0.051742 | 0.27359 | 1.093633 | | |
| 74 | hsa-miR-876-5p | 0.366114 | 0.301323 | 0.051742 | 0.27359 | 1.093633 | | |
| 75 | hsa-miR-30a-3p | 0.366114 | 0.301323 | 0.051742 | 0.27359 | 1.093633 | | |
| 76 | hsa-miR-195-3p | 0.366114 | 0.301323 | 0.051742 | 0.27359 | 1.093633 | | |
| 77 | hsa-miR-770-5p | 0.366114 | 0.301323 | 0.051742 | 0.27359 | 1.093633 | | |
| 78 | hsa-miR-875-5p | 0.366114 | 0.301323 | 0.051742 | 0.27359 | 1.093633 | | |
| 79 | hsa-let-7f-2-3p | 0.366114 | 0.301323 | 0.051742 | 0.27359 | 1.093633 | | |
| 80 | hsa-miR-193b-5p | 0.366114 | 0.301323 | 0.051742 | 0.27359 | 1.093633 | | |
| 81 | hsa-miR-124-3p | 0.366114 | 0.301323 | 0.051742 | 0.27359 | 1.093633 | | |
| 82 | hsa-miR-509-3p | 0.366114 | 0.301323 | 0.051742 | 0.27359 | 1.093633 | | |
| 83 | hsa-miR-508-5p | 0.366114 | 0.301323 | 0.051742 | 0.27359 | 1.093633 | | |
| 84 | hsa-miR-548e | 0.184781 | 0.301323 | −0.12959 | 0.27359 | 1.093633 | | |
| 85 | hsa-miR-873-5p | 0.184781 | 0.301323 | −0.12959 | 0.27359 | 1.093633 | | |
| 86 | hsa-miR-1285-3p | 0.184781 | 0.301323 | −0.12959 | 0.27359 | 1.093633 | | |
| 87 | hsa-miR-454-5p | 0.184781 | 0.301323 | −0.12959 | 0.27359 | 1.093633 | | |
| 88 | hsa-miR-514a-3p | 0.184781 | 0.301323 | −0.12959 | 0.27359 | 1.093633 | | |
| 89 | hsa-let-7i-3p | 0.184781 | 0.301323 | −0.12959 | 0.27359 | 1.093633 | | |
| 90 | hsa-miR-409-5p | 0.184781 | 0.301323 | −0.12959 | 0.27359 | 1.093633 | | |
| 91 | hsa-miR-338-5p | 0.184781 | 0.301323 | −0.12959 | 0.27359 | 1.093633 | | |
| 92 | hsa-miR-513c-5p | 0.184781 | 0.301323 | −0.12959 | 0.27359 | 1.093633 | | |
| 93 | hsa-miR-1307-3p | 0.184781 | 0.301323 | −0.12959 | 0.27359 | 1.093633 | | |
| 94 | hsa-miR-422a | 0.184781 | 0.301323 | −0.12959 | 0.27359 | 1.093633 | | |
| 95 | hsa-miR-10b-5p | 0.184781 | 0.301323 | −0.12959 | 0.27359 | 1.093633 | | |
| 96 | hsa-miR-1469 | 0.273552 | 0.301323 | −0.04082 | 0.27359 | 1.093633 | | |
| 97 | hsa-miR-877-5p | 0.111006 | 0.301323 | −0.20337 | 0.27359 | 1.093633 | | |
| 98 | hsa-miR-513b | 0.15378 | 0.301323 | −0.16059 | 0.27359 | 1.093633 | | |
| 99 | hsa-miR-198 | 0.15378 | 0.301323 | −0.16059 | 0.27359 | 1.093633 | | |
| 100 | hsa-miR-624-3p | 0.221548 | 0.301323 | −0.09282 | 0.27359 | 1.093633 | | |
| 101 | hsa-miR-24-1-5p | 0.221548 | 0.301323 | −0.09282 | 0.27359 | 1.093633 | | |
| 102 | hsa-miR-188-3p | 0.221548 | 0.301323 | −0.09282 | 0.27359 | 1.093633 | | |
| 103 | hsa-miR-487a | 0.221548 | 0.301323 | −0.09282 | 0.27359 | 1.093633 | | |
| 104 | hsa-miR-576-3p | 0.221548 | 0.301323 | −0.09282 | 0.27359 | 1.093633 | | |
| 105 | hsa-miR-374b-3p | 0.221548 | 0.301323 | −0.09282 | 0.27359 | 1.093633 | | |
| 106 | hsa-miR-145-3p | 0.221548 | 0.301323 | −0.09282 | 0.27359 | 1.093633 | | |
| 107 | hsa-miR-337-3p | 0.221548 | 0.301323 | −0.09282 | 0.27359 | 1.093633 | | |
| 108 | hsa-miR-369-5p | 0.221548 | 0.301323 | −0.09282 | 0.27359 | 1.093633 | | |
| 109 | hsa-miR-1255a | 0.221548 | 0.301323 | −0.09282 | 0.27359 | 1.093633 | | |
| 110 | hsa-miR-299-5p | 0.221548 | 0.301323 | −0.09282 | 0.27359 | 1.093633 | | |
| 111 | hsa-miR-206 | 0.221548 | 0.301323 | −0.09282 | 0.27359 | 1.093633 | | |
| 112 | hsa-miR-138-2-3p | 0.221548 | 0.301323 | −0.09282 | 0.27359 | 1.093633 | | |
| 113 | hsa-miR-520f | 0.221548 | 0.301323 | −0.09282 | 0.27359 | 1.093633 | | |
| 114 | hsa-miR-339-5p | 0.064931 | 0.301323 | −0.24944 | 0.27359 | 1.093633 | | |
| 115 | hsa-miR-1180 | 0.064931 | 0.301323 | −0.24944 | 0.27359 | 1.093633 | | |
| 116 | hsa-miR-29b-2-5p | 0.230534 | 0.301323 | −0.08384 | 0.27359 | 1.093633 | | |
| 117 | hsa-miR-122-5p | 0.287058 | 0.301323 | −0.02731 | 0.27359 | 1.093633 | | |
| 118 | hsa-miR-887 | 0.266054 | 0.301323 | −0.04832 | 0.27359 | 1.093633 | | |
| 119 | hsa-miR-452-5p | 0.461993 | 0.301323 | 0.147621 | 0.27359 | 1.093633 | | |
| 120 | hsa-miR-580 | 0.461993 | 0.301323 | 0.147621 | 0.27359 | 1.093633 | | |
| 121 | hsa-miR-19a-5p | 0.461993 | 0.301323 | 0.147621 | 0.27359 | 1.093633 | | |
| 122 | hsa-miR-190a | 0.461993 | 0.301323 | 0.147621 | 0.27359 | 1.093633 | | |
| 123 | hsa-miR-493-3p | 0.461993 | 0.301323 | 0.147621 | 0.27359 | 1.093633 | | |
| 124 | hsa-miR-431-3p | 0.461993 | 0.301323 | 0.147621 | 0.27359 | 1.093633 | | |
| 125 | hsa-miR-34a-3p | 0.461993 | 0.301323 | 0.147621 | 0.27359 | 1.093633 | | |
| 126 | hsa-miR-1827 | 0.461993 | 0.301323 | 0.147621 | 0.27359 | 1.093633 | | |
| 127 | hsa-miR-208a | 0.461993 | 0.301323 | 0.147621 | 0.27359 | 1.093633 | | |
| 128 | hsa-miR-27a-5p | 0.461993 | 0.301323 | 0.147621 | 0.27359 | 1.093633 | | |
| 129 | hsa-miR-1270 | 0.461993 | 0.301323 | 0.147621 | 0.27359 | 1.093633 | | |
| 130 | hsa-miR-218-5p | 0.461993 | 0.301323 | 0.147621 | 0.27359 | 1.093633 | | |
| 131 | hsa-miR-33a-3p | 0.461993 | 0.301323 | 0.147621 | 0.27359 | 1.093633 | | |
| 132 | hsa-miR-656 | 0.461993 | 0.301323 | 0.147621 | 0.27359 | 1.093633 | | |
| 133 | hsa-miR-148b-5p | 0.461993 | 0.301323 | 0.147621 | 0.27359 | 1.093633 | | |
| 134 | hsa-let-7a-3p | 0.461993 | 0.301323 | 0.147621 | 0.27359 | 1.093633 | | |
| 135 | hsa-miR-1185-5p | 0.461993 | 0.301323 | 0.147621 | 0.27359 | 1.093633 | | |
| 136 | hsa-miR-1284 | 0.461993 | 0.301323 | 0.147621 | 0.27359 | 1.093633 | | |
| 137 | hsa-let-7g-3p | 0.461993 | 0.301323 | 0.147621 | 0.27359 | 1.093633 | | |
| 138 | hsa-miR-1910 | 0.461993 | 0.301323 | 0.147621 | 0.27359 | 1.093633 | | |
| 139 | hsa-miR-551a | 0.461993 | 0.301323 | 0.147621 | 0.27359 | 1.093633 | | |
| 140 | hsa-miR-148a-5p | 0.461993 | 0.301323 | 0.147621 | 0.27359 | 1.093633 | | |
| 141 | hsa-miR-650 | 0.461993 | 0.301323 | 0.147621 | 0.27359 | 1.093633 | | |
| 142 | hsa-miR-187-5p | 0.461993 | 0.301323 | 0.147621 | 0.27359 | 1.093633 | | |
| 143 | hsa-miR-185-3p | 0.461993 | 0.301323 | 0.147621 | 0.27359 | 1.093633 | | |
| 144 | hsa-miR-1233 | 0.461993 | 0.301323 | 0.147621 | 0.27359 | 1.093633 | | |
| 145 | hsa-miR-363-5p | 0.461993 | 0.301323 | 0.147621 | 0.27359 | 1.093633 | | |
| 146 | hsa-miR-485-5p | 0.461993 | 0.301323 | 0.147621 | 0.27359 | 1.093633 | | |
| 147 | hsa-miR-491-3p | 0.461993 | 0.301323 | 0.147621 | 0.27359 | 1.093633 | | |
| 148 | hsa-miR-380-3p | 0.461993 | 0.301323 | 0.147621 | 0.27359 | 1.093633 | | |
| 149 | hsa-miR-135a-5p | 0.461993 | 0.301323 | 0.147621 | 0.27359 | 1.093633 | | |
| 150 | hsa-miR-526b-5p | 0.461993 | 0.301323 | 0.147621 | 0.27359 | 1.093633 | | |
| 151 | hsa-miR-335-3p | 0.461993 | 0.301323 | 0.147621 | 0.27359 | 1.093633 | | |
| 152 | hsa-miR-30c-2-3p | 0.461993 | 0.301323 | 0.147621 | 0.27359 | 1.093633 | | |
| 153 | hsa-miR-589-3p | 0.461993 | 0.301323 | 0.147621 | 0.27359 | 1.093633 | | |
| 154 | hsa-miR-130b-5p | 0.461993 | 0.301323 | 0.147621 | 0.27359 | 1.093633 | | |
| 155 | hsa-miR-149-3p | 0.461993 | 0.301323 | 0.147621 | 0.27359 | 1.093633 | | |
| 156 | hsa-miR-412 | 0.461993 | 0.301323 | 0.147621 | 0.27359 | 1.093633 | | |
| 157 | hsa-miR-379-3p | 0.461993 | 0.301323 | 0.147621 | 0.27359 | 1.093633 | | |
| 158 | hsa-miR-1323 | 0.461993 | 0.301323 | 0.147621 | 0.27359 | 1.093633 | | |
| 159 | hsa-miR-183-3p | 0.461993 | 0.301323 | 0.147621 | 0.27359 | 1.093633 | | |
| 160 | hsa-miR-491-5p | 0.461993 | 0.301323 | 0.147621 | 0.27359 | 1.093633 | | |
| 161 | hsa-miR-200b-5p | 0.461993 | 0.301323 | 0.147621 | 0.27359 | 1.093633 | | |
| 162 | hsa-miR-34c-3p | 0.461993 | 0.301323 | 0.147621 | 0.27359 | 1.093633 | | |
| 163 | hsa-miR-1909-5p | 0.461993 | 0.301323 | 0.147621 | 0.27359 | 1.093633 | | |
| 164 | hsa-miR-642a-5p | 0.461993 | 0.301323 | 0.147621 | 0.27359 | 1.093633 | | |
| 165 | hsa-miR-1179 | 0.461993 | 0.301323 | 0.147621 | 0.27359 | 1.093633 | | |
| 166 | hsa-miR-936 | 0.461993 | 0.301323 | 0.147621 | 0.27359 | 1.093633 | | |
| 167 | hsa-miR-520b | 0.461993 | 0.301323 | 0.147621 | 0.27359 | 1.093633 | | |
| 168 | hsa-miR-492 | 0.461993 | 0.301323 | 0.147621 | 0.27359 | 1.093633 | | |
| 169 | hsa-miR-146a-3p | 0.461993 | 0.301323 | 0.147621 | 0.27359 | 1.093633 | | |
| 170 | hsa-miR-648 | 0.461993 | 0.301323 | 0.147621 | 0.27359 | 1.093633 | | |
| 171 | hsa-miR-520e | 0.461993 | 0.301323 | 0.147621 | 0.27359 | 1.093633 | | |
| 172 | hsa-miR-631 | 0.461993 | 0.301323 | 0.147621 | 0.27359 | 1.093633 | | |
| 173 | hsa-miR-146b-3p | 0.461993 | 0.301323 | 0.147621 | 0.27359 | 1.093633 | | |
| 174 | hsa-miR-196a-3p | 0.461993 | 0.301323 | 0.147621 | 0.27359 | 1.093633 | | |
| 175 | hsa-miR-204-5p | 0.461993 | 0.301323 | 0.147621 | 0.27359 | 1.093633 | | |
| 176 | hsa-miR-548j | 0.461993 | 0.301323 | 0.147621 | 0.27359 | 1.093633 | | |
| 177 | hsa-miR-1254 | 0.461993 | 0.301323 | 0.147621 | 0.27359 | 1.093633 | | |
| 178 | hsa-miR-10a-3p | 0.461993 | 0.301323 | 0.147621 | 0.27359 | 1.093633 | | |
| 179 | hsa-miR-662 | 0.461993 | 0.301323 | 0.147621 | 0.27359 | 1.093633 | | |
| 180 | hsa-miR-490-5p | 0.461993 | 0.301323 | 0.147621 | 0.27359 | 1.093633 | | |
| 181 | hsa-miR-1908 | 0.461993 | 0.301323 | 0.147621 | 0.27359 | 1.093633 | | |
| 182 | hsa-miR-504 | 0.461993 | 0.301323 | 0.147621 | 0.27359 | 1.093633 | | |
| 183 | hsa-miR-323a-3p | 0.318639 | 0.301323 | 0.004267 | 0.27359 | 1.093633 | | |
| 184 | hsa-miR-455-5p | 0.405475 | 0.301323 | 0.091103 | 0.27359 | 1.093633 | | |
| 185 | hsa-miR-34c-5p | 0.405475 | 0.301323 | 0.091103 | 0.27359 | 1.093633 | | |
| 186 | hsa-miR-369-3p | 0.405475 | 0.301323 | 0.091103 | 0.27359 | 1.093633 | | |
| 187 | hsa-miR-424-3p | 0.405475 | 0.301323 | 0.091103 | 0.27359 | 1.093633 | | |
| 188 | hsa-miR-1208 | 0.405475 | 0.301323 | 0.091103 | 0.27359 | 1.093633 | | |
| 189 | hsa-miR-23b-5p | 0.405475 | 0.301323 | 0.091103 | 0.27359 | 1.093633 | | |
| 190 | hsa-miR-411-3p | 0.405475 | 0.301323 | 0.091103 | 0.27359 | 1.093633 | | |
| 191 | hsa-miR-937 | 0.405475 | 0.301323 | 0.091103 | 0.27359 | 1.093633 | | |
| 192 | hsa-miR-1301 | 0.405475 | 0.301323 | 0.091103 | 0.27359 | 1.093633 | | |
| 193 | hsa-let-7d-3p | 0.405475 | 0.301323 | 0.091103 | 0.27359 | 1.093633 | | |
| 194 | hsa-miR-449b-5p | 0.405475 | 0.301323 | 0.091103 | 0.27359 | 1.093633 | | |
| 195 | hsa-miR-512-3p | 0.405475 | 0.301323 | 0.091103 | 0.27359 | 1.093633 | | |
| 196 | hsa-miR-509-3-5p | 0.405475 | 0.301323 | 0.091103 | 0.27359 | 1.093633 | | |
| 197 | hsa-miR-583 | 0.405475 | 0.301323 | 0.091103 | 0.27359 | 1.093633 | | |
| 198 | hsa-miR-498 | 0.405475 | 0.301323 | 0.091103 | 0.27359 | 1.093633 | | |
| 199 | hsa-miR-1182 | 0.405475 | 0.301323 | 0.091103 | 0.27359 | 1.093633 | | |
| 200 | hsa-miR-483-3p | 0.405475 | 0.301323 | 0.091103 | 0.27359 | 1.093633 | | |
| 201 | hsa-miR-411-5p | 0.322396 | 0.301323 | 0.008024 | 0.27359 | 1.093633 | | |
| 202 | hsa-miR-26a-1-3p | 0.270391 | 0.301323 | −0.04398 | 0.27359 | 1.093633 | | |
| 203 | hsa-miR-33b-5p | 0.304414 | 0.301323 | −0.00996 | 0.27359 | 1.093633 | | |
| 204 | hsa-miR-641 | 0.304414 | 0.301323 | −0.00996 | 0.27359 | 1.093633 | | |
| 205 | hsa-miR-625-3p | 0.304414 | 0.301323 | −0.00996 | 0.27359 | 1.093633 | | |
| 206 | hsa-miR-1228-5p | 0.304414 | 0.301323 | −0.00996 | 0.27359 | 1.093633 | | |
| 207 | hsa-miR-532-5p | −3.65228 | 4.661727 | −0.51087 | 1.090914 | 1.092165 | | |
| 208 | hsa-miR-340-3p | −3.57728 | 4.630331 | −0.42253 | 1.168126 | 1.088134 | | |
| 209 | hsa-miR-409-3p | −1.31015 | 0.942363 | 0.002197 | 1.539315 | 1.057632 | | |
| 210 | hsa-miR-30e-3p | −4.19223 | 5.222565 | −0.80857 | 1.220657 | 1.050298 | | |
| 211 | hsa-miR-148b-3p | −2.19914 | 1.585119 | −0.74215 | 1.195364 | 1.048017 | | |
| 212 | hsa-miR-192-5p | −2.10472 | 1.727367 | −0.49192 | 1.371908 | 1.040761 | | |
| 213 | hsa-miR-579 | 0.552385 | 1.113035 | −0.16059 | 0.27359 | 1.028362 | | |
| 214 | hsa-miR-575 | −3.49217 | 5.069089 | −0.14572 | 1.568716 | 1.0083 | | |
| 215 | hsa-miR-32-3p | −1.99871 | 0.301323 | −0.69239 | 2.292785 | 1.007144 | | |
| 216 | hsa-miR-199b-5p | −3.9391 | 5.037471 | −0.72521 | 1.352483 | 1.00592 | | |
| 217 | hsa-miR-550a-3p | 0.9307 | 3.369231 | −0.89444 | 0.27359 | 1.002046 | | |
| 218 | hsa-miR-588 | 1.783329 | 3.793837 | −0.24944 | 0.27359 | 0.999536 | | |
| 219 | hsa-miR-423-3p | 0.393987 | 1.213692 | −0.34821 | 0.27359 | 0.998062 | | |
| 220 | hsa-miR-182-5p | 0.617726 | 1.231328 | −0.12959 | 0.27359 | 0.993167 | | |
| 221 | hsa-miR-10a-5p | −3.09647 | 4.416394 | −0.51046 | 0.808336 | 0.989914 | | |
| 222 | hsa-miR-106a-3p | 0.592433 | 1.251001 | −0.16059 | 0.27359 | 0.987839 | | |
| 223 | hsa-miR-154-3p | 0.41571 | 1.288572 | −0.34821 | 0.27359 | 0.978033 | | |
| 224 | hsa-miR-181a-5p | −0.19162 | 0.483912 | 0.098337 | 0.112242 | 0.972745 | | |
| 225 | hsa-miR-9-5p | 0.424619 | 1.319293 | −0.34821 | 0.27359 | 0.970356 | | |
| 226 | hsa-miR-25-3p | −0.37435 | 0.373783 | −0.10477 | 0.185836 | 0.963433 | | |
| 227 | hsa-miR-602 | 1.124326 | 1.443883 | −0.28154 | 1.477906 | 0.962329 | | |
| 228 | hsa-miR-142-3p | −0.2795 | 0.757408 | 0.549866 | 1.011262 | 0.93784 | | |
| 229 | hsa-miR-365a-3p | −3.46924 | 4.771805 | −0.65862 | 1.223364 | 0.937628 | | |
| 230 | hsa-miR-376a-5p | 0.565728 | 1.46537 | −0.24944 | 0.27359 | 0.937536 | | |
| 231 | hsa-miR-136-3p | 0.571301 | 1.484608 | −0.24944 | 0.27359 | 0.933617 | | |
| 232 | hsa-miR-200a-3p | 0.474762 | 1.492317 | −0.34821 | 0.27359 | 0.932071 | | |
| 233 | hsa-miR-26b-3p | 0.433376 | 1.508071 | −0.39416 | 0.27359 | 0.928952 | | |
| 234 | hsa-miR-340-5p | −4.15554 | 5.279581 | −0.66089 | 2.304447 | 0.92158 | | |
| 235 | hsa-miR-150-5p | 0.388566 | 0.187193 | 0.123564 | 0.389958 | 0.918311 | | |
| 236 | hsa-miR-192-3p | 0.704387 | 1.637367 | −0.16059 | 0.27359 | 0.905284 | | |
| 237 | hsa-miR-17-5p | −0.29863 | 0.400368 | −0.06589 | 0.114128 | 0.904737 | | |
| 238 | hsa-miR-622 | 0.064931 | 0.301323 | 1.457674 | 2.798587 | 0.89857 | | |
| 239 | hsa-miR-638 | 0.66987 | 1.677151 | −0.25495 | 0.389303 | 0.895079 | | |
| 240 | hsa-miR-125b-5p | −2.85635 | 4.47146 | −0.52637 | 0.738108 | 0.894499 | | |
| 241 | hsa-miR-582-5p | −3.92764 | 5.2781 | −0.67487 | 1.996934 | 0.89423 | | |
| 242 | hsa-miR-338-3p | −3.63586 | 5.722212 | −0.54126 | 1.229431 | 0.89032 | | |
| 243 | hsa-miR-155-3p | −1.32592 | 2.58209 | 1.365214 | 3.480133 | 0.887838 | | |
| 244 | hsa-miR-19a-3p | −1.32716 | 1.519757 | −0.076 | 1.309712 | 0.884379 | | |
| 245 | hsa-miR-223-5p | −3.26608 | 4.721784 | −0.5975 | 1.341956 | 0.880175 | | |
| 246 | hsa-miR-1261 | 0.15378 | 0.301323 | 1.528848 | 2.832913 | 0.877451 | | |
| 247 | hsa-miR-769-3p | 0.764925 | 1.846542 | −0.16059 | 0.27359 | 0.873075 | | |
| 248 | hsa-miR-557 | 1.531375 | 1.8639 | 0.11115 | 1.479382 | 0.849599 | | |
| 249 | hsa-miR-497-5p | −1.87841 | 3.10774 | −0.04578 | 1.232348 | 0.844511 | | |
| 250 | hsa-miR-144-3p | −3.43247 | 5.952893 | 0.242774 | 2.843169 | 0.835657 | | |
| 251 | hsa-miR-449a | −0.9867 | 4.635018 | 1.661522 | 1.715752 | 0.833986 | | |
| 252 | hsa-miR-601 | −0.03384 | 0.301323 | 0.994856 | 2.170491 | 0.832342 | | |
| 253 | hsa-miR-421 | −0.79161 | 2.11427 | 1.400155 | 3.204475 | 0.824165 | | |
| 254 | hsa-miR-127-3p | −0.35898 | 1.159038 | 0.952271 | 2.026267 | 0.82331 | | |
| 255 | hsa-miR-548d-5p | 1.287321 | 3.273812 | −0.62095 | 1.36812 | 0.822187 | | |
| 256 | hsa-miR-584-5p | −1.96313 | 0.657635 | −0.87538 | 1.989875 | 0.821717 | | |
| 257 | hsa-miR-9-3p | 0.064931 | 0.301323 | 1.065205 | 2.187107 | 0.80394 | | |
| 258 | hsa-miR-766-3p | 0.821535 | 1.116148 | 0.024951 | 0.8926 | 0.793115 | | |
| 259 | hsa-miR-503 | 0.76429 | 2.652187 | −0.39416 | 0.27359 | 0.791893 | | |
| 260 | hsa-miR-26b-5p | −0.45588 | 0.4401 | −0.11955 | 0.417364 | 0.784475 | | |
| 261 | hsa-miR-1306-3p | −0.60558 | 3.956496 | 1.157961 | 0.551452 | 0.782415 | | |
| 262 | hsa-miR-610 | 0.221548 | 0.301323 | 1.010296 | 1.75746 | 0.766228 | | |
| 263 | hsa-miR-126-5p | −2.37489 | 3.521532 | −0.55582 | 1.319844 | 0.751468 | | |
| 264 | hsa-miR-101-3p | −1.24892 | 1.461256 | −0.15607 | 1.468065 | 0.746147 | | |
| 265 | hsa-miR-140-3p | 0.26379 | 0.171984 | 0.069903 | 0.349243 | 0.743965 | | |
| 266 | hsa-miR-196a-5p | 1.511198 | 2.800027 | −0.85327 | 3.556877 | 0.743906 | | |
| 267 | hsa-miR-99b-3p | −0.03384 | 0.301323 | 0.670127 | 1.611841 | 0.73592 | | |
| 268 | hsa-miR-16-5p | −0.16945 | 0.495211 | 0.078387 | 0.183834 | 0.729965 | | |
| 269 | hsa-miR-342-3p | 0.090031 | 0.544182 | −0.29391 | 0.50907 | 0.729061 | | |
| 270 | hsa-miR-376a-3p | −2.37221 | 3.826934 | −0.23545 | 2.037723 | 0.728692 | | |
| 271 | hsa-miR-125b-2-3p | 0.405475 | 0.301323 | 1.084703 | 1.569392 | 0.72617 | | |
| 272 | hsa-miR-1299 | 0.15378 | 0.301323 | 0.827448 | 1.559853 | 0.723916 | | |
| 273 | hsa-miR-487b | −1.38145 | 1.208473 | −0.00482 | 2.632066 | 0.716894 | | |
| 274 | hsa-miR-539-5p | −0.48734 | 1.858449 | 1.173739 | 2.802148 | 0.712817 | | |
| 275 | hsa-miR-424-5p | −3.46198 | 5.74535 | −0.72496 | 1.955477 | 0.710839 | | |
| 276 | hsa-miR-7-5p | −3.19972 | 5.279069 | −0.71868 | 1.741847 | 0.706757 | | |
| 277 | hsa-miR-147b | 0.064931 | 0.301323 | 0.749093 | 1.644473 | 0.70322 | | |
| 278 | hsa-miR-455-3p | 1.369322 | 3.38225 | 0.091103 | 0.27359 | 0.699275 | | |
| 279 | hsa-miR-10b-3p | 0.184781 | 0.301323 | 0.792018 | 1.446 | 0.695049 | | |
| 280 | hsa-miR-326 | −1.01477 | 0.7661 | −0.1759 | 1.65798 | 0.692117 | | |
| 281 | hsa-miR-21-5p | −0.54088 | 0.865617 | −0.04218 | 0.591633 | 0.684447 | | |
| 282 | hsa-miR-885-3p | 1.24418 | 3.291033 | 0.051742 | 0.27359 | 0.66904 | | |
| 283 | hsa-miR-298 | 1.230091 | 3.455739 | −0.00996 | 0.27359 | 0.665025 | | |
| 284 | hsa-miR-616-3p | 1.397008 | 3.488045 | 0.147621 | 0.27359 | 0.664279 | | |
| 285 | hsa-miR-106b-3p | 1.419128 | 3.564576 | 0.147621 | 0.27359 | 0.66256 | | |
| 286 | hsa-miR-433 | 1.443886 | 3.650239 | 0.147621 | 0.27359 | 0.660715 | | |
| 287 | hsa-miR-659-3p | 1.264417 | 3.681282 | −0.04082 | 0.27359 | 0.660066 | | |
| 288 | hsa-miR-33a-5p | −2.75069 | 3.14524 | −0.41211 | 3.955556 | 0.658682 | | |
| 289 | hsa-miR-28-3p | 0.101327 | 0.301323 | 0.634618 | 1.319512 | 0.658045 | | |
| 290 | hsa-miR-129-5p | 1.539384 | 4.312531 | 0.051742 | 0.27359 | 0.648758 | | |
| 291 | hsa-miR-1470 | 1.733619 | 3.314439 | 0.04275 | 1.957682 | 0.641438 | | |
| 292 | hsa-miR-486-5p | −0.93614 | 1.271966 | 0.087751 | 1.998438 | 0.626159 | | |
| 293 | hsa-miR-193a-3p | −2.9872 | 3.878034 | −0.5008 | 4.06754 | 0.625858 | | |
| 294 | hsa-miR-502-5p | −0.28508 | 1.214603 | 0.693206 | 1.957408 | 0.616825 | | |
| 295 | hsa-miR-32-5p | −2.88963 | 3.506163 | −0.66278 | 3.87597 | 0.603307 | | |
| 296 | hsa-miR-933 | 1.196517 | 2.270312 | −0.03782 | 1.85786 | 0.598007 | | |
| 297 | hsa-miR-22-3p | −0.5874 | 0.585448 | −0.324 | 0.317385 | 0.583504 | | |
| 298 | hsa-miR-101-5p | 0.367397 | 1.951978 | 1.354034 | 1.45624 | 0.578976 | | |
| 299 | hsa-miR-149-5p | 1.227845 | 1.991457 | 0.200613 | 1.653876 | 0.563588 | | |
| 300 | hsa-miR-940 | 1.115825 | 1.615718 | 0.391137 | 0.983369 | 0.557648 | | |
| 301 | hsa-miR-1268a | 0.327376 | 0.764817 | −0.02961 | 0.51719 | 0.556925 | | |
| 302 | hsa-miR-1234 | 1.30743 | 1.95475 | 0.353838 | 1.47931 | 0.555373 | | |
| 303 | hsa-miR-29b-3p | −0.64339 | 1.199083 | 0.079341 | 1.45804 | 0.543993 | | |
| 304 | hsa-miR-142-5p | −1.04833 | 1.530449 | −0.08965 | 2.049719 | 0.535548 | | |
| 305 | hsa-miR-324-3p | 0.633977 | 0.861138 | 0.187062 | 0.817503 | 0.532473 | | |
| 306 | hsa-miR-1228-3p | 1.164218 | 1.824938 | 0.321592 | 1.350336 | 0.530743 | | |
| 307 | hsa-miR-339-3p | −1.2129 | 1.295292 | −0.50414 | 1.398868 | 0.526146 | | |
| 308 | hsa-miR-15b-5p | −0.19466 | 0.121216 | −0.11057 | 0.200954 | 0.522034 | | |
| 309 | hsa-miR-129-2-3p | 1.999632 | 3.281272 | 0.326515 | 3.156173 | 0.519808 | | |
| 310 | hsa-miR-486-3p | −1.51447 | 2.723989 | −0.35828 | 1.824378 | 0.508397 | | |
| 311 | hsa-miR-19b-1-5p | −1.84708 | 2.119538 | −0.71691 | 2.361296 | 0.504447 | | |
| 312 | hsa-miR-30b-5p | −0.53494 | 0.731713 | −0.23221 | 0.47538 | 0.501592 | | |
| 313 | hsa-miR-29c-3p | −0.39904 | 0.95615 | 0.063746 | 0.924389 | 0.492186 | | |
| 314 | hsa-miR-33b-3p | 1.27533 | 2.003179 | 0.441005 | 1.414215 | 0.488281 | | |
| 315 | hsa-miR-1281 | 1.401102 | 2.079847 | 0.542178 | 1.442687 | 0.487674 | | |
| 316 | hsa-miR-191-3p | 1.251735 | 2.134976 | 0.367859 | 1.505098 | 0.485637 | | |
| 317 | hsa-miR-1539 | 1.350064 | 2.015753 | 0.523959 | 1.442767 | 0.477722 | | |
| 318 | hsa-miR-654-3p | −0.98258 | 2.99318 | −2.34452 | 2.737045 | 0.475355 | | |
| 319 | hsa-miR-545-3p | 0.193881 | 1.797011 | 1.050989 | 1.810326 | 0.475203 | | |
| 320 | hsa-miR-19b-3p | −0.63448 | 0.987777 | −0.20646 | 0.826985 | 0.471706 | | |
| 321 | hsa-miR-598 | −2.5385 | 2.756566 | −1.29656 | 2.530234 | 0.469825 | | |
| 322 | hsa-miR-144-5p | −3.79503 | 5.828154 | −1.29535 | 4.944213 | 0.464092 | | |
| 323 | hsa-miR-627 | −2.43202 | 3.613617 | −0.86679 | 3.146146 | 0.463101 | | |
| 324 | hsa-miR-155-5p | −0.47891 | 1.182446 | −0.07125 | 0.578678 | 0.462958 | | |
| 325 | hsa-miR-296-5p | −1.38203 | 1.537312 | −0.65336 | 1.687331 | 0.451941 | | |
| 326 | hsa-miR-425-3p | 1.264991 | 2.083319 | 0.430835 | 1.608424 | 0.451904 | | |
| 327 | hsa-miR-301b | −2.4864 | 3.323783 | −1.0634 | 2.997112 | 0.450252 | | |
| 328 | hsa-miR-550a-5p | 1.087227 | 2.096641 | 0.251937 | 1.6378 | 0.447344 | | |
| 329 | hsa-let-7b-3p | 1.180342 | 2.188518 | 0.290966 | 1.80281 | 0.445654 | | |
| 330 | hsa-miR-1294 | 2.086686 | 3.31736 | 0.952298 | 1.992986 | 0.427237 | | |
| 331 | hsa-miR-483-5p | −3.20945 | 3.591957 | −1.56471 | 4.149657 | 0.424909 | | |
| 332 | hsa-miR-1275 | −0.06598 | 0.60499 | −0.52069 | 1.550267 | 0.421946 | | |
| 333 | hsa-miR-1224-5p | −0.61946 | 3.368546 | 0.245534 | 0.739157 | 0.421159 | | |
| 334 | hsa-miR-1246 | −0.68756 | 2.175805 | −1.60292 | 2.311889 | 0.407941 | | |
| 335 | hsa-miR-542-5p | −2.11264 | 3.468371 | −0.65938 | 3.678667 | 0.406676 | | |
| 336 | hsa-miR-15a-3p | −1.11965 | 2.26364 | −0.3021 | 1.843853 | 0.398076 | | |
| 337 | hsa-miR-663a | −2.70759 | 3.058807 | −1.35057 | 3.811537 | 0.395038 | | |
| 338 | hsa-miR-96-5p | −3.15741 | 4.786161 | −1.49909 | 3.647229 | 0.393275 | | |
| 339 | hsa-miR-29b-1-5p | 0.618364 | 1.00842 | 0.24216 | 0.934295 | 0.387297 | | |
| 340 | hsa-miR-876-3p | 2.580118 | 3.339418 | 1.300213 | 3.352852 | 0.382503 | | |
| 341 | hsa-miR-542-3p | −1.53565 | 3.733328 | −0.30233 | 2.839433 | 0.375282 | | |
| 342 | hsa-miR-320c | 1.328151 | 1.479988 | 0.758696 | 1.633588 | 0.365789 | | |
| 343 | hsa-miR-92b-3p | 0.701755 | 3.987748 | −0.42055 | 2.187272 | 0.363499 | | |
| 344 | hsa-miR-222-3p | 1.28283 | 1.780216 | 0.689848 | 1.486661 | 0.363027 | | |
| 345 | hsa-miR-146a-5p | 1.128504 | 1.736584 | 0.586956 | 1.25729 | 0.36177 | | |
| 346 | hsa-miR-432-5p | 1.686465 | 2.456291 | 0.745157 | 2.791798 | 0.358724 | | |
| 347 | hsa-miR-629-5p | −2.28114 | 3.460359 | −1.22721 | 2.438238 | 0.357349 | | |
| 348 | hsa-miR-150-3p | −3.05205 | 2.759784 | −1.93779 | 3.569689 | 0.352087 | | |
| 349 | hsa-miR-494 | −0.25353 | 1.905204 | 0.301531 | 1.301368 | 0.346205 | | |
| 350 | hsa-miR-30a-5p | −0.29894 | 1.881063 | −0.89216 | 1.603778 | 0.340457 | | |
| 351 | hsa-miR-501-5p | 0.800112 | 2.059136 | 1.506608 | 2.156761 | 0.335158 | | |
| 352 | hsa-miR-181c-3p | −1.10335 | 2.155438 | −0.44375 | 1.781644 | 0.33507 | | |
| 353 | hsa-miR-20a-5p | −0.14006 | 0.354009 | −0.05556 | 0.152939 | 0.333377 | | |
| 354 | hsa-miR-181d | 0.036609 | 1.269049 | −0.54637 | 2.265316 | 0.329892 | | |
| 355 | hsa-miR-345-5p | −0.70868 | 2.520935 | 0.019193 | 1.95122 | 0.325512 | | |
| 356 | hsa-miR-362-3p | −3.20974 | 4.138484 | −1.80309 | 4.557966 | 0.323498 | | |
| 357 | hsa-miR-23a-5p | −0.39255 | 2.540507 | 0.411449 | 2.594012 | 0.313176 | | |
| 358 | hsa-miR-107 | 0.762279 | 0.990972 | 0.423129 | 1.212722 | 0.307801 | | |
| 359 | hsa-miR-181b-5p | 0.5171 | 0.782726 | 0.251163 | 0.975957 | 0.302427 | | |
| 360 | hsa-miR-20a-3p | −3.13358 | 4.053548 | −1.86613 | 4.38415 | 0.300427 | | |
| 361 | hsa-miR-186-5p | 1.138451 | 1.461685 | 0.690655 | 1.553628 | 0.297015 | | |
| 362 | hsa-miR-193b-3p | −1.54611 | 3.665332 | −0.61868 | 2.600331 | 0.296035 | | |
| 363 | hsa-miR-574-3p | −0.00834 | 0.928762 | −0.20742 | 0.449742 | 0.288837 | | |
| 364 | hsa-miR-760 | 2.115634 | 1.97618 | 1.589355 | 1.681783 | 0.287744 | | |
| 365 | hsa-miR-140-5p | 0.302015 | 0.882988 | 0.5586 | 0.921371 | 0.284405 | | |
| 366 | hsa-miR-221-3p | 0.833492 | 1.725422 | 0.351979 | 1.683093 | 0.282535 | | |
| 367 | hsa-miR-92a-3p | 0.061104 | 0.468892 | −0.02363 | 0.146736 | 0.275281 | | |
| 368 | hsa-let-7f-1-3p | 1.110498 | 2.450502 | 0.595555 | 1.316128 | 0.273424 | | |
| 369 | hsa-miR-212-3p | −2.22976 | 3.264003 | −1.45688 | 2.395792 | 0.27311 | | |
| 370 | hsa-let-7f-5p | −0.77704 | 0.92763 | −0.53868 | 0.822112 | 0.272454 | | |
| 371 | hsa-miR-1914-3p | −3.53853 | 4.91357 | −2.19157 | 5.004069 | 0.271629 | | |
| 372 | hsa-miR-1825 | 1.12521 | 2.541844 | 0.578224 | 1.536218 | 0.268258 | | |
| 373 | hsa-let-7g-5p | −0.56544 | 0.95391 | −0.33118 | 0.881273 | 0.255304 | | |
| 374 | hsa-miR-1305 | −3.57207 | 4.945917 | −2.32712 | 4.925916 | 0.252222 | | |
| 375 | hsa-miR-634 | 1.96667 | 3.009501 | 1.239876 | 2.787823 | 0.250734 | | |
| 376 | hsa-miR-331-3p | 0.298743 | 0.478725 | 0.15804 | 0.658058 | 0.247545 | | |
| 377 | hsa-miR-451a | −0.36446 | 1.462645 | 0.007332 | 1.54885 | 0.246917 | | |
| 378 | hsa-miR-194-5p | −3.25449 | 4.384355 | −2.18277 | 4.314935 | 0.246392 | | |
| 379 | hsa-miR-106b-5p | −0.48673 | 0.851158 | −0.28722 | 0.794971 | 0.242401 | | |
| 380 | hsa-miR-572 | −0.11764 | 1.76539 | −0.42176 | 0.772663 | 0.239651 | | |
| 381 | hsa-miR-379-5p | 0.791534 | 2.081223 | 0.310637 | 1.932821 | 0.239607 | | |
| 382 | hsa-miR-154-5p | 0.958627 | 1.86612 | 0.546034 | 1.635098 | 0.235685 | | |
| 383 | hsa-let-7d-5p | 0.739733 | 1.100571 | 0.461647 | 1.272212 | 0.234396 | | |
| 384 | hsa-miR-146b-5p | −0.00672 | 0.59671 | −0.10996 | 0.328858 | 0.223083 | | |
| 385 | hsa-let-7i-5p | −0.49148 | 0.80892 | −0.32394 | 0.707307 | 0.221004 | | |
| 386 | hsa-miR-99b-5p | −1.37915 | 3.115689 | −0.85436 | 1.780877 | 0.214351 | | |
| 387 | hsa-miR-7-1-3p | −3.66728 | 4.976572 | −2.63328 | 4.759686 | 0.2124 | | |
| 388 | hsa-miR-139-5p | −1.04237 | 2.80321 | −1.63393 | 2.815386 | 0.210572 | | |
| 389 | hsa-miR-335-5p | −2.88269 | 4.275335 | −2.01499 | 4.050347 | 0.20844 | | |
| 390 | hsa-miR-183-5p | −0.82319 | 3.526812 | −0.25862 | 1.957947 | 0.205868 | | |
| 391 | hsa-let-7a-5p | 0.259276 | 0.756307 | 0.444343 | 1.077335 | 0.201857 | | |
| 392 | hsa-miR-664-3p | −1.01138 | 3.359845 | −1.64407 | 2.975777 | 0.199724 | | |
| 393 | hsa-miR-34b-5p | 0.808641 | 2.775781 | 0.332936 | 2.037033 | 0.197683 | | |
| 394 | hsa-miR-21-3p | 0.533775 | 0.771849 | 0.696674 | 0.90077 | 0.194783 | | |
| 395 | hsa-miR-423-5p | −3.71499 | 4.891737 | −2.7936 | 4.659542 | 0.192935 | | |
| 396 | hsa-miR-628-5p | −1.80397 | 3.097134 | −1.22591 | 2.961516 | 0.190821 | | |
| 397 | hsa-miR-29a-3p | −0.3431 | 0.835368 | −0.50343 | 0.857346 | 0.189432 | | |
| 398 | hsa-miR-200b-3p | −1.82477 | 2.746965 | −1.33703 | 2.490162 | 0.186266 | | |
| 399 | hsa-miR-376b | 1.101289 | 1.646874 | 0.77754 | 1.875396 | 0.18383 | | |
| 400 | hsa-miR-199a-3p | −0.07745 | 0.889408 | 0.098151 | 1.026596 | 0.183299 | | |
| 401 | hsa-miR-505-5p | −2.22447 | 3.121873 | −1.68646 | 2.921086 | 0.178061 | | |
| 402 | hsa-miR-671-5p | −2.05438 | 4.718371 | −1.27132 | 4.164059 | 0.176317 | | |
| 403 | hsa-miR-139-3p | −0.91694 | 2.79443 | −0.4435 | 2.65029 | 0.173908 | | |
| 404 | hsa-miR-624-5p | −1.37267 | 2.698812 | −1.02335 | 1.464817 | 0.167797 | | |
| 405 | hsa-miR-129-1-3p | 0.668339 | 3.420887 | 0.113211 | 3.232738 | 0.166865 | | |
| 406 | hsa-miR-16-2-3p | −2.46337 | 3.842737 | −1.86535 | 3.468504 | 0.16359 | | |
| 407 | hsa-miR-145-5p | −1.77804 | 3.293356 | −2.22128 | 2.152312 | 0.162785 | | |
| 408 | hsa-miR-630 | −1.3341 | 4.460925 | −0.81358 | 1.961734 | 0.16209 | | |
| 409 | hsa-miR-133b | 0.347828 | 3.258209 | 0.840931 | 2.847378 | 0.161525 | | |
| 410 | hsa-miR-431-5p | 0.966645 | 2.126756 | 0.640928 | 1.974952 | 0.15882 | | |
| 411 | hsa-let-7b-5p | 0.854819 | 1.204541 | 0.656172 | 1.312625 | 0.157834 | | |
| 412 | hsa-miR-543 | 0.686506 | 1.910423 | 0.375274 | 2.044105 | 0.157406 | | |
| 413 | hsa-miR-181a-3p | −2.7222 | 4.041121 | −2.12647 | 3.553019 | 0.156891 | | |
| 414 | hsa-miR-15b-3p | −0.54458 | 2.186429 | −0.82235 | 1.364557 | 0.156446 | | |
| 415 | hsa-miR-190b | 3.083119 | 4.776007 | 2.38654 | 4.149311 | 0.156091 | | |
| 416 | hsa-miR-330-3p | −1.81396 | 3.620399 | −1.29455 | 3.118402 | 0.154155 | | |
| 417 | hsa-miR-582-3p | 0.526743 | 1.830603 | 0.821323 | 2.016605 | 0.15314 | | |
| 418 | hsa-miR-30d-5p | −0.10287 | 0.191318 | −0.12823 | 0.142001 | 0.152133 | | |
| 419 | hsa-miR-223-3p | −0.65302 | 1.129143 | −0.48464 | 1.090426 | 0.151724 | | |
| 420 | hsa-miR-126-3p | −0.48393 | 0.357533 | −0.40923 | 0.632976 | 0.150825 | | |
| 421 | hsa-let-7e-5p | 0.48629 | 1.411062 | 0.747855 | 2.115797 | 0.148327 | | |
| 422 | hsa-miR-532-3p | −2.47835 | 3.558184 | −2.00081 | 3.324734 | 0.138763 | | |
| 423 | hsa-miR-23a-3p | −0.80881 | 0.955194 | −0.6729 | 1.017962 | 0.137753 | | |
| 424 | hsa-miR-30d-3p | −0.28383 | 1.469114 | −0.49395 | 1.647235 | 0.13485 | | |
| 425 | hsa-miR-186-3p | 1.107339 | 3.444947 | 1.537191 | 3.158388 | 0.130192 | | |
| 426 | hsa-miR-377-5p | 1.25903 | 3.555861 | 1.696786 | 3.2276 | 0.129066 | | |
| 427 | hsa-miR-623 | 0.925035 | 3.507421 | 1.344767 | 3.172136 | 0.125677 | | |
| 428 | hsa-miR-501-3p | −0.22358 | 2.46912 | −0.48689 | 1.811012 | 0.123041 | | |
| 429 | hsa-miR-22-5p | −2.5651 | 4.064847 | −2.11133 | 3.487385 | 0.120168 | | |
| 430 | hsa-miR-103a-3p | 0.503679 | 0.988108 | 0.374387 | 1.24984 | 0.115545 | | |
| 431 | hsa-miR-664-5p | −1.90045 | 2.580377 | −1.6116 | 2.437099 | 0.115135 | | |
| 432 | hsa-miR-130b-3p | 0.529246 | 0.803921 | 0.636302 | 1.269926 | 0.103244 | | |
| 433 | hsa-miR-378a-5p | −1.18528 | 3.152946 | −0.86741 | 3.009646 | 0.103162 | | |
| 434 | hsa-miR-125a-5p | −2.59129 | 3.72498 | −2.24025 | 3.169899 | 0.101827 | | |
| 435 | hsa-miR-1295a | 2.55578 | 2.823912 | 2.237356 | 4.157492 | 0.091221 | | |
| 436 | hsa-miR-381 | −0.42539 | 2.283614 | −0.11128 | 4.716474 | 0.089746 | | |
| 437 | hsa-miR-574-5p | 0.070778 | 1.203672 | 0.007128 | 0.229651 | 0.088814 | | |
| 438 | hsa-miR-320b | 0.609585 | 0.834529 | 0.539566 | 0.787158 | 0.086354 | | |
| 439 | hsa-miR-224-5p | −1.15377 | 1.189352 | −1.35396 | 3.660703 | 0.08255 | | |
| 440 | hsa-miR-202-3p | 0.817797 | 2.678342 | 1.013055 | 2.201857 | 0.080021 | | |
| 441 | hsa-miR-30c-1-3p | −0.21597 | 1.278423 | −0.33083 | 1.595168 | 0.079945 | | |
| 442 | hsa-miR-299-3p | 1.959681 | 3.735819 | 1.641907 | 4.217561 | 0.079909 | | |
| 443 | hsa-miR-188-5p | 0.076817 | 1.012454 | 0.001868 | 0.907193 | 0.078087 | | |
| 444 | hsa-miR-513a-5p | 0.347526 | 0.301323 | 0.420591 | 1.596179 | 0.077013 | | |
| 445 | hsa-miR-410 | −1.4495 | 0.514358 | −1.62516 | 4.188034 | 0.074711 | | |
| 446 | hsa-miR-1267 | 0.63107 | 3.985315 | 0.416081 | 1.838594 | 0.07383 | | |
| 447 | hsa-miR-132-5p | −2.30624 | 2.77761 | −2.10267 | 2.835077 | 0.072542 | | |
| 448 | hsa-miR-629-3p | −1.4592 | 2.144475 | −1.59253 | 1.55434 | 0.072093 | | |
| 449 | hsa-miR-320d | 0.488425 | 0.816299 | 0.543863 | 0.819158 | 0.067796 | | |
| 450 | hsa-miR-1271-5p | −1.18245 | 2.730096 | −1.03713 | 2.160764 | 0.059425 | | |
| 451 | hsa-miR-130a-3p | 0.42432 | 1.325263 | 0.512193 | 1.923677 | 0.054093 | | |
| 452 | hsa-miR-1202 | −0.84455 | 1.6033 | −0.76007 | 1.540564 | 0.053742 | | |
| 453 | hsa-miR-1225-5p | 0.187024 | 0.710061 | 0.147455 | 0.780693 | 0.053087 | | |
| 454 | hsa-miR-377-3p | −2.36517 | 3.77778 | −2.17639 | 3.61056 | 0.051102 | | |
| 455 | hsa-miR-1471 | −0.04378 | 4.742826 | 0.102249 | 1.168682 | 0.049406 | | |
| 456 | hsa-miR-92a-1-5p | 0.424525 | 0.86185 | 0.35335 | 2.068748 | 0.048574 | | |
| 457 | hsa-miR-493-5p | −0.65147 | 1.106977 | −0.76479 | 3.947007 | 0.044843 | | |
| 458 | hsa-miR-34a-5p | 0.797093 | 1.529786 | 0.865369 | 1.525975 | 0.044687 | | |
| 459 | hsa-miR-100-5p | −1.22871 | 3.968866 | −1.06984 | 3.734941 | 0.041247 | | |
| 460 | hsa-miR-337-5p | 1.043103 | 1.872902 | 1.137214 | 2.830918 | 0.040015 | | |
| 461 | hsa-miR-495 | −2.27875 | 3.605861 | −2.41228 | 3.214964 | 0.039153 | | |
| 462 | hsa-miR-143-3p | 0.288563 | 2.737132 | 0.20429 | 1.749909 | 0.037563 | | |
| 463 | hsa-miR-382-5p | 0.205856 | 2.178827 | 0.101603 | 3.708148 | 0.035418 | | |
| 464 | hsa-miR-1290 | −1.99372 | 2.635023 | −1.89669 | 3.519258 | 0.031532 | | |
| 465 | hsa-miR-1183 | 0.312768 | 4.402878 | 0.253212 | 0.904968 | 0.022441 | | |
| 466 | hsa-miR-24-3p | −0.56401 | 0.563673 | −0.57861 | 0.756204 | 0.022124 | | |
| 467 | hsa-miR-181a-2-3p | −0.63007 | 2.170118 | −0.5991 | 1.542299 | 0.016685 | | |
| 468 | hsa-miR-1226-5p | −0.79908 | 3.492881 | −0.74038 | 3.712035 | 0.016293 | | |
| 469 | hsa-miR-135a-3p | −1.89656 | 2.808929 | −1.92692 | 3.67155 | 0.00937 | | |
| 470 | hsa-miR-1181 | −1.45154 | 3.340725 | −1.42644 | 3.763091 | 0.007067 | | |
| 471 | hsa-miR-30b-3p | −1.3847 | 2.502301 | −1.38795 | 1.971448 | 0.001449 | | |

TABLE 3A
HC ratio calculations for pregnancy outcome prediction using 471 microRNAs from pregnant Blacks (See full version of table, Table 3B)

| | | | | | | | | (Column R) |
|---|---|---|---|---|---|---|---|---|
| | | | | | | (Column P) | | Shared |
| | | | | (Column N) | | \|HC ratio = | | microRNA with |
| (Column J) | | (Column L) | | Mean | | Compromised | (Column Q) | self-identified |
| Ratio | (Column K) | Mean Healthy | (Column M) | Compromised | (Column O) | minus Healthy/ | XX = \|HC | Non-Black |
| order | 471 MicroRNAs | BLACK | SD | BLACK | SD | (mean SD)\| | Ratio\| ≥ 1.5 | persons |
| 1 | hsa-miR-1267 | 1.008133 | 1.572933 | 3.68654 | 0.457825 | 2.63784 | xx | |
| 2 | hsa-miR-452-5p | 0.1 | 1.70E−17 | 3.464422 | 3.210483 | 2.095898 | xx | |
| 3 | hsa-miR-580 | 0.1 | 1.70E−17 | 2.18256 | 2.017471 | 2.064526 | xx | |
| 4 | hsa-miR-455-5p | 0.1 | 1.70E−17 | 3.704497 | 3.790497 | 1.90186 | xx | |
| 5 | hsa-miR-149-5p | 5.636273 | 0.432961 | 6.99795 | 1.008194 | 1.889703 | xx | |
| 6 | hsa-miR-18b-3p | 1.635137 | 0.24768 | 6.01141 | 4.468984 | 1.855665 | xx | x |
| 7 | hsa-miR-1539 | 5.78433 | 0.309926 | 7.177102 | 1.213145 | 1.828899 | xx | |
| 8 | hsa-miR-19a-5p | 0.1 | 1.70E−17 | 1.842585 | 1.940627 | 1.795899 | xx | |
| 9 | hsa-miR-190a | 0.608143 | 0.88013 | 14.27126 | 14.78784 | 1.744083 | xx | |
| 10 | hsa-miR-144-3p | 1298.11 | 330.7328 | 11483.14 | 11487.35 | 1.723634 | xx | |
| 11 | hsa-miR-548a- | 1.354785 | 1.533526 | 9.410058 | 7.952339 | 1.698374 | xx | |
| 12 | hsa-miR-32-5p | 54.08237 | 23.16953 | 401.513 | 400.6363 | 1.639574 | xx | |
| 13 | hsa-miR-186-3p | 2.292743 | 1.932089 | 8.987733 | 6.361054 | 1.614585 | xx | |
| 14 | hsa-miR-33b-5p | 1.265057 | 0.270846 | 8.21405 | 8.417689 | 1.599578 | xx | |
| 15 | hsa-miR-301a- | 372.5503 | 200.0697 | 1408.847 | 1121.465 | 1.568323 | xx | |
| 16 | hsa-miR-624-3p | 0.50149 | 0.695401 | 3.68011 | 3.380816 | 1.559593 | xx | |
| 17 | hsa-miR-590-5p | 580.1497 | 173.8664 | 2564.739 | 2375.112 | 1.557164 | xx | x |
| 18 | hsa-miR-191-3p | 6.86531 | 0.503358 | 8.409832 | 1.502668 | 1.539882 | xx | |
| 19 | hsa-miR-24-1-5p | 8.011073 | 4.359647 | 23.77917 | 16.20986 | 1.533153 | xx | |
| 20 | hsa-miR-144-5p | 313.3803 | 34.05616 | 1573.889 | 1614.138 | 1.529564 | xx | |
| 21 | hsa-miR-504 | 90.202 | 48.89069 | 44.30585 | 12.21383 | 1.502218 | xx | |
| 22 | hsa-miR-33a-5p | 34.4442 | 5.488233 | 229.4273 | 254.9026 | 1.497619 | xx | |
| 23 | hsa-miR-545-3p | 9.49608 | 2.191043 | 76.41258 | 87.51856 | 1.491847 | xx | |
| 24 | hsa-miR-19a-3p | 3543.693 | 2022.001 | 12238.33 | 9679.308 | 1.486096 | xx | |
| 25 | hsa-miR-551b- | 44.84363 | 17.70073 | 117.1409 | 80.33489 | 1.474918 | xx | |
| 26 | hsa-miR-141-3p | 82.59723 | 29.12215 | 313.6675 | 286.7378 | 1.463119 | xx | |
| 27 | hsa-miR-557 | 18.01197 | 2.037169 | 27.32418 | 10.77519 | 1.45363 | xx | |
| 28 | hsa-miR-101-3p | 2065.382 | 1232.121 | 7598.832 | 6393.937 | 1.451196 | xx | |
| 29 | hsa-miR-340-5p | 448.0383 | 415.4183 | 2036.804 | 1794.695 | 1.437723 | | |
| 30 | hsa-miR-628-5p | 45.10693 | 18.46749 | 106.8211 | 68.09535 | 1.425881 | | |
| 31 | hsa-miR-192-5p | 606.7603 | 183.9096 | 1559.788 | 1162.925 | 1.415211 | | |
| 32 | hsa-miR-362-3p | 167.9153 | 137.3452 | 700.0322 | 616.2536 | 1.412202 | | |
| 33 | hsa-miR-493-3p | 1.751764 | 1.205592 | 4.331805 | 2.485494 | 1.397985 | | |
| 34 | hsa-miR-193a- | 37.70363 | 32.35907 | 210.8888 | 216.6352 | 1.391078 | | |
| 35 | hsa-miR-18b-5p | 260.0957 | 143.133 | 689.5827 | 478.7648 | 1.381214 | | |
| 36 | hsa-miR-224-5p | 19.39167 | 15.47242 | 64.07994 | 49.30735 | 1.379698 | | |
| 37 | hsa-miR-132-3p | 206.0875 | 117.5854 | 963.7525 | 995.8154 | 1.360992 | | |
| 38 | hsa-miR-7-5p | 450.8163 | 230.8244 | 1296.672 | 1030.427 | 1.341296 | | |
| 39 | hsa-miR-129-1- | 1.873213 | 0.429905 | 3.094062 | 1.39485 | 1.338096 | | |
| 40 | hsa-miR-520f | 54.28884 | 47.80037 | 15.75792 | 10.47201 | 1.322442 | | |
| 41 | hsa-miR-582-3p | 5.582067 | 3.783832 | 20.0183 | 18.19959 | 1.313374 | | |
| 42 | hsa-miR-30d-3p | 12.78553 | 8.461298 | 39.27953 | 32.0727 | 1.307248 | | |
| 43 | hsa-miR-429 | 2.866653 | 4.791984 | 15.9349 | 15.25821 | 1.303553 | | |
| 44 | hsa-miR-542-3p | 34.54687 | 24.16941 | 117.974 | 104.4413 | 1.297358 | | |
| 45 | hsa-miR-185-5p | 1269.566 | 585.6775 | 3061.072 | 2177.344 | 1.296773 | | |
| 46 | hsa-miR-138-2- | 32.70445 | 24.67982 | 13.24969 | 5.467858 | 1.29063 | | |
| 47 | hsa-miR-296-5p | 15.18167 | 3.465018 | 29.16082 | 18.29344 | 1.284939 | | |
| 48 | hsa-miR-431-3p | 1.089472 | 0.487798 | 4.277557 | 4.47638 | 1.284436 | | |
| 49 | hsa-miR-29b-3p | 6179.653 | 2723.419 | 17123.35 | 14393.02 | 1.278735 | | |
| 50 | hsa-miR-1306- | 27.9432 | 4.481779 | 21.78588 | 5.159936 | 1.277224 | | |
| 51 | hsa-miR-106a- | 3.68549 | 2.72496 | 10.98227 | 8.704763 | 1.276808 | | |
| 52 | hsa-miR-212-3p | 28.9425 | 10.43888 | 117.0236 | 127.7442 | 1.274846 | | |
| 53 | hsa-miR-425-3p | 7.408413 | 1.0114 | 8.900028 | 1.334484 | 1.271687 | | |
| 54 | hsa-miR-34a-3p | 1.130767 | 1.78534 | 7.973982 | 8.998675 | 1.26914 | | |
| 55 | hsa-miR-154-3p | 13.95673 | 13.24418 | 71.19688 | 77.02247 | 1.268246 | | |
| 56 | hsa-miR-34c-5p | 0.806307 | 1.223359 | 4.499645 | 4.645382 | 1.258648 | | |
| 57 | hsa-miR-200a- | 10.41362 | 6.002765 | 29.60447 | 24.49442 | 1.258533 | | |
| 58 | hsa-miR-96-5p | 96.03277 | 15.5637 | 364.4783 | 412.0578 | 1.255528 | | |

TABLE 3B
HC ratio calculations for pregnancy outcome prediction using 471 microRNAs from pregnant Blacks (full version)

| | | | | | | | | (Column R) |
|---|---|---|---|---|---|---|---|---|
| | | | | | | | | Shared |
| | | | | | | (Column P) | | microRNA |
| | | | | (Column N) | | \|HC ratio = | | with self- |
| | | (Column L) | | Mean | | Compromised | (Column Q) | identified |
| (Column J) | (Column K) | Mean Healthy | (Column M) | Compromised | (Column O) | minus Healthy/ | XX = \|HC | Non-Black |
| Ratio order | 471 MicroRNAs | BLACK | SD | BLACK | SD | (mean SD)\| | Ratio\| ≥ 1.5 | persons |
| 1 | hsa-miR-1267 | 1.008133 | 1.572933 | 3.68654 | 0.457825 | 2.63784 | xx | |
| 2 | hsa-miR-452-5p | 0.1 | 1.70E−17 | 3.464422 | 3.210483 | 2.095898 | xx | |
| 3 | hsa-miR-580 | 0.1 | 1.70E−17 | 2.18256 | 2.017471 | 2.064526 | xx | |
| 4 | hsa-miR-455-5p | 0.1 | 1.70E−17 | 3.704497 | 3.790497 | 1.90186 | xx | |
| 5 | hsa-miR-149-5p | 5.636273 | 0.432961 | 6.99795 | 1.008194 | 1.889703 | xx | |
| 6 | hsa-miR-18b-3p | 1.635137 | 0.24768 | 6.01141 | 4.468984 | 1.855665 | xx | x |
| 7 | hsa-miR-1539 | 5.78433 | 0.309926 | 7.177102 | 1.213145 | 1.828899 | xx | |
| 8 | hsa-miR-19a-5p | 0.1 | 1.70E−17 | 1.842585 | 1.940627 | 1.795899 | xx | |
| 9 | hsa-miR-190a | 0.608143 | 0.88013 | 14.27126 | 14.78784 | 1.744083 | xx | |
| 10 | hsa-miR-144-3p | 1298.11 | 330.7328 | 11483.14 | 11487.35 | 1.723634 | xx | |
| 11 | hsa-miR-548a-5p | 1.354785 | 1.533526 | 9.410058 | 7.952339 | 1.698374 | xx | |
| 12 | hsa-miR-32-5p | 54.08237 | 23.16953 | 401.513 | 400.6363 | 1.639574 | xx | |
| 13 | hsa-miR-186-3p | 2.292743 | 1.932089 | 8.987733 | 6.361054 | 1.614585 | xx | |
| 14 | hsa-miR-33b-5p | 1.265057 | 0.270846 | 8.21405 | 8.417689 | 1.599578 | xx | |
| 15 | hsa-miR-301a-3p | 372.5503 | 200.0697 | 1408.847 | 1121.465 | 1.568323 | xx | |
| 16 | hsa-miR-624-3p | 0.50149 | 0.695401 | 3.68011 | 3.380816 | 1.559593 | xx | |
| 17 | hsa-miR-590-5p | 580.1497 | 173.8664 | 2564.739 | 2375.112 | 1.557164 | xx | x |
| 18 | hsa-miR-191-3p | 6.86531 | 0.503358 | 8.409832 | 1.502668 | 1.539882 | xx | |
| 19 | hsa-miR-24-1-5p | 8.011073 | 4.359647 | 23.77917 | 16.20986 | 1.533153 | xx | |
| 20 | hsa-miR-144-5p | 313.3803 | 34.05616 | 1573.889 | 1614.138 | 1.529564 | xx | |
| 21 | hsa-miR-504 | 90.202 | 48.89069 | 44.30585 | 12.21383 | 1.502218 | xx | |
| 22 | hsa-miR-33a-5p | 34.4442 | 5.488233 | 229.4273 | 254.9026 | 1.497619 | xx | |
| 23 | hsa-miR-545-3p | 9.49608 | 2.191043 | 76.41258 | 87.51856 | 1.491847 | xx | |
| 24 | hsa-miR-19a-3p | 3543.693 | 2022.001 | 12238.33 | 9679.308 | 1.486096 | xx | |
| 25 | hsa-miR-551b-3p | 44.84363 | 17.70073 | 117.1409 | 80.33489 | 1.474918 | xx | |
| 26 | hsa-miR-141-3p | 82.59723 | 29.12215 | 313.6675 | 286.7378 | 1.463119 | xx | |
| 27 | hsa-miR-557 | 18.01197 | 2.037169 | 27.32418 | 10.77519 | 1.45363 | xx | |
| 28 | hsa-miR-101-3p | 2065.382 | 1232.121 | 7598.832 | 6393.937 | 1.451196 | xx | |
| 29 | hsa-miR-340-5p | 448.0383 | 415.4183 | 2036.804 | 1794.695 | 1.437723 | | |
| 30 | hsa-miR-628-5p | 45.10693 | 18.46749 | 106.8211 | 68.09535 | 1.425881 | | |
| 31 | hsa-miR-192-5p | 606.7603 | 183.9096 | 1559.788 | 1162.925 | 1.415211 | | |
| 32 | hsa-miR-362-3p | 167.9153 | 137.3452 | 700.0322 | 616.2536 | 1.412202 | | |
| 33 | hsa-miR-493-3p | 1.751764 | 1.205592 | 4.331805 | 2.485494 | 1.397985 | | |
| 34 | hsa-miR-193a-3p | 37.70363 | 32.35907 | 210.8888 | 216.6352 | 1.391078 | | |
| 35 | hsa-miR-18b-5p | 260.0957 | 143.133 | 689.5827 | 478.7648 | 1.381214 | | |
| 36 | hsa-miR-224-5p | 19.39167 | 15.47242 | 64.07994 | 49.30735 | 1.379698 | | |
| 37 | hsa-miR-132-3p | 206.0875 | 117.5854 | 963.7525 | 995.8154 | 1.360992 | | |
| 38 | hsa-miR-7-5p | 450.8163 | 230.8244 | 1296.672 | 1030.427 | 1.341296 | | |
| 39 | hsa-miR-129-1-3p | 1.873213 | 0.429905 | 3.094062 | 1.39485 | 1.338096 | | |
| 40 | hsa-miR-520f | 54.28884 | 47.80037 | 15.75792 | 10.47201 | 1.322442 | | |
| 41 | hsa-miR-582-3p | 5.582067 | 3.783832 | 20.0183 | 18.19959 | 1.313374 | | |
| 42 | hsa-miR-30d-3p | 12.78553 | 8.461298 | 39.27953 | 32.0727 | 1.307248 | | |
| 43 | hsa-miR-429 | 2.866653 | 4.791984 | 15.9349 | 15.25821 | 1.303553 | | |
| 44 | hsa-miR-542-3p | 34.54687 | 24.16941 | 117.974 | 104.4413 | 1.297358 | | |
| 45 | hsa-miR-185-5p | 1269.566 | 585.6775 | 3061.072 | 2177.344 | 1.296773 | | |
| 46 | hsa-miR-138-2-3p | 32.70445 | 24.67982 | 13.24969 | 5.467858 | 1.29063 | | |
| 47 | hsa-miR-296-5p | 15.18167 | 3.465018 | 29.16082 | 18.29344 | 1.284939 | | |
| 48 | hsa-miR-431-3p | 1.089472 | 0.487798 | 4.277557 | 4.47638 | 1.284436 | | |
| 49 | hsa-miR-29b-3p | 6179.653 | 2723.419 | 17123.35 | 14393.02 | 1.278735 | | |
| 50 | hsa-miR-1306-3p | 27.9432 | 4.481779 | 21.78588 | 5.159936 | 1.277224 | | |
| 51 | hsa-miR-106a-3p | 3.68549 | 2.72496 | 10.98227 | 8.704763 | 1.276808 | | |
| 52 | hsa-miR-212-3p | 28.9425 | 10.43888 | 117.0236 | 127.7442 | 1.274846 | | |
| 53 | hsa-miR-425-3p | 7.408413 | 1.0114 | 8.900028 | 1.334484 | 1.271687 | | |
| 54 | hsa-miR-34a-3p | 1.130767 | 1.78534 | 7.973982 | 8.998675 | 1.26914 | | |
| 55 | hsa-miR-154-3p | 13.95673 | 13.24418 | 71.19688 | 77.02247 | 1.268246 | | |
| 56 | hsa-miR-34c-5p | 0.806307 | 1.223359 | 4.499645 | 4.645382 | 1.258648 | | |
| 57 | hsa-miR-200a-3p | 10.41362 | 6.002765 | 29.60447 | 24.49442 | 1.258533 | | |
| 58 | hsa-miR-96-5p | 96.03277 | 15.5637 | 364.4783 | 412.0578 | 1.255528 | | |
| 59 | hsa-miR-454-3p | 328.032 | 196.4543 | 818.4815 | 586.4243 | 1.252939 | | |
| 60 | hsa-let-7e-5p | 476.6923 | 187.7944 | 1068.093 | 756.4392 | 1.252657 | | |
| 61 | hsa-miR-106b-5p | 6006.483 | 2311.056 | 12892 | 8732.418 | 1.246984 | | |
| 62 | hsa-miR-18a-5p | 520.8037 | 411.4186 | 1460.614 | 1096.493 | 1.246506 | | |
| 63 | hsa-miR-19b-1-5p | 22.5342 | 15.00563 | 50.61987 | 30.11325 | 1.244963 | | |
| 64 | hsa-miR-1228-3p | 24.6391 | 2.439412 | 39.93022 | 22.33153 | 1.234601 | | |
| 65 | hsa-miR-582-5p | 352.3437 | 149.1587 | 956.4956 | 832.5654 | 1.230798 | | |
| 66 | hsa-miR-17-3p | 182.7591 | 158.9683 | 631.6045 | 577.5603 | 1.218813 | | |
| 67 | hsa-miR-15a-3p | 16.60062 | 12.37033 | 51.42005 | 44.79466 | 1.218208 | | |
| 68 | hsa-miR-33b-3p | 7.399523 | 1.220338 | 9.520732 | 2.277306 | 1.212936 | | |
| 69 | hsa-miR-301b | 36.3048 | 32.34816 | 104.8405 | 80.72308 | 1.212257 | | |
| 70 | hsa-miR-140-5p | 2016.63 | 845.7923 | 4172.938 | 2723.643 | 1.208207 | | |
| 71 | hsa-miR-449a | 6.908377 | 5.471758 | 18.89656 | 14.39584 | 1.206808 | | |
| 72 | hsa-miR-876-5p | 2.285097 | 1.904716 | 11.06494 | 12.68436 | 1.203618 | | |
| 73 | hsa-miR-190b | 10.81273 | 11.86103 | 2.19144 | 2.570878 | 1.194754 | | |
| 74 | hsa-miR-624-5p | 29.91903 | 18.34509 | 69.11202 | 47.4762 | 1.190891 | | |
| 75 | hsa-miR-132-5p | 76.46993 | 88.13096 | 307.9969 | 301.1394 | 1.189543 | | |
| 76 | hsa-miR-9-3p | 21.0064 | 11.61072 | 56.81661 | 48.78049 | 1.185941 | | |
| 77 | hsa-miR-34a-5p | 178.7823 | 63.4443 | 413.526 | 335.5427 | 1.176698 | | |
| 78 | hsa-miR-34b-5p | 19.37207 | 6.074508 | 50.10527 | 46.21896 | 1.175413 | | |
| 79 | hsa-miR-147b | 1.018407 | 0.80226 | 4.996193 | 5.969346 | 1.174843 | | |
| 80 | hsa-miR-660-5p | 570.891 | 363.1036 | 1346.169 | 956.8177 | 1.174733 | | |
| 81 | hsa-miR-30e-5p | 2725.333 | 1516.929 | 6889.431 | 5591.639 | 1.171571 | | |
| 82 | hsa-miR-148a-3p | 1182.201 | 737.4074 | 2979.048 | 2332.75 | 1.170524 | | |
| 83 | hsa-miR-1299 | 8.89661 | 3.14242 | 12.40008 | 2.890412 | 1.161468 | | |
| 84 | hsa-miR-588 | 11.26377 | 11.57469 | 2.839738 | 2.966298 | 1.15866 | | |
| 85 | hsa-miR-183-5p | 34.03443 | 4.937429 | 91.66865 | 95.1247 | 1.151969 | | |
| 86 | hsa-miR-136-5p | 32.0773 | 27.293 | 284.211 | 411.2908 | 1.149763 | | |
| 87 | hsa-miR-15a-5p | 9891.937 | 6427.276 | 23445.46 | 17245.85 | 1.145055 | | |
| 88 | hsa-miR-502-5p | 43.81267 | 31.14826 | 95.86398 | 59.95839 | 1.142646 | | |
| 89 | hsa-miR-199b-5p | 338.343 | 255.893 | 811.6319 | 580.7848 | 1.131353 | | |
| 90 | hsa-miR-99b-5p | 57.17727 | 38.97042 | 134.602 | 99.04643 | 1.121961 | | |
| 91 | hsa-miR-188-3p | 1.604447 | 2.605778 | 5.750668 | 4.790804 | 1.121118 | | |
| 92 | hsa-miR-20b-5p | 4036.763 | 1617.655 | 7830.154 | 5165.903 | 1.118407 | | |
| 93 | hsa-miR-376b | 15.278 | 19.61197 | 87.21086 | 109.2489 | 1.116442 | | |
| 94 | hsa-miR-487a | 6.881737 | 8.263959 | 29.99443 | 33.26955 | 1.112966 | | |
| 95 | hsa-miR-548am- | 45.5835 | 25.8876 | 102.223 | 75.95413 | 1.112305 | | |
| 96 | hsa-miR-20a-3p | 108.2719 | 65.14576 | 249.9249 | 190.1024 | 1.109924 | | |
| 97 | hsa-miR-424-5p | 777.7953 | 567.153 | 2010.216 | 1657.504 | 1.107965 | | |
| 98 | hsa-miR-579 | 3.250003 | 3.557073 | 9.962585 | 8.576222 | 1.106473 | | |
| 99 | hsa-miR-182-5p | 4.840547 | 3.598637 | 15.42556 | 15.54712 | 1.105729 | | |
| 100 | hsa-miR-1827 | 19.99206 | 11.71383 | 52.19957 | 46.64407 | 1.103792 | | |
| 101 | hsa-miR-99a-5p | 192.5437 | 83.75527 | 410.3311 | 311.0059 | 1.103388 | | |
| 102 | hsa-miR-627 | 47.19793 | 31.3448 | 110.24 | 84.1533 | 1.091656 | | |
| 103 | hsa-miR-10b-3p | 36.53927 | 23.94815 | 20.90535 | 4.793263 | 1.087902 | | |
| 104 | hsa-miR-1234 | 18.97527 | 1.528042 | 25.20087 | 9.921269 | 1.087506 | | |
| 105 | hsa-miR-29a-5p | 18.04698 | 10.71517 | 43.64147 | 36.50117 | 1.084137 | | |
| 106 | hsa-miR-20a-5p | 13056.53 | 3338.42 | 21784.01 | 12873.49 | 1.076674 | | |
| 107 | hsa-miR-126-5p | 220.0252 | 127.578 | 472.3843 | 343.0763 | 1.072376 | | |
| 108 | hsa-miR-208a | 7.56429 | 0.425428 | 12.12098 | 8.127838 | 1.065484 | | |
| 109 | hsa-miR-374a-5p | 3031.054 | 2300.906 | 7455.466 | 6070.472 | 1.057033 | | |
| 110 | hsa-miR-9-5p | 11.63566 | 8.634356 | 28.54157 | 23.62362 | 1.048169 | | |
| 111 | hsa-miR-369-3p | 2.061633 | 3.397649 | 9.124017 | 10.21743 | 1.037436 | | |
| 112 | hsa-miR-335-5p | 138.8968 | 117.5993 | 336.718 | 266.8738 | 1.029051 | | |
| 113 | hsa-miR-221-3p | 1028.044 | 346.0793 | 1618.203 | 801.2276 | 1.028773 | | |
| 114 | hsa-miR-548e | 10.16744 | 9.252776 | 26.0239 | 21.66414 | 1.025747 | | |
| 115 | hsa-miR-136-3p | 15.25555 | 20.76389 | 61.62085 | 70.29536 | 1.018354 | | |
| 116 | hsa-miR-373-5p | 5.762747 | 4.711466 | 10.56054 | 4.734051 | 1.015888 | | |
| 117 | hsa-miR-27a-5p | 1.917831 | 2.412213 | 5.828368 | 5.291689 | 1.01521 | | |
| 118 | hsa-miR-1270 | 1.192577 | 0.94622 | 2.783015 | 2.200341 | 1.010906 | | |
| 119 | hsa-miR-30a-3p | 4.677473 | 3.299333 | 8.699635 | 4.679869 | 1.008161 | | |
| 120 | hsa-miR-340-3p | 291.3493 | 244.5485 | 609.3466 | 392.086 | 0.998995 | | |
| 121 | hsa-miR-1294 | 0.1 | 1.70E−17 | 1.353907 | 2.517484 | 0.996159 | | |
| 122 | hsa-miR-324-5p | 402.1717 | 262.264 | 803.5611 | 545.1766 | 0.994227 | | |
| 123 | hsa-miR-298 | 21.42958 | 16.79502 | 9.558662 | 7.246907 | 0.987518 | | |
| 124 | hsa-miR-19b-3p | 11302.14 | 6955.939 | 23129.46 | 17102 | 0.983237 | | |
| 125 | hsa-miR-576-3p | 2.827263 | 3.503097 | 8.411562 | 7.923392 | 0.97743 | | |
| 126 | hsa-miR-218-5p | 1.815 | 1.594069 | 6.512625 | 8.045456 | 0.974659 | | |
| 127 | hsa-miR-501-5p | 17.60427 | 3.441415 | 23.14182 | 7.975945 | 0.970023 | | |
| 128 | hsa-miR-634 | 2.875107 | 1.312292 | 4.301303 | 1.634373 | 0.968007 | | |
| 129 | hsa-miR-195-3p | 4.45436 | 0.988187 | 10.65391 | 11.83451 | 0.966965 | | |
| 130 | hsa-miR-376a-3p | 183.3076 | 221.8073 | 596.0151 | 634.0029 | 0.964484 | | |
| 131 | hsa-miR-338-3p | 916.341 | 699.483 | 2243.518 | 2059.56 | 0.962056 | | |
| 132 | hsa-miR-876-3p | 2.439963 | 2.027786 | 6.77189 | 7.020446 | 0.957519 | | |
| 133 | hsa-miR-196a-5p | 65.19743 | 24.20754 | 104.3558 | 58.31384 | 0.949047 | | |
| 134 | hsa-miR-337-5p | 35.26824 | 35.39319 | 109.9924 | 122.3251 | 0.947564 | | |
| 135 | hsa-miR-486-5p | 586.651 | 363.0785 | 1440.293 | 1440.503 | 0.946607 | | |
| 136 | hsa-miR-873-5p | 12.62384 | 8.213764 | 32.17894 | 33.15986 | 0.945293 | | |
| 137 | hsa-miR-1908 | 19.63097 | 8.395014 | 13.80487 | 3.947736 | 0.944052 | | |
| 138 | hsa-miR-377-3p | 136.4046 | 154.1658 | 428.783 | 465.7138 | 0.943339 | | |
| 139 | hsa-miR-532-5p | 264.9617 | 207.185 | 522.8253 | 339.7915 | 0.942869 | | |
| 140 | hsa-miR-155-3p | 157.2815 | 256.3036 | 26.7993 | 21.27255 | 0.940154 | | |
| 141 | hsa-miR-194-5p | 334.6617 | 182.2838 | 657.6424 | 505.0221 | 0.939846 | | |
| 142 | hsa-miR-379-5p | 23.23865 | 25.01905 | 63.49565 | 60.72545 | 0.938999 | | |
| 143 | hsa-miR-770-5p | 7.07006 | 2.030097 | 9.538505 | 3.230468 | 0.938471 | | |
| 144 | hsa-miR-598 | 38.42457 | 4.737836 | 55.77089 | 32.39218 | 0.934356 | | |
| 145 | hsa-miR-200b-3p | 46.8328 | 35.73877 | 96.4324 | 70.60606 | 0.932807 | | |
| 146 | hsa-miR-376c | 281.237 | 290.3267 | 814.0394 | 852.3123 | 0.932582 | | |
| 147 | hsa-miR-125b-2- | 3.38324 | 3.118488 | 6.037832 | 2.590305 | 0.930001 | | |
| 148 | hsa-miR-27b-3p | 1407.188 | 965.8829 | 2678.947 | 1769.536 | 0.929846 | | |
| 149 | hsa-miR-181a-3p | 181.4669 | 222.1718 | 402.138 | 260.8043 | 0.913797 | | |
| 150 | hsa-miR-490-5p | 8.575117 | 6.092529 | 4.218613 | 3.547455 | 0.903841 | | |
| 151 | hsa-miR-33a-3p | 0.425753 | 0.564221 | 1.129043 | 0.992671 | 0.903454 | | |
| 152 | hsa-miR-21-3p | 187.846 | 92.24739 | 367.4585 | 306.1998 | 0.901562 | | |
| 153 | hsa-miR-129-2-3p | 2.266217 | 0.631388 | 3.047898 | 1.106577 | 0.899537 | | |
| 154 | hsa-miR-575 | 615.464 | 443.465 | 329.1812 | 193.1349 | 0.899412 | | |
| 155 | hsa-miR-362-5p | 210.4039 | 203.1629 | 421.2884 | 266.6432 | 0.897751 | | |
| 156 | hsa-miR-1226-5p | 25.48953 | 8.957313 | 34.90602 | 12.04039 | 0.896906 | | |
| 157 | hsa-miR-29c-3p | 14879.56 | 6440.149 | 26395.84 | 19305.45 | 0.894622 | | |
| 158 | hsa-miR-421 | 19.49304 | 22.48176 | 44.26052 | 33.07066 | 0.891679 | | |
| 159 | hsa-miR-374b-3p | 1.186263 | 1.007528 | 2.568615 | 2.093531 | 0.891535 | | |
| 160 | hsa-miR-1268a | 520.2263 | 322.8214 | 348.1637 | 63.763 | 0.890169 | | |
| 161 | hsa-miR-93-5p | 2357.955 | 1863.763 | 4381.103 | 2708.514 | 0.884963 | | |
| 162 | hsa-miR-875-5p | 0.620387 | 0.901336 | 2.69363 | 3.80917 | 0.880264 | | |
| 163 | hsa-miR-656 | 0.992543 | 1.54593 | 3.39428 | 3.913691 | 0.879818 | | |
| 164 | hsa-miR-548d-5p | 10.63489 | 4.628149 | 19.51122 | 15.58422 | 0.878307 | | |
| 165 | hsa-miR-195-5p | 289.1412 | 202.51 | 568.6113 | 435.8694 | 0.875561 | | |
| 166 | hsa-miR-181c-3p | 26.6853 | 15.0928 | 45.7548 | 28.51775 | 0.874536 | | |
| 167 | hsa-miR-27a-3p | 8601.15 | 5655.97 | 15705.32 | 10653.27 | 0.871184 | | |
| 168 | hsa-miR-497-5p | 36.1686 | 15.31979 | 59.41077 | 38.29806 | 0.866956 | | |
| 169 | hsa-miR-662 | 4.11999 | 1.633326 | 2.569832 | 1.95182 | 0.864767 | | |
| 170 | hsa-miR-148b-5p | 4.324451 | 3.882256 | 8.92008 | 6.83411 | 0.857684 | | |
| 171 | hsa-let-7a-3p | 1.523313 | 1.365024 | 3.099315 | 2.328518 | 0.853382 | | |
| 172 | hsa-miR-10a-3p | 6.550873 | 2.407515 | 4.399725 | 2.693591 | 0.843405 | | |
| 173 | hsa-miR-143-3p | 28.76578 | 19.07357 | 56.42163 | 46.95587 | 0.837683 | | |
| 174 | hsa-miR-7-1-3p | 334.1263 | 212.0399 | 616.1754 | 468.835 | 0.82849 | | |
| 175 | hsa-miR-1254 | 4.054643 | 2.363122 | 1.993332 | 2.64469 | 0.823238 | | |
| 176 | hsa-miR-1185-5p | 2.030057 | 3.342956 | 6.440427 | 7.391315 | 0.821736 | | |
| 177 | hsa-miR-130a-3p | 2433.883 | 1584.835 | 4538.946 | 3560.783 | 0.818196 | | |
| 178 | hsa-miR-503 | 10.64531 | 5.670913 | 17.23307 | 10.43322 | 0.818145 | | |
| 179 | hsa-miR-1284 | 0.1 | 1.70E−17 | 2.20755 | 5.162422 | 0.816497 | | |
| 180 | hsa-miR-15b-3p | 22.24109 | 18.36317 | 43.49992 | 33.90878 | 0.813393 | | |
| 181 | hsa-miR-381 | 56.88544 | 58.87612 | 126.1442 | 112.6181 | 0.807709 | | |
| 182 | hsa-miR-543 | 30.84928 | 30.22573 | 65.99608 | 56.87934 | 0.806998 | | |
| 183 | hsa-let-7g-3p | 3.42548 | 2.360384 | 6.533988 | 5.346364 | 0.806698 | | |
| 184 | hsa-miR-193a-5p | 19.74152 | 11.91064 | 32.48219 | 19.76778 | 0.804375 | | |
| 185 | hsa-miR-192-3p | 10.75833 | 7.336629 | 18.64859 | 12.33679 | 0.802123 | | |
| 186 | hsa-miR-130b-3p | 791.807 | 585.7731 | 1360.248 | 844.6789 | 0.794772 | | |
| 187 | hsa-miR-1285-3p | 3.815257 | 3.902323 | 7.893895 | 6.383187 | 0.793084 | | |
| 188 | hsa-miR-1910 | 2.850717 | 3.507741 | 5.787593 | 3.91509 | 0.791309 | | |
| 189 | hsa-miR-454-5p | 9.127203 | 7.617936 | 15.67453 | 8.982961 | 0.788792 | | |
| 190 | hsa-miR-25-5p | 3.930079 | 4.420997 | 7.998482 | 5.947288 | 0.784778 | | |
| 191 | hsa-miR-30a-5p | 78.8286 | 64.43019 | 140.9674 | 94.4098 | 0.782408 | | |
| 192 | hsa-miR-125b-5p | 246.8471 | 178.7685 | 462.258 | 374.8524 | 0.778189 | | |
| 193 | hsa-miR-616-3p | 3.454333 | 3.406183 | 1.739227 | 1.017038 | 0.775501 | | |
| 194 | hsa-miR-145-3p | 3.368953 | 3.148319 | 6.8969 | 5.95479 | 0.775108 | | |
| 195 | hsa-miR-501-3p | 19.37154 | 20.92876 | 36.23172 | 22.60968 | 0.774496 | | |
| 196 | hsa-miR-514a-3p | 3.84076 | 5.157551 | 9.97037 | 10.70331 | 0.772923 | | |
| 197 | hsa-miR-495 | 88.53167 | 104.1574 | 212.3229 | 217.4844 | 0.769746 | | |
| 198 | hsa-miR-25-3p | 6030.313 | 2217.078 | 8779.755 | 4967.81 | 0.76534 | | |
| 199 | hsa-miR-411-5p | 9.038327 | 13.81282 | 23.63657 | 24.57336 | 0.760599 | | |
| 200 | hsa-miR-551a | 0.424057 | 0.561283 | 0.91942 | 0.747851 | 0.756781 | | |
| 201 | hsa-let-7i-3p | 2.87879 | 2.946215 | 5.841027 | 4.887115 | 0.756316 | | |
| 202 | hsa-miR-30b-3p | 24.20297 | 15.53749 | 39.97932 | 26.2189 | 0.755637 | | |
| 203 | hsa-miR-769-3p | 9.441827 | 6.560658 | 14.80909 | 7.65954 | 0.754878 | | |
| 204 | hsa-miR-28-5p | 711.7587 | 437.8647 | 1154.231 | 737.1785 | 0.753117 | | |
| 205 | hsa-miR-628-3p | 13.84826 | 10.29626 | 25.64031 | 21.12319 | 0.750621 | | |
| 206 | hsa-miR-431-5p | 39.17785 | 43.05046 | 80.89494 | 68.61648 | 0.74717 | | |
| 207 | hsa-miR-425-5p | 2889.868 | 2947.54 | 5091.319 | 2974.502 | 0.743477 | | |
| 208 | hsa-miR-29c-5p | 208.012 | 130.2171 | 343.9953 | 237.4238 | 0.739762 | | |
| 209 | hsa-miR-142-5p | 6344.022 | 4678.484 | 11705.34 | 9845.562 | 0.738268 | | |
| 210 | hsa-miR-424-3p | 4.344583 | 3.676928 | 7.60428 | 5.15879 | 0.737845 | | |
| 211 | hsa-miR-365a-3p | 206.0242 | 199.2049 | 394.486 | 314.1666 | 0.734212 | | |
| 212 | hsa-miR-148a-5p | 1.75694 | 2.038959 | 3.516951 | 2.76961 | 0.732031 | | |
| 213 | hsa-miR-1208 | 6.430113 | 2.510058 | 8.844777 | 4.107281 | 0.729799 | | |
| 214 | hsa-miR-410 | 111.5335 | 124.6606 | 235.565 | 216.2834 | 0.727577 | | |
| 215 | hsa-miR-1915-3p | 242.471 | 112.95 | 341.4607 | 161.4085 | 0.721608 | | |
| 216 | hsa-let-7d-5p | 7019.867 | 3572.69 | 10500.68 | 6107.19 | 0.719185 | | |
| 217 | hsa-let-7i-5p | 14205 | 4546.668 | 20131.53 | 11969.34 | 0.717671 | | |
| 218 | hsa-miR-92a-1-5p | 12.22182 | 5.884461 | 18.62822 | 12.15098 | 0.710423 | | |
| 219 | hsa-miR-363-3p | 2425.627 | 1428.773 | 3863.479 | 2631.647 | 0.708228 | | |
| 220 | hsa-miR-485-3p | 14.7532 | 15.40457 | 27.18437 | 19.72967 | 0.707638 | | |
| 221 | hsa-miR-641 | 4.414043 | 3.555341 | 7.511283 | 5.206503 | 0.706984 | | |
| 222 | hsa-miR-125a-5p | 204.0793 | 115.5344 | 316.2467 | 202.71 | 0.704913 | | |
| 223 | hsa-miR-221-5p | 182.7801 | 119.4804 | 297.1518 | 206.248 | 0.702252 | | |
| 224 | hsa-miR-299-3p | 14.15081 | 9.534297 | 24.58692 | 20.24373 | 0.700927 | | |
| 225 | hsa-miR-1181 | 116.8189 | 71.71704 | 78.40652 | 38.05673 | 0.699846 | | |
| 226 | hsa-miR-191-5p | 4.820003 | 4.989985 | 8.669722 | 6.11738 | 0.693183 | | |
| 227 | hsa-miR-625-5p | 358.7367 | 161.328 | 541.3216 | 368.4545 | 0.689283 | | |
| 228 | hsa-miR-154-5p | 39.65204 | 40.03139 | 74.54746 | 62.08308 | 0.683457 | | |
| 229 | hsa-miR-629-3p | 47.30073 | 23.2381 | 59.1317 | 11.58939 | 0.679404 | | |
| 230 | hsa-miR-107 | 8223.777 | 6067.449 | 13156.11 | 8592.961 | 0.672878 | | |
| 231 | hsa-miR-650 | 3.161577 | 2.656657 | 6.310913 | 6.75038 | 0.66957 | | |
| 232 | hsa-miR-26b-5p | 13151.16 | 7778.707 | 20773.85 | 15098.09 | 0.666412 | | |
| 233 | hsa-miR-382-5p | 36.48117 | 30.68769 | 61.40942 | 44.39999 | 0.663977 | | |
| 234 | hsa-miR-455-3p | 1.02228 | 0.874728 | 1.898947 | 1.795904 | 0.656524 | | |
| 235 | hsa-miR-532-3p | 92.10393 | 79.90847 | 151.3951 | 102.2108 | 0.651124 | | |
| 236 | hsa-miR-17-5p | 5472.84 | 3255.521 | 8176.176 | 5082.163 | 0.648462 | | |
| 237 | hsa-miR-542-5p | 66.1532 | 38.45464 | 99.13132 | 63.35747 | 0.647823 | | |
| 238 | hsa-miR-31-5p | 501.6527 | 371.7484 | 855.0319 | 720.8526 | 0.646859 | | |
| 239 | hsa-miR-361-5p | 831.897 | 670.7455 | 1309.945 | 811.9549 | 0.644835 | | |
| 240 | hsa-miR-539-5p | 21.27419 | 19.16675 | 37.54004 | 31.52993 | 0.641693 | | |
| 241 | hsa-miR-23b-5p | 2.186717 | 1.466044 | 3.173117 | 1.613825 | 0.640547 | | |
| 242 | hsa-let-7b-5p | 11822.19 | 4441.573 | 15965.63 | 8610.029 | 0.634932 | | |
| 243 | hsa-miR-187-5p | 3.04236 | 2.675683 | 4.924757 | 3.259119 | 0.634359 | | |
| 244 | hsa-miR-185-3p | 0.269977 | 0.294409 | 0.763571 | 1.270528 | 0.630816 | | |
| 245 | hsa-miR-199a-3p | 3810.253 | 1887.753 | 5451.463 | 3324.975 | 0.629693 | | |
| 246 | hsa-miR-502-3p | 104.5753 | 93.50873 | 169.0489 | 113.1025 | 0.624105 | | |
| 247 | hsa-miR-126-3p | 4494.71 | 3198.316 | 7082.994 | 5144.949 | 0.620449 | | |
| 248 | hsa-miR-24-3p | 7818.943 | 5735.524 | 12116.46 | 8234.611 | 0.615244 | | |
| 249 | hsa-miR-22-3p | 6905.223 | 5172.377 | 10548.84 | 6746.466 | 0.611404 | | |
| 250 | hsa-miR-1233 | 19.28533 | 9.61116 | 26.42618 | 13.77636 | 0.610655 | | |
| 251 | hsa-miR-22-5p | 108.7826 | 108.564 | 178.9204 | 122.1679 | 0.60796 | | |
| 252 | hsa-miR-550a-3p | 50.55793 | 45.70892 | 80.38178 | 53.59877 | 0.600635 | | |
| 253 | hsa-miR-409-3p | 89.7205 | 93.51277 | 152.0789 | 114.5698 | 0.599362 | | |
| 254 | hsa-miR-548j | 1.99244 | 2.249581 | 1.07865 | 0.802375 | 0.598823 | | |
| 255 | hsa-miR-363-5p | 0.707043 | 1.05143 | 1.331285 | 1.034313 | 0.59858 | | |
| 256 | hsa-miR-487b | 143.2004 | 159.5325 | 262.32 | 239.9184 | 0.596417 | | |
| 257 | hsa-miR-652-3p | 350.4593 | 202.8118 | 496.2308 | 286.5722 | 0.595735 | | |
| 258 | hsa-miR-186-5p | 1057.622 | 853.8807 | 1695.87 | 1292.561 | 0.594703 | | |
| 259 | hsa-miR-411-3p | 1.53688 | 2.488749 | 3.374304 | 3.791407 | 0.585152 | | |
| 260 | hsa-let-7f-2-3p | 1.94057 | 1.773632 | 3.009828 | 1.905167 | 0.581308 | | |
| 261 | hsa-miR-151a-3p | 370.4393 | 146.4765 | 493.5205 | 279.6075 | 0.577732 | | |
| 262 | hsa-miR-937 | 238.7363 | 91.6735 | 289.659 | 87.63988 | 0.567974 | | |
| 263 | hsa-miR-30c-1-3p | 11.29205 | 6.77279 | 15.14351 | 6.909011 | 0.563004 | | |
| 264 | hsa-miR-485-5p | 1.421593 | 2.289067 | 2.8547 | 2.811765 | 0.561911 | | |
| 265 | hsa-miR-337-3p | 17.23728 | 22.70078 | 31.95729 | 30.12215 | 0.557334 | | |
| 266 | hsa-miR-151a-5p | 1309.25 | 669.1002 | 1768.537 | 999.8741 | 0.550382 | | |
| 267 | hsa-miR-432-5p | 58.86417 | 43.92841 | 86.99262 | 58.3962 | 0.549789 | | |
| 268 | hsa-miR-885-3p | 3.89961 | 4.610653 | 2.118063 | 1.872639 | 0.549581 | | |
| 269 | hsa-miR-29b-1-5p | 415.613 | 212.3941 | 556.6301 | 304.3726 | 0.545767 | | |
| 270 | hsa-miR-1301 | 3.333757 | 3.932864 | 5.344283 | 3.443814 | 0.545104 | | |
| 271 | hsa-miR-491-3p | 1.953843 | 1.631648 | 2.839458 | 1.632747 | 0.542591 | | |
| 272 | hsa-miR-505-5p | 63.48963 | 38.41235 | 86.93075 | 48.25967 | 0.540915 | | |
| 273 | hsa-miR-26a-1-3p | 2.643253 | 1.365117 | 3.604598 | 2.209526 | 0.537869 | | |
| 274 | hsa-miR-508-5p | 5.463243 | 4.060878 | 3.795992 | 2.158792 | 0.536122 | | |
| 275 | hsa-miR-1825 | 11.80468 | 9.89897 | 8.349237 | 3.006213 | 0.535512 | | |
| 276 | hsa-miR-625-3p | 6.72876 | 1.923049 | 8.351405 | 4.168249 | 0.532775 | | |
| 277 | hsa-miR-409-5p | 18.04407 | 23.87256 | 32.00876 | 28.57859 | 0.532484 | | |
| 278 | hsa-miR-572 | 30.5718 | 16.45414 | 22.46117 | 14.38704 | 0.525961 | | |
| 279 | hsa-miR-493-5p | 48.44273 | 61.59503 | 84.18154 | 75.02485 | 0.523186 | | |
| 280 | hsa-miR-30e-3p | 390.0133 | 289.0365 | 559.2558 | 361.1204 | 0.52062 | | |
| 281 | hsa-miR-369-5p | 18.44486 | 25.53079 | 33.43566 | 32.08937 | 0.520332 | | |
| 282 | hsa-miR-377-5p | 1.92696 | 2.158412 | 3.261167 | 2.979375 | 0.51937 | | |
| 283 | hsa-miR-103a-3p | 11523.88 | 8273.218 | 16344.53 | 10326.85 | 0.518348 | | |
| 284 | hsa-miR-486-3p | 17.4404 | 4.676391 | 21.99785 | 12.99213 | 0.515884 | | |
| 285 | hsa-miR-654-3p | 71.84219 | 89.65051 | 122.4087 | 107.6913 | 0.512476 | | |
| 286 | hsa-miR-146b-5p | 7368.29 | 5131.347 | 10746.21 | 8254.191 | 0.504712 | | |
| 287 | hsa-miR-1255a | 3.161873 | 1.395644 | 5.455855 | 7.712905 | 0.503699 | | |
| 288 | hsa-miR-30b-5p | 6804.067 | 4791.251 | 9708.432 | 6836.939 | 0.499539 | | |
| 289 | hsa-miR-23b-3p | 1333.595 | 1122.345 | 1896.278 | 1165.335 | 0.491925 | | |
| 290 | hsa-miR-148b-3p | 811.776 | 702.9443 | 1182.256 | 812.741 | 0.488861 | | |
| 291 | hsa-miR-1305 | 82.0401 | 49.77637 | 111.4723 | 71.17207 | 0.48669 | | |
| 292 | hsa-miR-1207-5p | 579.1353 | 153.6938 | 694.168 | 319.7199 | 0.485971 | | |
| 293 | hsa-miR-324-3p | 495.003 | 174.2826 | 638.5113 | 420.0319 | 0.482937 | | |
| 294 | hsa-miR-378a-5p | 59.31367 | 55.69239 | 82.38462 | 40.66101 | 0.478882 | | |
| 295 | hsa-miR-181c-5p | 220.1181 | 173.7281 | 315.3771 | 224.2531 | 0.478711 | | |
| 296 | hsa-miR-223-5p | 136.0065 | 98.99465 | 187.8518 | 120.4292 | 0.472558 | | |
| 297 | hsa-miR-629-5p | 38.0754 | 25.00279 | 54.20122 | 43.50886 | 0.470747 | | |
| 298 | hsa-miR-1228-5p | 15.86768 | 7.295844 | 19.73537 | 9.309247 | 0.465845 | | |
| 299 | hsa-miR-339-3p | 15.92968 | 11.03274 | 21.56479 | 13.25298 | 0.464068 | | |
| 300 | hsa-miR-664-5p | 40.7797 | 22.38181 | 52.62555 | 28.78599 | 0.46302 | | |
| 301 | hsa-miR-106b-3p | 0.96648 | 0.881506 | 1.307765 | 0.593744 | 0.462681 | | |
| 302 | hsa-miR-338-5p | 5.859857 | 3.243918 | 8.384382 | 7.806198 | 0.456923 | | |
| 303 | hsa-let-7f-5p | 19679.93 | 12211.84 | 26694.1 | 18614.08 | 0.455082 | | |
| 304 | hsa-miR-505-3p | 213.1429 | 167.1484 | 291.1117 | 176.1873 | 0.454184 | | |
| 305 | hsa-miR-92a-3p | 3167.25 | 1813.958 | 4055.534 | 2103.237 | 0.453531 | | |
| 306 | hsa-miR-345-5p | 167.0847 | 261.0021 | 74.82009 | 146.3026 | 0.453049 | | |
| 307 | hsa-miR-671-5p | 3454.906 | 5851.571 | 1383.638 | 3293.553 | 0.452978 | | |
| 308 | hsa-let-7d-3p | 9.428113 | 4.615789 | 11.62526 | 5.10862 | 0.451882 | | |
| 309 | hsa-miR-30c-5p | 2311.067 | 1898.189 | 3233.48 | 2234.92 | 0.446353 | | |
| 310 | hsa-miR-323a-3p | 13.39341 | 18.47769 | 21.61584 | 18.66188 | 0.442786 | | |
| 311 | hsa-miR-1295a | 7.081507 | 4.579548 | 5.230067 | 3.805959 | 0.441581 | | |
| 312 | hsa-miR-26a-5p | 8084.227 | 7119.212 | 11516 | 8491.006 | 0.439683 | | |
| 313 | hsa-miR-23a-3p | 12706.69 | 10130.47 | 17312.42 | 10837.09 | 0.439319 | | |
| 314 | hsa-miR-376a-5p | 25.29827 | 35.96121 | 43.34448 | 47.8772 | 0.4305 | | |
| 315 | hsa-miR-30d-5p | 1614.978 | 1372.173 | 2225.656 | 1466.07 | 0.430321 | | |
| 316 | hsa-miR-610 | 5.25813 | 0.660453 | 5.849747 | 2.09053 | 0.430113 | | |
| 317 | hsa-miR-380-3p | 1.786143 | 2.920486 | 3.387602 | 4.601482 | 0.425808 | | |
| 318 | hsa-miR-423-3p | 6.781883 | 3.316862 | 8.372432 | 4.206707 | 0.422818 | | |
| 319 | hsa-miR-93-3p | 6.52924 | 6.759195 | 9.344452 | 6.577218 | 0.422184 | | |
| 320 | hsa-miR-663a | 81.0552 | 12.02709 | 97.896 | 70.4208 | 0.40852 | | |
| 321 | hsa-miR-140-3p | 2526.506 | 1997.048 | 3431.999 | 2472.778 | 0.405158 | | |
| 322 | hsa-miR-299-5p | 23.13157 | 30.51451 | 35.92983 | 32.84214 | 0.404007 | | |
| 323 | hsa-miR-484 | 95.85107 | 88.1117 | 129.3314 | 78.00853 | 0.403086 | | |
| 324 | hsa-miR-374b-5p | 2377.32 | 1958.474 | 3279.88 | 2544.662 | 0.400859 | | |
| 325 | hsa-miR-636 | 3.607733 | 1.946871 | 2.897012 | 1.605341 | 0.400157 | | |
| 326 | hsa-miR-342-5p | 1461.325 | 589.5351 | 1787.203 | 1041.509 | 0.399595 | | |
| 327 | hsa-miR-1260a | 1269.511 | 1421.848 | 1826.906 | 1373.046 | 0.398867 | | |
| 328 | hsa-miR-146a-5p | 3298.612 | 2579.438 | 4457.899 | 3296.583 | 0.394582 | | |
| 329 | hsa-miR-135a-5p | 6.99037 | 5.415104 | 9.830608 | 9.013422 | 0.393698 | | |
| 330 | hsa-miR-135a-3p | 96.92643 | 70.39155 | 76.0676 | 36.10367 | 0.391733 | | |
| 331 | hsa-miR-1281 | 18.02117 | 16.32073 | 13.73981 | 5.917603 | 0.385043 | | |
| 332 | hsa-let-7b-3p | 8.251073 | 1.808078 | 8.939083 | 1.790619 | 0.382366 | | |
| 333 | hsa-miR-101-5p | 11.7366 | 8.373966 | 15.53989 | 11.68932 | 0.37913 | | |
| 334 | hsa-miR-331-3p | 1630.77 | 1141.028 | 2077.104 | 1235.323 | 0.375647 | | |
| 335 | hsa-miR-526b-5p | 3.216077 | 3.920491 | 4.875167 | 4.9157 | 0.375522 | | |
| 336 | hsa-miR-513a-5p | 74.3619 | 73.65251 | 99.27915 | 59.94056 | 0.373032 | | |
| 337 | hsa-miR-16-2-3p | 56.6879 | 40.17095 | 73.91741 | 52.30452 | 0.372629 | | |
| 338 | hsa-miR-199a-5p | 1097.387 | 644.1913 | 1374.666 | 848.7248 | 0.371459 | | |
| 339 | hsa-miR-194-3p | 3.014393 | 1.355708 | 2.566053 | 1.068384 | 0.369904 | | |
| 340 | hsa-miR-204-5p | 6.57603 | 9.106743 | 4.375868 | 2.823361 | 0.368842 | | |
| 341 | hsa-miR-335-3p | 15.08942 | 11.41621 | 19.47365 | 12.42643 | 0.367764 | | |
| 342 | hsa-miR-15b-5p | 22693.23 | 12881.56 | 28005.05 | 16366.02 | 0.363231 | | |
| 343 | hsa-miR-142-3p | 30288.4 | 12765.49 | 36881.8 | 23864.18 | 0.360003 | | |
| 344 | hsa-miR-200c-3p | 91.1941 | 76.02312 | 119.5142 | 81.70223 | 0.359107 | | |
| 345 | hsa-let-7a-5p | 26857.33 | 14360.66 | 33090.28 | 20668.14 | 0.355876 | | |
| 346 | hsa-miR-30c-2-3p | 3.284037 | 4.446397 | 4.901672 | 4.646709 | 0.355794 | | |
| 347 | hsa-miR-139-3p | 22.16177 | 12.51684 | 27.16137 | 16.11636 | 0.349217 | | |
| 348 | hsa-miR-320d | 2079.06 | 953.234 | 2463.833 | 1257.818 | 0.348045 | | |
| 349 | hsa-miR-127-3p | 77.51037 | 80.35192 | 104.2827 | 74.2444 | 0.346352 | | |
| 350 | hsa-miR-339-5p | 1.048453 | 1.642769 | 1.684834 | 2.041732 | 0.345437 | | |
| 351 | hsa-miR-222-3p | 227.7308 | 156.4323 | 289.5964 | 205.484 | 0.341878 | | |
| 352 | hsa-miR-589-3p | 1.486937 | 2.402245 | 2.165263 | 1.573932 | 0.341195 | | |
| 353 | hsa-miR-92b-3p | 4.238993 | 0.470578 | 4.441385 | 0.723554 | 0.338977 | | |
| 354 | hsa-miR-29b-2-5p | 6.892323 | 4.291383 | 8.724758 | 6.728381 | 0.332572 | | |
| 355 | hsa-miR-601 | 49.13867 | 20.21229 | 39.52145 | 37.68331 | 0.332226 | | |
| 356 | hsa-miR-326 | 64.82767 | 48.84782 | 79.39585 | 40.1261 | 0.327471 | | |
| 357 | hsa-miR-665 | 12.23184 | 13.18676 | 9.379102 | 4.44625 | 0.323568 | | |
| 358 | hsa-miR-28-3p | 2.45242 | 1.091067 | 2.962616 | 2.085632 | 0.321212 | | |
| 359 | hsa-miR-181d | 160.6319 | 147.5842 | 127.6226 | 57.99943 | 0.321128 | | |
| 360 | hsa-miR-654-5p | 15.48453 | 14.94002 | 12.31072 | 4.948324 | 0.319163 | | |
| 361 | hsa-miR-193b-3p | 63.96133 | 43.31425 | 80.15432 | 60.34848 | 0.312417 | | |
| 362 | hsa-miR-550a-5p | 8.675293 | 5.58982 | 10.08398 | 3.515567 | 0.309417 | | |
| 363 | hsa-miR-483-3p | 7.261147 | 11.09941 | 4.815728 | 5.022977 | 0.303357 | | |
| 364 | hsa-miR-32-3p | 32.6775 | 14.21091 | 37.97591 | 21.09347 | 0.300156 | | |
| 365 | hsa-miR-320b | 1218.731 | 701.2434 | 1444.679 | 806.308 | 0.299755 | | |
| 366 | hsa-miR-433 | 3.65934 | 4.005434 | 4.842422 | 3.89638 | 0.299446 | | |
| 367 | hsa-miR-206 | 12.85865 | 8.125677 | 15.39674 | 9.03612 | 0.295783 | | |
| 368 | hsa-miR-188-5p | 335.6299 | 284.04 | 263.3468 | 218.402 | 0.287727 | | |
| 369 | hsa-miR-130b-5p | 7.622113 | 7.225717 | 9.432301 | 5.422103 | 0.286245 | | |
| 370 | hsa-miR-659-3p | 9.898013 | 7.151982 | 11.58232 | 5.362551 | 0.269175 | | |
| 371 | hsa-miR-193b-5p | 28.56514 | 32.8286 | 36.86319 | 29.15931 | 0.267731 | | |
| 372 | hsa-miR-622 | 21.36708 | 25.4598 | 15.53146 | 19.1582 | 0.261581 | | |
| 373 | hsa-miR-1225-5p | 1502.368 | 1613.329 | 1138.597 | 1178.249 | 0.260621 | | |
| 374 | hsa-miR-513c-5p | 48.43014 | 39.38422 | 59.64882 | 48.11767 | 0.256421 | | |
| 375 | hsa-miR-342-3p | 11714.18 | 7616.215 | 13893.04 | 9501.925 | 0.254568 | | |
| 376 | hsa-miR-602 | 18.7729 | 5.243699 | 20.71983 | 10.17346 | 0.252567 | | |
| 377 | hsa-miR-760 | 8.924253 | 5.261754 | 7.794998 | 3.771681 | 0.250017 | | |
| 378 | hsa-miR-766-3p | 61.13087 | 20.9282 | 66.68048 | 23.75935 | 0.248374 | | |
| 379 | hsa-miR-320a | 412.4623 | 272.2151 | 481.0802 | 284.2021 | 0.246642 | | |
| 380 | hsa-miR-181b-5p | 660.247 | 397.9675 | 760.4503 | 416.863 | 0.245949 | | |
| 381 | hsa-miR-494 | 1455.917 | 1338.21 | 1765.69 | 1181.018 | 0.245926 | | |
| 382 | hsa-miR-584-5p | 138.0114 | 69.22222 | 154.0336 | 61.47383 | 0.245182 | | |
| 383 | hsa-miR-449b-5p | 3.77337 | 3.336613 | 4.89187 | 6.023952 | 0.238981 | | |
| 384 | hsa-miR-330-3p | 61.9249 | 47.04076 | 71.96511 | 38.22322 | 0.235509 | | |
| 385 | hsa-miR-645 | 4.914527 | 2.548388 | 5.995105 | 6.692568 | 0.233867 | | |
| 386 | hsa-miR-149-3p | 4.991453 | 3.285212 | 5.981364 | 5.192961 | 0.23352 | | |
| 387 | hsa-miR-196a-3p | 12.18244 | 8.50423 | 10.40664 | 6.755128 | 0.232749 | | |
| 388 | hsa-let-7g-5p | 29099.47 | 14398.27 | 33266.97 | 21525.72 | 0.232018 | | |
| 389 | hsa-miR-595 | 46.5403 | 30.30505 | 40.96673 | 18.50656 | 0.228371 | | |
| 390 | hsa-miR-16-5p | 31403.27 | 20422.46 | 36411.59 | 23868.71 | 0.226154 | | |
| 391 | hsa-miR-664-3p | 80.12683 | 60.89015 | 94.12007 | 63.63035 | 0.224754 | | |
| 392 | hsa-miR-1224-5p | 52.56527 | 30.24894 | 46.88165 | 20.43599 | 0.224272 | | |
| 393 | hsa-miR-1471 | 48.13967 | 33.73182 | 41.24935 | 28.36042 | 0.221938 | | |
| 394 | hsa-miR-1307-3p | 11.62689 | 4.697584 | 12.74788 | 5.410026 | 0.221811 | | |
| 395 | hsa-miR-29a-3p | 26971.7 | 14119.13 | 30848.99 | 20864.87 | 0.221661 | | |
| 396 | hsa-miR-1470 | 5.10126 | 0.819391 | 5.316963 | 1.135327 | 0.2207 | | |
| 397 | hsa-miR-21-5p | 36727.6 | 20643.8 | 41693.24 | 24429.86 | 0.220335 | | |
| 398 | hsa-miR-513b | 47.5851 | 41.36009 | 56.74912 | 41.96157 | 0.219967 | | |
| 399 | hsa-miR-146b-3p | 2.037323 | 1.677813 | 1.678778 | 1.610345 | 0.218083 | | |
| 400 | hsa-miR-877-5p | 13.13662 | 5.565019 | 12.20105 | 3.399027 | 0.208739 | | |
| 401 | hsa-let-7f-1-3p | 6.353057 | 2.06283 | 6.60365 | 0.365618 | 0.206381 | | |
| 402 | hsa-miR-124-3p | 16.08747 | 14.44674 | 21.16739 | 35.17461 | 0.204747 | | |
| 403 | hsa-miR-412 | 3.187337 | 5.347424 | 4.30951 | 5.891885 | 0.199687 | | |
| 404 | hsa-miR-150-3p | 66.83743 | 31.77815 | 72.95885 | 29.5385 | 0.199666 | | |
| 405 | hsa-miR-423-5p | 203.6791 | 156.7286 | 233.8702 | 147.3453 | 0.198577 | | |
| 406 | hsa-miR-379-3p | 0.682 | 1.008054 | 0.909532 | 1.287777 | 0.198213 | | |
| 407 | hsa-miR-520d-3p | 1.652043 | 1.492903 | 1.336978 | 1.691729 | 0.197866 | | |
| 408 | hsa-miR-1182 | 18.64594 | 22.6303 | 15.06861 | 15.44819 | 0.187892 | | |
| 409 | hsa-miR-181a-2- | 53.3324 | 47.23221 | 62.13176 | 46.51568 | 0.187724 | | |
| 410 | hsa-miR-198 | 20.98891 | 30.64731 | 28.19938 | 49.40523 | 0.180143 | | |
| 411 | hsa-miR-498 | 6.030483 | 4.965282 | 5.227623 | 4.075037 | 0.177618 | | |
| 412 | hsa-miR-26b-3p | 26.98183 | 35.86881 | 31.71766 | 18.30138 | 0.17485 | | |
| 413 | hsa-miR-1183 | 32.92744 | 33.76232 | 38.22102 | 30.41969 | 0.164955 | | |
| 414 | hsa-miR-1202 | 1020.729 | 369.1553 | 1081.489 | 417.6954 | 0.154438 | | |
| 415 | hsa-miR-631 | 5.03589 | 4.305834 | 4.497333 | 2.900213 | 0.149474 | | |
| 416 | hsa-miR-1323 | 4.93872 | 5.834835 | 5.662525 | 4.076047 | 0.146063 | | |
| 417 | hsa-miR-183-3p | 4.814904 | 6.573417 | 5.606068 | 4.733106 | 0.139948 | | |
| 418 | hsa-miR-623 | 12.69251 | 6.379778 | 13.56803 | 6.403392 | 0.13698 | | |
| 419 | hsa-miR-491-5p | 2.94761 | 2.824584 | 3.293925 | 2.239753 | 0.136766 | | |
| 420 | hsa-miR-630 | 458.9939 | 623.7433 | 558.9466 | 954.8763 | 0.126633 | | |
| 421 | hsa-miR-320c | 1962.412 | 1101.79 | 2076.385 | 738.4427 | 0.123869 | | |
| 422 | hsa-miR-574-5p | 223.9447 | 102.2467 | 231.5458 | 23.98219 | 0.120435 | | |
| 423 | hsa-miR-1271-5p | 60.76607 | 38.27068 | 65.6363 | 42.68175 | 0.120323 | | |
| 424 | hsa-miR-155-5p | 2012.349 | 1339.343 | 1863.061 | 1166.854 | 0.119135 | | |
| 425 | hsa-miR-202-3p | 22.92909 | 14.36097 | 21.48376 | 10.72969 | 0.115209 | | |
| 426 | hsa-miR-520e | 4.302847 | 3.654586 | 3.751648 | 5.934293 | 0.114966 | | |
| 427 | hsa-miR-371a-5p | 22.7806 | 11.31218 | 21.57705 | 9.819408 | 0.11391 | | |
| 428 | hsa-miR-145-5p | 187.3063 | 137.004 | 202.8244 | 135.7907 | 0.113771 | | |
| 429 | hsa-miR-197-3p | 224.2353 | 138.0016 | 237.1594 | 104.5676 | 0.10656 | | |
| 430 | hsa-miR-887 | 18.97241 | 17.95691 | 17.23777 | 14.9985 | 0.105272 | | |
| 431 | hsa-miR-574-3p | 113.7205 | 115.6945 | 122.3799 | 50.16735 | 0.104416 | | |
| 432 | hsa-miR-940 | 163.2496 | 145.4333 | 154.0243 | 33.51787 | 0.103103 | | |
| 433 | hsa-miR-133b | 41.17953 | 6.969282 | 43.1708 | 34.0301 | 0.097136 | | |
| 434 | hsa-miR-200b-5p | 1.545344 | 1.422017 | 1.638708 | 1.035515 | 0.075982 | | |
| 435 | hsa-miR-181a-5p | 3718.717 | 2509.147 | 3894.045 | 2213.54 | 0.074249 | | |
| 436 | hsa-miR-10a-5p | 266.1613 | 250.7402 | 249.835 | 194.2736 | 0.073375 | | |
| 437 | hsa-miR-512-3p | 4.657813 | 5.350185 | 4.950648 | 2.781017 | 0.072027 | | |
| 438 | hsa-miR-765 | 54.09848 | 73.51626 | 59.2968 | 72.19761 | 0.07135 | | |
| 439 | hsa-miR-1261 | 18.9986 | 5.993089 | 19.7949 | 17.1759 | 0.068738 | | |
| 440 | hsa-miR-10b-5p | 26.08557 | 30.18625 | 24.45054 | 21.67934 | 0.063049 | | |
| 441 | hsa-miR-1290 | 434.8957 | 631.9971 | 468.0871 | 450.26 | 0.061337 | | |
| 442 | hsa-miR-129-5p | 6.462117 | 3.701531 | 6.751317 | 5.917741 | 0.060129 | | |
| 443 | hsa-miR-933 | 5.8796 | 2.353735 | 5.783698 | 1.080333 | 0.055853 | | |
| 444 | hsa-miR-223-3p | 45758.1 | 24596.6 | 44363.92 | 25499.81 | 0.05566 | | |
| 445 | hsa-miR-150-5p | 34689.27 | 16071.64 | 33770.65 | 17624.76 | 0.054523 | | |
| 446 | hsa-miR-483-5p | 239.3901 | 228.6052 | 226.9273 | 253.8112 | 0.051668 | | |
| 447 | hsa-miR-509-3p | 2.047347 | 3.372903 | 2.162538 | 1.392412 | 0.048346 | | |
| 448 | hsa-miR-583 | 7.834733 | 10.61972 | 7.374138 | 9.416405 | 0.045976 | | |
| 449 | hsa-miR-1914-3p | 145.074 | 59.96223 | 142.492 | 55.57492 | 0.044696 | | |
| 450 | hsa-miR-1180 | 19.89897 | 6.924458 | 19.55078 | 8.665175 | 0.044669 | | |
| 451 | hsa-miR-1246 | 2580.713 | 3454.018 | 2697.936 | 2182.698 | 0.041593 | | |
| 452 | hsa-miR-1275 | 509.8963 | 389.7724 | 496.8028 | 281.3516 | 0.03902 | | |
| 453 | hsa-miR-34c-3p | 4.223877 | 2.832373 | 4.346084 | 3.635872 | 0.037787 | | |
| 454 | hsa-miR-451a | 47185.1 | 19149.64 | 46291.28 | 31561 | 0.035252 | | |
| 455 | hsa-miR-100-5p | 163.1732 | 127.3883 | 167.6265 | 132.1868 | 0.034312 | | |
| 456 | hsa-miR-1909-5p | 2.897347 | 2.969052 | 2.980363 | 1.917515 | 0.033978 | | |
| 457 | hsa-miR-99b-3p | 5.426177 | 4.884456 | 5.565555 | 3.347455 | 0.033863 | | |
| 458 | hsa-miR-139-5p | 20.2524 | 11.21228 | 20.64685 | 12.26414 | 0.033604 | | |
| 459 | hsa-miR-638 | 349.611 | 291.3912 | 340.0301 | 312.2274 | 0.031745 | | |
| 460 | hsa-miR-509-3-5p | 2.972853 | 1.492912 | 2.908362 | 2.713286 | 0.030665 | | |
| 461 | hsa-miR-23a-5p | 27.95575 | 17.71157 | 28.31583 | 8.302653 | 0.027684 | | |
| 462 | hsa-miR-642a-5p | 2693.82 | 2291.67 | 2752.384 | 2152.738 | 0.026354 | | |
| 463 | hsa-miR-1179 | 2.935237 | 2.740039 | 3.041697 | 6.079273 | 0.024142 | | |
| 464 | hsa-miR-648 | 1.626567 | 1.323217 | 1.595998 | 1.309175 | 0.023225 | | |
| 465 | hsa-miR-146a-3p | 0.86922 | 1.332328 | 0.841158 | 1.14916 | 0.022617 | | |
| 466 | hsa-miR-936 | 7.103187 | 7.90074 | 7.249663 | 6.260051 | 0.020688 | | |
| 467 | hsa-miR-520b | 7.7247277 | 4.98155 | 7.857498 | 9.277179 | 0.018693 | | |
| 468 | hsa-miR-422a | 10.74906 | 7.171814 | 10.6277 | 9.684227 | 0.014399 | | |
| 469 | hsa-miR-1469 | 22.97945 | 21.48178 | 23.18769 | 13.99185 | 0.011741 | | |
| 470 | hsa-miR-122-5p | 2.136077 | 3.526588 | 2.11293 | 1.895057 | 0.008539 | | |
| 471 | hsa-miR-492 | 11.66558 | 10.57342 | 11.60271 | 6.371008 | 0.007421 | | |

TABLE 4
Non-Black race population: Top 25-microRNAs of 471 for pregnancy outcome prediction from microarray ordered by p value^{a}

| | | | Area | | | | | | | | |
|---|---|---|---|---|---|---|---|---|---|---|---|
| | | | under | Significance | | | | | | | |
| | | | the ROC | level | | | | | | P value | |
| P value | | Sample | curve | P (Area = | | Associated | | | “HC | ROC | HC |
| rank | MicroRNA | size | (AUC) | 0.5) | γ^{b} | criterion | Sens.^{c} | Spec.^{d} | Ratio”^{e} | curve <0.05 | Ratio ≥1.5 |
| 1 | hsa-miR-655 | 8 | 1 | 0.0001 | 1 | >0.13261 | 100 | 100 | 2.9853472 | x | x |
| 2 | hsa-miR-27a-3p | 8 | 1 | 0.0001 | 1 | >−0.1287 | 100 | 100 | 2.8648008 | x | x |
| 3 | hsa-miR-136-5p | 8 | 0.833 | 0.0253 | 0.666 | >−2.6669 | 100 | 66.67 | 1.852514 | x | x |
| 4 | hsa-miR-18a-5p | 8 | 0.833 | 0.0453 | 0.833 | >−0.1343 | 100 | 83.33 | 4.8214506 | x | x |
| 5 | hsa-miR-301a-3p | 8 | 0.806 | 0.0563 | 0.656 | >−1.6367 | 100 | 66.67 | 1.4701679 | | x |
| 6 | hsa-miR-625-5p | 8 | 0.806 | 0.0881 | 0.5 | >−2.6429 | 100 | 50 | 2.3075302 | | x |
| 7 | hsa-miR-20b-5p | 8 | 0.778 | 0.0965 | 0.5 | >−0.3118 | 100 | 50 | 2.1682291 | | x |
| 8 | hsa-miR-185-5p | 8 | 0.778 | 0.1102 | 0.666 | >−0.6039 | 100 | 66.67 | 4.1412891 | | x |
| 9 | hsa-miR-454-3p | 8 | 0.778 | 0.1102 | 0.666 | >−0.2482 | 100 | 66.67 | 3.9656328 | | x |
| 10 | hsa-miR-195-5p | 8 | 0.778 | 0.1102 | 0.666 | >−0.4243 | 100 | 66.67 | 2.3363489 | | x |
| 11 | hsa-miR-548am-5p | 8 | 0.778 | 0.121 | 0.666 | >−0.4144 | 100 | 66.67 | 1.5528394 | | x |
| 12 | hsa-miR-551b-3p | 8 | 0.778 | 0.1337 | 0.5 | >−1.3302 | 100 | 50 | 2.2702556 | | x |
| 13 | hsa-miR-18b-5p | 8 | 0.75 | 0.1588 | 0.666 | >−0.4996 | 100 | 66.67 | 2.9148195 | | x |
| 14 | hsa-miR-374a-5p | 8 | 0.75 | 0.1649 | 0.666 | >−0.2085 | 100 | 66.67 | 4.3369903 | | x |
| 15 | hsa-miR-363-3p | 8 | 0.75 | 0.1768 | 0.5 | >−1.4304 | 100 | 50 | 2.2182013 | | x |
| 16 | hsa-miR-590-5p | 8 | 0.75 | 0.2155 | 0.5 | >0.1802 | 66.67 | 83.33 | 1.5180919 | | x |
| 17 | hsa-miR-765 | 8 | 0.778 | 0.2335 | 0.666 | >0.5549 | 66.67 | 100 | 1.6948 | | x |
| 18 | hsa-miR-29c-5p | 8 | 0.722 | 0.2521 | 0.5 | >−0.5920 | 100 | 50 | 2.2756439 | | x |
| 19 | hsa-miR-148a-3p | 8 | 0.722 | 0.2523 | 0.5 | >−3.7966 | 100 | 50 | 2.2114932 | | x |
| 20 | hsa-miR-502-3p | 8 | 0.722 | 0.3233 | 0.5 | >−0.0498 | 66.67 | 83.33 | 3.1000913 | | x |
| 21 | hsa-miR-324-5p | 8 | 0.722 | 0.3233 | 0.5 | >0.0155 | 66.67 | 83.33 | 2.3973067 | | x |
| 22 | hsa-miR-141-3p | 8 | 0.722 | 0.3233 | 0.5 | >0.8865 | 66.67 | 83.33 | 1.9848975 | | x |
| 23 | hsa-miR-28-5p | 8 | 0.694 | 0.4268 | 0.5 | >0.1550 | 66.67 | 83.33 | 1.7616641 | | x |
| 24 | hsa-miR-484 | 8 | 0.667 | 0.4292 | 0.666 | >−0.9825 | 100 | 66.67 | 1.4990728 | | x |
| 25 | hsa-miR-652-3p | 8 | 0.667 | 0.4561 | 0.5 | >−0.2192 | 100 | 50 | 4.5751514 | | x |

^{a}All listed microRNAs demonstrate “HC Ratio” ≥1.5;
^{b}Youden index J;
^{c}Sensitivity;
^{d}Specificity;
^{e}=|Difference Compromised-Healthy/(mean SD)|

TABLE 5
Black race population: Top 25 microRNAs of 471 for pregnancy outcome prediction from microarray ordered by p value^{a}

| | | | | Area | | | | | | | |
|---|---|---|---|---|---|---|---|---|---|---|---|
| | | | | under | Significance | | | | | | |
| P | | | | the ROC | level | | | | | | |
| value | | | Sample | curve | P (Area = | | Associated | | | “HC | |
| rank | MicroRNA | | size | (AUC) | 0.5) | γ^{b} | criterion | Sens.^{c} | Spec.^{d} | Ratio”^{e} | P value^{f} |
| 1 | hsa-miR-1267 | 9 | 1 | 0.0001 | 1 | >2.8244 | 100 | 100 | 2.64 | x | x |
| 2 | hsa-miR-98-3p | 9 | 0.944 | 0.0001 | 0.8333 | >4.5488 | 83.33 | 100 | 2.25 | x | x |
| 3 | hsa-miR-149-5p | 9 | 0.944 | 0.0001 | 0.8333 | >6.112 | 83.33 | 100 | 1.89 | x | x |
| 4 | hsa-miR-452-5p | 9 | 0.833 | 0.0016 | 0.6667 | >0.1 | 66.67 | 100 | 2.09 | x | x |
| 5 | hsa-miR-455-5p | 9 | 0.833 | 0.0016 | 0.6667 | >0.1 | 66.67 | 100 | 1.9 | x | x |
| 6 | hsa-miR-198-5p | 9 | 0.833 | 0.0016 | 0.6667 | >0.1 | 66.67 | 100 | 1.795 | x | x |
| 7 | hsa-miR-563 | 9 | 0.889 | 0.0017 | 0.8333 | >3.8547 | 83.33 | 100 | 2.41 | x | x |
| 8 | hsa-miR-186-3p | 9 | 0.861 | 0.0108 | 0.8333 | >3.7453 | 83.33 | 100 | 1.61 | x | x |
| 9 | hsa-miR-1539 | 9 | 0.833 | 0.0455 | 0.8333 | >5.9896 | 83.33 | 100 | 1.829 | x | x |
| 10 | hsa-miR-391-3p | 9 | 0.833 | 0.0455 | 0.8333 | >7.4441 | 83.33 | 100 | 1.539882 | x | x |
| 11 | hsa-miR-24-1- | 9 | 0.833 | 0.0455 | 0.8333 | >12.6289 | 83.33 | 100 | 1.5331525 | x | x |
| 12 | hsa-miR-551b- | 9 | 0.833 | 0.0455 | 0.8333 | >62.7273 | 83.33 | 100 | 1.474918 | x | x |
| 13 | hsa-miR-624-3p | 9 | 0.778 | 0.0661 | 0.6667 | >1.3044 | 66.67 | 100 | 1.56 | | x |
| 14 | hsa-miR-501a- | 9 | 0.778 | 0.1102 | 0.6667 | >601.907 | 66.67 | 100 | 1.57 | | x |
| 15 | hsa-miR-557 | 9 | 0.778 | 0.1102 | 0.6667 | >20.1292 | 66.67 | 100 | 1.4536301 | | x |
| 16 | hsa-miR-548a- | 9 | 0.722 | 0.2278 | 0.6667 | >3.06423 | 66.67 | 100 | 1.698 | | x |
| 17 | hsa-miR-141-3p | 9 | 0.722 | 0.2402 | 0.6667 | >114.69 | 66.67 | 100 | 1.4631186 | | x |
| 18 | hsa-miR-18b-5p | 9 | 0.667 | 0.4292 | 0.6667 | >1.83022 | 66.67 | 100 | 1.855 | | x |
| 19 | hsa-miR-144-3p | 9 | 0.667 | 0.4292 | 0.6667 | >1670.41 | 66.67 | 100 | 1.724 | | x |
| 20 | hsa-miR-32-5p | 9 | 0.667 | 0.4292 | 0.6667 | >75.5787 | 66.67 | 100 | 1.64 | | x |
| 21 | hsa-miR-33b-5p | 9 | 0.667 | 0.4292 | 0.6667 | >1.57673 | 66.67 | 100 | 1.6 | | x |
| 22 | hsa-miR-590-5p | 9 | 0.667 | 0.4292 | 0.6667 | >720.79 | 66.67 | 100 | 1.5571643 | | x |
| 23 | hsa-miR-144-5p | 9 | 0.667 | 0.4292 | 0.6667 | >352.682 | 66.67 | 100 | 1.5295642 | | x |
| 24 | hsa-miR-33a-5p | 9 | 0.667 | 0.4292 | 0.6667 | >40.7681 | 66.67 | 100 | 1.497619 | | x |
| 25 | hsa-miR-545-3p | 9 | 0.667 | 0.4292 | 0.6667 | >12.0259 | 66.67 | 100 | 1.491847 | | x |

^{a}All listed microRNAs demonstrate “HC Ratio” ≥1.5;
^{b}Youden index J;
^{c}Sensitivity;
^{d}Specificity;
^{e}=|Difference Compromised-Healthy/(mean SD)|;
^{f}ROC curve <0.05)

TABLE 6
Population details: Training and Validation sets for “Unmet
Need” demonstration (Cluster ID importance) PCR study

| | | | | | |
|---|---|---|---|---|---|
| | | | | | Gest. age |
| Population groups | | Gest. Age sample | Maternal | | delivery |
| (Mean ± SD) | # Samples | (pregnancy weeks) | Age (years) | BMI | (weeks) |

Training set: 36 pregnant women

| | | | | | |
|---|---|---|---|---|---|
| Healthy pregnancy | | | | | |
| Non-Black | 17 | 12.7 ± 0.8 | 33.3 ± 7.8 | 26.8 ± 5.6 | 39.9 ± 0.9 |
| Black | 9 | 12.5 ± 0.4 | 31.4 ± 6.3 | 30.3 ± 8.1 | 39.6 ± 0.6 |
| Compromised pregnancy | | | | | |
| Non-Black | 3 | 12.6 ± 0.1 | 28.0 ± 6.2 | 29.6 ± 6.8 | 33.4 ± 4.2 |
| Black | 7 | 12.4 ± 0.5 | 35.0 ± 6.4 | 32.8 ± 10.9 | 33.7 ± 5.2 |
| Total: | 36 | | | | |

Validation set: 35 pregnant women

| | | | | | |
|---|---|---|---|---|---|
| Healthy pregnancy | | | | | |
| Non-Black | 15 | 12.7 ± 0.7 | 35.0 ± 3.8 | 24.3 ± 4.1 | 39.8 ± 0.9 |
| Black | 9 | 13.5 ± 2.8 | 34.2 ± 6.4 | 26.8 ± 2.6 | 40.0 ± 4.3 |
| Compromised pregnancy | | | | | |
| Non-Black | 4 | 12.4 ± 0.2 | 35.4 ± 4.5 | 30.1 ± 5.3 | 33.5 ± 4.5 |
| Black | 7 | 12.4 ± 0.8 | 33.9 ± 8.8 | 34.5 ± 11.8 | 34.6 ± 5.3 |
| Total: | 35 | | | | |

TABLE 7
MicroRNA ROC curves for pregnancy outcome prediction developed on a training set of non-Black patients. Each microRNA
Youden Index J Associated Criterion Value determines to the “cut-off” used to assign a risk score to a
patient. (Detailed Score method described in Winger, Reed: U.S. Pat. No. 10,323,282 B2 issued on Jun. 18, 2019)

| | | | | | | Area | | | | | |
|---|---|---|---|---|---|---|---|---|---|---|---|
| | | | | | | under the | | | | | |
| | | | Sample | Positive | Negative | ROC curve | | Youden | Associated | | |
| # | microRNA | Population | size | group ^{a} | group ^{b} | (AUC) | Significance^{c} | index J | criterion | Sens. | Specificity |
| 1 | miR-33a | Non-Black | 13 | 2 (15.38%) | 11 | 0.909 | <0.0001 | 0.91 | ≤23.1170072 | 100 | 90.91 |
| 2 | miR-155 | Non-Black | 18 | 3 (16.67%) | 15 | 0.844 | 0.0003 | 0.8 | ≤13.4318116 | 100 | 80 |
| 3 | miR-575 | Non-Black | 15 | 3 (20.00%) | 12 | 0.861 | 0.0024 | 0.67 | ≤27.6650842 | 100 | 66.67 |
| 4 | miR-1267 | Non-Black | 18 | 3 (16.67%) | 15 | 0.667 | 0.2086 | 0.53 | ≤14.7490935 | 100 | 53.33 |
| 5 | miR-340 | Non-Black | 15 | 2 (13.33%) | 13 | 0.731 | 0.2786 | 0.54 | ≤23.4352585 | 100 | 53.85 |
| 6 | miR-30e | Non-Black | 18 | 3 (16.67%) | 15 | 0.644 | 0.3453 | 0.53 | ≤12.0797695 | 100 | 53.33 |
| 7 | miR-133b | Non-Black | 18 | 3 (16.67%) | 15 | 0.644 | 0.4855 | 0.47 | ≤21.8467334 | 66.67 | 80 |
| 8 | miR-1267 | Non-Black | 16 | 3 (18.75%) | 13 | 0.667 | 0.5304 | 0.51 | ≤9.27266064 | 66.67 | 84.62 |
| 9 | miR-223 | Non-Black | 18 | 3 (16.67%) | 15 | 0.6 | 0.5784 | 0.33 | ≤14.8672543 | 100 | 33.33 |
| 10 | miR-301a | Non-Black | 17 | 3 (17.65%) | 14 | 0.548 | 0.8722 | 0.33 | ≤27.8489938 | 66.67 | 0 |
| 11 | miR-16 | Non-Black | 18 | 3 (16.67%) | 15 | 0.511 | 0.9679 | 0.33 | >13.8445097 | 33.33 | 100 |
| 12 | miR-1229 | Non-Black | | | | | | | | | |
| 13 | miR-148a | Non-Black | 1 | | | | | | | | |

^{a} Unhealthy pregnancy outcome;
^{b} Healthy pregnancy outcome;
^{c}Significance level P (Area = 0.5)

TABLE 8
MicroRNA ROC curves for pregnancy outcome prediction developed on a training set of Black patients. Each microRNA
Youden Index J. Assoc. Criterion Value determines to the “cut-off” used to assign a risk score to a
patient. (Detailed Score method described in Winger, Reed: U.S. Pat. No. 10,323,282 B2 issued on Jun. 18, 2019)

| | | | | | | Area | Significance | | | | |
|---|---|---|---|---|---|---|---|---|---|---|---|
| | | | | | | under the | level P | | | | |
| | | | Sample | Positive | Negative | ROC curve | (Area = | Youden | Associated | | |
| # | microRNA | Population | size | group ^{a} | group ^{b} | (AUC) | 0.5) | index J | criterion | Sensitivity | Specificity |
| 1 | miR-148a | Black | 13 | 6 (46.15%) | 7 (53.85%) | 0.786 | 0.0408 | 0.5238 | ≤29.108930 | 66.67 | 85.71 |
| 2 | miR-30e | Black | 16 | 7 (43.75%) | 9 (56.25%) | 0.762 | 0.0838 | 0.6349 | >13.754576 | 85.71 | 77.78 |
| 3 | miR-155 | Black | 16 | 7 (43.75%) | 9 (56.25%) | 0.73 | 0.1091 | 0.6667 | ≤17.871195 | 100 | 66.67 |
| 4 | miR-223 | Black | 16 | 7 (43.75%) | 9 (56.25%) | 0.73 | 0.1109 | 0.6349 | ≤13.005543 | 85.71 | 77.78 |
| 5 | miR-340 | Black | 13 | 6 (46.15%) | 7 (53.85%) | 0.738 | 0.1267 | 0.5476 | >24.700879 | 83.33 | 71.43 |
| 6 | miR-575 | Black | 11 | 5 (45.45%) | 6 (54.55%) | 0.767 | 0.1763 | 0.6333 | >31.169559 | 80 | 83.33 |
| 7 | miR-1267 | Black | 16 | 7 (43.75%) | 9 (56.25%) | 0.698 | 0.1831 | 0.4921 | >17.194837 | 71.43 | 77.78 |
| 8 | miR-133b | Black | 14 | 6 (42.86%) | 8 (57.14%) | 0.667 | 0.3116 | 0.4583 | >22.929899 | 83.33 | 62.5 |
| 9 | miR-126 | Black | 15 | 6 (40.00%) | 9 (60.00%) | 0.611 | 0.5062 | 0.3889 | >15.905575 | 50 | 88.89 |
| 10 | miR-301a | Black | 13 | 6 (46.15%) | 7 (53.85%) | 0.595 | 0.5806 | 0.2857 | >21.814630 | 100 | 28.57 |
| 11 | miR-1229 | Black | 8 | 4 (50.00%) | 4 (50.00%) | 0.625 | 0.6171 | 0.5 | ≤32.828576 | 75 | 75 |
| 12 | miR-16 | Black | 16 | 7 (43.75%) | 9 (56.25%) | 0.571 | 0.6476 | 0.3016 | ≤11.998493 | 85.71 | 44.44 |
| 13 | miR-33 | Black | 10 | 4 (40.00%) | 6 (60.00%) | 0.583 | 0.7443 | 0.5 | >34.527199 | 50 | 100 |

^{a} Unhealthy pregnancy outcome;
^{b} Healthy pregnancy outcome;
^{c}Significance level P (Area = 0.5)

TABLE 9
PCR study: Non-Black population using Non-Black score* for
pregnancy outcome prediction (see ROC curve in FIG. 1A)

| | (1) | | (2) | | (3) | | (4) | |
|---|---|---|---|---|---|---|---|---|
| Non-Black | miR33a | <23.12 | miR155 | <13.43 | miR575 | <27.67 | miR1267 | <14.7 |
| Patient # 1 | 44.4 | | 17.2 | | 32.4 | | 20.6 | |
| Patient # 2 | 40.9 | | 16.6 | | 32 | | 19.2 | |
| Patient # 3 | 40.2 | | 17.3 | | 36.6 | | 19.1 | |
| Patient # 4 | | | 16.3 | | 31.2 | | 17.8 | |
| Patient # 5 | 29.7 | | 15.7 | | 24.4 | 1 | 14.1 | 1 |
| Patient # 6 | 40.9 | | 15.8 | | 26.5 | 1 | 17 | |
| Patient # 7 | 30.1 | | 14.1 | | 25.3 | 1 | 12.2 | 1 |
| Patient # 8 | 24.3 | | 12.7 | 1 | 22.5 | 1 | 12.8 | 1 |
| Patient # 9 | | | 7.88 | 1 | | | 9.41 | 1 |
| Patient # 10 | 34 | | 15.6 | | 26.2 | 1 | 9.52 | 1 |
| Patient # 11 | | | 8.14 | 1 | | | 7.28 | 1 |
| Patient # 12 | 20.8 | 1 | 11.6 | 1 | 24.9 | 1 | 8.54 | 1 |
| Patient # 13 | 19.1 | 1 | 13.2 | 1 | 27.9 | | 13.5 | 1 |
| Patient # 14 | 20.6 | 1 | 5.14 | 1 | 19.5 | 1 | | |
| Patient # 15 | 15.7 | 1 | 16.7 | | | | 12.3 | 1 |
| Patient # 16 | | | | | | | | |
| Patient # 17 | | | | | | | | |
| Patient # 18 | | | | | | | | |
| Patient # 19 | | | | | | | | |

| | | | | | | Outcome | |
|---|---|---|---|---|---|---|---|
| | | | | | | (1 = | |
| | | | | | | Unhealthy, | |
| | (5) | | (6) | | | 2 = | |
| Non-Black | miR340 | <23.4 | miR30e-3p | <12.08 | Score | Healthy) | P.O. |
| Patient # 1 | 27.1 | 0 | 19.7 | | 0 | 0 | Healthy |
| Patient # 2 | 28.7 | 0 | 18.8 | | 0 | 0 | Healthy |
| Patient # 3 | 25.6 | 0 | 18.6 | | 0 | 0 | Healthy |
| Patient # 4 | 25 | 0 | 18.6 | | 0 | 0 | Healthy |
| Patient # 5 | 23.9 | 0 | 18.2 | | 2 | 0 | Healthy |
| Patient # 6 | 24.2 | 0 | 17.7 | | 1 | 0 | Healthy |
| Patient # 7 | 24.2 | 0 | 16.2 | | 2 | 1 | Early PE |
| Patient # 8 | 20.3 | 1 | 14.4 | | 4 | 1 | Late PE |
| Patient # 9 | | | 11.4 | 1 | 3 | 1 | Late PE |
| Patient # 10 | 22.5 | 1 | 9.9 | 1 | 4 | 0 | Healthy |
| Patient # 11 | 23.2 | 1 | 9.26 | 1 | 4 | 0 | Healthy |
| Patient # 12 | 19.7 | 1 | 6.99 | 1 | 6 | 1 | Early PE |
| Patient # 13 | | | 6.34 | 1 | 4 | 0 | Healthy |
| Patient # 14 | 21.1 | 1 | 3.28 | 1 | 5 | 0 | Healthy |
| Patient # 15 | | | 2.55 | 1 | 3 | 0 | Healthy |
| Patient # 16 | | | | | 0 | 0 | Healthy |
| Patient # 17 | | | | | 0 | 0 | Healthy |
| Patient # 18 | | | | | 0 | 0 | Healthy |
| Patient # 19 | | | | | 0 | 0 | Healthy |

*Score: Black on Black score; Outcome: 1 = Unhealthy, 0 = Healthy; P.O.: pregnancy outcome. Detailed Score method described in Winger, Reed: U.S. Pat. No. 10,323,282 B2 issued on Jun. 18, 2019.

TABLE 10
PCR study. Black population using non-Black score* for
pregnancy outcome prediction (See ROC curve in FIG. 1B)

| | (1) | | (2) | | (3) | | (4) | |
|---|---|---|---|---|---|---|---|---|
| Black | miR33a | <23.12 | miR155 | <13.43 | miR575 | <27.67 | miR1267 | <14.7 |
| Patient # 1 | | 0 | 16.57 | 0 | 39.11 | 0 | 20.5 | 0 |
| Patient # 2 | | 0 | 17.54 | 0 | | 0 | 18.21 | 0 |
| Patient # 3 | | 0 | 18.23 | 0 | 34.43 | 0 | 28.62 | 0 |
| Patient # 4 | 41.88 | 0 | 16.93 | 0 | 37.84 | 0 | 16.73 | 0 |
| Patient # 5 | | 0 | 10.98 | 1 | | 0 | 15.98 | 0 |
| Patient # 6 | | 0 | 15.32 | 0 | 29.52 | 0 | 16.92 | 0 |
| Patient # 7 | | 0 | 15.16 | 0 | 30.16 | 0 | 14.47 | 1 |
| Patient # 8 | 29.26 | 0 | 16.21 | 0 | 29.68 | 0 | 13.91 | 1 |
| Patient # 9 | 26.38 | 0 | 15.49 | 0 | 31.5 | 0 | 10.31 | 1 |
| Patient # 10 | | 0 | 7.755 | 1 | | 0 | 10.68 | 1 |
| Patient # 11 | 20.85 | 1 | 9.275 | 1 | 23.41 | 1 | 5.895 | 1 |
| Patient # 12 | 17.31 | 1 | 13.29 | 1 | | 0 | 12.2 | 1 |
| Patient # 13 | | 0 | | 0 | | 0 | | 0 |
| Patient # 14 | | 0 | | 0 | | 0 | | 0 |
| Patient # 15 | | 0 | | 0 | | 0 | | 0 |
| Patient # 16 | | 0 | | 0 | | 0 | | 0 |

| | (5) | | (6) | | | | |
|---|---|---|---|---|---|---|---|
| Black | miR340 | <23.4 | miR30e-3p | <12.08 | Score | Outcome | P.O. |
| Patient # 1 | 26.52 | 0 | 20.03 | 0 | 0 | 1 | Early PE |
| Patient # 2 | 27.55 | 0 | 19.58 | 0 | 0 | 1 | Early PE |
| Patient # 3 | 24.98 | 0 | 19.43 | 0 | 0 | 1 | Late PE |
| Patient # 4 | 25.29 | 0 | 18.28 | 0 | 0 | 0 | Healthy |
| Patient # 5 | | 0 | 17.66 | 0 | 1 | 0 | Healthy |
| Patient # 6 | 22.71 | 1 | 17.59 | 0 | 1 | 1 | Early PE |
| Patient # 7 | 24.39 | 0 | 17.5 | 0 | 1 | 0 | Healthy |
| Patient # 8 | 21.4 | 1 | 14.58 | 0 | 2 | 1 | Late PE |
| Patient # 9 | 18.1 | 1 | 11.02 | 1 | 3 | 0 | Healthy |
| Patient # 10 | | 0 | 10.14 | 1 | 3 | 0 | Healthy |
| Patient # 11 | 20.12 | 1 | 5.125 | 1 | 6 | 1 | Late PE |
| Patient # 12 | | 0 | | 0 | 3 | 0 | Healthy |
| Patient # 13 | | 0 | | 0 | 0 | 0 | Healthy |
| Patient # 14 | | 0 | | 0 | 0 | 0 | Healthy |
| Patient # 15 | | 0 | | 0 | 0 | 0 | Healthy |
| Patient # 16 | | 0 | | 0 | 0 | 1 | Late PE |

*Score: Black on Non-Black score; Outcome: 1 = Unhealthy, 0 = Healthy; P.O.: pregnancy outcome. Detailed score method described in Winger, Reed: U.S. Pat. No. 10,323,282 B2 issued on Jun. 18, 2019 which is incorporated herein in its entirety by reference.

TABLE 11
PCR study. Black population using Black score* for pregnancy
outcome prediction (See ROC curve in FIG. 1C)

| | (1) | | | | (3) | | (4) | | (5) | |
|---|---|---|---|---|---|---|---|---|---|---|
| Black | miR148a | <29.1 | (2)miR30e-3p | >13.75 | miR155 | <17.87 | miR223 | <13.00 | miR340 | >24.70 |
| Patient # 1 | 32.4 | | 18.3 | 1 | 16.9 | 1 | 11.5 | 1 | 25.3 | 1 |
| Patient # 2 | 31.8 | | 19.6 | 1 | 17.5 | | 12.7 | 1 | 27.5 | 1 |
| Patient # 3 | | | 10.1 | | 7.75 | 1 | 7.82 | 1 | | |
| Patient # 4 | 25.7 | 1 | 19.4 | 1 | 18.2 | | 13.2 | | 25 | |
| Patient # 5 | 26.4 | 1 | 14.6 | 1 | 16.2 | 1 | 14.5 | | 21.4 | |
| Patient # 6 | 22.8 | 1 | 11 | | 15.5 | 1 | 11.4 | 1 | 18.1 | |
| Patient # 7 | 29.2 | | 17.5 | 1 | 15.2 | 1 | 10.7 | 1 | 24.4 | |
| Patient # 8 | 35.2 | | 20 | 1 | 16.6 | 1 | 13.1 | | 26.5 | 1 |
| Patient # 9 | 27.5 | 1 | 17.6 | 1 | 15.3 | 1 | 11 | 1 | 22.7 | |
| Patient # 10 | | | 5.13 | | 9.27 | 1 | 14.1 | | 20.1 | |
| Patient # 11 | | | 17.7 | 1 | 11 | 1 | 11.4 | 1 | | |
| Patient # 12 | | | 1.62 | | 13.3 | 1 | 17.8 | | | |
| Patient # 13 | | | | | | | 16.4 | | | |
| Patient # 14 | | | | | | | | | | |
| Patient # 15 | | | | | | | | | | |
| Patient # 16 | | | | | | | | | | |

| | (6) | | (7) | | (8) | | | | |
|---|---|---|---|---|---|---|---|---|---|
| Black | miR575 | >31.169 | miR1267 | >17.19 | miR133b | >22.93 | Score | Outcome | P.O. |
| Patient # 1 | 37.8 | 1 | 16.7 | | 31.6 | 1 | 6 | 0 | Healthy |
| Patient # 2 | | | 18.2 | 1 | 28.2 | 1 | 11 | 1 | PE early |
| Patient # 3 | | | 10.7 | | 27.2 | 1 | 8 | 0 | Healthy |
| Patient # 4 | 34.4 | 1 | 28.6 | 1 | 26.2 | 1 | 8 | 1 | Late PE |
| Patient # 5 | 29.7 | | 13.9 | | 25.8 | 1 | 9 | 1 | Early PE |
| Patient # 6 | 31.5 | 1 | 10.3 | | 24.4 | 1 | 9 | 0 | Healthy |
| Patient # 7 | 30.2 | | 14.5 | | 23.8 | 1 | 9 | 0 | Healthy |
| Patient # 8 | 39.1 | 1 | 20.5 | 1 | 21.7 | | 9 | 1 | Early PE |
| Patient # 9 | 29.5 | | 16.9 | | 20.8 | | 9 | 1 | Early PE |
| Patient # 10 | 23.4 | | 5.7 | | 16.7 | | 5 | 1 | Late PE |
| Patient # 11 | | | 16 | | | | 4 | 0 | Healthy |
| Patient # 12 | | | 12.2 | | | | 4 | 0 | Healthy |
| Patient # 13 | | | | | | | 1 | 0 | Healthy |
| Patient # 14 | | | | | | | 0 | 0 | Healthy |
| Patient # 15 | | | | | | | 0 | 0 | Healthy |
| Patient # 16 | | | | | | | 0 | 1 | Late PE |

*Score: Black on Black score; Outcome: 1 = Unhealthy, 0 = Healthy; P.O.: pregnancy outcome. Detailed Score method described in Winger, Reed: U.S. Pat. No. 10,323,282 B2 issued on Jun. 18, 2019.

TABLE 12
PCR study. Non-Black population using Black score for
pregnancy outcome prediction (see ROC curve in FIG. 1D)

| | (1) | | | | (3) | | (4) | | (5) | |
|---|---|---|---|---|---|---|---|---|---|---|
| Non-Black | miR148a | <29.1 | (2)miR30e-3p | >13.75 | miR155 | <17.87 | miR223 | <13.00 | miR340 | >24.70 |
| Patient # 1 | 36.97 | 0 | 18.84 | 1 | 16.6 | 1 | 14.68 | 0 | 28.7 | 1 |
| Patient # 2 | 29.65 | 0 | 18.63 | 1 | 17.25 | 1 | 12.52 | 1 | 25.57 | 1 |
| Patient # 3 | 27.64 | 1 | 18.56 | 1 | 16.29 | 1 | 11.8 | 1 | 25.01 | 1 |
| Patient # 4 | | 0 | 6.34 | 0 | 13.19 | 1 | 19.95 | 0 | | 0 |
| Patient # 5 | | 0 | 2.547 | 0 | 16.74 | 1 | 14.94 | 0 | | 0 |
| Patient # 6 | 26.81 | 1 | 17.7 | 1 | 15.82 | 1 | 11.13 | 1 | 24.15 | 0 |
| Patient # 7 | | 0 | 9.283 | 0 | 8.137 | 1 | 6.827 | 1 | 23.2 | 0 |
| Patient # 8 | 27.05 | 1 | 18.21 | 1 | 15.73 | 1 | 11.17 | 1 | 23.86 | 0 |
| Patient # 9 | 27.44 | 1 | 18.21 | 1 | 14.08 | 1 | 10.99 | 1 | 24.19 | 0 |
| Patient # 10 | 33.02 | 0 | 19.72 | 1 | 17.24 | 1 | 13.39 | 0 | 27.15 | 1 |
| Patient # 11 | 25.06 | 1 | 14.43 | 1 | 12.75 | 1 | 7.939 | 1 | 20.32 | 0 |
| Patient # 12 | 31.87 | 0 | 6.985 | 0 | 11.57 | 1 | 14.6 | 0 | 19.74 | 0 |
| Patient # 13 | 29.26 | 0 | 9.901 | 0 | 15.55 | 1 | 13.3 | 0 | 22.48 | 0 |
| Patient # 14 | | 0 | 3.28 | 0 | 5.142 | 1 | 16.02 | 0 | 21.12 | 0 |
| Patient # 15 | | 0 | 11.35 | 0 | 7.883 | 1 | 9.173 | 1 | | 0 |
| Patient #16 | | 0 | | 0 | | 0 | | | | 0 |
| Patient #17 | | 0 | | 0 | | 0 | | | | 0 |
| Patient #18 | | 0 | | 0 | | 0 | | | | 0 |
| Patient #19 | | 0 | | 0 | | 0 | | | | 0 |

| | (6) | | (7) | | 8) | | | | |
|---|---|---|---|---|---|---|---|---|---|
| Non-Black | miR575 | >31.169 | miR1267 | >17.19 | miR133b | >22.93 | Score | Outcome | P.O. |
| Patient # 1 | 32.02 | 1 | 19.2 | 1 | 33.79 | 1 | 6 | 0 | Healthy |
| Patient # 2 | 36.59 | 1 | 19.11 | 1 | 29.92 | 1 | 7 | 0 | Healthy |
| Patient # 3 | 31.15 | 0 | 17.78 | 1 | 28.67 | 1 | 7 | 0 | Healthy |
| Patient # 4 | 27.94 | 0 | 13.47 | 0 | 26.6 | 1 | 2 | 0 | Healthy |
| Patient # 5 | | 0 | 12.31 | 0 | 25.6 | 1 | 2 | 0 | Healthy |
| Patient # 6 | 26.55 | 0 | 16.98 | 0 | 24.87 | 1 | 5 | 0 | Healthy |
| Patient # 7 | | 0 | 7.284 | 0 | 24.72 | 1 | 3 | 0 | Healthy |
| Patient # 8 | 24.39 | 0 | 14.07 | 0 | 24.17 | 1 | 5 | 0 | Healthy |
| Patient # 9 | 25.27 | 0 | 12.22 | 0 | 24.02 | 1 | 5 | 1 | Early PE |
| Patient # 10 | 32.44 | 1 | 20.6 | 1 | 22.32 | 0 | 5 | 0 | Healthy |
| Patient # 11 | 22.47 | 0 | 12.81 | 0 | 20.71 | 0 | 4 | 1 | Late PE |
| Patient # 12 | 24.94 | 0 | 8.545 | 0 | 20.41 | 0 | 1 | 1 | Early PE |
| Patient # 13 | 26.21 | 0 | 9.518 | 0 | 15.9 | 0 | 1 | 0 | Healthy |
| Patient # 14 | 19.5 | 0 | | 0 | 11.77 | 0 | 1 | 0 | Healthy |
| Patient # 15 | | 0 | 9.411 | 0 | | 0 | 2 | 1 | Late PE |
| Patient #16 | | 0 | | 0 | | 0 | 0 | 0 | Healthy |
| Patient #17 | | 0 | | 0 | | 0 | 0 | 0 | Healthy |
| Patient #18 | | 0 | | 0 | | 0 | 0 | 0 | Healthy |
| Patient #19 | | 0 | | 0 | | 0 | 0 | 0 | Healthy |

*Score: Black on Black score; Outcome: 1 = Unhealthy, 0 = Healthy; P.O.: pregnancy outcome. Detailed Score method described in Winger, Reed: U.S. Pat. No. 10,323,282 B2 issued on Jun. 18, 2019.

TABLE 13A
C1C2ratio calculations for race assessment taken from healthy
first trimester pregnancy samples in Blacks and non-Blacks using 175
microRNAs with low HC ratios <1.0 (low pregnancy outcome prediction
ability to focus on race sensitivity)
(See Table 13B for full table of 175 microRNAs)

| | | Cluster 1: | | Cluster 2: | | C1C2 | | |
|---|---|---|---|---|---|---|---|---|
| C1C2 | | Mean order | | Mean order | | Calculation = | | |
| calculation | | ranking of 175 | | ranking of 175 | | \|(Difference | | |
| Order | 175 microRNAs | miRs in NON- | | miRs in BLACKS (3 | | C2 − C1 | | T test |
| (Highest to | with HC | BLACKS (6 healthy | | healthy pregnant | | expression)/ | | p |
| lowest) | Ratio <1.0 | pregnant samples) | SD | samples) | SD | (Mean SD)\| | C1C2 >2.3 | value* |
| 1 | hsa-miR-150-5p | 55.6 | 25.9 | 2.3 | 1.7 | 3.85700298 | x | 0.01 |
| 2 | hsa-let-7g-5p | 82.9 | 40.7 | 4.5 | 3.3 | 3.55936353 | x | 0.01 |
| 3 | hsa-miR-16-5p | 82.1 | 40.9 | 4.5 | 4.2 | 3.44196763 | x | 0.02 |
| 4 | hsa-miR-29a-3p | 76.9 | 37.9 | 5.5 | 3.8 | 3.42060233 | x | 0.02 |
| 5 | hsa-let-7f-5p | 88.4 | 42.1 | 8.0 | 5.6 | 3.37086159 | x | 0.02 |
| 6 | hsa-miR-15b-5p | 79.6 | 41.8 | 6.3 | 4.5 | 3.16610454 | x | 0.02 |
| 7 | hsa-let-7i-5p | 85.6 | 41.2 | 9.5 | 6.9 | 3.1642313 | x | 0.02 |
| 8 | hsa-miR-26b-5p | 80.6 | 37.6 | 10.5 | 7.0 | 3.13838514 | x | 0.02 |
| 9 | hsa-miR-223-3p | 75.9 | 48.4 | 1.0 | 0.8 | 3.04200685 | x | 0.04 |
| 10 | hsa-miR-21-5p | 80.1 | 48.3 | 2.5 | 3.1 | 3.02246197 | x | 0.03 |
| 11 | hsa-miR-23a-3p | 85.0 | 45.0 | 11.3 | 8.1 | 2.7802185 | x | 0.03 |
| 12 | hsa-miR-142-3p | 77.6 | 51.8 | 3.3 | 2.5 | 2.73790201 | x | 0.05 |
| 13 | hsa-miR-19b-3p | 86.9 | 46.9 | 12.8 | 8.7 | 2.67015274 | x | 0.03 |
| 14 | hsa-let-7a-5p | 78.6 | 53.1 | 4.5 | 3.3 | 2.62528426 | x | 0.03 |
| 15 | hsa-miR-22-3p | 86.7 | 40.6 | 18.0 | 12.4 | 2.59663764 | x | 0.03 |
| 16 | hsa-miR-29c-3p | 78.4 | 50.1 | 9.0 | 6.1 | 2.47481324 | x | 0.05 |
| 17 | hsa-miR-342-3p | 69.7 | 38.1 | 12.3 | 9.0 | 2.44274309 | x | 0.04 |
| 18 | hsa-miR-25-3p | 79.4 | 38.4 | 17.5 | 12.8 | 2.42183473 | x | 0.03 |
| 19 | hsa-miR-24-3p | 82.3 | 45.2 | 16.5 | 11.4 | 2.32744139 | x | 0.04 |
| 20 | hsa-miR-17-5p | 77.0 | 36.8 | 20.0 | 13.6 | 2.26567742 | x | 0.04 |
| 21 | hsa-miR-30b-5p | 75.1 | 42.9 | 18.3 | 12.4 | 2.0542632 | x | 0.07 |
| 22 | hsa-miR-103a-3p | 66.7 | 45.0 | 11.8 | 8.7 | 2.04632663 | x | 0.08 |
| 23 | hsa-miR-146b-5p | 68.1 | 38.5 | 17.5 | 12.4 | 1.98823854 | x | 0.07 |
| 24 | hsa-miR-142-5p | 89.6 | 58.6 | 19.5 | 14.1 | 1.92989965 | x | 0.09 |
| 25 | hsa-miR-181a-5p | 78.1 | 44.2 | 22.8 | 15.5 | 1.85513326 | x | 0.08 |
| 26 | hsa-miR-126-3p | 76.9 | 45.2 | 22.0 | 14.7 | 1.83099019 | x | 0.09 |
| 27 | hsa-miR-338-3p | 112.6 | 64.4 | 34.8 | 24.4 | 1.75396275 | x | 0.09 |
| 28 | hsa-let-7b-5p | 59.7 | 47.7 | 11.8 | 8.5 | 1.70778028 | x | 0.13 |
| 29 | hsa-miR-1202 | 92.3 | 52.5 | 31.3 | 21.1 | 1.65895314 | x | 0.10 |
| 30 | hsa-miR-199a-3p | 73.6 | 47.2 | 22.8 | 15.8 | 1.6134873 | x | 0.12 |
| 31 | hsa-miR-155-5p | 82.3 | 49.4 | 28.3 | 19.2 | 1.57518212 | x | 0.12 |
| 32 | hsa-miR-140-3p | 65.0 | 30.1 | 28.3 | 19.3 | 1.4879003 | | |
| 33 | hsa-miR-194-5p | 112.1 | 66.5 | 42.0 | 28.1 | 1.48368344 | | |
| 34 | hsa-miR-107 | 56.0 | 43.2 | 16.0 | 10.8 | 1.48268974 | | |
| 35 | hsa-let-7d-5p | 57.3 | 44.0 | 16.5 | 11.6 | 1.46765887 | | |
| 36 | hsa-miR-130a-3p | 76.3 | 52.4 | 26.0 | 17.8 | 1.4330449 | | |
| 37 | hsa-miR-331-3p | 71.0 | 41.9 | 28.8 | 19.8 | 1.37140534 | | |
| 38 | hsa-miR-30d-5p | 69.3 | 37.3 | 30.5 | 20.8 | 1.33561327 | | |
| 39 | hsa-miR-92a-3p | 59.1 | 36.1 | 24.3 | 17.0 | 1.31383063 | | |
| 40 | hsa-miR-320d | 62.0 | 36.8 | 26.8 | 18.0 | 1.28693323 | | |

*P values calculated using Graphpad T test calculator from https://www.graphpad.com/quickcalcs/ttest1.cfm

TABLE 13B
C1C2ratio calculations for race assessment taken from healthy first trimester pregnancy samples in Blacks and non-Blacks
using 175 microRNAs with low HC ratios <1.0 (low pregnancy outcome prediction ability to focus on race sensitivity)

| | | Mean order ranking | | Mean order ranking | | | | |
|---|---|---|---|---|---|---|---|---|
| C1C2ratio Order | 175 microRNAs | of 175 miRs in NON- | | of 175 miRs in | | C1C2ratio | | |
| (Highest | with HC | BLACKS (6 healthy | | BLACKS (3 healthy | | (non-Blacks = C1 | | T test |
| to lowest) | Ratio <1.0 | pregnant samples) | SD | pregnant samples) | SD | Blacks = C2) | C1C2ratio >2.3 | P value* |
| 1 | hsa-miR-150-5p | 55.6 | 25.9 | 2.3 | 1.7 | 3.85700298 | x | 0.01 |
| 2 | hsa-let-7g-5p | 82.9 | 40.7 | 4.5 | 3.3 | 3.55936353 | x | 0.01 |
| 3 | hsa-miR-16-5p | 82.1 | 40.9 | 4.5 | 4.2 | 3.44196763 | x | 0.02 |
| 4 | hsa-miR-29a-3p | 76.9 | 37.9 | 5.5 | 3.8 | 3.42060233 | x | 0.02 |
| 5 | hsa-let-7f-5p | 88.4 | 42.1 | 8.0 | 5.6 | 3.37086159 | x | 0.02 |
| 6 | hsa-miR-15b-5p | 79.6 | 41.8 | 6.3 | 4.5 | 3.16610454 | x | 0.02 |
| 7 | hsa-let-7i-5p | 85.6 | 41.2 | 9.5 | 6.9 | 3.1642313 | x | 0.02 |
| 8 | hsa-miR-26b-5p | 80.6 | 37.6 | 10.5 | 7.0 | 3.13838514 | x | 0.02 |
| 9 | hsa-miR-223-3p | 75.9 | 48.4 | 1.0 | 0.8 | 3.04200685 | x | 0.04 |
| 10 | hsa-miR-21-5p | 80.1 | 48.3 | 2.5 | 3.1 | 3.02246197 | x | 0.03 |
| 11 | hsa-miR-23a-3p | 85.0 | 45.0 | 11.3 | 8.1 | 2.7802185 | x | 0.03 |
| 12 | hsa-miR-142-3p | 77.6 | 51.8 | 3.3 | 2.5 | 2.73790201 | x | 0.05 |
| 13 | hsa-miR-19b-3p | 86.9 | 46.9 | 12.8 | 8.7 | 2.67015274 | x | 0.03 |
| 14 | hsa-let-7a-5p | 78.6 | 53.1 | 4.5 | 3.3 | 2.62528426 | x | 0.03 |
| 15 | hsa-miR-22-3p | 86.7 | 40.6 | 18.0 | 12.4 | 2.59663764 | x | 0.03 |
| 16 | hsa-miR-29c-3p | 78.4 | 50.1 | 9.0 | 6.1 | 2.47481324 | x | 0.05 |
| 17 | hsa-miR-342-3p | 69.7 | 38.1 | 12.3 | 9.0 | 2.44274309 | x | 0.04 |
| 18 | hsa-miR-25-3p | 79.4 | 38.4 | 17.5 | 12.8 | 2.42183473 | x | 0.03 |
| 19 | hsa-miR-24-3p | 82.3 | 45.2 | 16.5 | 11.4 | 2.32744139 | x | 0.04 |
| 20 | hsa-miR-17-5p | 77.0 | 36.8 | 20.0 | 13.6 | 2.26567742 | x | 0.04 |
| 21 | hsa-miR-30b-5p | 75.1 | 42.9 | 18.3 | 12.4 | 2.0542632 | x | 0.07 |
| 22 | hsa-miR-103a-3p | 66.7 | 45.0 | 11.8 | 8.7 | 2.04632663 | x | 0.08 |
| 23 | hsa-miR-146b-5p | 68.1 | 38.5 | 17.5 | 12.4 | 1.98823854 | x | 0.07 |
| 24 | hsa-miR-142-5p | 89.6 | 58.6 | 19.5 | 14.1 | 1.92989965 | x | 0.09 |
| 25 | hsa-miR-181a-5p | 78.1 | 44.2 | 22.8 | 15.5 | 1.85513326 | x | 0.08 |
| 26 | hsa-miR-126-3p | 76.9 | 45.2 | 22.0 | 14.7 | 1.83099019 | x | 0.09 |
| 27 | hsa-miR-338-3p | 112.6 | 64.4 | 34.8 | 24.4 | 1.75396275 | x | 0.09 |
| 28 | hsa-let-7b-5p | 59.7 | 47.7 | 11.8 | 8.5 | 1.70778028 | x | 0.13 |
| 29 | hsa-miR-1202 | 92.3 | 52.5 | 31.3 | 21.1 | 1.65895314 | x | 0.10 |
| 30 | hsa-miR-199a-3p | 73.6 | 47.2 | 22.8 | 15.8 | 1.6134873 | x | 0.12 |
| 31 | hsa-miR-155-5p | 82.3 | 49.4 | 28.3 | 19.2 | 1.57518212 | x | 0.12 |
| 32 | hsa-miR-140-3p | 65.0 | 30.1 | 28.3 | 19.3 | 1.4879003 | | |
| 33 | hsa-miR-194-5p | 112.1 | 66.5 | 42.0 | 28.1 | 1.48368344 | | |
| 34 | hsa-miR-107 | 56.0 | 43.2 | 16.0 | 10.8 | 1.48268974 | | |
| 35 | hsa-let-7d-5p | 57.3 | 44.0 | 16.5 | 11.6 | 1.46765887 | | |
| 36 | hsa-miR-130a-3p | 76.3 | 52.4 | 26.0 | 17.8 | 1.4330449 | | |
| 37 | hsa-miR-331-3p | 71.0 | 41.9 | 28.8 | 19.8 | 1.37140534 | | |
| 38 | hsa-miR-30d-5p | 69.3 | 37.3 | 30.5 | 20.8 | 1.33561327 | | |
| 39 | hsa-miR-92a-3p | 59.1 | 36.1 | 24.3 | 17.0 | 1.31383063 | | |
| 40 | hsa-miR-320d | 62.0 | 36.8 | 26.8 | 18.0 | 1.28693323 | | |
| 41 | hsa-miR-125a-5p | 107.3 | 64.0 | 48.8 | 33.0 | 1.20661919 | | |
| 42 | hsa-miR-1246 | 78.0 | 60.9 | 29.0 | 22.0 | 1.18196886 | | |
| 43 | hsa-miR-483-5p | 112.3 | 72.8 | 54.5 | 39.0 | 1.03305998 | | |
| 44 | hsa-miR-181a-3p | 114.6 | 70.7 | 57.0 | 42.8 | 1.01436945 | | |
| 45 | hsa-miR-10a-5p | 100.1 | 70.6 | 47.8 | 33.7 | 1.00436441 | | |
| 46 | hsa-miR-320b | 59.4 | 35.8 | 32.0 | 21.4 | 0.95973362 | | |
| 47 | hsa-miR-125b-5p | 97.1 | 69.7 | 48.5 | 33.7 | 0.94108797 | | |
| 48 | hsa-miR-378a-5p | 85.6 | 67.7 | 40.8 | 28.6 | 0.93028798 | | |
| 49 | hsa-miR-1275 | 70.4 | 40.0 | 40.5 | 27.8 | 0.88285377 | | |
| 50 | hsa-miR-130b-3p | 64.3 | 41.0 | 35.8 | 24.3 | 0.87479863 | | |
| 51 | hsa-miR-1290 | 101.0 | 68.7 | 53.5 | 40.9 | 0.86682517 | | |
| 52 | hsa-miR-150-3p | 116.1 | 72.2 | 69.0 | 46.5 | 0.79425197 | | |
| 53 | hsa-miR-365a-3p | 102.7 | 69.8 | 58.5 | 44.2 | 0.77581617 | | |
| 54 | hsa-miR-584-5p | 94.4 | 67.6 | 54.3 | 36.5 | 0.7721553 | | |
| 55 | hsa-miR-181b-5p | 60.4 | 38.6 | 36.3 | 24.4 | 0.76741398 | | |
| 56 | hsa-miR-145-5p | 89.9 | 61.2 | 52.8 | 35.6 | 0.76731343 | | |
| 57 | hsa-miR-1914-3p | 96.0 | 75.6 | 53.5 | 35.9 | 0.76224281 | | |
| 58 | hsa-miR-486-5p | 72.0 | 57.6 | 39.0 | 30.7 | 0.74735488 | | |
| 59 | hsa-miR-146a-5p | 49.1 | 46.2 | 25.5 | 17.4 | 0.74439873 | | |
| 60 | hsa-miR-671-5p | 98.0 | 77.9 | 50.0 | 54.8 | 0.72347847 | | |
| 61 | hsa-miR-223-5p | 94.7 | 72.7 | 55.3 | 37.4 | 0.71670868 | | |
| 62 | hsa-miR-423-5p | 98.1 | 73.8 | 57.3 | 42.3 | 0.70439906 | | |
| 63 | hsa-miR-29b-1-5p | 68.0 | 55.2 | 39.3 | 26.7 | 0.70201592 | | |
| 64 | hsa-miR-100-5p | 101.1 | 69.5 | 62.0 | 47.1 | 0.67159735 | | |
| 65 | hsa-miR-320c | 49.6 | 51.3 | 27.3 | 18.3 | 0.64122886 | | |
| 66 | hsa-miR-1268a | 63.0 | 51.9 | 38.8 | 25.9 | 0.62365561 | | |
| 67 | hsa-miR-663a | 97.6 | 73.8 | 62.0 | 42.9 | 0.60932486 | | |
| 68 | hsa-miR-22-5p | 98.6 | 72.1 | 64.0 | 43.9 | 0.59613616 | | |
| 69 | hsa-miR-1225-3p | 56.1 | 58.8 | 31.5 | 24.0 | 0.59512093 | | |
| 70 | hsa-miR-532-3p | 93.9 | 61.2 | 64.0 | 43.9 | 0.56820135 | | |
| 71 | hsa-miR-376a-3p | 92.1 | 72.4 | 58.5 | 47.1 | 0.56287749 | | |
| 72 | hsa-miR-324-3p | 54.1 | 33.2 | 38.0 | 25.4 | 0.55171079 | | |
| 73 | hsa-miR-502-5p | 77.0 | 64.5 | 50.0 | 34.1 | 0.54782409 | | |
| 74 | hsa-miR-1305 | 99.6 | 75.0 | 66.8 | 45.2 | 0.54593048 | | |
| 75 | hsa-miR-135a-3p | 95.9 | 73.0 | 63.5 | 45.7 | 0.54491882 | | |
| 76 | hsa-miR-345-5p | 90.9 | 72.5 | 60.0 | 46.0 | 0.5207865 | | |
| 77 | hsa-miR-186-5p | 52.1 | 43.7 | 35.5 | 24.4 | 0.48875691 | | |
| 78 | hsa-miR-630 | 78.6 | 71.3 | 51.5 | 44.1 | 0.46949385 | | |
| 79 | hsa-miR-574-5p | 65.3 | 52.3 | 47.0 | 31.4 | 0.43691209 | | |
| 80 | hsa-miR-629-5p | 111.0 | 67.9 | 85.3 | 58.1 | 0.40845673 | | |
| 81 | hsa-miR-188-5p | 72.3 | 67.1 | 51.0 | 42.5 | 0.38854007 | | |
| 82 | hsa-miR-377-3p | 87.9 | 69.9 | 64.5 | 50.7 | 0.38721497 | | |
| 83 | hsa-miR-542-3p | 90.4 | 69.6 | 68.0 | 47.8 | 0.38214292 | | |
| 84 | hsa-miR-330-3p | 95.0 | 72.9 | 73.3 | 50.0 | 0.35393185 | | |
| 85 | hsa-miR-1181 | 77.0 | 73.6 | 58.0 | 39.5 | 0.33597495 | | |
| 86 | hsa-miR-200b-3p | 98.6 | 67.8 | 78.5 | 53.2 | 0.33176627 | | |
| 87 | hsa-miR-601 | 89.9 | 54.0 | 74.3 | 50.0 | 0.29993094 | | |
| 88 | hsa-miR-1271-5p | 90.0 | 67.9 | 73.3 | 50.0 | 0.28416487 | | |
| 89 | hsa-miR-21-3p | 61.0 | 39.8 | 50.8 | 34.7 | 0.27504839 | | |
| 90 | hsa-miR-16-2-3p | 94.7 | 65.4 | 78.3 | 55.7 | 0.27183714 | | |
| 91 | hsa-miR-629-3p | 91.3 | 68.8 | 75.5 | 50.7 | 0.26422571 | | |
| 92 | hsa-miR-193b-3p | 88.1 | 74.3 | 74.5 | 52.7 | 0.21490991 | | |
| 93 | hsa-miR-664-5p | 93.3 | 58.2 | 81.5 | 55.4 | 0.20757356 | | |
| 94 | hsa-miR-326 | 79.7 | 56.0 | 69.3 | 47.8 | 0.20158728 | | |
| 95 | hsa-miR-664-3p | 78.7 | 64.2 | 68.5 | 47.1 | 0.18366381 | | |
| 96 | hsa-miR-497-5p | 93.3 | 61.5 | 82.8 | 55.2 | 0.18054027 | | |
| 97 | hsa-miR-30a-5p | 77.6 | 61.6 | 68.0 | 46.3 | 0.17731339 | | |
| 98 | hsa-miR-486-3p | 105.4 | 68.7 | 96.5 | 64.5 | 0.13412305 | | |
| 99 | hsa-miR-638 | 51.4 | 53.6 | 45.8 | 33.6 | 0.1301481 | | |
| 100 | hsa-miR-133b | 83.7 | 65.7 | 77.5 | 53.0 | 0.10470386 | | |
| 101 | hsa-miR-181a-2-3p | 83.7 | 63.8 | 79.5 | 55.6 | 0.07057664 | | |
| 102 | hsa-miR-940 | 54.9 | 56.8 | 53.5 | 37.2 | 0.02888818 | | |
| 103 | hsa-miR-139-5p | 97.6 | 65.4 | 96.3 | 64.3 | 0.02038286 | | |
| 104 | hsa-miR-155-3p | 86.4 | 66.5 | 86.5 | 74.2 | −0.0010155 | | |
| 105 | hsa-miR-513a-5p | 75.4 | 56.7 | 76.0 | 54.7 | −0.0102611 | | |
| 106 | hsa-miR-181c-3p | 86.9 | 62.0 | 88.8 | 59.2 | −0.0312404 | | |
| 107 | hsa-miR-339-3p | 100.0 | 50.5 | 102.5 | 69.2 | −0.0417715 | | |
| 108 | hsa-miR-381 | 74.1 | 62.3 | 77.3 | 58.1 | −0.0516254 | | |
| 109 | hsa-miR-539-5p | 92.6 | 63.6 | 97.8 | 65.8 | −0.0800021 | | |
| 110 | hsa-miR-1224-5p | 69.0 | 65.0 | 73.8 | 53.2 | −0.0803344 | | |
| 111 | hsa-miR-421 | 97.4 | 65.3 | 104.0 | 71.6 | −0.0959827 | | |
| 112 | hsa-miR-654-3p | 71.6 | 65.8 | 78.5 | 60.7 | −0.1095402 | | |
| 113 | hsa-miR-30b-3p | 87.3 | 59.4 | 94.0 | 63.0 | −0.1096971 | | |
| 114 | hsa-miR-1226-5p | 81.6 | 73.0 | 89.0 | 60.0 | −0.1117619 | | |
| 115 | hsa-miR-143-3p | 83.7 | 65.5 | 92.3 | 63.2 | −0.1325989 | | |
| 116 | hsa-miR-127-3p | 62.6 | 57.6 | 69.8 | 50.6 | −0.1326759 | | |
| 117 | hsa-miR-15a-3p | 89.9 | 58.9 | 99.0 | 67.1 | −0.1451746 | | |
| 118 | hsa-miR-1471 | 65.7 | 71.9 | 76.8 | 58.4 | −0.1693598 | | |
| 119 | hsa-miR-574-3p | 53.1 | 55.3 | 62.5 | 42.9 | −0.1905497 | | |
| 120 | hsa-miR-23a-5p | 79.7 | 68.4 | 92.8 | 62.7 | −0.19889 | | |
| 121 | hsa-miR-222-3p | 41.1 | 45.0 | 49.0 | 33.1 | −0.2009865 | | |
| 122 | hsa-miR-622 | 84.0 | 51.6 | 97.5 | 69.4 | −0.2231093 | | |
| 123 | hsa-miR-766-3p | 57.0 | 43.0 | 68.0 | 45.8 | −0.2475827 | | |
| 124 | hsa-miR-1183 | 68.0 | 74.4 | 88.0 | 63.8 | −0.2895919 | | |
| 125 | hsa-miR-139-3p | 73.9 | 63.7 | 92.3 | 62.9 | −0.290532 | | |
| 126 | hsa-miR-1261 | 78.7 | 49.8 | 95.8 | 64.4 | −0.2983336 | | |
| 127 | hsa-miR-493-5p | 65.7 | 59.8 | 88.3 | 66.1 | −0.3581465 | | |
| 128 | hsa-miR-572 | 64.0 | 55.4 | 85.0 | 60.5 | −0.3624095 | | |
| 129 | hsa-miR-623 | 77.9 | 60.0 | 101.8 | 68.5 | −0.3718577 | | |
| 130 | hsa-miR-382-5p | 63.0 | 55.5 | 84.3 | 58.6 | −0.3725689 | | |
| 131 | hsa-miR-99b-3p | 89.9 | 54.2 | 115.8 | 78.4 | −0.3903932 | | |
| 132 | hsa-miR-26b-3p | 74.0 | 56.1 | 100.8 | 71.0 | −0.421171 | | |
| 133 | hsa-miR-202-3p | 69.6 | 56.8 | 97.5 | 66.5 | −0.4531205 | | |
| 134 | hsa-miR-101-5p | 76.9 | 60.8 | 108.8 | 73.6 | −0.4743609 | | |
| 135 | hsa-miR-543 | 62.9 | 56.4 | 92.8 | 69.4 | −0.475117 | | |
| 136 | hsa-miR-1825 | 66.9 | 72.6 | 103.0 | 71.7 | −0.5008794 | | |
| 137 | hsa-miR-298 | 62.1 | 50.4 | 93.3 | 66.6 | −0.5316709 | | |
| 138 | hsa-miR-30c-1-3p | 74.1 | 51.9 | 107.8 | 73.2 | −0.537183 | | |
| 139 | hsa-miR-431-5p | 55.0 | 51.5 | 89.8 | 67.0 | −0.5863763 | | |
| 140 | hsa-miR-1281 | 56.9 | 61.4 | 95.5 | 69.5 | −0.5902242 | | |
| 141 | hsa-miR-376a-5p | 66.9 | 51.9 | 104.3 | 74.7 | −0.590943 | | |
| 142 | hsa-miR-379-5p | 61.6 | 57.7 | 100.0 | 70.9 | −0.5977252 | | |
| 143 | hsa-miR-501-5p | 57.0 | 63.8 | 95.3 | 63.8 | −0.5993322 | | |
| 144 | hsa-miR-501-3p | 62.9 | 58.6 | 101.3 | 68.9 | −0.6024197 | | |
| 145 | hsa-miR-154-5p | 53.4 | 48.3 | 88.5 | 67.0 | −0.608182 | | |
| 146 | hsa-miR-92a-1-5p | 65.7 | 51.6 | 104.0 | 71.2 | −0.6234082 | | |
| 147 | hsa-miR-548b-5p | 66.9 | 56.1 | 107.5 | 72.8 | −0.6304894 | | |
| 148 | hsa-miR-423-3p | 73.1 | 53.0 | 115.3 | 77.1 | −0.6474573 | | |
| 149 | hsa-miR-602 | 55.0 | 58.7 | 95.3 | 64.0 | −0.6559588 | | |
| 150 | hsa-miR-933 | 66.4 | 70.4 | 115.0 | 77.4 | −0.657004 | | |
| 151 | hsa-miR-550a-5p | 64.3 | 64.0 | 109.8 | 74.1 | −0.6582102 | | |
| 152 | hsa-miR-28-3p | 81.4 | 50.2 | 126.5 | 84.3 | −0.6699499 | | |
| 153 | hsa-miR-610 | 74.6 | 47.1 | 116.5 | 77.7 | −0.6721302 | | |
| 154 | hsa-miR-196a-5p | 58.3 | 69.9 | 108.3 | 73.6 | −0.6963265 | | |
| 155 | hsa-miR-659-3p | 64.0 | 52.4 | 107.8 | 72.9 | −0.697993 | | |
| 156 | hsa-let-7b-3p | 60.6 | 64.3 | 108.5 | 72.8 | −0.6992656 | | |
| 157 | hsa-let-7f-1-3p | 62.3 | 67.4 | 112.5 | 75.7 | −0.7017772 | | |
| 158 | hsa-miR-92b-3p | 63.6 | 75.6 | 121.0 | 80.7 | −0.7346224 | | |
| 159 | hsa-miR-769-3p | 61.6 | 50.8 | 109.5 | 74.5 | −0.7648789 | | |
| 160 | hsa-miR-192-3p | 62.1 | 50.0 | 110.0 | 73.8 | −0.7731786 | | |
| 161 | hsa-miR-337-5p | 55.4 | 52.0 | 108.3 | 75.5 | −0.8284917 | | |
| 162 | hsa-miR-299-3p | 47.6 | 53.8 | 103.5 | 69.2 | −0.9094092 | | |
| 163 | hsa-miR-129-5p | 57.1 | 47.9 | 114.8 | 77.1 | −0.9217343 | | |
| 164 | hsa-miR-377-5p | 62.4 | 50.5 | 126.0 | 84.1 | −0.9439717 | | |
| 165 | hsa-miR-1470 | 51.7 | 59.8 | 118.0 | 78.8 | −0.9565807 | | |
| 166 | hsa-miR-125b-2-3p | 63.6 | 39.8 | 121.8 | 81.5 | −0.9589703 | | |
| 167 | hsa-miR-106a-3p | 63.4 | 48.3 | 129.0 | 86.0 | −0.9762021 | | |
| 168 | hsa-miR-885-3p | 58.4 | 47.1 | 122.5 | 82.9 | −0.9858061 | | |
| 169 | hsa-miR-760 | 42.0 | 60.5 | 108.0 | 72.7 | −0.9915031 | | |
| 170 | hsa-miR-634 | 52.0 | 62.1 | 125.5 | 83.7 | −1.0084598 | | |
| 171 | hsa-miR-129-2-3p | 46.1 | 64.2 | 126.5 | 84.4 | −1.0817029 | | |
| 172 | hsa-miR-616-3p | 51.9 | 42.0 | 122.5 | 82.3 | −1.1368657 | | |
| 173 | hsa-miR-876-3p | 43.0 | 62.1 | 126.5 | 84.4 | −1.1396534 | | |
| 174 | hsa-miR-1295a | 33.0 | 44.0 | 113.0 | 76.2 | −1.3303823 | | |
| 175 | hsa-miR-1294 | 38.4 | 42.1 | 130.5 | 87.0 | −1.4267729 | | |

*P values calculated using Graphpad T test calculator from https://www.graphpad.com/quickcalcs/ttest1.cfm, last accessed Feb. 23. 2023

TABLE 14
ROC curve Youden Index J Associated Criterion Value “Cut-off” values for microRNAs using only Healthy
pregnancy outcomes to focus Black vs. non-Black pregnancy group ethnicity assessment* (“Patient Cluster”)

| | | | | | Area | | | | | | | |
|---|---|---|---|---|---|---|---|---|---|---|---|---|
| | | | | | under | | | | | | | |
| | | | | Negative | the | | | | | | C1C2 | |
| | | | Positive | group | ROC | | | 95% | | | calculation | C1C2 |
| | | Sample | group | (non- | curve | Youden | Associated | Confidence | | | (from | ROC |
| # | MicroRNA | size | (Black) | Black) | (AUC) | index J | criterion | interval | Sensitivity | Specificity | Table 13) | P value |
| 1 | hsa-let-7g-5p | 9 | 3 | 6 | 1 | 1 | ≤7 | ≤4 to ≤7 | 100 | 100 | 3.559364 | 0.01 |
| 2 | hsa-miR-150-5p | 9 | 3 | 6 | 1 | 1 | ≤4 | ≤3 to ≤4 | 100 | 100 | 3.857003 | 0.01 |
| 3 | hsa-let-7f-5p | 9 | 3 | 6 | 1 | 1 | ≤13 | ≤10 to ≤13 | 100 | 100 | 3.370862 | 0.02 |
| 4 | hsa-let-7i-5p | 9 | 3 | 6 | 1 | 1 | ≤15 | ≤14 to ≤15 | 100 | 100 | 3.164231 | 0.02 |
| 5 | hsa-miR-15b-5p | 9 | 3 | 6 | 1 | 1 | ≤10 | ≤9 to ≤10 | 100 | 100 | 3.166105 | 0.02 |
| 6 | hsa-miR-16-5p | 9 | 3 | 6 | 1 | 1 | ≤10 | ≤5 to ≤10 | 100 | 100 | 3.441968 | 0.02 |
| 7 | hsa-miR-26b-5p | 9 | 3 | 6 | 1 | 1 | ≤15 | ≤14 to ≤15 | 100 | 100 | 3.138385 | 0.02 |
| 8 | hsa-miR-29a-3p | 9 | 3 | 6 | 1 | 1 | ≤8 | ≤6 to ≤8 | 100 | 100 | 3.420602 | 0.02 |
| 9 | hsa-let-7a-5p | 9 | 3 | 6 | 1 | 1 | ≤8 | ≤5 to ≤8 | 100 | 100 | 2.625284 | 0.03 |
| 10 | hsa-miR-19b-3p | 9 | 3 | 6 | 1 | 1 | ≤19 | ≤17 to ≤19 | 100 | 100 | 2.670153 | 0.03 |
| 11 | hsa-miR-21-5p | 9 | 3 | 6 | 1 | 1 | 57 | ≤2 to ≤7 | 100 | 100 | 3.022462 | 0.03 |
| 12 | hsa-miR-223-3p | 9 | 3 | 6 | 1 | 1 | ≤2 | ≤1 to ≤2 | 100 | 100 | 2.596638 | 0.03 |
| 13 | hsa-miR-23a-3p | 9 | 3 | 6 | 1 | 1 | ≤18 | ≤16 to ≤18 | 100 | 100 | 2.780219 | 0.03 |
| 14 | hsa-miR-25-3p | 9 | 3 | 6 | 1 | 1 | ≤27 | ≤16 to ≤27 | 100 | 100 | 2.421835 | 0.03 |
| 15 | hsa-miR-17-5p | 9 | 3 | 6 | 1 | 1 | ≤30 | ≤26 to ≤30 | 100 | 100 | 2.265677 | 0.04 |
| 16 | hsa-miR-22-3p | 9 | 3 | 6 | 1 | 1 | ≤28 | ≤23 to ≤28 | 100 | 100 | 3.042007 | 0.04 |
| 17 | hsa-miR-24-3p | 9 | 3 | 6 | 1 | 1 | ≤26 | ≤20 to ≤26 | 100 | 100 | 2.327441 | 0.04 |
| 18 | hsa-miR-342-3p | 9 | 3 | 6 | 1 | 1 | ≤21 | ≤16 to ≤21 | 100 | 100 | 2.442743 | 0.04 |
| 19 | hsa-miR-142-3p | 9 | 3 | 6 | 1 | 1 | ≤6 | ≤4 to ≤6 | 100 | 100 | 2.737902 | 0.05 |
| 20 | hsa-miR-29c-3p | 9 | 3 | 6 | 1 | 1 | ≤13 | ≤12 to ≤13 | 100 | 100 | 2.474813 | 0.05 |

*ROC curve calculations using MedCalc Statistical Software version 19.0.7 (MedCalc Software bvba, Ostend, Belgium; https://www.medcalc.org; 2019)

TABLE 15
Sequential microRNA quantification with Unhealthy pregnancy outcome (Column A) and Healthy pregnancy outcome (Column B)*

| Column A | | | | | Column B | | | | |
|---|---|---|---|---|---|---|---|---|---|
| xx = | | | | | xx = | | | | |
| Top marker | | | | | Top marker | | | | |
| candidate | | | | | candidate | | | | |
| microRNAs | | | | | microRNAs | | | | |
| (Demon- | | | | | (Demon- | | | | |
| strates | | | | | strates | | | | |
| reverse | | | | | reverse | | | | |
| behavior | | Change in | | | behavior | | Change in | | |
| in Unhealthy | | microRNA | | | in Unhealthy | | microRNA | | |
| vs Healthy | Unhealthy | expression | Pattern | | vs Healthy | Healthy | expression | Pattern | |
| pregnancy) | pregnancy | level | of change | Order | pregnancy) | pregnancy | level | of change | Order |
| xx | hsa-miR-29a-3p | 17825 | Most increasing | 1 | | hsa-miR-1268b | 65 | Most increasing | 1 |
| | hsa-miR-671-5p | 14704 | Most increasing | 2 | | hsa-miR-6165 | 44 | Most increasing | 2 |
| xx | hsa-let-7i-5p | 14334 | Most increasing | 3 | | hsa-miR-6763-5p | 30 | Most increasing | 3 |
| xx | hsa-let-7a-5p | 12883 | Most increasing | 4 | | hsa-miR-6793-5p | 25 | Most increasing | 4 |
| xx | hsa-let-7g-5p | 12060 | Most increasing | 5 | | hsa-miR-3912-5p | 24 | Most increasing | 5 |
| xx | hsa-miR-29c-3p | 11726 | Most increasing | 6 | | hsa-miR-602 | 21 | Most increasing | 6 |
| xx | hsa-miR-15b-5p | 10943 | Most increasing | 7 | | hsa-miR-4472 | 21 | Most increasing | 7 |
| xx | hsa-miR-150-5p | 10384 | Most increasing | 8 | | hsa-miR-4481 | 17 | Most increasing | 8 |
| xx | hsa-miR-26b-5p | 10349 | Most increasing | 9 | | hsa-miR-5196-5p | 17 | Most increasing | 9 |
| | hsa-miR-342-3p | 9864 | Most increasing | 10 | | hsa-miR-4711-3p | 15 | Most increasing | 10 |
| | hsa-miR-146b-5p | 9801 | Most increasing | 11 | | hsa-miR-6832-5p | 13 | Most increasing | 11 |
| xx | hsa-miR-16-5p | 9502 | Most increasing | 12 | | hsa-miR-4648 | 12 | Most increasing | 12 |
| xx | hsa-miR-20a-5p | 8162 | Most increasing | 13 | | hsa-miR-4707-5p | 11 | Most increasing | 13 |
| xx | hsa-let-7f-5p | 8148 | Most increasing | 14 | | hsa-miR-5092 | 11 | Most increasing | 14 |
| | hsa-let-7b-5p | 8071 | Most increasing | 15 | | hsa-miR-7154-5p | 10 | Most increasing | 15 |
| xx | hsa-miR-15a-5p | 7531 | Most increasing | 16 | | hsa-miR-5087 | 10 | Most increasing | 16 |
| | hsa-miR-4516 | 6251 | Most increasing | 17 | | hsa-miR-4456 | 10 | Most increasing | 17 |
| xx | hsa-miR-19b-3p | 6007 | Most increasing | 18 | | hsa-miR-6751-5p | 10 | Most increasing | 18 |
| xx | hsa-miR-23a-3p | 5608 | Most increasing | 19 | | hsa-miR-1261 | 10 | Most increasing | 19 |
| | hsa-miR-3960 | 5261 | Most increasing | 20 | | hsa-miR-455-5p | 9 | Most increasing | 20 |
| xx | hsa-miR-29b-3p | 5060 | Most increasing | 21 | | hsa-miR-4440 | 9 | Most increasing | 21 |
| | hsa-miR-155-5p | 4969 | Most increasing | 22 | | hsa-miR-6787-5p | 8 | Most increasing | 22 |
| xx | hsa-miR-223-3p | 4820 | Most increasing | 23 | | hsa-miR-7156-5p | 8 | Most increasing | 23 |
| xx | hsa-miR-107 | 4815 | Most increasing | 24 | | hsa-miR-504-3p | 8 | Most increasing | 24 |
| xx | hsa-let-7d-5p | 4653 | Most increasing | 25 | | hsa-miR-1179 | 8 | Most increasing | 25 |
| xx | hsa-miR-103a-3p | 4565 | Most increasing | 26 | | hsa-miR-4755-5p | 8 | Most increasing | 26 |
| xx | hsa-miR-21-5p | 4540 | Most increasing | 27 | | hsa-miR-5007-5p | 8 | Most increasing | 27 |
| xx | hsa-miR-106b-5p | 4519 | Most increasing | 28 | | hsa-miR-3189-3p | 8 | Most increasing | 28 |
| | hsa-miR-4459 | 4512 | Most increasing | 29 | | hsa-miR-219a-2-3p | 7 | Most increasing | 29 |
| | hsa-miR-30b-5p | 4429 | Most increasing | 30 | | hsa-miR-6760-5p | 7 | Most increasing | 30 |
| | hsa-miR-381-3p | −299 | Most decreasing | 30 | | hsa-miR-144-3p | −19500 | Most decreasing | 30 |
| | hsa-miR-3653-3p | −307 | Most decreasing | 29 | | hsa-miR-26a-5p | −20071 | Most decreasing | 29 |
| | hsa-miR-376b-3p | −310 | Most decreasing | 28 | xx | hsa-miR-106b-5p | −21380 | Most decreasing | 28 |
| | hsa-miR-409-3p | −315 | Most decreasing | 27 | | hsa-miR-24-3p | −21671 | Most decreasing | 27 |
| | hsa-miR-27b-3p | −315 | Most decreasing | 26 | xx | hsa-miR-107 | −21991 | Most decreasing | 26 |
| | hsa-miR-144-5p | −366 | Most decreasing | 25 | | hsa-miR-142-5p | −22283 | Most decreasing | 25 |
| | hsa-miR-337-5p | −367 | Most decreasing | 24 | xx | hsa-let-7i-5p | −24437 | Most decreasing | 24 |
| | hsa-miR-425-5p | −396 | Most decreasing | 23 | xx | hsa-miR-19a-3p | −24506 | Most decreasing | 23 |
| | hsa-miR-486-5p | −404 | Most decreasing | 22 | xx | hsa-miR-103a-3p | −26612 | Most decreasing | 22 |
| | hsa-miR-132-3p | −419 | Most decreasing | 21 | xx | hsa-miR-20a-5p | −27082 | Most decreasing | 21 |
| | hsa-miR-410-3p | −479 | Most decreasing | 20 | | hsa-miR-27a-3p | −28059 | Most decreasing | 20 |
| | hsa-miR-487b-3p | −519 | Most decreasing | 19 | xx | hsa-miR-23a-3p | −29522 | Most decreasing | 19 |
| | hsa-miR-126-5p | −522 | Most decreasing | 18 | xx | hsa-miR-29b-3p | −29702 | Most decreasing | 18 |
| | hsa-miR-495-3p | −530 | Most decreasing | 17 | | hsa-miR-7977 | −30229 | Most decreasing | 17 |
| | hsa-miR-338-3p | −585 | Most decreasing | 16 | xx | hsa-miR-15b-5p | −34221 | Most decreasing | 16 |
| | hsa-miR-142-3p | −628 | Most decreasing | 15 | xx | hsa-miR-26b-5p | −34324 | Most decreasing | 15 |
| | hsa-miR-221-3p | −656 | Most decreasing | 14 | xx | hsa-miR-150-5p | −34499 | Most decreasing | 14 |
| | hsa-miR-136-5p | −984 | Most decreasing | 13 | | hsa-miR-7975 | −37025 | Most decreasing | 13 |
| | hsa-miR-4284 | −1139 | Most decreasing | 12 | xx | hsa-miR-19b-3p | −38620 | Most decreasing | 12 |
| | hsa-miR-377-3p | −1249 | Most decreasing | 11 | xx | hsa-miR-29c-3p | −40587 | Most decreasing | 11 |
| | hsa-miR-5100 | −1331 | Most decreasing | 10 | xx | hsa-let-7f-5p | −41212 | Most decreasing | 10 |
| | hsa-miR-376a-3p | −1788 | Most decreasing | 9 | xx | hsa-miR-29a-3p | −42751 | Most decreasing | 9 |
| | hsa-miR-199a-5p | −1863 | Most decreasing | 8 | xx | hsa-let-7g-5p | −42862 | Most decreasing | 8 |
| | hsa-miR-376c-3p | −2315 | Most decreasing | 7 | xx | hsa-miR-15a-5p | −44417 | Most decreasing | 7 |
| | hsa-miR-130a-3p | −2601 | Most decreasing | 6 | xx | hsa-let-7a-5p | −46085 | Most decreasing | 6 |
| | hsa-miR-126-3p | −3776 | Most decreasing | 5 | xx | hsa-miR-21-5p | −46571 | Most decreasing | 5 |
| | hsa-miR-199a-3p | −3923 | Most decreasing | 4 | | hsa-miR-142-3p | −46695 | Most decreasing | 4 |
| | hsa-miR-7977 | −4945 | Most decreasing | 3 | xx | hsa-miR-16-5p | −51359 | Most decreasing | 3 |
| | hsa-miR-144-3p | −6484 | Most decreasing | 2 | xx | hsa-miR-223-3p | −53400 | Most decreasing | 2 |
| | hsa-miR-7975 | −7220 | Most decreasing | 1 | | hsa-miR-451a | −54692 | Most decreasing | 1 |

*Top 2.4% Most sequentially differentially expressed (most increasing and most decreasing) microRNAs in the first trimester: ↑Unhealthy pregnancy ↓Healthy pregnancy, out of 1,240 microRNAs from two sequential blood draws at 7 weeks pregnant and 11 weeks pregnant.

TABLE 16
MicroRNA features comparison table

| | | | C. Top 2.4% | D: Top 2.4% most | |
|---|---|---|---|---|---|
| | | B. Top 20 “race | decreasing microRNA | increasing microRNA | |
| | | identifying” microRNAs | expression levels in 1^{st} | expression levels in 1st | E. Neanderthal |
| | | in first trimester (Black | trimester Healthy | trimester Unhealthy | introgressed |
| | | vs Non-Black; see Table | Pregnancy (see Table | pregnancy (see Table | enhancer SNP gene |
| # | A. MicroRNA | 13) | 15, Column B) | 15, Column A) | related microRNAs** |
| 1 | hsa-let-7a-5p | xx | x | x | x |
| 2 | hsa-let-7f-5p | xx | x | x | x |
| 3 | hsa-let-7g-5p | xx | x | x | x |
| 4 | hsa-let-7i-5p | xx | x | x | x |
| 5 | hsa-miR-150-5p | xx | x | x | |
| 6 | hsa-miR-15b-5p | xx | x | x | |
| 7 | hsa-miR-16-5p | xx | x | x | x |
| 8 | hsa-miR-19b-3p | xx | x | x | x |
| 9 | hsa-miR-21-5p | xx | x | x | |
| 10 | hsa-miR-223-3p | xx | x | x | |
| 11 | hsa-miR-23a-3p | xx | x | x | x |
| 12 | hsa-miR-26b-5p | xx | x | x | |
| 13 | hsa-miR-29a-3p | xx | x | x | x |
| 14 | hsa-miR-29c-3p | xx | x | x | x |
| 15 | hsa-miR-142-3p | xx | x | | |
| 16 | hsa-miR-24-3p | xx | x | | x |
| 17 | hsa-miR-342-3p | xx | | x | |
| 18 | hsa-miR-17-5p | xx | | | x |
| 19 | hsa-miR-22-3p | xx | | | x |
| 20 | hsa-miR-25-3p | xx | | | x |
| 21 | hsa-miR-103a-3p | | x | x | |
| 22 | hsa-miR-106b-5p | | x | x | |
| 23 | hsa-miR-107 | | x | x | |
| 24 | hsa-miR-15a-5p | | x | x | x |
| 25 | hsa-miR-29b-3p | | x | x | x |
| 26 | hsa-miR-20a-5p | | x | x | |
| 27 | hsa-miR-26b-5p | | x | x | |
| 28 | hsa-miR-142-3p | | x | | |
| 29 | hsa-miR-144-3p | | x | | |
| 30 | hsa-miR-26a-5p | | x | | |
| 31 | hsa-miR-27a-3p | | x | | |
| 32 | hsa-miR-451a | | x | | |
| 33 | hsa-miR-7975 | | x | | |
| 34 | hsa-miR-7977 | | x | | |
| 35 | hsa-let-7b-5p | | | x | x |
| 36 | hsa-let-7d-5p | | | x | x |
| 37 | hsa-miR-146b-5p | | | x | |
| 38 | hsa-miR-155-5p | | | x | |
| 39 | hsa-miR-30b-5p | | | x | x |
| 40 | hsa-miR-4459 | | | x | |
| 41 | hsa-miR-4516 | | | x | |
| 42 | hsa-miR-671-5p | | | x | |

**Neanderthal gene information resource: Supplementary file entitled “Document S8. Table S7” from Silvert M, Quintana-Murci L, Rotival M. Am J Hum Genet. 2019 Jun. 6; 104(6): 1241-1250. doi: 10.1016/j.ajhg.2019.04.016. Epub 2019 May 30. PMID: 31155285; PMCID: PMC6557732.)

TABLE 17
KEGG pathways associated the most differentially expressed microRNAs between
Black and non-Black populations in the first trimester of pregnancy

| | | | | Number of microRNAs out of the top |
|---|---|---|---|---|
| | | | | 20 C1C2 microRNAs |
| # | KEGG pathway | p-value | #genes | (taken from Table 13) |
| 1 | MAPK signaling pathway (hsa04010) | 7.62E−31 | 113 | 20 |
| 2 | PI3K-Akt signaling pathway (hsa04151) | 1.60E−28 | 136 | 20 |
| 3 | Prostate cancer (hsa05215) | 1.95E−28 | 48 | 20 |
| 4 | Focal adhesion (hsa04510) | 6.13E−24 | 87 | 20 |
| 5 | Neurotrophin signaling pathway (hsa04722) | 6.13E−24 | 58 | 20 |
| 6 | Chronic myeloid leukemia (hsa05220) | 2.21E−23 | 38 | 20 |
| 7 | Small cell lung cancer (hsa05222) | 5.30E−23 | 42 | 20 |
| 8 | Pathways in cancer (hsa05200) | 4.18E−21 | 132 | 20 |
| 9 | mTOR signaling pathway (hsa04150) | 5.59E−20 | 34 | 20 |
| 10 | ErbB signaling pathway (hsa04012) | 1.39E−19 | 45 | 20 |
| 11 | Glioma (hsa05214) | 1.66E−18 | 38 | 20 |
| 12 | Transcriptional misregulation in cancer (hsa05202) | 3.38E−18 | 76 | 20 |
| 13 | Melanoma (hsa05218) | 1.18E−17 | 35 | 20 |
| 14 | p53 signaling pathway (hsa04115) | 1.37E−15 | 34 | 18 |
| 15 | Insulin signaling pathway (hsa04910) | 8.39E−13 | 56 | 20 |
| 16 | TGF-beta signaling pathway (hsa04350) | 9.92E−13 | 40 | 19 |
| 17 | Acute myeloid leukemia (hsa05221) | 1.14E−12 | 28 | 20 |
| 18 | Long-term potentiation (hsa04720) | 1.95E−12 | 32 | 20 |
| 19 | Endometrial cancer (hsa05213) | 5.86E−12 | 26 | 19 |
| 20 | Endocytosis (hsa04144) | 9.20E−12 | 78 | 20 |

TABLE 18
Study population characteristics. Self-identified racially distinct populations
of first trimester pregnant women with healthy pregnancy outcomes were randomly
divided into training and validation sets for microRNA analysis by racial group

| | | | | | | |
|---|---|---|---|---|---|---|
| | | Gest. Age sample | Maternal | | | Gest. age |
| | | (pregnancy | Age | | | delivery |
| Racial groups (Mean ± SD) | # Samples | weeks) | (years) | BMI | Gravida | (weeks) |

Training set: 193 pregnant women

| | | | | | | |
|---|---|---|---|---|---|---|
| White | 119 | 11.9 ± 2.9 | 30.1 ± 5.6 | 26.5 ± 6.6 | 2.3 ± 1.2 | 39.1 ± 1.3 |
| Black | 38 | 12.1 ± 3.0 | 25.4 ± 5.7 | 32.1 ± 8.8 | 3.5 ± 2.5 | 37.0 ± 4.0 |
| Hispanic | 23 | 11.9 ± 2.2 | 28.5 ± 6.7 | 29.1 ± 6.1 | 2.5 ± 1.2 | 38.3 ± 1.9 |
| Asian/AmerInd | 13 | 12.6 ± 2.8 | 30.8 ± 5.2 | 24.9 ± 6.1 | 2.5 ± 1.2 | 37.7 ± 1.7 |
| Total: | 193 | | | | | |

Validation set: 72 pregnant women

| | | | | | | |
|---|---|---|---|---|---|---|
| White | 42 | 11.9 ± 2.9 | 30.2 ± 5.1 | 26.2 ± 6.1 | 2.2 ± 1.0 | 38.7 ± 2.6 |
| Black | 8 | 10.8 ± 8.4 | 27.3 ± 4.3 | 35.5 ± 9.0 | 3.3 ± 1.6 | 35.5 ± 5.1 |
| Hispanic | 11 | 10.2 ± 2.2 | 27.3 ± 4.5 | 26.9 ± 4.5 | 2.9 ± 1.7 | 38.5 ± 1.9 |
| Asian/Amerind | 5 | 12.7 ± 2.8 | 33.2 ± 5.5 | 25.1 ± 1.4 | 2.4 ± 0.5 | 38.0 ± 1.9 |
| Mixed/unknown | 6 | 12.2 ± 2.1 | 31.2 ± 2.6 | 30.5 ± 1.4 | 3.5 ± 3.2 | 38.8 ± 12 |
| Total: | 72 | | | | | |

TABLE 19
Development a scoring system on a Training set: Selection of microRNAs for Cluster group (race) assessment panel

| | | | | | E | |
|---|---|---|---|---|---|---|
| | | B | | D | Asian/AmerInd | |
| | | Black % signal | C | Hispanic % signal | % signal (of 30 | |
| | A | (of 29 Healthy | White % signal (of 114 | (of 20 total | total Healthy | F |
| # | MicroRNAs | samples) | total Healthy samples) | Healthy samples) | samples) | Ficher's exact test P values * |
| 1 | hsa-miR-582-5p | 0.21 | 0.02 | 0.00 | 0.00 | 0.0001 (Black to non-Black) |
| 2 | hsa-miR-6737-3p | 0.28 | 0.09 | 0.25 | 0.27 | 0.0013 (white to non-white) |
| 3 | hsa-miR-193a-3p | 0.03 | 0.19 | 0.15 | 0.13 | 0.05 (Black to non-Black) |
| 4 | hsa-miR-223-5p | 0.59 | 0.68 | 0.85 | 0.80 | 0.18 (Black to non-Black) |
| 5 | hsa-miR-148a-3p | 0.07 | 0.01 | 0.05 | 0.03 | |
| 6 | hsa-miR-16-5p | 1.00 | 0.99 | 1.00 | 1.00 | |
| 7 | hsa-miR-181a-5p | 0.48 | 0.57 | 0.65 | 0.63 | |
| 8 | hsa-miR-210-3p | 0.66 | 0.74 | 0.70 | 0.67 | |
| 9 | hsa-miR-301a-3p | 0.79 | 0.70 | 0.70 | 0.70 | |
| 10 | hsa-miR-33a-5p | 0.00 | 0.00 | 0.00 | 0.00 | |
| 11 | hsa-miR-340-5p | 0.83 | 0.78 | 0.75 | 0.70 | |
| 12 | hsa-miR-575 | 0.76 | 0.73 | 0.65 | 0.63 | |
| 13 | hsa-miR-671-5p | 0.10 | 0.18 | 0.20 | 0.17 | |
| 14 | hsa-miR-30e-3p | 0.97 | 0.88 | 0.80 | 0.80 | |
| 15 | hsa-miR-7-5p | 0.55 | 0.39 | 0.35 | 0.33 | |
| 16 | hsa-miR-1267 | 0.45 | 0.54 | 0.50 | 0.43 | |
| 17 | hsa-miR-132-3p | 0.62 | 0.74 | 0.75 | 0.73 | |
| 18 | hsa-miR-133b | 0.03 | 0.07 | 0.10 | 0.07 | |
| 19 | hsa-miR-1-3p | 0.03 | 0.01 | 0.00 | 0.00 | |
| 20 | hsa-miR-146a-5p | 1.00 | 1.00 | 1.00 | 1.00 | |
| 21 | hsa-miR-155-5p | 1.00 | 1.00 | 1.00 | 1.00 | |
| 22 | hsa-miR-196a-5p | 0.62 | 0.63 | 0.60 | 0.67 | |
| 23 | hsa-miR-199a-5p | 0.00 | 0.03 | 0.10 | 0.10 | |
| 24 | hsa-miR-221-5p | 0.00 | 0.00 | 0.00 | 0.00 | |
| 25 | hsa-miR-424-5p | 0.00 | 0.00 | 0.00 | 0.00 | |
| 26 | hsa-miR-1244 | 0.41 | 0.24 | 0.45 | 0.43 | |
| 27 | hsa-miR-199b-5p | 0.00 | 0.04 | 0.00 | 0.03 | |
| 28 | hsa-miR-219-5p | 0.00 | 0.00 | 0.05 | 0.03 | |
| 29 | hsa-miR-513-5p | 0.00 | 0.00 | 0.00 | 0.00 | |
| 30 | hsa-miR-1229-5p | 0.07 | 0.11 | 0.05 | 0.03 | |
| 31 | hsa-miR-144-3p | 1.00 | 0.89 | 0.95 | 0.93 | |
| 32 | hsa-mir-223-3p | 1.00 | 1.00 | 1.00 | 1.00 | |
| 33 | hsa-miR-181a-3p | 0.76 | 0.78 | 0.70 | 0.63 | |
| 34 | hsa-miR-210-5p | 0.00 | 0.12 | 0.15 | 0.13 | |
| 35 | hsa-miR-30e-5p | 0.97 | 0.99 | 1.00 | 1.00 | |
| 36 | hsa-miR-221-3p | 0.72 | 0.78 | 0.65 | 0.63 | |
| 37 | hsa-miR-551b-3p | 0.00 | 0.00 | 0.00 | 0.00 | |
| 38 | hsa-miR-4485-5p | 0.83 | 0.60 | 0.65 | 0.53 | |
| 39 | hsa-miR-24-1-5p | 0.00 | 0.00 | 0.00 | 0.00 | |
| 40 | hsa-miR-1238-3p | 0.83 | 0.88 | 0.95 | 0.97 | |
| 41 | hsa-miR-6757-3p | 0.41 | 0.34 | 0.15 | 0.13 | |
| 42 | hsa-miR-6819-3p | 0.41 | 0.46 | 0.45 | 0.43 | |
| 43 | hsa-miR-6889-3p | 0.00 | 0.03 | 0.15 | 0.10 | |
| 44 | hsa-miR-6752-3p | 0.00 | 0.01 | 0.00 | 0.00 | |
| 45 | hsa-miR-1237-3p | 0.62 | 0.44 | 0.60 | 0.57 | |

* P values calculated using Graphpad T test calculator from https://www.graphpad.com/quickcalcs/ttest1.cfm, last accessed Jun. 25, 2024

TABLE 20
Application of a microRNA panel constructed using the Training set data from Table
19 for Cluster (race) assessment using the Validation set data from Table 19

| | | | | | | | | F. |
|---|---|---|---|---|---|---|---|---|
| | | | | | | | | Final |
| | | | | | | | | “Pregnancy Race |
| | | | | | | F. | | Type” |
| | | B. | | | E. | miR-223-5p | | designations: |
| | | Patient | C. | D. | miR-193a-3p | signal = | | Cluster 1 = |
| | | self- | miR582-5p | miR-6737-3p | signal = Cluster | Cluster 2 | G. | NonBlack |
| | A. | identified | signal = | signal = | 2 designation | designation | Final Cluster | Cluster 2 = |
| # | Patient ID # | race | Cluster 2 | Cluster2 | nullified | nullified | designation | Black |
| 1 | Patient # 1 | White | | | 24.32563229 | 21.6934869 | Cluster1 | Non-Black |
| 2 | Patient # 2 | White | | | | 21.41845978 | Cluster1 | Non-Black |
| 3 | Patient # 3 | White | | | | 27.25074729 | Cluster1 | Non-Black |
| 4 | Patient # 4 | White | | 23.39263363 | | | Cluster2 | Black |
| 5 | Patient # 5 | Black | 25.10551897 | | | | Cluster2 | Black |
| 6 | Patient # 6 | White | | | | 21.4672349 | Cluster1 | Non-Black |
| 7 | Patient # 7 | White | | | 23.21486135 | 20.38856461 | Cluster1 | Non-Black |
| 8 | Patient # 8 | White | | | 23.76143593 | 22.25261256 | Cluster1 | Non-Black |
| 9 | Patient # 9 | Multi Race | | 25.96068999 | | 22.37890851 | Cluster 2 | Non-Black |
| | | | | | | (Cluster 2 | nullified = | |
| | | | | | | nullified) | Cluster1 | |
| 10 | Patient # 10 | Hispanic | | | | 21.23499989 | Cluster1 | Non-Black |
| 11 | Patient # 11 | White | | | | 23.0782084 | Cluster1 | Non-Black |
| 12 | Patient # 12 | Black | | | | 22.89509772 | Cluster2 | Non-Black |
| 13 | Patient # 13 | Hispanic | | | 24.81579744 | 21.39288909 | Cluster1 | Non-Black |
| 14 | Patient # 14 | Black | 25.47973252 | | 23.42584525 | 21.95538153 | Cluster2 | Black |
| 15 | Patient # 15 | White | | | | 21.9357383 | Cluster1 | Non-Black |
| 16 | Patient # 16 | Hispanic | | | | 22.92485098 | Cluster1 | Non-Black |
| 17 | Patient # 17 | White | | | | 21.92284447 | Cluster1 | Non-Black |
| 18 | Patient # 18 | Hispanic | | | | 23.32055658 | Cluster1 | Non-Black |
| 19 | Patient # 19 | White | | | | 21.14138317 | Cluster1 | Non-Black |
| 20 | Patient # 20 | White | | | | 21.26511608 | Cluster1 | Non-Black |
| 21 | Patient # 21 | Black | 25.4274781 | | | 23.66552055 | Cluster2 | Black |
| 22 | Patient # 22 | Multi Race | | | | 21.4365215 | Cluster1 | Non-Black |
| 23 | Patient # 23 | White | | | 24.87959948 | 21.46495594 | Cluster1 | Non-Black |
| 24 | Patient # 24 | White | | | | 22.39409252 | Cluster1 | Non-Black |
| 25 | Patient # 25 | AI/AN | | | | 21.56718559 | Cluster1 | Non-Black |
| 26 | Patient # 26 | White | | 24.96340054 | — | 22.06458281 | Cluster 2 | Non-Black |
| | | | | | | (Cluster 2 | nullified = | |
| | | | | | | nullified) | Cluster1 | |
| 27 | Patient # 27 | White | | | | 21.45199166 | Cluster1 | Non-Black |
| 28 | Patient # 28 | White | | | 25.42600827 | 22.44944664 | Cluster1 | Non-Black |
| 29 | Patient # 29 | White | | 24.20924579 | | 22.26952408 | Cluster 2 | Non-Black |
| | | | | | | (Cluster 2 | nullified = | |
| | | | | | | nullified) | Cluster1 | |
| 30 | Patient # 30 | Multi Race | | 25.98435596 | | | Cluster2 | Black |
| 31 | Patient # 31 | White | | 24.62070349 | | 22.67528003 | Cluster 2 | Non-Black |
| | | | | | | (Cluster 2 | nullified = | |
| | | | | | | nullified) | Cluster1 | |
| 32 | Patient # 32 | White | | | | 22.5323726 | Cluster1 | Non-Black |
| 33 | Patient # 33 | White | | 22.29462629 | 22.05994311 | | Cluster 2 | Non-Black |
| | | | | | (Cluster 2 | | nullified = | |
| | | | | | nullified) | | Cluster1 | |
| 34 | Patient # 34 | Multi Race | | | | 20.61725765 | Cluster1 | Non-Black |
| 35 | Patient # 35 | White | | 24.31128368 | — | 2.03123514 | Cluster 2 | Non-Black |
| | | | | | | (Cluster 2 | nullified = | |
| | | | | | | nullified) | Cluster1 | |
| 36 | Patient # 36 | Hispanic | | | | 22.30919395 | Cluster1 | Non-Black |
| 37 | Patient # 37 | White | | 25.42980887 | | | Cluster2 | Black |
| 38 | Patient # 38 | Hispanic | | 22.30480146 | | | Cluster2 | Black |
| 39 | Patient # 39 | White | | | | 23.63248557 | Cluster1 | Non-Black |
| 40 | Patient # 40 | Hispanic | | | 26.47514267 | 21.4101689 | Cluster1 | Non-Black |
| 41 | Patient # 41 | White | | | | 22.51341849 | Cluster1 | Non-Black |

TABLE 21
Application of non-Black microRNA pregnancy prediction score panel to a “self-
identified” non-Black validation set population (See ROC curve, FIG. 2A)

| | Self | | | | | | | | |
|---|---|---|---|---|---|---|---|---|---|
| | ID | (1)miR- | | (2) miR- | | (3) miR- | | (4) miR- | |
| # | Race | 210-3p | ≤21.486 | 196a-5p | >23.216 | 133b | >24.874 | 221-3p | >20.116 |
| 1 | White | 22.826 | | | | | | | |
| 2 | White | | | | | | | 18.972 | |
| 3 | White | | | | | 23.545 | | 20.697 | x |
| 4 | White | 22.285 | | 24.307 | x | | | | |
| 5 | White | | | | | | | | |
| 6 | White | | | | | | | 19.219 | |
| 7 | White | 22.843 | | | | | | 19.971 | |
| 8 | White | 22.166 | | 22.832 | | | | | |
| 9 | White | 22.851 | | | | | | 20.000 | |
| 10 | White | | | 24.528 | x | 24.784 | | 20.164 | x |
| 11 | White | 23.413 | | 24.846 | x | | | 20.058 | |
| 12 | White | 23.398 | | | | | | 20.813 | x |
| 13 | White | 25.240 | | 25.891 | x | | | 20.536 | x |
| 14 | White | | | 24.298 | x | | | | |
| 15 | White | | | 22.340 | | | | | |
| 16 | White | 21.285 | x | 21.586 | | | | 20.409 | x |
| 17 | White | 21.908 | | | | | | 20.913 | x |
| 18 | White | 21.544 | | 22.777 | | | | 20.325 | x |
| 19 | White | 24.773 | | | | | | 20.257 | x |
| 20 | White | 24.893 | | | | | | | |
| 21 | White | | | 20.647 | | | | 20.575 | x |
| 22 | White | 20.049 | x | 20.783 | | | | | |
| 23 | White | | | | | | | 22.562 | x |
| 24 | White | 20.530 | x | 25.034 | x | | | | |
| 25 | White | 20.361 | x | | | | | | |
| 26 | White | 20.335 | x | 21.325 | | | | 20.177 | x |
| 27 | White | 20.029 | x | 22.125 | | | | 19.335 | |
| 28 | White | | | | | | | | |
| 29 | White | 19.827 | x | 20.541 | | | | 19.575 | |
| 30 | White | 18.511 | x | 21.122 | | | | | |
| 31 | White | 20.298 | x | 20.796 | | | | 20.353 | x |
| 32 | White | | | | | | | 18.660 | |
| 33 | White | | | | | | | 19.361 | |
| 34 | White | | | | | | | 19.551 | |
| 35 | White | 22.517 | | 23.613 | x | | | 19.923 | |
| 36 | White | | | | | | | | |
| 37 | White | 23.238 | | | | | | 21.387 | x |
| 38 | White | 21.964 | | 22.321 | | | | | |
| 39 | White | 21.388 | x | | | | | 20.245 | x |
| 40 | White | | | | | | | | |
| 41 | White | 20.529 | x | | | | | | |
| 42 | White | 19.824 | x | | | | | | |

| | (5) -miR- | | (6) -miR- | | (7) miR- | | | |
|---|---|---|---|---|---|---|---|---|
| # | 575 | >23.126 | 16-5p | <7.41 | 30e-3p | >21.511 | Score* | PO** |
| 1 | 18.966 | | 7.177 | x | 23.99 | x | 2 | 0 |
| 2 | | | 14.731 | | 22.58 | x | 1 | 1 |
| 3 | 21.185 | | 10.099 | | 22.38 | x | 2 | 0 |
| 4 | 22.044 | | 9.155 | | 22.38 | x | 2 | 0 |
| 5 | | | 8.350 | | 22.19 | x | 1 | 0 |
| 6 | | | 14.985 | | 21.97 | x | 1 | 0 |
| 7 | 23.048 | x | 9.199 | | 21.94 | x | 2 | 0 |
| 8 | | | 5.605 | x | 21.74 | x | 2 | 0 |
| 9 | 22.201 | | 9.705 | | 21.72 | x | 1 | 0 |
| 10 | 20.835 | | 7.355 | x | 21.68 | x | 4 | 0 |
| 11 | | | 9.482 | | 21.66 | x | 2 | 0 |
| 12 | 20.501 | | 9.108 | | 21.64 | x | 2 | 0 |
| 13 | 20.144 | | 9.547 | | 21.62 | x | 3 | 0 |
| 14 | | | 6.282 | x | 21.56 | x | 3 | 0 |
| 15 | | | 9.733 | | 21.47 | x | 1 | 0 |
| 16 | 22.749 | | 8.366 | | 21.42 | | 2 | 0 |
| 17 | 17.673 | | 8.034 | | 21.27 | | 1 | 0 |
| 18 | 18.216 | | 7.729 | | 21.26 | | 1 | 0 |
| 19 | 20.306 | | 9.812 | | 21.17 | | 1 | 0 |
| 20 | 22.941 | | 7.348 | x | 21.12 | | 1 | 0 |
| 21 | 21.912 | | 8.150 | | 21.05 | | 1 | 0 |
| 22 | 28.307 | x | 5.578 | x | 20.86 | | 3 | 0 |
| 23 | | | 5.681 | x | 20.67 | | 2 | 0 |
| 24 | | | 6.045 | x | 20.59 | | 3 | 0 |
| 25 | | | 7.215 | x | 20.55 | | 2 | 0 |
| 26 | 24.320 | x | 7.228 | x | 20.42 | | 4 | 0 |
| 27 | | | 5.151 | x | 20.26 | | 2 | 0 |
| 28 | 20.934 | | 6.084 | x | 20.12 | | 1 | 0 |
| 29 | | | 6.814 | x | 20.01 | | 2 | 1 |
| 30 | 20.174 | | 6.243 | x | 19.72 | | 2 | 0 |
| 31 | 23.966 | x | 7.041 | x | 19.57 | | 4 | 0 |
| 32 | | | 14.178 | | | | 0 | 1 |
| 33 | | | 14.138 | | | | 0 | 0 |
| 34 | 19.214 | | 10.599 | | | | 0 | 0 |
| 35 | 22.788 | | 9.469 | | | | 1 | 0 |
| 36 | | | 8.276 | | | | 0 | 0 |
| 37 | 18.272 | | 7.732 | | | | 1 | 1 |
| 38 | 26.759 | x | 6.730 | x | | | 2 | 0 |
| 39 | 16.538 | | 6.641 | x | | | 3 | 0 |
| 40 | | | 6.567 | x | | | 1 | 0 |
| 41 | | | 6.021 | x | | | 2 | 0 |
| 42 | | | 5.901 | x | | | 2 | 0 |

Score: Self ID Non-Black using Non-Black score;
**PO = Pregnancy outcome; 1 = Unhealthy, 0 = Healthy

TABLE 22
Application of Black microRNA pregnancy prediction score* panel to a “self-
identified” non-Black validation set population (see ROC curve, FIG. 2B)

| | Self ID | | | (2) | | (6) | | |
|---|---|---|---|---|---|---|---|---|
| | non- | (5) miR- | | miR- | | miR- | | (4) |
| Pt # | Black | 340-5p | <22.734 | 575 | <21.693 | 196a-5p | <22.94 | 1244 |
| 1 | White | | x | 28.307 | | 20.7829 | x | |
| 2 | White | | | 16.537 | x | | | |
| 3 | White | | | 22.749 | | 21.5858 | x | |
| 4 | White | 23.678 | | 18.272 | x | | | 24.209 |
| 5 | White | 22.528 | x | | | 24.2984 | | |
| 6 | White | 21.600 | x | | | | | |
| 7 | White | | x | 20.173 | x | 21.1217 | x | |
| 8 | White | | | 26.759 | | 22.3212 | x | |
| 9 | White | 23.287 | | | | | | |
| 10 | White | | | | | | | |
| 11 | White | 23.121 | | | | | | |
| 12 | White | | | | | | | |
| 13 | White | | | | | | | |
| 14 | White | | | | | 22.8317 | x | |
| 15 | White | 24.029 | | | | 25.0336 | | |
| 16 | White | | | | | | | |
| 17 | White | 20.572 | x | 20.934 | x | | | 24.058 |
| 18 | White | 22.764 | x | | | | | |
| 19 | White | 21.970 | x | | | 22.1250 | x | |
| 20 | White | 22.926 | x | 17.672 | x | | | 23.643 |
| 21 | White | | | 19.213 | x | | | |
| 22 | White | 22.969 | | 20.500 | x | | | |
| 23 | White | | | | | | | |
| 24 | White | 23.756 | | 21.185 | x | | | |
| 25 | White | 21.361 | x | | | 20.5413 | x | |
| 26 | White | 22.527 | x | 22.788 | | 23.6127 | | |
| 27 | White | 21.155 | x | 18.215 | x | 22.7770 | x | |
| 28 | White | 25.713 | | 18.965 | x | | | |
| 29 | White | 21.742 | x | 20.305 | x | | | |
| 30 | White | 23.797 | | | | | | |
| 31 | White | 21.874 | x | 23.047 | | | | |
| 32 | White | 21.747 | x | 24.319 | | 21.3249 | x | |
| 33 | White | 20.555 | x | 23.965 | | 20.7955 | x | |
| 34 | White | 22.039 | x | 22.940 | | | | |
| 35 | White | 21.810 | x | 22.043 | | 24.3070 | | |
| 36 | White | | | | | | | |
| 37 | White | 22.356 | x | 20.143 | x | 25.8914 | | 23.701 |
| 38 | White | 22.141 | x | | | 24.8461 | | |
| 39 | White | 21.805 | x | 20.835 | x | 24.5283 | | 23.640 |
| 40 | White | 21.938 | x | | | 22.3400 | x | |
| 41 | White | 22.203 | x | 22.200 | | | | |
| 42 | White | 20.866 | x | 21.911 | | 20.6472 | x | 23.336 |

| | | | | (1) miR- | | | |
|---|---|---|---|---|---|---|---|
| Pt # | >23.133 | (3) 223-3p | (>8.71 | 6737-3p | <24.314 | Score* | PO** |
| 1 | | 11.01638 | x | 25.42981 | | 2 | 0 |
| 2 | | 10.75827 | x | 24.9634 | | 2 | 0 |
| 3 | | 9.656324 | x | 24.6207 | | 2 | 0 |
| 4 | x | 8.810635 | x | 24.31128 | x | 4 | 1 |
| 5 | | 9.814296 | x | 24.20925 | x | 3 | 0 |
| 6 | | 11.09603 | x | 23.39263 | x | 3 | 0 |
| 7 | | 10.34563 | x | 22.29463 | x | 4 | 0 |
| 8 | | 11.62935 | x | | | 2 | 0 |
| 9 | | 11.28421 | x | | | 1 | 1 |
| 10 | | 10.96907 | x | | | 1 | 0 |
| 11 | | 10.96744 | x | | | 1 | 0 |
| 12 | | 10.69779 | x | | | 1 | 0 |
| 13 | | 10.55155 | x | | | 1 | 1 |
| 14 | | 10.49151 | x | | | 2 | 0 |
| 15 | | 10.45292 | x | | | 1 | 0 |
| 16 | | 10.43118 | x | | | 1 | 0 |
| 17 | x | 9.812025 | x | | | 4 | 0 |
| 18 | | 9.744193 | x | | | 1 | 0 |
| 19 | | 9.712846 | x | | | 3 | 0 |
| 20 | x | 9.632874 | x | | | 3 | 0 |
| 21 | | 9.23048 | x | | | 2 | 0 |
| 22 | | 9.121497 | x | | | 2 | 0 |
| 23 | | 9.089585 | x | | | 1 | 0 |
| 24 | | 8.827747 | x | | | 2 | 0 |
| 25 | | 8.812075 | x | | | 3 | 1 |
| 26 | | 8.695218 | | | | 1 | 0 |
| 27 | | 8.681577 | | | | 3 | 0 |
| 28 | | 8.677639 | | | | 1 | 0 |
| 29 | | 8.601219 | | | | 2 | 0 |
| 30 | | 8.548696 | | | | 0 | 0 |
| 31 | | 8.38839 | | | | 1 | 0 |
| 32 | | 8.324411 | | | | 2 | 0 |
| 33 | | 8.321612 | | | | 2 | 0 |
| 34 | | 8.309739 | | | | 1 | 0 |
| 35 | | 8.248101 | | | | 1 | 0 |
| 36 | | 8.229777 | | | | 0 | 0 |
| 37 | x | 8.214922 | | | | 3 | 0 |
| 38 | | 7.974719 | | | | 1 | 0 |
| 39 | x | 7.963829 | | | | 3 | 0 |
| 40 | | 7.85452 | | | | 2 | 0 |
| 41 | | 7.853451 | | | | 1 | 0 |
| 42 | x | 7.442359 | | | | 3 | 0 |

Score: Self ID Non-Black using Black score;
**PO = Pregnancy outcome; 1 = Unhealthy, 0 = Healthy

TABLE 23
Application of non-Black microRNA pregnancy prediction score panel to a “self-
identified” Black/Black mixed race validation set population (see ROC curve, FIG. 2 C)

| Patient | SelfID | (1) miR-210- | | (2)miR-196a- | | | | (4) miR-221- |
|---|---|---|---|---|---|---|---|---|
| ID # | Black *unknown | 3p | ≤21.486 | 5p | <23.216 | (3) miR-133b | <24.874 | 3p |
| 1 | Black | 32.000 | x | | | | | 21.954 |
| 2 | Multi Race | 31.188 | | 31.854 | | | | 21.816 |
| 3 | Black | | | 23.052 | | | | 21.649 |
| 4 | Multi Race | | | | | | | 21.363 |
| 5 | Other | 19.198 | | 32.02 | | | | 21.304 |
| 6 | Black | 31.123 | x | 23.62 | x | | | 21.024 |
| 7 | Black | 31.261 | | 32.001 | | | | 20.693 |
| 8 | Black | 21.362 | | 32.011 | | | | 20.68 |
| 9 | Black | 19.679 | | 20.984 | | | | 20.366 |
| 10 | Multi Race | 31.958 | | 23.143 | | | | 20.127 |
| 11 | Multi Race | 22.275 | x | 23.752 | x | 36.62 | x | 19.643 |
| 12 | Black | 21.741 | x | 22.77 | | | | |
| 13 | Black | 20.391 | | 21.632 | | | | |

| Patient | | | | (6) miR-16- | | (7) miR- | | | |
|---|---|---|---|---|---|---|---|---|---|
| ID # | >20.116 | (5) miR-575 | >23.126 | 5p | 16-5p <7.41 | 30a-3p | >21.511 | Score* | P.O.** |
| 1 | x | 20.322 | | 7.132 | x | 20.978 | | 3 | 0 |
| 2 | x | | | 8.448 | | 30.959 | | 1 | 0 |
| 3 | x | 21.6 | | 8.489 | | 31.809 | x | 2 | 0 |
| 4 | x | 20.1 | | 8.992 | | 21.283 | | 1 | 0 |
| 5 | x | 20.714 | | 8.714 | x | 19.69 | | 2 | 0 |
| 6 | x | 21.453 | | 8.74 | | 21.698 | x | 4 | 0 |
| 7 | x | 21.918 | | 8.304 | | 20.837 | | 1 | 1 |
| 8 | x | 21.416 | | 8.458 | | 20.543 | | 1 | 0 |
| 9 | x | 21.836 | | 7.239 | x | 20.003 | | 2 | 1 |
| 10 | x | | | 8.34 | x | 20.732 | | 2 | 0 |
| 11 | | 32.058 | | 8.878 | | 30.857 | | 3 | 0 |
| 12 | | 21.774 | | 6.418 | x | 19.715 | | 2 | 0 |
| 13 | | | | 6.003 | x | 21.214 | | 1 | 0 |

Self ID Black (or mixed/unknown) using Non-Black Score
**PO = Pregnancy outcome; 1 = Unhealthy, 0 = Healthy

TABLE 24
Application of Black microRNA pregnancy prediction score
panel to a “self- identified” Black and Black
mixed race validation set population (see ROC curve, FIG. 2D)

| Patient | Self ID | (1) miR- | | (2) -miR- | | (3) -mir- | |
|---|---|---|---|---|---|---|---|
| # | race | 6737-3p | <24.314 | 575 | <21.693 | 223-3p | >8.71 |
| 1 | Multi | | | 22.058 | | 7.355 | |
| 2 | Black | | | 21.493 | x | 8.931 | x |
| 3 | Multi | | | | | 8.006 | |
| 4 | Black | | | 21.600 | x | 9.105 | x |
| 5 | Black | | | 21.774 | | 10.821 | x |
| 6 | Other | | | 20.714 | x | 9.250 | x |
| 7 | Black | | | 21.418 | x | 9.084 | x |
| 8 | Black | | | 21.915 | | 9.892 | x |
| 9 | Black | | | | | 9.844 | x |
| 10 | Multi | 25.9606 | | | | 11.054 | x |
| 11 | Black | | | 21.838 | | 8.877 | x |
| 12 | Multi | | | 20.100 | x | 8.111 | |
| 13 | Black | | | 20.322 | x | 10.899 | x |
| 14 | Multi | 25.9843 | | | | 10.152 | x |

| | | | | | | | Self ID | |
|---|---|---|---|---|---|---|---|---|
| | | | | | | | Black | |
| | | | | | | | using | |
| Patient | (4)miR- | | (5) miR- | | (6) miR- | | Black | |
| # | 1244 | >23.133 | 340-5p | <22.734 | 196a-5p | <22.94 | score | PO** |
| 1 | | | 21.237 | x | 23.753 | | 1 | 0 |
| 2 | | | 23.442 | | 23.520 | | 2 | 0 |
| 3 | | | 22.581 | x | 23.143 | | 1 | 0 |
| 4 | 23.742 | x | 22.712 | | 23.052 | x | 4 | 0 |
| 5 | | | | | 22.770 | x | 2 | 0 |
| 6 | | | | | 22.020 | x | 3 | 0 |
| 7 | | | 22.474 | x | 22.011 | x | 4 | 0 |
| 8 | | | 22.569 | x | 22.001 | x | 3 | 1 |
| 9 | | | | | 21.832 | x | 2 | 0 |
| 10 | | | 22.511 | x | 21.594 | x | 3 | 0 |
| 11 | | | 21.448 | x | 20.984 | x | 3 | 1 |
| 12 | | | 23.483 | | | | 1 | 0 |
| 13 | 27.192 | x | | | | | 3 | 0 |
| 14 | | | | | | | 1 | 0 |

Self ID Black (or mixed/unknown) using Black score;
**PO = Pregnancy outcome; 1 = Unhealthy, 0 = Healthy

TABLE 25
Application of non-Black microRNA pregnancy prediction score panel to a “microRNA
designated” non-black validation set population (See ROC curve, FIG. 3E)

| | Patient | | | | | | | | |
|---|---|---|---|---|---|---|---|---|---|
| | Self- | | | | | | | | |
| | Identified | Race-type by | (1) miR-210- | | (2) miR- [symbol] - | | (3) miR- | | |
| Patient # | race | microRNA score | 3p | ≤21.486 | 5p | >23.216 | 133b | >24.834 | (4) miR-221-3p |
| 1 | White | Non-black type | 22.82591622 | | | | | | |
| 2 | Hispanic | Non-black type | 25.22753675 | | | | | | 19.74163334 |
| 3 | White | Non-black type | | | | | | | 18.97166813 |
| 4 | White | Non-black type | 22.84285833 | | | | | | 19.97128631 |
| 5 | Hispanic | Non-black type | 22.6643771 | | 25.83869234 | x | | | |
| 6 | White | Non-black type | 22.15589073 | | 22.33178824 | | | | |
| 7 | White | Non-black type | 22.85078654 | | | | | | 19.5999528 |
| 8 | White | Non-black type | | | 24.52831533 | x | 24.788562 | | 20.16358496 |
| 9 | White | Non-black type | 23.41269879 | | 24.3461422 | x | | | 20.36773598 |
| 10 | White | Hon-black type | 25.23982094 | | | x | | | 20.53808887 |
| 11 | White | Non-black type | | | 24.29846562 | x | | | |
| 12 | White | Non-black type | | | 22.34637457 | | | | |
| 13 | White | Non-black type | 21.28463692 | x | 21.58523865 | | | | 20.40913219 |
| 14 | White | Non-black type | 21.90835977 | | | | | | 20.91345621 |
| 15 | White | Non-black type | 21.54372524 | | 22.7775254 | | | | 28.32482466 |
| 16 | White | Non-black type | 24.77386202 | | | | | | |
| 17 | White | Non-black type | | | 28.54725153 | | | | 26.57514595 |
| 18 | AI/AN | Non-black type | 21.47045917 | x | | | | | |
| 19 | Multi Race | Non-black type | 21.18329386 | x | 23.59858665 | | | | 21.83648716 |
| 20 | Multi Race | Non-black type | 22.27484988 | | 23.75818783 | x | 25.515288 | x | 19.52266971 |
| 21 | Hispanic | Non-black type | 22.93315185 | | 22.49825429 | | | | 12.85149998 |
| 22 | Hispanic | Non-black type | 19.73598891 | x | | | | | |
| 23 | Multi Race | Non-black type | 21.35829168 | x | 23.14939891 | | | | 26.12655748 |
| 24 | White | Non-black type | 20.52961059 | x | 25.88364125 | x | | | |
| 25 | Black | Non-black type | 21.36194551 | x | 22.24141285 | | | | 20.68084697 |
| 26 | White | Non-black type | 28.33518191 | x | 21.32295273 | | | | 2.17521657 |
| 27 | Hispanic | Non-black type | 20.93545887 | x | 20.95218519 | | 5.527822 | x | 19.42614544 |
| 28 | Hispanic | Non-black type | 21.97544154 | | 20.45348863 | | | | |
| 29 | White | Non-black type | 18.51115941 | x | 21.12175388 | | | | |
| 30 | White | Non-black type | 20.29802988 | x | | | | | 20.55342049 |
| 31 | White | Non-black type | 22.51705425 | | 23.61271983 | x | | | 13.92325211 |
| 32 | White | Non-black type | 23.23755164 | | | | | | 21.3872298 |
| 33 | White | Non-black type | 21.32249194 | x | | | | | 20.24539641 |
| 34 | White | Non-black type | | | | | | | |

| | | | | | | | | | Pregnancy |
|---|---|---|---|---|---|---|---|---|---|
| | | | | | | | | Non-black | [symbol] |
| | | | | | | | | using Non- | 0 = Healthy; |
| Patient # | >20.116 | (5)miR-575 | >23.125 | (6) miR-16-5p | <7.41 | (7) miR-30e-3p | >21.511 | black score | 1 = Compromised |
| 1 | | 18.955824 | | 7.177834534 | x | 28.221233 | x | 2 | 0 |
| 2 | | | | 8.725432948 | | 23.587325 | x | 1 | 0 |
| 3 | | | | 14.75060014 | | 22.58415 | x | 1 | 1 |
| 4 | | 23.047945 | | 9.190696354 | | 31.938278 | x | 1 | 0 |
| 5 | | 22.668587 | | 8.35234475 | | 21.392568 | x | 2 | 0 |
| 6 | | | | 5.602927315 | x | 21.737483 | x | 2 | 0 |
| 7 | | 22.20069 | | 9.304869611 | | 24.724995 | x | 1 | 0 |
| 8 | x | 28.385383 | | 7.354612194 | x | 21.682473 | x | 4 | 0 |
| 9 | | | | [symbol] | | 21.55494 | x | 2 | 0 |
| 10 | x | 29.143203 | | 9.54718221 | | 21.615124 | x | 3 | 0 |
| 11 | | | | 6.281916208 | x | 21.587775 | x | 3 | 0 |
| 12 | | | | 9.73387813 | | 21.258956 | | 0 | 0 |
| 13 | x | 22.349453 | | 8.355129301 | | 21.419816 | | 2 | 0 |
| 14 | x | 17.67289 | | 8.083600528 | | 21.265062 | | 1 | 0 |
| 15 | x | 18.215338 | | 7.728643292 | | 21.253365 | | 1 | 0 |
| 16 | x | 20.32571 | | 9.812407683 | | 21.17425 | | 1 | 0 |
| 17 | x | 21.311575 | | 8.158848858 | | 21.045437 | | 1 | 0 |
| 18 | | 23.634237 | x | 9.308453935 | | 20.98955 | | 2 | 0 |
| 19 | x | | | 8.848116514 | | 20.959278 | | 2 | 0 |
| 20 | | 22.458277 | | 8.377943121 | | 26.35715 | | 2 | 0 |
| 21 | | 23.885047 | | 8.807383422 | | 20.799858 | | 0 | 0 |
| 22 | | | | 7.100308291 | x | 20.355582 | | 2 | 0 |
| 23 | x | | | 5.339512915 | x | 20.73235 | | 3 | 0 |
| 24 | | | | 6.04452528 | x | 20.500922 | | 3 | 0 |
| 25 | x | 21.418217 | | 8.457862007 | | 20.54299 | | 2 | 0 |
| 26 | x | 24.319823 | x | 7.227640771 | x | 20.417011 | | 4 | 0 |
| 27 | | 23.784516 | x | 7.877122131 | x | 20.338687 | | 4 | 0 |
| 28 | | 23.525623 | x | 8.461589882 | | 20.31431 | | 1 | 0 |
| 29 | | 22.173502 | | 6.243809432 | x | 19.721489 | | 2 | 0 |
| 30 | x | 23.965882 | x | 7.541061928 | x | 19.570462 | | 4 | 0 |
| 31 | | 22.788388 | | 9.46941633 | | | | 1 | 0 |
| 32 | x | 18.273122 | | 7.781741882 | | | | 1 | 1 |
| 33 | x | 16.537353 | | 5.54133273 | x | | | 3 | 0 |
| 34 | | | | 6.566783374 | x | | | 1 | 0 |

Score: “MicroRNA designated” Non-Black using Non-Black Score; Pregnancy outcome: 1 = Unhealthy, 0 = Healthy [symbol]
indicates data missing or illegible when filed

TABLE 26
Application of non-Black microRNA pregnancy prediction score* panel to a “microRNA
designated” Black validation set population (see ROC curve, FIG. 3F)

| | | Race | | | | | | | |
|---|---|---|---|---|---|---|---|---|---|
| | | type by | | | | | | | |
| Patient | Black + | microRNA | (1) miR- | | (2) miR- | | (3) miR- | | (4) miR- |
| # | Unknown | score | 210-3p | ≤21.486 | 196a-5p | >23.216 | 133b | >24.874 | 221-3p |
| 1 | Black | Black | 22.008 | x | | | | | 21.954 |
| 2 | Multi Race | Black | 21.183 | | 21.594 | | | | 21.816 |
| 3 | Black | Black | | | 23.052 | | | | 21.549 |
| 4 | Multi Race | Black | | | | | | | 21.363 |
| 5 | Other | Black | 19.198 | | 22.020 | | | | 21.304 |
| 6 | Black | Black | 22.223 | x | 23.920 | x | | | 21.024 |
| 7 | Black | Black | 21.261 | | 22.001 | | | | 20.693 |
| 8 | Black | Black | 21.362 | | 22.011 | | | | 20.680 |
| 9 | Black | Black | 19.679 | | 20.984 | | | | 20.365 |
| 10 | Multi Race | Black | 21.358 | | 23.143 | | | | 20.127 |
| 11 | Multi Race | Black | 22.275 | x | 23.753 | x | 25.52 | x | 19.543 |
| 12 | Black | Black | 21.741 | x | 22.770 | | | | |
| 13 | Black | Black | 20.381 | | 21.832 | | | | |
| 14 | Multi Race | Black | 20.834 | | | | | | |

| Patient | | (5) miR- | | (6) miR- | 16-5p | (7) miR- | | | |
|---|---|---|---|---|---|---|---|---|---|
| # | >20.116 | 575 | >23.126 | 16-5p | <7.41 | 30a-3p | >21.511 | Score | PO |
| 1 | x | 20.322 | | 7.132 | x | 20.976 | | 3 | 0 |
| 2 | x | | | 8.448 | | 20.959 | | 1 | 0 |
| 3 | x | 21.600 | | 8.489 | | 21.809 | x | 2 | 0 |
| 4 | x | 20.100 | | 8.992 | | 21.263 | | 1 | 0 |
| 5 | x | 20.714 | | 5.714 | x | 19.590 | | 2 | 0 |
| 6 | x | 21.493 | | 8.740 | | 21.698 | x | 4 | 0 |
| 7 | x | 21.915 | | 8.304 | | 20.897 | | 1 | 1 |
| 8 | x | 21.418 | | 8.458 | | 20.543 | | 1 | 0 |
| 9 | x | 21.838 | | 7.239 | x | 20.003 | | 2 | 1 |
| 10 | x | | | 5.340 | x | 20.732 | | 2 | 0 |
| 11 | | 22.058 | | 8.878 | | 20.857 | | 3 | 0 |
| 12 | | 21.774 | | 6.415 | x | 19.715 | | 2 | 0 |
| 13 | | | | 6.003 | x | 21.214 | | 1 | 0 |
| 14 | | | | 5.990 | x | | | 1 | 0 |

*Score: “MicroRNA-designated” Black using Non-Black Score; PO = Pregnancy outcome; 1 = Unhealthy, 0 = Healthy

TABLE 27
Application of Black microRNA pregnancy prediction score* panel to a “microRNA
designated” non-Black validation set population (see ROC curve, FIG. 3G)

| | | | (1) hsa- | | | | | | |
|---|---|---|---|---|---|---|---|---|---|
| | | | miR- | | | | | | |
| Patient | Self ID | Race type | 6737-3p | | miR-575 | | 223-3p | | miR- |
| # | Race | by mir score | <24.314 | <24.314 | <21.693 | <21.693 | >8.71 | >8.71 | 1244 |
| 1 | White | Non-Black | 24.31128 | | 18.27212 | x | 8.810635 | x | 24.20949 |
| 2 | White | Non-Black | | | | | 11.28421 | x | |
| 3 | Multi Race | Non-Black | 25.96069 | | | | 11.05419 | x | |
| 4 | Hispanic | Non-Black | | | 23.5256 | | 7.543606 | | 23.7936 |
| 5 | White | Non-Black | | | | | 7.85452 | | |
| 6 | White | Non-Black | | | 23.04795 | | 8.38839 | | |
| 7 | White | Non-Black | | | 24.31961 | | 8.324411 | | |
| 8 | White | Non-Black | | | 21.91157 | | 7.442359 | | 23.33635 |
| 9 | White | Non-Black | | | 20.1436 | x | 8.214922 | | 23.70179 |
| 10 | Hispanic | Non-Black | | | 23.73462 | | 7.660142 | | 25.63048 |
| 11 | White | Non-Black | | | | | 7.974719 | | |
| 12 | White | Non-Black | | | 23.96583 | | 8.321612 | | |
| 13 | Hispanic | Non-Black | | | | | 7.680044 | | |
| 14 | White | Non-Black | | | 18.96582 | x | 8.677639 | | |
| 15 | Hispanic | Non-Black | | | 22.66969 | | 8.84807 | x | 25.76036 |
| 16 | White | Non-Black | | | 22.20069 | | 7.853451 | | |
| 17 | White | Non-Black | | | 20.30571 | x | 8.601219 | | |
| 18 | Multi Race | Non-Black | | | | | 8.00649 | | |
| 19 | White | Non-Black | | | 18.21583 | x | | | |
| 20 | White | Non-Black | | | 22.78819 | | | | |
| 21 | AI/AN | Non-Black | | | 23.63424 | | 7.808486 | | 25.10579 |
| 22 | White | Non-Black | 24.9634 | | 16.53786 | x | 10.75827 | x | |
| 23 | White | Non-Black | | | 20.83519 | x | 7.963829 | | 23.64038 |
| 24 | White | Non-Black | | | 17.67289 | x | 9.632874 | x | 23.64329 |
| 25 | White | Non-Black | 24.20925 | x | | | 9.814296 | x | |
| 26 | White | Non-Black | 24.6207 | | 22.74915 | | 9.656324 | x | |
| 27 | White | Non-Black | | | | | 10.45292 | x | |
| 28 | Multi Race | Non-Black | | | 22.05828 | | 7.354759 | | |
| 29 | Hispanic | Non-Black | | | | | 9.082567 | x | |
| 30 | Hispanic | Non-Black | | | 20.80505 | x | 8.148619 | | 25.83988 |
| 31 | White | Non-Black | | | | | 10.49151 | x | |
| 32 | Black | Non-Black | | | 21.41822 | x | 9.083947 | x | |
| 33 | White | Non-Black | 22.29463 | x | 20.1735 | x | 10.34563 | x | |

| Patient | | miR-340- | | miR- | | | |
|---|---|---|---|---|---|---|---|
| # | >23.133 | 5p | <22.734 | 196a-5p | <22.94 | Score* | Outcome |
| 1 | x | 23.67836 | | | | 3 | 1 |
| 2 | | 23.28719 | | | | 1 | 1 |
| 3 | | 22.511 | x | 21.59351 | x | 3 | 0 |
| 4 | x | 20.19024 | x | 21.45349 | x | 3 | 0 |
| 5 | | 21.93806 | x | 22.34007 | x | 2 | 0 |
| 6 | | 21.87418 | x | | | 1 | 0 |
| 7 | | 21.74773 | x | 21.32495 | x | 2 | 0 |
| 8 | x | 20.86647 | x | 20.64725 | x | 3 | 0 |
| 9 | x | 22.35626 | x | 25.89141 | | 3 | 0 |
| 10 | x | 20.50649 | x | 20.96419 | x | 3 | 0 |
| 11 | | 22.14165 | x | 24.84614 | | 1 | 0 |
| 12 | | 20.55578 | x | 20.79555 | x | 2 | 0 |
| 13 | | 24.31184 | | | | 0 | 0 |
| 14 | | 25.71359 | | | | 1 | 0 |
| 15 | x | 22.00653 | x | 25.03869 | | 3 | 0 |
| 16 | | 22.20326 | x | | | 1 | 0 |
| 17 | | 21.74206 | x | | | 2 | 0 |
| 18 | | 22.58145 | 1 | 23.1434 | | 1 | 0 |
| 19 | | 21.15586 | 1 | 22.77703 | 1 | 3 | 0 |
| 20 | | 22.52731 | 1 | 23.61272 | | 1 | 0 |
| 21 | x | 20.58967 | 1 | | | 2 | 0 |
| 22 | | | | | | 2 | 0 |
| 23 | 1 | 21.80529 | 1 | 24.52831 | | 3 | 0 |
| 24 | 1 | 22.92661 | | | | 3 | 0 |
| 25 | | 22.52836 | 1 | 24.29847 | | 3 | 0 |
| 26 | | | | 21.58584 | 1 | 2 | 0 |
| 27 | | 24.02914 | | 25.03364 | | 1 | 0 |
| 28 | | 21.23701 | 1 | 23.75319 | | 1 | 0 |
| 29 | | 24.30534 | | 23.00191 | | 1 | 0 |
| 30 | x | 21.32871 | x | 22.49606 | x | 4 | 0 |
| 31 | | | | 22.83179 | x | 2 | 0 |
| 32 | | 22.47423 | x | 22.0114 | x | 4 | 0 |
| 33 | | | | 21.12176 | x | 4 | 0 |

TABLE 28
Application of Black patient microRNA pregnancy prediction panel to a “microRNA
designated” Black patient validation set population (see ROC curve, FIG. 3H)

| | Patient | Race-type | | | | | | | |
|---|---|---|---|---|---|---|---|---|---|
| | self - | based on | (1) | | | | | | (4) |
| | identified | microRNA | miR- | | (2) | | (3) miR- | | miR- |
| Patient# | Race | score* | 6737-3p | <24.314 | miR-575 | <21.693 | 223-3p | >8.71 | 1244 |
| 1 | White | Black type | 25.43 | | 28.307 | | 11.016 | x | |
| 2 | Black | Black type | | | 21.915 | | 9.8925 | x | |
| 3 | Black | Black type | | | 21.838 | | 8.8773 | x | |
| 4 | Black | Black type | | | 21.6 | x | 9.1051 | x | 23.74 |
| 5 | Hispanic | Black type | 22.305 | x | 19.06 | x | 9.759 | x | |
| 6 | White | Black type | 23.393 | x | | | 11.096 | x | |
| 7 | Multi | Black type | 25.984 | | | | 10.152 | x | |

| | | (5) | | (6) | | | |
|---|---|---|---|---|---|---|---|
| | | miR- | | miR- | | | |
| Patient# | >23.133 | 340-5p | <22.734 | 196a-5p | <22.94 | Score* | PO* |
| 1 | | | | 20.782 | x | 2 | 0 |
| 2 | | 22.56 | x | 22.001 | x | 3 | 1 |
| 3 | | 21.44 | x | 20.983 | x | 3 | 1 |
| 4 | | 22.71 | | 23.051 | | 2 | 0 |
| 5 | | | | | | 3 | 0 |
| 6 | | 21.6 | x | | | 3 | 0 |
| 7 | | | | | | 1 | 0 |

*Score: “MicroRNA-designated” Non-Black using Non -Black Score; PO = Pregnancy outcome; 1 = Unhealthy, 0 = Healthy

TABLE 29
Comparison of ROC curves for pregnancy outcome prediction using “self-identified”
race versus “microRNA designated” race in validation set populations

| Self-identified (ID) race: | Non-Black microRNA Risk score | Black microRNA Risk Score |
|---|---|---|
| Non-Black | ROC: 0.72, p = 0.07 (FIG. 2A) | ROC: 0.57, p = 0.72 (FIG. 2B) |
| Black | ROC: 0.60, p = 0.62 (FIG. 2C) | ROC: 0.71, p = 0.07 (FIG. 2D) |

| MicroRNA designated race: | Non-Black microRNA Risk score | Black microRNA Risk Score |
|---|---|---|
| Non-Black | ROC: 0.78, p < 0.001 (FIG. 3E) | ROC: 0.54, p = 0.89 (FIG. 3G) |
| Black | ROC: 0.50, p = 1.00 (FIG. 3F) | ROC: 0.80, p = 0.014 (FIG. 3H) |

TABLE 30
Analysis of the top 20 differentially expressed immune cell microRNAs between pregnant Blacks and Non-Blacks
in early pregnancy (see Table 13) top 22 genes with associated pathways in common (see Table 31). Note:
Non-Black populations are known to carry significant Neanderthal DNA while Black populations do not.

| | | Top C1C2microRNA- associated with |
|---|---|---|
| | | Neanderthal SNP regulated gene |
| # | C1C2 regulatory microRNAs* | sets** |
| 1. | hsa-miR-25-3p | ADAM10 |
| 2. | bsa-miR-22-3p | CCN1 |
| 3. | hsa-miR-23a-3p, hsa-miR-23b-3p | CXCL12 |
| 4. | hsa-let-7c-5p, hsa-let-7d-5p, hsa-let-7e-5p, hsa-let-7g-5p, hsa-let-7i-5p, | CYP19A1 |
| 5. | hsa-let-7f-5p, hsa-let-7g-5p, hsa-miR-24-3p | DLC1 |
| 6. | hsa-miR-25-3p | DUSP10 |
| 7. | hsa-let-7e-5p | EDN1 |
| 8. | hsa-miR-17-5p | HIF1A |
| 9. | hsa-miR-19a-3p, hsa-miR-19b-3p, hsa-miR-29a-3p, hsa-miR-29b-3p, hsa-miR-29c-3p | IGF1 |
| 10. | hsa-miR-19a-3p, hsa-miR-19b-3p. | IGFBP3 |
| 11. | hsa-miR-25-3p | ITGA5 |
| 12. | hsa-miR-23b-3p | LPAR1 |
| 13. | hsa-miR-25-3p | MIA3 |
| 14. | hsa-miR-17-5p | MINK1 |
| 15. | hsa-let-7a-5p, hsa-let-7b-5p, hsa-let-7c-5p, hsa-let-7d-5p, hsa-let-7e-5p, hsa-let-7f-5p, hsa-let-7g-5p, hsa- | PARD6B |
| | let-7i-5p, hsa-miR-18a-5p, | |
| 16. | hsa-miR-23b-3p, hsa-miR-24-3p, hsa-miR-24-3p | PTGER4 |
| 17. | hsa-let-7b-5p, hsa-let-7c-5p, hsa-let-7d-5p, hsa-let-7e-5p, hsa-let-7f-5p, | RNF20 |
| | hsa-let-7g-5p | |
| 18. | hsa-miR-30b-5p | SNAI1 |
| 19. | hsa-miR-17-5p | TGFBR2 |
| 20. | hsa-miR-23a-3p, hsa-miR-23b-3p, | TRFAIP6 |
| 21. | hsa-miR-29a-3p, hsa-miR-29c-3p | TAM1 |
| 22. | hsa-miR-15a-5p, hsa-miR-15b-5p, hsa-miR-16-5p, hsa-miR-29a-3p, hsa-miR-29b-3p, hsa-miR-29c-3p | VEGFA |

*See Table 13 of our specification for complete list of C1C2 microRNAs. MicroRNAs selected using gene sets analysis using mirDip online analysis tool
**Neanderthal gene information resource: Supplementary Data File #7 from Silvert M, Quintana-Murci L, Rotival M. Impact and Evolutionary Determinants of Neanderthal Introgression on Transcriptional and Post-Transcriptional Regulation. Am J Hum Genet. 2019 Jun. 6; 104(6): 1241-1250. doi: 10.1016/j.ajhg.2019.04.016. Epub 2019 May 30. PMID: 31155285; PMCID: PMC6557732.)

TABLE 31
Reactome Pathways Analysis of 22 Neanderthal introgressed genes (from Table 30) regulated
by our top 10 C1C2 race identifying microRNAs (from Table 32) in early pregnancy **

| | | Entities | Entities | Entities | Entities |
|---|---|---|---|---|---|
| # | Pathway name | found | Total | ratio | pValue |
| 1 | Regulation of gene expression by Hypoxia-Inducible Factor | 3 | 15 | 0.001 | 6.73E−06 |
| 2 | Signal Transduction | 19 | 3,028 | 0.2 | 1.24E−05 |
| 3 | Regulation of Insulin-like Growth Factor (IGF) transport and uptake | 5 | 127 | 0.008 | 1.25E−05 |
| | by Insulin-like Growth Factor Binding Proteins (IGFBPs) | | | | |
| 4 | Signaling by Receptor Tyrosine Kinases | 8 | 623 | 0.041 | 8.69E−05 |
| 5 | Post-translational protein phosphorylation | 4 | 109 | 0.007 | 1.31E−04 |
| 6 | Epithelial-Mesenchymal Transition (EMT) during gastrulation | 2 | 8 | 0.001 | 1.73E−04 |
| 7 | RUNX2 regulates genes involved in cell migration | 2 | 14 | 0.001 | 5.25E−04 |
| | TP53 Regulates Transcription of Death Receptors and Ligands | 2 | 18 | 0.001 | 8.63E−04 |
| 9 | STAT3 nuclear events downstream of ALK signaling | 2 | 18 | 0.001 | 8.63E−04 |
| 10 | Transcription of E2F targets under negative control by p107 (RBL1) | 2 | 20 | 0.00 | 1.06E−03 |
| | and p130 (RBL2) in complex with HDAC1 | 3 | 86 | 0.006 | |
| 11 | Cellular response to hypoxia | 2 | 21 | 0.001 | 1.13E−03 |
| 12 | TFAP2 (AP-2) family regulates transcription of growth factors and | 4 | 211 | 0.0014 | 1.17E−03 |
| | their receptors | | | | |
| 13 | Interleukin-4 and Interleukin-13 signaling | 8 | 211 | 0.014 | 1.54E−03 |
| 14 | Cellular responses to stress | 8 | 1,007 | 0.066 | 2.12E−03 |
| 15 | Cellular responses to stimuli | 8 | 1,025 | 0.068 | 2.37E−03 |
| 16 | G0 and Early G1 | 2 | 38 | 0.003 | 3.73E−03 |
| 17 | TGFBR2 MSI Frameshift Mutants in Cancer | 1 | 2 | 0 | 4.73E−03 |
| 18 | Signaling by ALK | 2 | 43 | 0.003 | 4.75E−03 |
| 19 | Nuclear signaling by ERBB4 | 2 | 47 | 0.003 | 5.64E−03 |
| 20 | Transcriptional regulation by the AP-2 (TFAP2) family of | 2 | 52 | 0.003 | 6.85E−03 |
| | transcription factors | | | | |

** Reactome: Marc Gillespie et al: The reactome pathway knowledgebase 2022, Nucleic Acids Research, 2021;, gkab1028, https://doi.org/10.1093/nar/gkab1028; https://reactome.org/ (last accessed Apr. 18, 2023)

TABLE 32*
C1C2ratio calculations for race assessment taken from healthy first trimester pregnancy samples in Blacks
and non-Blacks using 175 microRNAs with low HC ratios <1.0 (low pregnancy outcome prediction ability)

| | | Mean order | | Mean order | | | | |
|---|---|---|---|---|---|---|---|---|
| | | ranking | | ranking | | | | |
| | | of 175 miRs | | of 175 miRs | | | | |
| C1C2ratio | | in NON- | | in BLACKS | | C1C2ratio | | |
| Order | 175 microRNAs | BLACKS (6 healthy | | (3 healthy | | (non- | | |
| (Highest to | with HC | pregnant | | pregnant | | Blacks = C1 | | |
| lowest) | Ratio <1.0 | samples) | SD | samples) | SD | Blacks = C2) | C1C2ratio >2.3 | P value* |
| 1 | hsa-miR-150-5p | 55.6 | 25.9 | 2.3 | 1.7 | 3.85700298 | x | 0.01 |
| 2 | hsa-let-7g-5p | 82.9 | 40.7 | 4.5 | 3.3 | 3.55936353 | x | 0.01 |
| 3 | hsa-miR-16-5p | 82.1 | 40.9 | 4.5 | 4.2 | 3.44196763 | x | 0.02 |
| 4 | hsa-miR-29a-3p | 76.9 | 37.9 | 5.5 | 3.8 | 3.42060233 | x | 0.02 |
| 5 | hsa-let-7f-5p | 88.4 | 42.1 | 8.0 | 5.6 | 3.37086159 | x | 0.02 |
| 6 | hsa-miR-15b-5p | 79.6 | 41.8 | 6.3 | 4.5 | 3.16610454 | x | 0.02 |
| 7 | hsa-let-7i-5p | 85.6 | 41.2 | 9.5 | 6.9 | 3.1642313 | x | 0.02 |
| 8 | hsa-miR-26b-5p | 80.6 | 37.6 | 10.5 | 7.0 | 3.13838514 | x | 0.02 |
| 9 | hsa-miR-223-3p | 75.9 | 48.4 | 1.0 | 0.8 | 3.04200685 | x | 0.04 |
| 10 | hsa-miR-21-5p | 80.1 | 48.3 | 2.5 | 3.1 | 3.02246197 | x | 0.03 |
| 11 | hsa-miR-23a-3p | 85.0 | 45.0 | 11.3 | 8.1 | 2.7802185 | x | 0.03 |
| 12 | hsa-miR-142-3p | 77.6 | 51.8 | 3.3 | 2.5 | 2.73790201 | x | 0.05 |
| 13 | hsa-miR-19b-3p | 86.9 | 46.9 | 12.8 | 8.7 | 2.67015274 | x | 0.03 |
| 14 | hsa-let-7a-5p | 78.6 | 53.1 | 4.5 | 3.3 | 2.62528426 | x | 0.03 |
| 15 | hsa-miR-22-3p | 86.7 | 40.6 | 18.0 | 12.4 | 2.59663764 | x | 0.03 |
| 16 | hsa-miR-29c-3p | 78.4 | 50.1 | 9.0 | 6.1 | 2.47481324 | x | 0.05 |
| 17 | hsa-miR-342-3p | 69.7 | 38.1 | 12.3 | 9.0 | 2.44274309 | x | 0.04 |
| 18 | hsa-miR-25-3p | 79.4 | 38.4 | 17.5 | 12.8 | 2.42183473 | x | 0.03 |
| 19 | hsa-miR-24-3p | 82.3 | 45.2 | 16.5 | 11.4 | 2.32744139 | x | 0.04 |
| 20 | hsa-miR-17-5p | 77.0 | 36.8 | 20.0 | 13.6 | 2.26567742 | x | 0.04 |
| 21 | hsa-miR-30b-5p | 75.1 | 42.9 | 18.3 | 12.4 | 2.0542632 | x | 0.07 |
| 22 | hsa-miR-103a-3p | 66.7 | 45.0 | 11.8 | 8.7 | 2.04632663 | x | 0.08 |
| 23 | hsa-miR-146b-5p | 68.1 | 38.5 | 17.5 | 12.4 | 1.98823854 | x | 0.07 |
| 24 | hsa-miR-142-5p | 89.6 | 58.6 | 19.5 | 14.1 | 1.92989965 | x | 0.09 |
| 25 | hsa-miR-181a-5p | 78.1 | 44.2 | 22.8 | 15.5 | 1.85513326 | x | 0.08 |
| 26 | hsa-miR-126-3p | 76.9 | 45.2 | 22.0 | 14.7 | 1.83099019 | x | 0.09 |
| 27 | hsa-miR-338-3p | 112.6 | 64.4 | 34.8 | 24.4 | 1.75396275 | x | 0.09 |
| 28 | hsa-let-7b-5p | 59.7 | 47.7 | 11.8 | 8.5 | 1.70778028 | x | 0.13 |
| 29 | hsa-miR-1202 | 92.3 | 52.5 | 31.3 | 21.1 | 1.65895314 | x | 0.10 |
| 30 | hsa-miR-199a-3p | 73.6 | 47.2 | 22.8 | 15.8 | 1.6134873 | x | 0.12 |
| 31 | hsa-miR-155-5p | 82.3 | 49.4 | 28.3 | 19.2 | 1.57518212 | x | 0.12 |
| 32 | hsa-miR-140-3p | 65.0 | 30.1 | 28.3 | 19.3 | 1.4879003 | | |
| 33 | hsa-miR-194-5p | 112.1 | 66.5 | 42.0 | 28.1 | 1.48368344 | | |
| 34 | hsa-miR-107 | 56.0 | 43.2 | 16.0 | 10.8 | 1.48268974 | | |
| 35 | hsa-let-7d-5p | 57.3 | 44.0 | 16.5 | 11.6 | 1.46765887 | | |
| 36 | hsa-miR-130a-3p | 76.3 | 52.4 | 26.0 | 17.8 | 1.4330449 | | |
| 37 | hsa-miR-331-3p | 71.0 | 41.9 | 28.8 | 19.8 | 1.37140534 | | |
| 38 | hsa-miR-30d-5p | 69.3 | 37.3 | 30.5 | 20.8 | 1.33561327 | | |
| 39 | hsa-miR-92a-3p | 59.1 | 36.1 | 24.3 | 17.0 | 1.31383063 | | |
| 40 | hsa-miR-320d | 62.0 | 36.8 | 26.8 | 18.0 | 1.28693323 | | |
| 41 | hsa-miR-125a-5p | 107.3 | 64.0 | 48.8 | 33.0 | 1.20661919 | | |
| 42 | hsa-miR-1246 | 78.0 | 60.9 | 29.0 | 22.0 | 1.18196886 | | |
| 43 | hsa-miR-483-5p | 112.3 | 72.8 | 54.5 | 39.0 | 1.03305998 | | |
| 44 | hsa-miR-181a-3p | 114.6 | 70.7 | 57.0 | 42.8 | 1.01436945 | | |
| 45 | hsa-miR-10a-5p | 100.1 | 70.6 | 47.8 | 33.7 | 1.00436441 | | |
| 46 | hsa-miR-320b | 59.4 | 35.8 | 32.0 | 21.4 | 0.95973362 | | |
| 47 | hsa-miR-125b-5p | 97.1 | 69.7 | 48.5 | 33.7 | 0.94108797 | | |
| 48 | hsa-miR-378a-5p | 85.6 | 67.7 | 40.8 | 28.6 | 0.93028798 | | |
| 49 | hsa-miR-1275 | 70.4 | 40.0 | 40.5 | 27.8 | 0.88285377 | | |
| 50 | hsa-miR-130b-3p | 64.3 | 41.0 | 35.8 | 24.3 | 0.87479863 | | |
| 51 | hsa-miR-1290 | 101.0 | 68.7 | 53.5 | 40.9 | 0.86682517 | | |
| 52 | hsa-miR-150-3p | 116.1 | 72.2 | 69.0 | 46.5 | 0.79425197 | | |
| 53 | hsa-miR-365a-3p | 102.7 | 69.8 | 58.5 | 44.2 | 0.77581617 | | |
| 54 | hsa-miR-584-5p | 94.4 | 67.6 | 54.3 | 36.5 | 0.7721553 | | |
| 55 | hsa-miR-181b-5p | 60.4 | 38.6 | 36.3 | 24.4 | 0.76741398 | | |
| 56 | hsa-miR-145-5p | 89.9 | 61.2 | 52.8 | 35.6 | 0.76731343 | | |
| 57 | hsa-miR-1914-3p | 96.0 | 75.6 | 53.5 | 35.9 | 0.76224281 | | |
| 58 | hsa-miR-486-5p | 72.0 | 57.6 | 39.0 | 30.7 | 0.74735488 | | |
| 59 | hsa-miR-146a-5p | 49.1 | 46.2 | 25.5 | 17.4 | 0.74439873 | | |
| 60 | hsa-miR-671-5p | 98.0 | 77.9 | 50.0 | 54.8 | 0.72347847 | | |
| 61 | hsa-miR-223-5p | 94.7 | 72.7 | 55.3 | 37.4 | 0.71670868 | | |
| 62 | hsa-miR-423-5p | 98.1 | 73.8 | 57.3 | 42.3 | 0.70439906 | | |
| 63 | hsa-miR-29b-1-5p | 68.0 | 55.2 | 39.3 | 26.7 | 0.70201592 | | |
| 64 | hsa-miR-100-5p | 101.1 | 69.5 | 62.0 | 47.1 | 0.67159735 | | |
| 65 | hsa-miR-320c | 49.6 | 51.3 | 27.3 | 18.3 | 0.64122886 | | |
| 66 | hsa-miR-1268a | 63.0 | 51.9 | 38.8 | 25.9 | 0.62365561 | | |
| 67 | hsa-miR-663a | 97.6 | 73.8 | 62.0 | 42.9 | 0.60932486 | | |
| 68 | hsa-miR-22-5p | 98.6 | 72.1 | 64.0 | 43.9 | 0.59613616 | | |
| 69 | hsa-miR-1225-3p | 56.1 | 58.8 | 31.5 | 24.0 | 0.59512093 | | |
| 70 | hsa-miR-532-3p | 93.9 | 61.2 | 64.0 | 43.9 | 0.56820135 | | |
| 71 | hsa-miR-376a-3p | 92.1 | 72.4 | 58.5 | 47.1 | 0.56287749 | | |
| 72 | hsa-miR-324-3p | 54.1 | 33.2 | 38.0 | 25.4 | 0.55171079 | | |
| 73 | hsa-miR-502-5p | 77.0 | 64.5 | 50.0 | 34.1 | 0.54782409 | | |
| 74 | hsa-miR-1305 | 99.6 | 75.0 | 66.8 | 45.2 | 0.54593048 | | |
| 75 | hsa-miR-135a-3p | 95.9 | 73.0 | 63.5 | 45.7 | 0.54491882 | | |
| 76 | hsa-miR-345-5p | 90.9 | 72.5 | 60.0 | 46.0 | 0.5207865 | | |
| 77 | hsa-miR-186-5p | 52.1 | 43.7 | 35.5 | 24.4 | 0.48875691 | | |
| 78 | hsa-miR-630 | 78.6 | 71.3 | 51.5 | 44.1 | 0.46949385 | | |
| 79 | hsa-miR-574-5p | 65.3 | 52.3 | 47.0 | 31.4 | 0.43691209 | | |
| 80 | hsa-miR-629-5p | 111.0 | 67.9 | 85.3 | 58.1 | 0.40845673 | | |
| 81 | hsa-miR-188-5p | 72.3 | 67.1 | 51.0 | 42.5 | 0.38854007 | | |
| 82 | hsa-miR-377-3p | 87.9 | 69.9 | 64.5 | 50.7 | 0.38721497 | | |
| 83 | hsa-miR-542-3p | 90.4 | 69.6 | 68.0 | 47.8 | 0.38214292 | | |
| 84 | hsa-miR-330-3p | 95.0 | 72.9 | 73.3 | 50.0 | 0.35393185 | | |
| 85 | hsa-miR-1181 | 77.0 | 73.6 | 58.0 | 39.5 | 0.33597495 | | |
| 86 | hsa-miR-200b-3p | 98.6 | 67.8 | 78.5 | 53.2 | 0.33176627 | | |
| 87 | hsa-miR-601 | 89.9 | 54.0 | 74.3 | 50.0 | 0.29993094 | | |
| 88 | hsa-miR-1271-5p | 90.0 | 67.9 | 73.3 | 50.0 | 0.28416487 | | |
| 89 | hsa-miR-21-3p | 61.0 | 39.8 | 50.8 | 34.7 | 0.27504839 | | |
| 90 | hsa-miR-16-2-3p | 94.7 | 65.4 | 78.3 | 55.7 | 0.27183714 | | |
| 91 | hsa-miR-629-3p | 91.3 | 68.8 | 75.5 | 50.7 | 0.26422571 | | |
| 92 | hsa-miR-193b-3p | 88.1 | 74.3 | 74.5 | 52.7 | 0.21490991 | | |
| 93 | hsa-miR-664-5p | 93.3 | 58.2 | 81.5 | 55.4 | 0.20757356 | | |
| 94 | hsa-miR-326 | 79.7 | 56.0 | 69.3 | 47.8 | 0.20158728 | | |
| 95 | hsa-miR-664-3p | 78.7 | 64.2 | 68.5 | 47.1 | 0.18366381 | | |
| 96 | hsa-miR-497-5p | 93.3 | 61.5 | 82.8 | 55.2 | 0.18054027 | | |
| 97 | hsa-miR-30a-5p | 77.6 | 61.6 | 68.0 | 46.3 | 0.17731339 | | |
| 98 | hsa-miR-486-3p | 105.4 | 68.7 | 96.5 | 64.5 | 0.13412305 | | |
| 99 | hsa-miR-638 | 51.4 | 53.6 | 45.8 | 33.6 | 0.1301481 | | |
| 100 | hsa-miR-133b | 83.7 | 65.7 | 77.5 | 53.0 | 0.10470386 | | |
| 101 | hsa-miR-181a-2-3p | 83.7 | 63.8 | 79.5 | 55.6 | 0.07057664 | | |
| 102 | hsa-miR-940 | 54.9 | 56.8 | 53.5 | 37.2 | 0.02888818 | | |
| 103 | hsa-miR-139-5p | 97.6 | 65.4 | 96.3 | 64.3 | 0.02038286 | | |
| 104 | hsa-miR-155-3p | 86.4 | 66.5 | 86.5 | 74.2 | −0.0010155 | | |
| 105 | hsa-miR-513a-5p | 75.4 | 56.7 | 76.0 | 54.7 | −0.0102611 | | |
| 106 | hsa-miR-181c-3p | 86.9 | 62.0 | 88.8 | 59.2 | −0.0312404 | | |
| 107 | hsa-miR-339-3p | 100.0 | 50.5 | 102.5 | 69.2 | −0.0417715 | | |
| 108 | hsa-miR-381 | 74.1 | 62.3 | 77.3 | 58.1 | −0.0516254 | | |
| 109 | hsa-miR-539-5p | 92.6 | 63.6 | 97.8 | 65.8 | −0.0800021 | | |
| 110 | hsa-miR-1224-5p | 69.0 | 65.0 | 73.8 | 53.2 | −0.0803344 | | |
| 111 | hsa-miR-421 | 97.4 | 65.3 | 104.0 | 71.6 | −0.0959827 | | |
| 112 | hsa-miR-654-3p | 71.6 | 65.8 | 78.5 | 60.7 | −0.1095402 | | |
| 113 | hsa-miR-30b-3p | 87.3 | 59.4 | 94.0 | 63.0 | −0.1096971 | | |
| 114 | hsa-miR-1226-5p | 81.6 | 73.0 | 89.0 | 60.0 | −0.1117619 | | |
| 115 | hsa-miR-143-3p | 83.7 | 65.5 | 92.3 | 63.2 | −0.1325989 | | |
| 116 | hsa-miR-127-3p | 62.6 | 57.6 | 69.8 | 50.6 | −0.1326759 | | |
| 117 | hsa-miR-15a-3p | 89.9 | 58.9 | 99.0 | 67.1 | −0.1451746 | | |
| 118 | hsa-miR-1471 | 65.7 | 71.9 | 76.8 | 58.4 | −0.1693598 | | |
| 119 | hsa-miR-574-3p | 53.1 | 55.3 | 62.5 | 42.9 | −0.1905497 | | |
| 120 | hsa-miR-23a-5p | 79.7 | 68.4 | 92.8 | 62.7 | −0.19889 | | |
| 121 | hsa-miR-222-3p | 41.1 | 45.0 | 49.0 | 33.1 | −0.2009865 | | |
| 122 | hsa-miR-622 | 84.0 | 51.6 | 97.5 | 69.4 | −0.2231093 | | |
| 123 | hsa-miR-766-3p | 57.0 | 43.0 | 68.0 | 45.8 | −0.2475827 | | |
| 124 | hsa-miR-1183 | 68.0 | 74.4 | 88.0 | 63.8 | −0.2895919 | | |
| 125 | hsa-miR-139-3p | 73.9 | 63.7 | 92.3 | 62.9 | −0.290532 | | |
| 126 | hsa-miR-1261 | 78.7 | 49.8 | 95.8 | 64.4 | −0.2983336 | | |
| 127 | hsa-miR-493-5p | 65.7 | 59.8 | 88.3 | 66.1 | −0.3581465 | | |
| 128 | hsa-miR-572 | 64.0 | 55.4 | 85.0 | 60.5 | −0.3624095 | | |
| 129 | hsa-miR-623 | 77.9 | 60.0 | 101.8 | 68.5 | −0.3718577 | | |
| 130 | hsa-miR-382-5p | 63.0 | 55.5 | 84.3 | 58.6 | −0.3725689 | | |
| 131 | hsa-miR-99b-3p | 89.9 | 54.2 | 115.8 | 78.4 | −0.3903932 | | |
| 132 | hsa-miR-26b-3p | 74.0 | 56.1 | 100.8 | 71.0 | −0.421171 | | |
| 133 | hsa-miR-202-3p | 69.6 | 56.8 | 97.5 | 66.5 | −0.4531205 | | |
| 134 | hsa-miR-101-5p | 76.9 | 60.8 | 108.8 | 73.6 | −0.4743609 | | |
| 135 | hsa-miR-543 | 62.9 | 56.4 | 92.8 | 69.4 | −0.475117 | | |
| 136 | hsa-miR-1825 | 66.9 | 72.6 | 103.0 | 71.7 | −0.5008794 | | |
| 137 | hsa-miR-298 | 62.1 | 50.4 | 93.3 | 66.6 | −0.5316709 | | |
| 138 | hsa-miR-30c-1-3p | 74.1 | 51.9 | 107.8 | 73.2 | −0.537183 | | |
| 139 | hsa-miR-431-5p | 55.0 | 51.5 | 89.8 | 67.0 | −0.5863763 | | |
| 140 | hsa-miR-1281 | 56.9 | 61.4 | 95.5 | 69.5 | −0.5902242 | | |
| 141 | hsa-miR-376a-5p | 66.9 | 51.9 | 104.3 | 74.7 | −0.590943 | | |
| 142 | hsa-miR-379-5p | 61.6 | 57.7 | 100.0 | 70.9 | −0.5977252 | | |
| 143 | hsa-miR-501-5p | 57.0 | 63.8 | 95.3 | 63.8 | −0.5993322 | | |
| 144 | hsa-miR-501-3p | 62.9 | 58.6 | 101.3 | 68.9 | −0.6024197 | | |
| 145 | hsa-miR-154-5p | 53.4 | 48.3 | 88.5 | 67.0 | −0.608182 | | |
| 146 | hsa-miR-92a-1-5p | 65.7 | 51.6 | 104.0 | 71.2 | 0.6234082 | | |
| 147 | hsa-miR-548b-5p | 66.9 | 56.1 | 107.5 | 72.8 | −0.6304894 | | |
| 148 | hsa-miR-423-3p | 73.1 | 53.0 | 115.3 | 77.1 | −0.6474573 | | |
| 149 | hsa-miR-602 | 55.0 | 58.7 | 95.3 | 64.0 | −0.6559588 | | |
| 150 | hsa-miR-933 | 66.4 | 70.4 | 115.0 | 77.4 | −0.657004 | | |
| 151 | hsa-miR-550a-5p | 64.3 | 64.0 | 109.8 | 74.1 | −0.6582102 | | |
| 152 | hsa-miR-28-3p | 81.4 | 50.2 | 126.5 | 84.3 | −0.6699499 | | |
| 153 | hsa-miR-610 | 74.6 | 47.1 | 116.5 | 77.7 | −0.6721302 | | |
| 154 | hsa-miR-196a-5p | 58.3 | 69.9 | 108.3 | 73.6 | −0.6963265 | | |
| 155 | hsa-miR-659-3p | 64.0 | 52.4 | 107.8 | 72.9 | −0.697993 | | |
| 156 | hsa-let-7b-3p | 60.6 | 64.3 | 108.5 | 72.8 | −0.6992656 | | |
| 157 | hsa-let-7f-1-3p | 62.3 | 67.4 | 112.5 | 75.7 | −0.7017772 | | |
| 158 | hsa-miR-92b-3p | 63.6 | 75.6 | 121.0 | 80.7 | −0.7346224 | | |
| 159 | hsa-miR-769-3p | 61.6 | 50.8 | 109.5 | 74.5 | −0.7648789 | | |
| 160 | hsa-miR-192-3p | 62.1 | 50.0 | 110.0 | 73.8 | −0.7731786 | | |
| 161 | hsa-miR-337-5p | 55.4 | 52.0 | 108.3 | 75.5 | −0.8284917 | | |
| 162 | hsa-miR-299-3p | 47.6 | 53.8 | 103.5 | 69.2 | −0.9094092 | | |
| 163 | hsa-miR-129-5p | 57.1 | 47.9 | 114.8 | 77.1 | −0.9217343 | | |
| 164 | hsa-miR-377-5p | 62.4 | 50.5 | 126.0 | 84.1 | −0.9439717 | | |
| 165 | hsa-miR-1470 | 51.7 | 59.8 | 118.0 | 78.8 | −0.9565807 | | |
| 166 | hsa-miR-125b-2-3p | 63.6 | 39.8 | 121.8 | 81.5 | −0.9589703 | | |
| 167 | hsa-miR-106a-3p | 63.4 | 48.3 | 129.0 | 86.0 | −0.9762021 | | |
| 168 | hsa-miR-885-3p | 58.4 | 47.1 | 122.5 | 82.9 | −0.9858061 | | |
| 169 | hsa-miR-760 | 42.0 | 60.5 | 108.0 | 72.7 | −0.9915031 | | |
| 170 | hsa-miR-634 | 52.0 | 62.1 | 125.5 | 83.7 | −1.0084598 | | |
| 171 | hsa-miR-129-2-3p | 46.1 | 64.2 | 126.5 | 84.4 | −1.0817029 | | |
| 172 | hsa-miR-616-3p | 51.9 | 42.0 | 122.5 | 82.3 | −1.1368657 | | |
| 173 | hsa-miR-876-3p | 43.0 | 62.1 | 126.5 | 84.4 | −1.1396534 | | |
| 174 | hsa-miR-1295a | 33.0 | 44.0 | 113.0 | 76.2 | −1.3303823 | | |
| 175 | hsa-miR-1294 | 38.4 | 42.1 | 130.5 | 87.0 | −1.4267729 | | |

*Also, see related Table 13
*P values calculated using Graphpad T test calculator from https://www.graphpad.com/quickcalcs/ttest1.cfm, last accessed Feb. 23, 2023

While certain embodiments have been described in terms of the preferred embodiments, it is understood that variations and modifications will occur to those skilled in the art. Therefore, it is intended that the appended claims cover all such equivalent variations that come within the scope of the following claims.

1-22. (canceled)

23. A method comprising:
a) classifying a pregnant human as a member of a particular patient cluster by quantifying the expression of a first panel of microRNAs (miRNAs) in one or more test biological samples of the pregnant human being as compared to that of a control sample of the particular patient cluster;
b) comparing the expression of a second panel miRNAs associated with an increased risk of developing a placental bed disorder in the particular patient cluster, wherein a difference in the expression in a test biological sample of the pregnant human being relative to a control biological sample of the particular patient cluster indicates the pregnant human being is at risk of developing a placental bed disorder; and,
c) treating the pregnant human being identified in step b) as being at risk of developing a placental bed disorder using immunotherapy.

24. The method of claim 1 wherein expression of the miRNAs in the pregnant human being is higher than that of the control sample of the particular patient cluster, thereby identifying the pregnant human being a member of that particular patient cluster.

25. The method of claim 1 wherein expression of the miRNAs in the pregnant human being is lower than that of the control sample of the particular patient cluster, thereby identifying the pregnant human being a member of that particular patient cluster.

26. The method of claim 1 wherein the miRNAs of the first panel are selected from the group consisting of hsa-let-7g-5p, hsa-miR-150-5p, hsa-let-7f-5p, hsa-let-7i-5p, hsa-miR-15b-5p, hsa-miR-16-5p, hsa-miR-26b-5p, hsa-miR-29a-3p, hsa-let-7a-5p, hsa-miR-19b-3p, hsa-miR-21-5p, hsa-miR-223-3p, hsa-miR-23a-3p, hsa-miR-25-3p, hsa-miR-17-5p, hsa-miR-22-3p, hsa-miR-24-3p, hsa-miR-342-3p, hsa-miR-142-3p, and hsa-miR-29c-3p.

27. The method of claim 1 wherein the biological sample is obtained during the first trimester of pregnancy.

28. The method of claim 1 wherein the placental bed disorder is selected from the group consisting of preeclampsia, preterm birth, HELLP Syndrome, gestational diabetes, miscarriage, implantation failure, fetal growth restriction, and premature rupture of the membranes (P.R.O.M.).

29. The method of claim 28, wherein the placental bed disorder is preeclampsia.

30. The method of claim 1 wherein the control biological sample is representative of a pregnant human being without a placental bed disorder.

31. The method of claim 1 wherein the biological samples are derived from peripheral blood.

32. The method of claim 31 wherein the biological sample comprises mononuclear cells.

33. The method of claim 31, further comprising the additional step of isolating mononuclear cells from the biological sample.

34. The method of claim 1, further comprising the step of extracting RNA from the biological sample.

35. The method of claim 1 comprising calculating a ratio (HC ratio) of expression of said at least one miRNA of the first and/or second panels, wherein said ratio comprises: a numerator equal to the difference between the mean value of expression of the at least one miRNA in the first population and the mean value of the second population and the denominator comprises the average of the two standard deviations of the values for the first and second populations; wherein the at least one miRNA is selected from the group consisting of the miRNAs presented herein as being capable of distinguishing the first and second populations.

36. The method of any one of claim 35 wherein said miRNA exhibits a signal consistency of at least about 85%; a mean signal strength of at least 5.0; and a p value of less than 0.05 (p<0.05).

37. The method of claim 35 wherein the at least one miRNA exhibits a HC ratio of greater than or equal to about any of 1.0. 1.1, 1.2, 1.3, 1.4, or 1.5; or greater than or equal to about 1.3.

38. The method of claim 1, the method comprising the use of a reagent for quantifying a panel miRNAs, the panel comprising miRNAs selected from the group consisting of hsa-let-7g-5p, hsa-miR-150-5p, hsa-let-7f-5p, hsa-let-7i-5p, hsa-miR-15b-5p, hsa-miR-16-5p, hsa-miR-26b-5p, hsa-miR-29a-3p, hsa-let-7a-5p, hsa-miR-19b-3p, hsa-miR-21-5p, hsa-miR-223-3p, hsa-miR-23a-3p, hsa-miR-25-3p, hsa-miR-17-5p, hsa-miR-22-3p, hsa-miR-24-3p, hsa-miR-342-3p, hsa-miR-142-3p, and hsa-miR-29c-3p.

39. A microarray, solid support, or collection of solid supports, comprising a panel of miRNAs selected from the group consisting of hsa-let-7g-5p, hsa-miR-150-5p, hsa-let-7f-5p, hsa-let-7i-5p, hsa-miR-15b-5p, hsa-miR-16-5p, hsa-miR-26b-5p, hsa-miR-29a-3p, hsa-let-7a-5p, hsa-miR-19b-3p, hsa-miR-21-5p, hsa-miR-223-3p, hsa-miR-23a-3p, hsa-miR-25-3p, hsa-miR-17-5p, hsa-miR-22-3p, hsa-miR-24-3p, hsa-miR-342-3p, hsa-miR-142-3p, and hsa-miR-29c-3p; and/or a binding partner for at least one of said miRNAs.

40. The microarray, solid support, or collection of solid supports of claim 39 comprising a nucleic acid amplification primer, a pair of nucleic acid amplification primers, and/or an oligonucleotide probe corresponding to at least one of said miRNAs.

41. The solid support or collection of solid supports of claim 39 wherein said solid support is a bead or collection of beads, respectively.

42. A kit comprising a component, microarray, solid support, or collection of solid supports or any one of claim 39, optionally further including instructions for use.
